(19) United States
(12) Patent Application Publication
(10) Pub. No.: US 2026/0306649 A1
(43) Pub. Date: Oct. 8, 2026

(54) METHODS FOR TREATING PATIENTS WITH LOCALLY ADVANCED OR METASTATIC UROTHELIAL CANCER WITH ANTIBODY DRUG CONJUGATES (ADC) THAT BIND 191P4D12 PROTEINS IN COMBINATION WITH PEMBROLIZUMAB

(71) Applicants: AGENSYS, INC., Northbrook, IL (US); SEAGEN INC., Bothell, WA (US); MERCK SHARP & DOHME LLC, Rahway, NJ (US)

(72) Inventors: Anne-Sophie CARRET, Kirkland, WA (US); Oyewale O. ABIDOYE, Atherton, CA (US)

(73) Assignees: AGENSYS, INC., Northbrook, IL (US); SEAGEN INC., Bothell, WA (US); MERCK SHARP & DOHME LLC, Rahway, NJ (US)

(21) Appl. No.: 18/996,742

(22) PCT Filed: Jul. 24, 2023
(86) PCT No.: PCT/US2023/070812

§ 371 Date: Jan. 17, 2025

Related U.S. Application Data
(60) Provisional application No. 63/392,067, filed on Jul. 25, 2022.
(60) Provisional application No. 63/402,830, filed on Aug. 31, 2022.
(60) Provisional application No. 63/504,183, filed on May 24, 2023.

(51) Int. Cl. A61K 47/68; A61K 9/00; A61K 39/00; A61K 47/22; A61K 47/26; A61P 35/04; C07K 16/28

(52) U.S. Cl. CPC A61K 47/68031; A61K 9/0019; A61K 47/22; A61K 47/26; A61K 47/6889; A61P 35/04; C07K 16/2803; C07K 16/2818; A61K 2039/505; A61K 2039/54; A61K 2039/545

(57) ABSTRACT

Provided herein are methods for treating cancers with antibody drug conjugates (ADC) that bind to 191P4D12 protein (Nectin-4) and pembrolizumab.

### CROSS-REFERENCE TO RELATED APPLICATIONS

100011 This application claims the benefit of U.S. Application No. 63/392,067, filed Jul. 25, 2022, U.S. Application No. 63/402,830, filed Aug. 31, 2022, and U.S. Application No. 63/504,183, filed May 24, 2023, the disclosure of each of which is incorporated by reference herein in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14369-295-228_SEQLISTING.xml”, was created on Jul. 18, 2023 and is 62,806 bytes in size.

### 1. FIELD

Provided herein are methods for treating cancers with antibody drug conjugates (ADC) that bind to 191P4D12 protein (Nectin-4). In particular, provided herein are methods for treating patients with unresectable locally advanced or metastatic urothelial cancer who are unable to receive cisplatin-based chemotherapy with antibody drug conjugates (ADC) that bind to 191P4D12 proteins in combination with pembrolizumab.

### 2. BACKGROUND

Cancer is the leading cause of death in the US for people 35 to 65 years of age and it is the second leading cause of death worldwide. It was estimated in 2019 that there would be approximately 1.7 million new cancer cases and approximately 610000 deaths from cancer in the US (National Cancer Institute. 2019. Cancer Stat Facts: Cancer of Any Site. seer.cancer.gov/statfacts/html/all.html. Accessed 5 Jun. 2019). Globally there were an estimated 18.1 million new cancer cases in 2018 and approximately 9.6 million deaths attributed to cancer in 2018 (World Health Organization. Press Release. September 2018. who.int/cancer/PRGlobocanFinal.pdf. Accessed 5 Jun. 2019). Most deaths now occur in patients with metastatic cancers. In fact, in the last 20 years, advances in treatment, including surgery, radiotherapy and adjuvant chemotherapy cured most patients with localized cancer. Patients whose cancer presented or recurred as metastatic disease obtained only modest benefit from conventional therapies in terms of overall survival (OS) and were rarely cured.

New therapeutic strategies for advanced and/or metastatic cancers include targeting molecular pathways important for cancer cell survival and novel cytotoxic compounds. The benefit of these novel drugs is reflected in prolonged survival; however, the outcome for most patients with distant metastases is still poor and novel therapies are needed.

191P4D12 (which is also known as Nectin-4) is a 66 kDa type I transmembrane protein that belongs to the nectin family of adhesion molecules. It is composed of an extracellular domain (ECD) containing 3 immunoglobulin (Ig)-like subdomains, a transmembrane helix, and an intracellular region (Takai et al., Annu Rev Cell Dev Biol (2008); 24: 309-42). Nectins are thought to mediate Ca²⁺-independent cell-cell adhesion via both homophilic and heterophilic trans-interactions at adherens junctions where they can recruit cadherins and modulate cytoskeletal rearrangements (Rikitake et al., Cell Mol Life Sci (2008); 65(2): 253-63.). Sequence identity of Nectin-4 to other Nectin family members is low and ranges between 25%-30% in the ECD (Reymond et al., Biol Chem (2001); 276(46): 43205-15).

The 3 Ig-like subdomains in the ECD of Nectin-4 are designated V, C1 and C2. The C1 domain is responsible for cis-interaction (homodimerization), while V domains of most Nectin molecules contribute to trans-interaction and cell-cell adhesion (Mandai et al., Curr Top Dev Biol (2015); 112: 197-231; Takai et al., Nat Rev Mol Cell Biol (2008); 9(8): 603-15.).

Nectin-4 was originally identified by bioinformatics and cloned from human trachea (Reymond et al., J Biol Chem (2001) 276(46): 43205-15.). Nectin-4 was identified as markedly upregulated in urothelial cancer using suppression subtractive hybridization on a pool of urothelial cancer specimens. Characterization of expression in multiple tumor specimens, both at the ribonucleic acid (RNA) level and by immunohistochemistry (IHC), also demonstrated high levels of Nectin-4 in breast, pancreatic, lung, and other cancers (Challita-Eid et al., Cancer Res (2016); 76(10): 3003-13.).

Nectin-4 has been found to be expressed in multiple cancers, particularly urothelial, breast, lung, pancreatic, and ovarian cancers. Higher levels of expression are associated with disease progression and/or poor prognosis (Fabre-Lafay et al., BMC Cancer (2007); 7: 73).

### PD-1

PD-1 is recognized as an important molecule in immune regulation and the maintenance of peripheral tolerance. PD-1 is moderately expressed on naive T, B and NKT cells and up-regulated by T/B cell receptor signaling on lymphocytes, monocytes and myeloid cells (Sharpe, Arlene H. et al., The function of programmed cell death 1 and its ligands in regulating autoimmunity and infection. Nature Immunology (2007); 8:239-245).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong, Haidong et al., Tumor-associated B7-H1 promotes T-cell apoptosis: a potential mechanism of immune evasion. Nat Med. 2002 August; 8(8):793-800; Yang, Wanhua et al., PD-1 interaction contributes to the functional suppression of T-cell responses to human uveal melanoma cells in vitro. Invest Ophthalmol Vis Sci. 2008 June; 49(6 (2008): 49: 2518-2525; Ghebeh, Hazem et al., The B7-H1 (PD-L1) T lymphocyte-inhibitory molecule is expressed in breast cancer patients with infiltrating ductal carcinoma: correlation with important high-risk prognostic factors. Neoplasia (2006) 8: 190-198; Hamanishi, Junzo et al., Programmed cell death 1 ligand 1 and tumor-infiltrating CD8+ T lymphocytes are prognostic factors of human ovarian cancer. Proc. Natl. Acad. Sci. USA (2007): 104: 3360-3365; Thompson, R Houston, and Eugene D Kwon, Significance of B7-H1 overexpression in kidney cancer. Clinical genitourin Cancer (2006): 5: 206-211; Nomi, Takeo et al., Clinical significance and therapeutic potential of the programmed death-1 ligand/programmed death-1 pathway in human pancreatic cancer. Clinical Cancer Research (2007); 13:2151-2157; Ohigashi, Yuichiro et al., Clinical significance of programmed death-1 ligand-1 and programmed death-1 ligand 2 expression in human esophageal cancer. Clin. Cancer Research (2005): 11: 2947-2953; Inman, Brant A et al., PD-L1 (B7-H1) expression by urothelial carcinoma of the bladder and BCG-induced granulomata: associations with localized stage progression. Cancer (2007): 109: 1499-1505; Shimauchi, Takatoshi et al., Augmented expression of programmed death-1 in both neoplasmatic and nonneoplastic CD4+ T-cells in adult T-cell Leukemia/Lymphoma. Int. J. Cancer (2007): 121:2585-2590; Gao, Qiang et al., Overexpression of PD-L1 significantly associates with tumor aggressiveness and postoperative recurrence in human hepatocellular carcinoma. Clinical Cancer Research (2009) 15: 971-979; Nakanishi, Juro et al., Overexpression of B7-H1 (PD-L1) significantly associates with tumor grade and postoperative prognosis in human urothelial cancers. Cancer Immunol Immunother. (2007) 56: 1173-1182; Hino et al., Tumor cell expression of programmed cell death-1 is a prognostic factor for malignant melanoma. Cancer (2010): 00: 1-9). Similarly, PD-1 expression on tumor infiltrating lymphocytes was found to mark dysfunctional T cells in breast cancer and melanoma (Ghebeh, Hazem et al., Foxp3+ tregs and B7-H1+/PD-1+ T lymphocytes co-infiltrate the tumor tissues of high-risk breast cancer patients: implication for immunotherapy. BMC Cancer. 2008 Feb. 23; 8:57; Ahmadzadeh, Mojgan et al., Tumor antigen-specific CD8 T cells infiltrating the tumor express high levels of PD-1 and are functionally impaired. Blood (2009) 114: 1537-1544) and to correlate with poor prognosis in renal cancer (Thompson, R Houston et al., PD-1 is expressed by tumor infiltrating cells and is associated with poor outcome for patients with renal carcinoma. Clinical Cancer Research (2007) 15: 1757-1761). Thus, it has been proposed that PD-L1-expressing tumor cells interact with PD-1-expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 have been approved for treating cancer. Pembrolizumab (KEYTRUDA®, Merck & Co., Inc., Rahway, NJ, USA) is a potent humanized immunoglobulin G4 (IgG4) mAb with high specificity of binding to the programmed cell death 1 (PD-1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. Keytruda® (pembrolizumab) is indicated for the treatment of patients across a number of indications and is indicated for the first-line treatment of patients with unresectable or metastatic CRC that is microsatellite instability-high or mismatch repair deficient (MSI-H/dMMR). Pembrolizumab is the current standard of care for first line MSI-H/dMMR mCRC.

### Urothelial Cancer

According to the International Agency for Research on Cancer (IARC), urothelial cancer kills more than 165000 patients annually and is the ninth most common cancer overall worldwide. Approximately 151000 new cases of urothelial cancer are diagnosed annually in Europe, with 52000 deaths per year. Over 22000 new cases are diagnosed annually in Japan, with 7600 deaths per year (Cancer Fact Sheets: All cancers excluding Non-Melanoma Skin. International Agency for Research on Cancer 2017. Retrieved from gco.iarc.fr/today/fact-sheets-cancers?cancer=29&type=0&sex=0. Accessed 19 Dec. 2017). In the U.S., the National Cancer Institute estimates more than 79,000 new cases of bladder cancer were diagnosed in 2017, and more than 16,800 people died from the disease (National Cancer Institute 2018).

Metastatic urothelial cancer has a 5-year mortality rate of approximately 85% (American Cancer Society (ACS) 2016).

Urothelial cancer is the most common type of bladder cancer (90 percent of cases), and can also be found in the urothelial cells that line the renal pelvis (where urine collects inside the kidney), ureter (tube that connects the kidneys to the bladder) and urethra.

First-line therapy for metastatic urothelial cancer in patients with sufficient renal function consists of cis-diamminedichloroplatinum (II) (cisplatin)-based combinations, like methotrexate, vinblastine, doxorubicin, and cisplatin (MVAC) or gemcitabine plus cisplatin, which demonstrate an objective response rate (ORR) up to 55%, including approximately 12% complete responses (CRs) (von der Maase 2000). Despite initial chemosensitivity, patients are not cured and the outcome of metastatic urothelial cancer after these regimens is poor: median time to progression is 7 months and median overall survival (OS) is 14 months. Long-term survival is poor (approximately 15%) and the prognosis is particularly grim for patients with visceral metastases, for whom the five year survival rate is 7% (von der Maase 2005; Bellmunt 2011).

Almost half of urothelial cancer patients are unfit for cisplatin-containing chemotherapy due to impaired renal function, poor performance status, or comorbidity (Dash et al. Cancer (2006);107(3): 506-13). In these patients, no standard first-line treatment has been defined, but current options typically include cis-Diammine (cyclobutane 1,1 dicarboxylato) platinum (carboplatin)-based regimens or single-agent taxane or gemcitabine (Cathomas 2015). In this setting, long term survival is even lower (De Santis et al. J Clin Oncol (2009); 27(33): 5634-9). In April 2017, the Food and Drug Administration (FDA) approved the anti-programmed death-ligand 1 (PD-L1) immune checkpoint inhibitor (CPI) atezolizumab (TECENTRIQ®) as first line treatment for locally advanced or metastatic urothelial carcinoma (la/mUC) patients ineligible for cisplatin. The accelerated approval was based on an open-label single arm study that showed long durations of response, indicating activity in this difficult-to-treat population, with an objective response rate (ORR) of 23% that was similar across varying levels of target expression (Balar 2017). The median OS for these patients was 15.9 months, although this is a single arm study and any OS benefit will need to be confirmed in a randomized experience (Balar et al., Lancet (2017); 389(10064): 67-76).

Pembrolizumab (Keytruda®) received accelerated approval from the FDA in May 2017 as first line treatment for patients with la/mUC ineligible for cisplatin. The study on which approval was based resulted in an ORR of 29% and median response duration not reached at the time of the analysis (median follow-up time of 7.8 months) (Pembrolizumab Prescribing Information, Merck Sharp and Dohme Corp., 2017). In May 2018, the FDA issued an alert regarding decreased survival of patients with low expression of PD-L1 being treated in the first line setting with pembrolizumab or atezolizumab, compared with platinum-based chemotherapy. Subsequently prescribing information for these two CPIs were revised to require high PD-L1 expression in first-line metastatic urothelial cancer patients who are eligible for platinum-containing chemotherapy. This development has further limited the options for metastatic urothelial cancer patients with low expression of PD-L1.

Other options for first line cisplatin-ineligible patients typically include carboplatin-based regimens or single-agent taxane or gemcitabine (Cathomas et al., Hematol Oncol Clin North Am (2015); 29(2): 329-40.).

Few options are available for second-line treatment of metastatic disease. In the European Union, the small-molecule tubulin inhibitor vinflunine (Javlor®) was authorized in 2009 based on modest activity (overall response rate 9%), moderate survival benefit of 2 months (6.9 months for vinflunine+best supportive care (BSC) vs 4.6 months for BSC alone, hazard ratio 0.88), and a favorable safety profile (Bellmunt et al. Clin Oncol (2009); 27(27): 4454-61). In May 2016, the FDA provided accelerated approval of atezolizumab as the first salvage therapy following platinum agents for la/mUC in the US, followed by EU approval in September 2017. In February 2017, nivolumab (Opdivo®) became the second immunotherapy granted accelerated approval by the FDA, which was followed by EU approval in June 2017. In March and May 2017, the FDA granted accelerated approval for avelumab (Bavencio®) and durvalumab (Imfinzi™), respectively, both PD-L1 blocking antibodies indicated for the treatment of patients with locally advanced or metastatic urothelial carcinoma who have disease progression during or following platinum-containing chemotherapy or have disease progression within 12 months of neoadjuvant or adjuvant treatment with platinum-containing chemotherapy. Pembrolizumab received regular approval from the FDA in May 2017 as second-line treatment (Keytruda Prescribing Information, Merck, May 2017). The approval was based on the first randomized experience reported for a CPI in the locally advanced or metastatic post-platinum urothelial cancer setting, a phase 3 study in 542 patients showing an OS of 10.3 months as compared to 7.4 months with taxane chemotherapy or vinflunine. Additionally, ORR was 21% for pembrolizumab and 11% for chemotherapy. No statistically significant difference in progression-free survival (PFS) between the two arms was observed (Bellmunt et al., N Engl J Med (2017);376(11): 1015-26). EU approval for the same indication was granted in September 2017 and Japanese approval in January 2018. Other programmed cell death protein 1 (PD-1) and PD-L1 inhibitors are currently being evaluated in clinical trials for urothelial cancer, as first and second-line therapy (Mullane et al., Curr Opin Urol (2016);26(6): 556-63).

While CPIs offer a new approach to treatment of metastatic urothelial cancer, tumor responses have occurred in a minority of patients and the improvement in long-term survival is only a few months. For example, in May 2017, Roche announced that a confirmatory phase 3 trial of second-line atezolizumab had failed to meet its primary endpoint of OS (Roche, press release “Roche provides update on phase III study of Tecentriq (atezolizumab) in people with previously treated advanced bladder cancer,” 10 May 2017). Most patients with locally advanced or metastatic urothelial cancer do not respond to CPIs and many who do respond ultimately develop disease progression (Rosenberg et al., Lancet (2016); 387(10031): 1909-20). Novel treatments are still needed, particularly for patients who have not responded to CPIs or who have progressed following CPI therapy.

The lack of approved first line therapies for patients with metastatic urothelial cancer and the limited activity observed with second-line chemotherapy adequately demonstrate that this population has significant unmet medical need.

### Bladder Cancer

Of all new cases of cancer in the United States, bladder cancer represents approximately 5 percent in men (fifth most common neoplasm) and 3 percent in women (eighth most common neoplasm). The incidence is increasing slowly, concurrent with an increasing older population. American Cancer Society (cancer.org) estimates that there are 81,400 new cases annually, including 62,100 in men and 19,300 in women, which accounts for 4.5% of all cancer cases. The age-adjusted incidence in the United States is 20 per 100,000 for men and women. There are an estimated 17,980 deaths from bladder cancer in annually (13,050 in men and 4,930 in women), which accounts for 3% of cancer related deaths. Bladder cancer incidence and mortality strongly increase with age and will be an increasing problem as the population becomes more elderly. Globally, approximately 580,000 people will be diagnosed with bladder cancer in 2020, and bladder cancer will be attributed to approximately 210,000 deaths worldwide.

Most bladder cancers recur in the bladder. Bladder cancer is managed with a combination of transurethral resection of the bladder (TUR) and intravesical chemotherapy or immunotherapy. The multifocal and recurrent nature of bladder cancer points out the limitations of TUR. Most muscle-invasive cancers are not cured by TUR alone. Radical cystectomy and urinary diversion is the most effective means to eliminate the cancer but carry an undeniable impact on urinary and sexual function. There continues to be a significant need for treatment modalities that are beneficial for bladder cancer patients.

There is a significant need for additional therapeutic methods for urothelial and bladder cancers. These include the use of antibodies and antibody drug conjugates, including in combination with other agents, as treatment modalities.

### 3. SUMMARY

Provided herein are methods for the treatment of various cancers in human subjects, including methods for treating patients with unresectable locally advanced or metastatic urothelial cancer who are unable to receive cisplatin-based chemotherapy with antibody drug conjugates (ADC) that bind to 191P4D12 proteins in combination with pembrolizumab.

In certain embodiments, the human subject treated with the methods provided herein is ineligible to receive cisplatin treatment and has not received previous treatment with an immune checkpoint inhibitor (CPI) (e.g., a PD-1 inhibitor, PD-L1 inhibitor, or PD-L2 inhibitor (including, but not limited to, atezolizumab, pembrolizumab, nivolumab, durvalumab, or avelumab)).

Embodiment 1. A method of treating cancer in a human subject, comprising administering to the subject:
(a) an effective amount of an antibody drug conjugate (ADC) comprising an anti-191P4D12 antibody or antigen binding fragment thereof and
(b) an effective amount of an anti-PD-1 antibody;
wherein the anti-191P4D12 antibody or antigen binding fragment thereof binds to 191P4D12 and is conjugated to one or more units of monomethyl auristatin E (MMAE); wherein the anti-PD-1 antibody comprises: (i) a light chain variable region comprising light chain CDR1, CDR2 and CDR3 of SEQ ID NOs: 24, 25 and 26, respectively and (ii) a heavy chain variable region comprising heavy chain CDR1, CDR2 and CDR3 of SEQ ID NOs: 29, 30 and 31, respectively;
wherein the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23;
wherein the subject has urothelial or bladder cancer;
wherein the subject has not received an immune checkpoint inhibitor (CPI) therapy; and
wherein the subject is ineligible to receive cisplatin treatment (cisplatin ineligible).

Embodiment 2. The method of embodiment 1, wherein the subject has visceral metastases.

Embodiment 3. The method of embodiment 1, wherein the subject has lymph nodes only disease.

Embodiment 4. The method of any one of embodiments 1 to 3, wherein the disease site of origin is in the upper tract.

Embodiment 5. The method of any one of embodiments 1 to 3, wherein the disease site of origin is in the lower tract.

Embodiment 6. The method of any one of embodiments 1 to 5, wherein the subject has a PD-L1 expression combined positive score (CPS) greater than or equal to 10.

Embodiment 7. The method of any one of embodiments 1 to 5, wherein the subject has a PD-L1 expression CPS less than 10.

Embodiment 8. The method of any one of embodiments 1 to 6, wherein the subject has a Nectin-4H-score between 0 and 300.

Embodiment 9. The method of any one of embodiments 1 to 7, wherein the subject has a Nectin-4H-score between 0 and 200.

Embodiment 10. The method of any one of embodiments 1 to 9, wherein the subject has an ECOG performance status score of 1 to 2.

Embodiment 11. The method of any one of embodiments 1 to 9, wherein the subject has one or more of the conditions selected from the group consisting of: ECOG performance status score of 2, impaired renal function, and no less than Grade 2 hearing loss.

Embodiment 12. The method of any one of embodiments 1 to 9, wherein the subject has NYHA Class III heart failure.

Embodiment 13. The method of embodiment 11, wherein the subject has an ECOG performance status score of 2, and
wherein the subject (i) has hemoglobin ≥10 g/dL; (ii) has GFR ≥50 mL/min; and (iii) does not have NYHA Class III heart failure.

Embodiment 14. The method of embodiment 11, wherein the impaired renal function is determined by creatinine clearance (CrCl) less than 60 mL/min.

Embodiment 15. The method of embodiment 11, wherein the impaired renal function is determined by CrCl less than 60 but no less than 30 mL/min.

Embodiment 16. The method of embodiment 11, wherein the impaired renal function is determined by CrCl less than 30 but no less than 15 mL/min.

Embodiment 17. The method of any one of embodiments 1 to 16, wherein the subject has one or more of the conditions selected from the group consisting of:
(i) absolute neutrophil count no less than 1500/μL;
(ii) platelet count no less than 100,000/μL;
(iii) hemoglobin no less than 9 g/dL;
(iv) serum bilirubin no more than either of 1.5 times of upper limit of normal (ULN) or 3 times ULN for patients with Gilbert's disease;
(v) CrCl no less than 30 mL/min, and
(vi) alanine aminotransferase and aspartate aminotransferase no more than 3 fold of ULN.

Embodiment 18. The method of embodiment 17, wherein the subject has all of conditions (i) to (vi) of embodiment 15.

Embodiment 19. The method of any one of embodiments 14 to 18, wherein the CrCl is measured by 24 hour urine collection or estimated by the Cockcroft-Gault criteria.

Embodiment 20. The method of any one of embodiments 1 to 19, wherein the subject has no more than Grade 2 sensory or motor neuropathy.

Embodiment 21. The method of any one of embodiments 1 to 20, wherein the subject has no active central nervous system metastases.

Embodiment 22. The method of any one of embodiments 1 to 21, wherein the subject has no uncontrolled diabetes.

Embodiment 23. The method of embodiment 22, wherein the uncontrolled diabetes is determined by hemoglobin A1c (HbA1c) no less than 8% or HbA1c between 7 and 8% with associated diabetes symptoms that are not otherwise explained.

Embodiment 24. The method of embodiment 23, wherein the associated diabetes symptoms comprise or consist of polyuria, polydipsia, or both polyuria and polydipsia.

Embodiment 25. The method of any one of embodiments 1 to 24, wherein the subject has locally advanced or metastatic urothelial cancer.

Embodiment 26. The method of any one of embodiments 1 to 25, wherein the subject has locally advanced or metastatic bladder cancer.

Embodiment 27. The method of any one of embodiments 1 to 26, wherein the anti-191P4D12 antibody or antigen binding fragment thereof comprises CDR-H1 comprising the amino acid sequence of SEQ ID NO:9, CDR-H2 comprising the amino acid sequence of SEQ ID NO:10, CDR-H3 comprising the amino acid sequence of SEQ ID NO:11; CDR-L1 comprising the amino acid sequence of SEQ ID NO:12, CDR-L2 comprising the amino acid sequence of SEQ ID NO:13, and CDR-L3 comprising the amino acid sequence of SEQ ID NO:14, or
wherein the anti-191P4D12 antibody or antigen binding fragment thereof comprises CDR-H1 comprising the amino acid sequence of SEQ ID NO:16, CDR-H2 comprising the amino acid sequence of SEQ ID NO:17, CDR-H3 comprising the amino acid sequence of SEQ ID NO:18; CDR-L1 comprising the amino acid sequence of SEQ ID NO:19, CDR-L2 comprising the amino acid sequence of SEQ ID NO:20, and CDR-L3 comprising the amino acid sequence of SEQ ID NO:21.

Embodiment 28. The method of any one of embodiments 1 to 26, wherein the anti-191P4D12 antibody or antigen binding fragment thereof comprises CDR-H1 consisting of the amino acid sequence of SEQ ID NO:9, CDR-H2 consisting of the amino acid sequence of SEQ ID NO:10, CDR-H3 consisting of the amino acid sequence of SEQ ID NO:11; CDR-L1 consisting of the amino acid sequence of SEQ ID NO:12, CDR-L2 consisting of the amino acid sequence of SEQ ID NO:13, and CDR-L3 consisting of the amino acid sequence of SEQ ID NO:14, or
wherein the anti-191P4D12 antibody or antigen binding fragment thereof comprises CDR-H1 consisting of the amino acid sequence of SEQ ID NO:16, CDR-H2 consisting of the amino acid sequence of SEQ ID NO:17, CDR-H3 consisting of the amino acid sequence of SEQ ID NO:18; CDR-L1 consisting of the amino acid sequence of SEQ ID NO:19, CDR-L2 consisting of the amino acid sequence of SEQ ID NO:20, and CDR-L3 consisting of the amino acid sequence of SEQ ID NO:21.

Embodiment 29. The method of any one of embodiments 1 to 28, wherein the anti-191P4D12 antibody or antigen binding fragment thereof comprises a heavy chain variable region comprising the amino acid sequence of SEQ ID NO:22 and a light chain variable region comprising the amino acid sequence of SEQ ID NO:23.

Embodiment 30. The method of any one of embodiments 1 to 29, wherein the anti-191P4D12 antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

Embodiment 31. The method of any one of embodiments 1 to 30, wherein the anti-191P4D12 antigen binding fragment is an Fab, F(ab′)2, Fv or scFv.

Embodiment 32. The method of any one of embodiments 1 to 30, wherein the anti-191P4D12 antibody is a fully human antibody.

Embodiment 33. The method of any one of embodiments 1 to 30 and 32, wherein the anti-191P4D12 antibody is an IgG1 and light chain is a kappa light chain

Embodiment 34. The method of any one of embodiments 1 to 33, wherein the anti-191P4D12 antibody or antigen binding fragment thereof is recombinantly produced.

Embodiment 35. The method of any one of embodiments 1 to 34, wherein the anti-191P4D12 antibody or antigen binding fragment is conjugated to each unit of MMAE via a linker.

Embodiment 36. The method of embodiment 35, wherein the linker is an enzyme-cleavable linker, and wherein the linker forms a bond with a sulfur atom of the antibody or antigen binding fragment thereof.

Embodiment 37. The method of embodiment 35 or 36, wherein the linker has a formula of: -Aa-Ww-Yy-; wherein -A- is a stretcher unit, a is 0 or 1; —W— is an amino acid unit, w is an integer ranging from 0 to 12; and —Y— is a spacer unit, y is 0, 1, or 2.

Embodiment 38. The method of embodiment 37, wherein the stretcher unit has the structure of Formula (1) below; the amino acid unit is valine-citrulline; and the spacer unit is a PAB group comprising the structure of Formula (2) below:

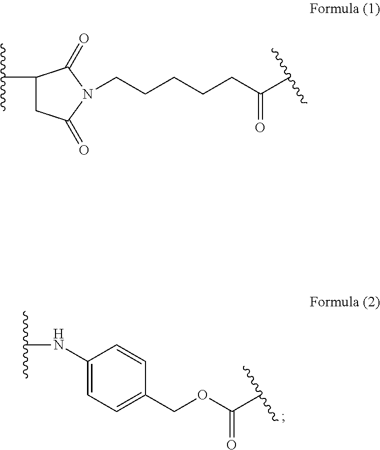 [SMILES: *C(=O)CCCCCN1C(=O)CC(*)C1=O ; *Nc1ccc(COC(*)=O)cc1]

Embodiment 39. The method of embodiment 37 or 38, wherein the stretcher unit forms a bond with a sulfur atom of the antibody or antigen binding fragment thereof, and wherein the spacer unit is linked to MMAE via a carbamate group.

Embodiment 40. The method of any one of embodiments 1 to 39, wherein the ADC comprises from 1 to 20 units of MMAE per antibody or antigen binding fragment thereof.

Embodiment 41. The method of any one of embodiments 1 to 40, wherein the ADC comprises from 1 to 10 units of MMAE per antibody or antigen binding fragment thereof.

Embodiment 42. The method of any one of embodiments 1 to 41, wherein the ADC comprises from 2 to 8 units of MMAE per antibody or antigen binding fragment thereof.

Embodiment 43. The method of any one of embodiments 1 to 42, wherein the ADC comprises from 3 to 5 units of MMAE per antibody or antigen binding fragment thereof.

Embodiment 44. The method of any one of embodiments 1 to 43, wherein the ADC has the following structure:

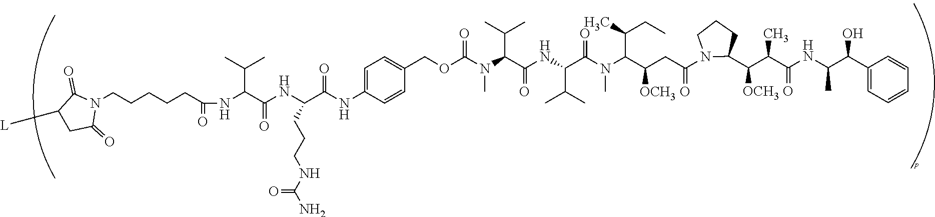

wherein L- represents the anti-191P4D12 antibody or antigen binding fragment thereof and p is from 1 to 10.

Embodiment 45. The method of embodiment 44, wherein p is from 2 to 8.

Embodiment 46. The method of embodiment 44 or 45, wherein p is from 3 to 5.

Embodiment 47. The method of any one of embodiments 44 to 46, wherein p is from 3 to 4.

Embodiment 48. The method of any one of embodiments 44 to 47, wherein p is about 4.

Embodiment 49. The method of any one of embodiments 44 to 47, wherein the average p value of the effective amount of the antibody drug conjugates is about 3.8.

Embodiment 50. The method of any one of embodiments 1 to 49, wherein the ADC is administered to the subject at a dose of about 1 to about 10 mg/kg of the subject's body weight, about 1 to about 5 mg/kg of the subject's body weight, about 1 to about 2.5 mg/kg of the subject's body weight, or about 1 to about 1.25 mg/kg of the subject's body weight.

Embodiment 51. The method of any one of embodiments 1 to 50, wherein the ADC is administered to the subject at a dose of about 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, about 2.25 mg/kg, or about 2.5 mg/kg of the subject's body weight.

Embodiment 52. The method of any one of embodiments 1 to 51, wherein the ADC is administered to the subject at a dose of about 1 mg/kg of the subject's body weight.

Embodiment 53. The method of any one of embodiments 1 to 51, wherein the ADC is administered to the subject at a dose of about 1.25 mg/kg of the subject's body weight.

Embodiment 54. The method of any one of embodiments 1 to 53, wherein the ADC is administered to the subject by an intravenous (IV) injection or infusion.

Embodiment 55. The method of any one of embodiments 1 to 54, wherein the ADC is administered to the subject by an IV injection or infusion up to 2 days of a 21-day treatment cycle.

Embodiment 56. The method of any one of embodiments 1 to 55, wherein the ADC is administered to the subject by an IV injection or infusion on days 1 and 8 of a 21-day treatment cycle.

Embodiment 57. The method of any one of embodiments 1 to 56, wherein the ADC is administered to the subject by an IV injection or infusion over about 30 minutes up to 2 days of a 21-day treatment cycle.

Embodiment 58. The method of any one of embodiments 1 to 57, wherein the ADC is administered by an IV injection or infusion over about 30 minutes on days 1 and 8 of a 21-day treatment cycle.

Embodiment 59. The method of any one of embodiments 1 to 58, wherein the ADC is formulated in a pharmaceutical composition comprising L-histidine, polysorbate-20 (TWEEN-20), and trehalose dehydrate.

Embodiment 60. The method of any one of embodiments 1 to 59, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.

Embodiment 61. The method of any one of embodiments 1 to 59, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

Embodiment 62. The method of any one of embodiments 1 to 61, wherein the ADC has the following structure:

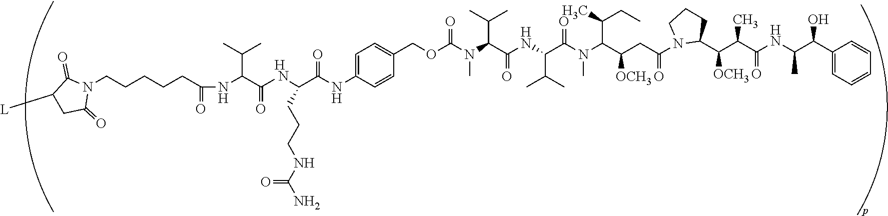

wherein L- represents the antibody or antigen binding fragment thereof and p is from about 3 to about 4, the anti-191P4D12 antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered at a dose of about 1.25 mg/kg of the subject's body weight, and wherein the dose is administered by an IV injection or infusion over about 30 minutes on days 1 and 8 of a 21-day treatment cycle.

Embodiment 63. The method of any one of embodiments 1 to 60, wherein the anti-PD-1 antibody is administered to the subject at a dose of about 100 mg to about 400 mg.

Embodiment 64. The method of any one of embodiments 1 to 61, wherein the anti-PD-1 antibody is administered to the subject at a dose of about 200 mg.

Embodiment 65. The method of embodiment 62, wherein:
(a) the anti-PD-1 antibody is administered to the subject at a dose of about 200 mg; and
(b) after step (a), the anti-PD-1 antibody is administered to the subject at a dose of about 400 mg every 42 days.

Embodiment 66. The method of any one of embodiments 1 to 65, wherein the anti-PD-1 antibody is administered to the subject by an IV infusion on 1 day of the 21-day treatment cycle.

Embodiment 67. The method of any one of embodiments 1 to 66, wherein the anti-PD-1 antibody is administered to the subject by an IV infusion on day 1 in each treatment cycle of 21 days.

Embodiment 68. The method of any one of embodiments 1 to 67, wherein the anti-PD-1 antibody is administered to the subject by an IV infusion over about 30 minutes on 1 day of the 21-day treatment cycle.

Embodiment 69. The method of any one of embodiments 1 to 66, wherein the anti-PD-1 antibody is administered to the subject by an IV infusion over about 30 minutes on day 1 in each treatment cycle of 21 days.

Embodiment 70. The method of any one of embodiments 1 to 69, whereby the subject has a complete response following the treatment.

Embodiment 71. The method of any one of embodiments 1 to 69, wherein the subject has a partial response following the treatment.

Embodiment 72. The method of any one of embodiments 1 to 69, wherein the subject has a complete response or a partial response following the treatment.

Embodiment 73. The method of any one of embodiments 1 to 69, wherein the subject has a stable disease following the treatment.

Embodiment 74. The method of any one of embodiments 1 to 69, wherein the subject has a duration of response of at least or about 6 months following the treatment.

Embodiment 75. The method of any one of embodiments 1 to 69, wherein the subject has a duration of response of at least or about 12 months following the treatment.

Embodiment 76. The method of any one of embodiments 1 to 69, wherein the subject has a duration of response of at least or about 24 months following the treatment.

Embodiment 77. The method of any one of embodiments 1 to 69, wherein the subject has a duration of response of at least or about 27 months following the treatment.

Embodiment 78. The method of any one of embodiments 1 to 69, wherein the subject has a progression free survival of at least or about 6 months following the treatment.

Embodiment 79. The method of any one of embodiments 1 to 69, wherein the subject has a progression free survival of at least or about 8 months following the treatment.

Embodiment 80. The method of any one of embodiments 1 to 69, wherein the subject has a progression free survival of at least or about 12 months following the treatment.

Embodiment 81. The method of any one of embodiments 1 to 69, wherein the subject has a progression free survival of at least or about 20 months following the treatment.

Embodiment 82. The method of any one of embodiments 1 to 69, wherein the subject has a progression free survival of at least or about 29 months following the treatment.

Embodiment 83. The method of any one of embodiments 1 to 69, wherein the subject has an overall survival of at least or about 22 months following the treatment.

Embodiment 84. The method of any one of embodiments 1 to 69, wherein the subject has an overall survival of at least or about 27 months following the treatment.

Embodiment 85. The method of any one of embodiments 1 to 69, wherein the subject has a overall survival of at least or about 30 months following the treatment.

Embodiment 86. The method of any one of embodiments 1 to 69, wherein the subject has an overall survival ranging from 19 to 25 months following the treatment.

Embodiment 87. The method of any one of embodiments 1 to 69, wherein the subject has an overall survival ranging from 28 to 32 months following the treatment.

Embodiment 88. The method of any one of embodiments 1 to 69, wherein a population of the subjects is treated by the methods, and wherein percentage of the subjects having complete response in the treated population is at least or about 1⁰%.

Embodiment 89. The method of any one of embodiments 1 to 69, wherein a population of the subjects is treated by the methods, and wherein percentage of the subjects having partial response in the treated population is at least or about 54%.

Embodiment 90. The method of any one of embodiments 1 to 69, wherein a population of the subjects is treated by the methods, and wherein objective response rate in the treated population is at least or about 65%.

Embodiment 91. The method of any one of embodiments 1 to 69, wherein a population of the subjects is treated by the methods, and wherein objective response rate in the treated population ranges from 53% to 75%.

Embodiment 92. The method of any one of embodiments 1 to 69, wherein a population of the subjects is treated by the methods, and wherein percentage of the subjects having stable disease in the treated population is at least or about 22%.

Embodiment 93. The method of any one of embodiments 1 to 69, wherein a population of the subjects is treated by the methods, and wherein duration of response in the treated population is at least or about 6 months.

Embodiment 94. The method of any one of embodiments 1 to 69, wherein a population of the subjects is treated by the methods, and wherein duration of response in the treated population is at least or about 12 months.

Embodiment 95. The method of any one of embodiments 1 to 69, wherein a population of the subjects is treated by the methods, and wherein duration of response in the treated population is at least or about 24 months.

Embodiment 96. The method of any one of embodiments 1 to 69, wherein a population of the subjects is treated by the methods, and wherein duration of response in the treated population is at least or about 27 months.

Embodiment 97. The method of any one of embodiments 1 to 69, wherein a population of the subjects is treated by the methods, and wherein progression free survival in the treated population is at least or about 6 months.

Embodiment 98. The method of any one of embodiments 1 to 69, wherein a population of the subjects is treated by the methods, and wherein progression free survival in the treated population is at least or about 12 months.

Embodiment 99. The method of any one of embodiments 1 to 69, wherein a population of the subjects is treated by the methods, and wherein progression free survival in the treated population is at least or about 20 months.

Embodiment 100. The method of any one of embodiments 1 to 69, wherein a population of the subjects is treated by the methods, and wherein progression free survival in the treated population is at least or about 29 months.

Embodiment 101. The method of any one of embodiments 1 to 69, wherein a population of the subjects is treated by the methods, and wherein median overall survival in the treated population is at least or about 22 months.

Embodiment 102. The method of any one of embodiments 1 to 69, wherein a population of the subjects is treated by the methods, and wherein median overall survival in the treated population is at least or about 27 months.

Embodiment 103. The method of any one of embodiments 1 to 69, wherein a population of the subjects is treated by the methods, and wherein median overall survival in the treated population is at least or about 30 months.

Embodiment 104. The method of any one of embodiments 1 to 69, wherein a population of the subjects is treated by the methods, and wherein overall survival in the treated population ranges from 19 to 25 months.

Embodiment 105. The method of any one of embodiments 1 to 69, wherein a population of the subjects is treated by the methods, and wherein overall survival in the treated population ranges from 30 to 32 months.

Embodiment 106. The method of any one of embodiments 1 to 70 and 72, wherein the complete response rate is at least or about 10% for a population of subjects treated with the method.

Embodiment 107. The method of any one of embodiments 1 to 69, 71, and 72, wherein the partial response rate is at least or about 54% for a population of subjects treated with the method.

Embodiment 108. The method of any one of embodiments 1 to 72, wherein objective response rate is at least or about 65% for a population of subjects treated with the method.

Embodiment 109. The method of any one of embodiments 1 to 72, wherein objective response rate is from 53% to 75% for a population of subjects treated with the method.

Embodiment 110. The method of any one of embodiments 1 to 69 and 73, wherein the stable disease rate is at least or about 22% for a population of subjects treated with the method.

Embodiment 111. The method of any one of embodiments 1 to 69 and 74 to 77, wherein the duration of response is at least or about 6 months for a population of subjects treated with the method.

Embodiment 112. The method of any one of embodiments 1 to 69 and 74 to 77, wherein the duration of response is at least or about 12 months for a population of subjects treated with the method.

Embodiment 113. The method of any one of embodiments 1 to 69 and 74 to 77, wherein the duration of response is at least or about 24 months for a population of subjects treated with the method.

Embodiment 114. The method of any one of embodiments 1 to 69 and 74 to 77, wherein the duration of response is at least or about 27 months for a population of subjects treated with the method.

Embodiment 115. The method of any one of embodiments 1 to 69 and 78 to 82, wherein the progression free survival is at least or about 6 months for a population of subjects treated with the method.

Embodiment 116. The method of any one of embodiments 1 to 69 and 78 to 82, wherein the progression free survival is at least or about 12 months for a population of subjects treated with the method.

Embodiment 117. The method of any one of embodiments 1 to 69 and 78 to 82, wherein the progression free survival is at least or about 20 months for a population of subjects treated with the method.

Embodiment 118. The method of any one of embodiments 1 to 69 and 78 to 82, wherein the progression free survival is at least or about 29 months for a population of subjects treated with the method.

Embodiment 119. The method of any one of embodiments 1 to 69 and 83 to 87, wherein the median overall survival is at least or about 22 months for a population of subjects treated with the method.

Embodiment 120. The method of any one of embodiments 1 to 69 and 83 to 87, wherein the median overall survival is at least or about 27 months for a population of subjects treated with the method.

Embodiment 121. The method of any one of embodiments 1 to 69 and 83 to 87, wherein the median overall survival is at least or about 30 months for a population of subjects treated with the method.

Embodiment 122. The method of any one of embodiments 1 to 69, 79, and 80, wherein the overall survival is from 19 to 25 months for a population of subjects treated with the method.

Embodiment 123. The method of any one of embodiments 1 to 69, 79, and 80, wherein the overall survival is from 30 to 32 months for a population of subjects treated with the method.

### 4. BRIEF DESCRIPTION OF THE DRAWINGS

FIGS. 1A-1E depict the nucleotide and amino acid sequences of nectin-4 protein (FIG. 1A), the nucleotide and amino acid sequences of the heavy chain (FIG. 1B) and light chain (FIG. 1C) of Ha22-2(2.4)6.1, and the amino acid sequences of the heavy chain (FIG. 1D) and light chain of Ha22-2(2.4)6.1 (FIG. 1E).

FIG. 2 depicts the overall study design of the clinical study described in Section 6.1.

FIG. 3 depicts the study stages of the clinical study, which is a phase 1b/2 study of enfortumab vedotin combined with pembrolizumab as first-line treatment in patients with unresectable locally advanced or metastatic urothelial cancer (la/mUC) who are unable to receive cisplatin-based chemotherapy, as described in Section 6.1.

FIG. 4 depicts the European Organization for the Research and Treatment (EORTC) Core Quality of Life (QLQ-C-30) assessment (EORTC-QLQ-C-30, current version, Version 3), as described in Section 6.1.

FIG. 5 depicts the EuroQol-5 Dimensions (EQ-5D-5L) described in Section 6.1.

FIG. 6 depicts tumor reduction (i.e., tumor size (% change from baseline)) for individual patients in the clinical study, as described in Section 6.1.

FIG. 7 depicts the percent change from baseline in sum of diameters of target lesions over time (in months) per blinded independent central review in the clinical study, as described in Section 6.1.

FIG. 8 depicts ORR subgroup analysis of patients administered a combination of enfortumab vedotin and pembrolizumab (i.e., the EV+pembro arm) in the clinical study described in Section 6.1.

FIG. 9 depicts ORR subgroup analysis of patients administered enfortumab vedotin monotherapy (i.e., the EV Mono arm) in the clinical study described in Section 6.1.

FIG. 10 depicts the H-score of Nectin-4 expression at baseline and best response per blinded independent central review in the clinical study, as described in Section 6.1.

FIG. 11 depicts the H-score of Nectin-4 expression at baseline and best overall response per blinded independent central review in the clinical study, as described in Section 6.1.

FIG. 12 depicts duration of response (DOR) per blinded independent central review in the clinical study described in Section 6.1.

FIG. 13 depicts progression-free survival per blinded independent central review in the clinical study described in Section 6.1.

FIG. 14 depicts overall survival in the clinical study described in Section 6.1.

FIG. 15 depicts duration of response (DOR) per blinded independent central review in the clinical study described in Section 6.2.

FIG. 16 depicts progression-free survival per blinded independent central review in the clinical study described in Section 6.2.

FIG. 17 depicts overall survival in the clinical study described in Section 6.2.

### 5. DETAILED DESCRIPTION

Before the present disclosure is further described, it is to be understood that the disclosure is not limited to the particular embodiments set forth herein, and it is also to be understood that the terminology used herein is for describing particular embodiments only, and is not intended to be limiting.

### 5.1 Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dubel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgAQ1, and IgA2) of immunoglobulin molecule. Antibodies may be agonistic antibodies or antagonistic antibodies.

The term “monoclonal antibody” refers to an antibody obtained from a population of substantially homogeneous antibodies, that is, the individual antibodies comprising the population are identical except for possible naturally occurring mutations that can be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell, for example, a cancer cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “antigen binding fragment,” “antigen binding domain,” “antigen binding region,” and similar terms refer to that portion of an antibody, which comprises the amino acid residues that interact with an antigen and confer on the binding agent its specificity and affinity for the antigen (e.g., the CDRs). “Antigen-binding fragment” as used herein include “antibody fragment,” which comprise a portion of an intact antibody, such as the antigen-binding or variable region of the intact antibody. Examples of antibody fragments include, without limitation, Fab, Fab′, F(ab′)₂, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see, e.g., Woolven et al., 1999, Immunogenetics 50: 98-101; and Streltsov et al., 2004, Proc Natl Acad Sci USA. 101:12444-49); and multispecific antibodies formed from antibody fragments.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well-known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody or antigen binding fragment thereof described herein terms such as “bind to,” “that specifically bind to,” and analogous terms are also used interchangeably herein and refer to binding molecules of antigen binding domains that specifically bind to an antigen, such as a polypeptide. An antibody or antigen binding fragment that binds to or specifically binds to an antigen may be cross-reactive with related antigens. In certain embodiments, an antibody or antigen binding fragment that binds to or specifically binds to an antigen does not cross-react with other antigens. An antibody or antigen binding fragment that binds to or specifically binds to an antigen can be identified, for example, by immunoassays, Octet®, Biacore®, or other techniques known to those of skill in the art. In some embodiments, an antibody or antigen binding fragment binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of an antibody or antigen binding fragment to a “non-target” protein is less than about 10% of the binding of the binding molecule or antigen binding domain to its particular target antigen, for example, as determined by fluorescence activated cell sorting (FACS) analysis or RIA. With regard terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding fragment that binds to an antigen includes one that is capable of binding the antigen with sufficient affinity such that the binding molecule is useful, for example, as a diagnostic agent in targeting the antigen. In certain embodiments, an antibody or antigen binding fragment that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding fragment binds to an epitope of an antigen that is conserved among the antigen from different species (e.g., between human and cyno species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using biolayer interferometry (BLI) or surface plasmon resonance (SPR) assays by Octet®, using, for example, a Octet®QK384 system, or by Biacore®, using, for example, a Biacore®-2000 or a Biacore®-3000. An “on-rate” or “rate of association” or “association rate” or “kon” may also be determined with the same biolayer interferometry (BLI) or surface plasmon resonance (SPR) techniques described above using, for example, the Octet®QK384, the Biacore®-2000, or the Biacore®-3000 system.

In certain embodiments, the antibodies or antigen binding fragments can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

In certain embodiments, the antibodies or antigen binding fragments can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate comprising the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

In certain embodiments, the antibodies or antigen binding fragments can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies comprising variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1: 755-68). Also available for the preparation of human monoclonal antibodies are methods described in Cole et al., Monoclonal Antibodies and Cancer Therapy 77 (1985); Boerner et al., 1991, J. Immunol. 147(1):86-95; and van Dijk and van de Winkel, 2001, Curr. Opin. Pharmacol. 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6(5):561-66; Bruggemann and Taussing, 1997, Curr. Opin. Biotechnol. 8(4):455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

In certain embodiments, the antibodies or antigen binding fragments can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies or antigen binding fragments can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well-known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail in the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a p sheet configuration, connected by three hypervariable regions, which form loops connecting, and in some cases form part of, the p sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while p and E contain approximately 550 amino acids. When combined with a light chain, these distinct types of heavy chains give rise to five well-known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Pluckthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted below in Table 1

TABLE 1

| | Kabat | AbM | Chothia | Contact | IMGT |
|---|---|---|---|---|---|
| CDR-L1 | L24--L34 | L24--L34 | L24--L34 | L30--L36 | L27--L38 |
| CDR-L2 | L50--L56 | L50--L56 | L50--L56 | L46--L55 | L56--L65 |
| CDR-L3 | L89--L97 | L89--L97 | L89--L97 | L89--L96 | L105- |
| | | | | | L117 |
| CDR-H1 | H31--H35B | H26-- | H26-- | H30-- | H27--H38 |
| | (Kabat | H35B | H32 . . . 34 | H35B | |
| | Numbering) | | | | |
| CDR-H1 | H31--H35 | H26--H35 | H26--H32 | H30--H35 | |
| | (Chothia | | | | |
| | Numbering) | | | | |
| CDR-H2 | H50--H65 | H50--H58 | H52--H56 | H47--H58 | H56--H65 |
| CDR-H3 | H95--H102 | H95-- | H95-- | H93-- | H105- |
| | | H102 | H102 | H101 | H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule comprising a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations comprising a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

The abbreviation “MMAE” refers to monomethyl auristatin E.

Unless otherwise indicated by context, a hyphen (-) designates the point of attachment to the pendant molecule.

The term “Chemotherapeutic Agent” refers to all chemical compounds that are effective in inhibiting tumor growth. Non-limiting examples of chemotherapeutic agents include alkylating agents; for example, nitrogen mustards, ethyleneimine compounds and alkyl sulphonates; antimetabolites, for example, folic acid, purine or pyrimidine antagonists; mitotic inhibitors, for example, anti-tubulin agents such as vinca alkaloids, auristatins and derivatives of podophyllotoxin; cytotoxic antibiotics; compounds that damage or interfere with DNA expression or replication, for example, DNA minor groove binders; and growth factor receptor antagonists. In addition, chemotherapeutic agents include cytotoxic agents (as defined herein), antibodies, biological molecules and small molecules.

As used herein, the term “conservative substitution” refers to substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al., MOLECULAR BIOLOGY OF THE GENE, The Benjamin/Cummings Pub. Co., p. 224 (4th Edition 1987)). Such exemplary substitutions are preferably made in accordance with those set forth in Table 2 and Table 3. For example, such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g. Table 3 herein; pages 13-15 “Biochemistry” 2nd ED. Lubert Stryer ed (Stanford University); Henikoffel al., PNAS 1992 Vol 89 10915-10919; Lei et al., J Biol Chem 1995 May 19; 270(20):11882-11886). Other substitutions are also permissible and may be determined empirically or in accord with known conservative substitutions.

TABLE 2
Amino Acid Abbreviations

| SINGLE LETTER | THREE LETTER | FULL NAME |
|---|---|---|
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gln | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

TABLE 3
Amino Acid Substitution or Similarity Matrix
Adapted from the GCG Software 9.0 BLOSUM62 amino acid substitution matrix
(block substitution matrix). The higher the value, the more likely a substitution
is found in related, natural proteins.

| A | C | D | E | F | G | H | I | K | L | M | N | P | Q | R | S | T | V | W | Y | . |
|---|---|---|---|---|---|---|---|---|---|---|---|---|---|---|---|---|---|---|---|---|
| 4 | 0 | −2 | −1 | −2 | 0 | −2 | −1 | −1 | −1 | −1 | −2 | −1 | −1 | −1 | 1 | 0 | 0 | −3 | −2 | A |
| | 9 | −3 | −4 | −2 | −3 | −3 | −1 | −3 | −1 | −1 | −3 | −3 | −3 | −3 | −1 | −1 | −1 | −2 | −2 | C |
| | | 6 | 2 | −3 | −1 | −1 | −3 | −1 | −4 | −3 | 1 | −1 | 0 | −2 | 0 | −1 | −3 | −4 | −3 | D |
| | | | 5 | −3 | −2 | 0 | −3 | 1 | −3 | −2 | 0 | −1 | 2 | 0 | 0 | −1 | −2 | −3 | −2 | E |
| | | | | 6 | −3 | −1 | 0 | −3 | 0 | 0 | −3 | −4 | −3 | −3 | −2 | −2 | −1 | 1 | 3 | F |
| | | | | | 6 | −2 | −4 | −2 | −4 | −3 | 0 | −2 | −2 | −2 | 0 | −2 | −3 | −2 | −3 | G |
| | | | | | | 8 | −3 | −1 | −3 | −2 | 1 | −2 | 0 | 0 | −1 | −2 | −3 | −2 | 2 | H |
| | | | | | | | 4 | −3 | 2 | 1 | −3 | −3 | −3 | −3 | −2 | −1 | 3 | −3 | −1 | I |
| | | | | | | | | 5 | −2 | −1 | 0 | −1 | 1 | 2 | 0 | −1 | −2 | −3 | −2 | K |
| | | | | | | | | | 4 | 2 | −3 | −3 | −2 | −2 | −2 | −1 | 1 | −2 | −1 | L |
| | | | | | | | | | | 5 | −2 | −2 | 0 | −1 | −1 | −1 | 1 | −1 | −1 | M |
| | | | | | | | | | | | 6 | −2 | 0 | 0 | 1 | 0 | −3 | −4 | −2 | N |
| | | | | | | | | | | | | 7 | −1 | −2 | −1 | −1 | −2 | −4 | −3 | P |
| | | | | | | | | | | | | | 5 | 1 | 0 | −1 | −2 | −2 | −1 | Q |
| | | | | | | | | | | | | | | 5 | −1 | −1 | −3 | −3 | −2 | R |
| | | | | | | | | | | | | | | | 4 | 1 | −2 | −3 | −2 | S |
| | | | | | | | | | | | | | | | | 5 | 0 | −2 | −2 | T |
| | | | | | | | | | | | | | | | | | 4 | −3 | −1 | V |
| | | | | | | | | | | | | | | | | | | 11 | 2 | W |
| | | | | | | | | | | | | | | | | | | | 7 | Y |

The term “homology” or “homologous” is intended to mean a sequence similarity between two polynucleotides or between two polypeptides. Similarity can be determined by comparing a position in each sequence, which can be aligned for purposes of comparison. If a given position of two polypeptide sequences is not identical, the similarity or conservativeness of that position can be determined by assessing the similarity of the amino acid of the position, for example, according to Table 3. A degree of similarity between sequences is a function of the number of matching or homologous positions shared by the sequences. The alignment of two sequences to determine their percent sequence similarity can be done using software programs known in the art, such as, for example, those described in Ausubel et al., Current Protocols in Molecular Biology, John Wiley and Sons, Baltimore, MD (1999). Preferably, default parameters are used for the alignment, examples of which are set forth below. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

The term “homologs” of to a given amino acid sequence or a nucleic acid sequence is intended to indicate that the corresponding sequences of the “homologs” having substantial identity or homology to the given amino acid sequence or nucleic acid sequence.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The term “cytotoxic agent” refers to a substance that inhibits or prevents the expression activity of cells, function of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to auristatins (e.g., auristatin E, auristatin F, MMAE and MMAF), auromycins, maytansinoids, ricin, ricin A-chain, combrestatin, duocarmycins, dolastatins, doxorubicin, daunorubicin, taxols, cisplatin, cc1065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷. Antibodies may also be conjugated to an anti-cancer pro-drug activating enzyme capable of converting the pro-drug to its active form.

The term “effective amount” or “therapeutically effective amount” as used herein refers to the amount of binding molecule (e.g., an antibody) or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., a cancer).

The term “cancer” or “cancer cell” is used herein to denote a tissue or cell found in a neoplasm which possesses characteristics which differentiate it from normal tissue or tissue cells. Among such characteristics include but are not limited to: degree of anaplasia, irregularity in shape, indistinctness of cell outline, nuclear size, changes in structure of nucleus or cytoplasm, other phenotypic changes, presence of cellular proteins indicative of a cancerous or pre-cancerous state, increased number of mitoses, and ability to metastasize. Words pertaining to “cancer” include carcinoma, sarcoma, tumor, epithelioma, leukemia, lymphoma, polyp, and scirrus, transformation, neoplasm, and the like.

As used herein, a “locally advanced” cancer refers to a cancer that has spread from where it started to nearby tissue or lymph nodes.

As used herein, a “metastatic” cancer refers to a cancer that has spread from where it started to different part of the body.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

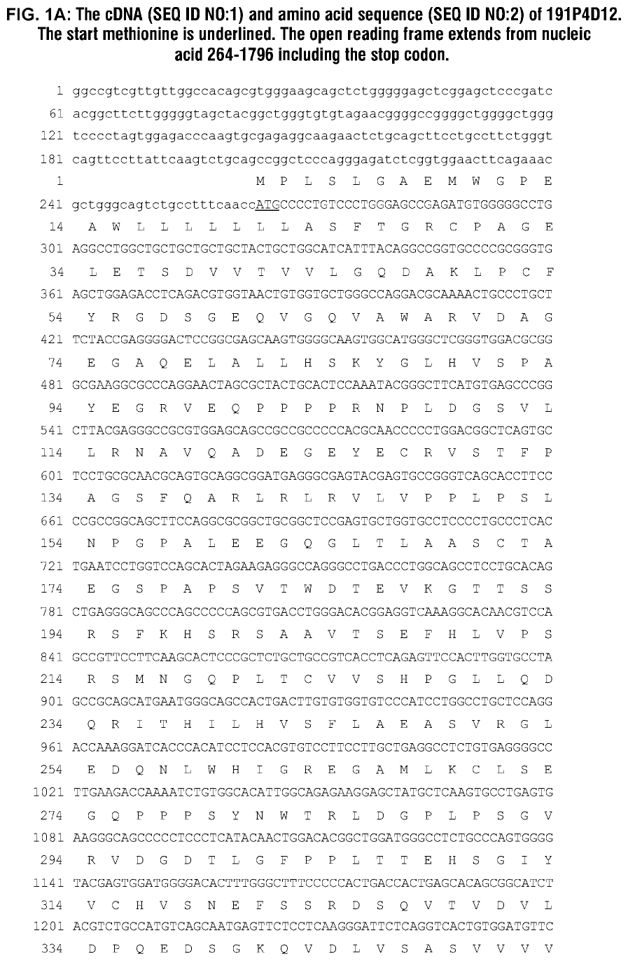

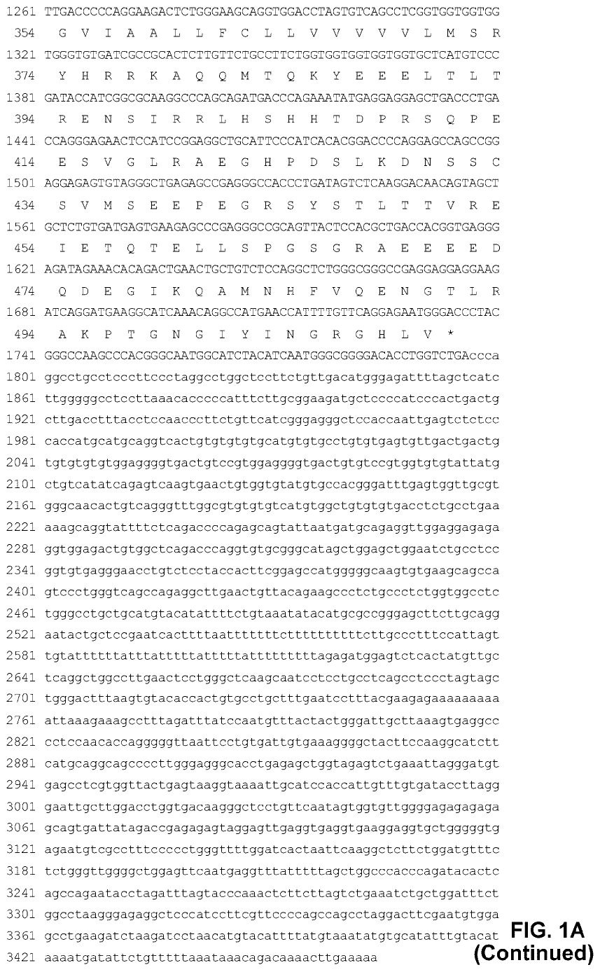

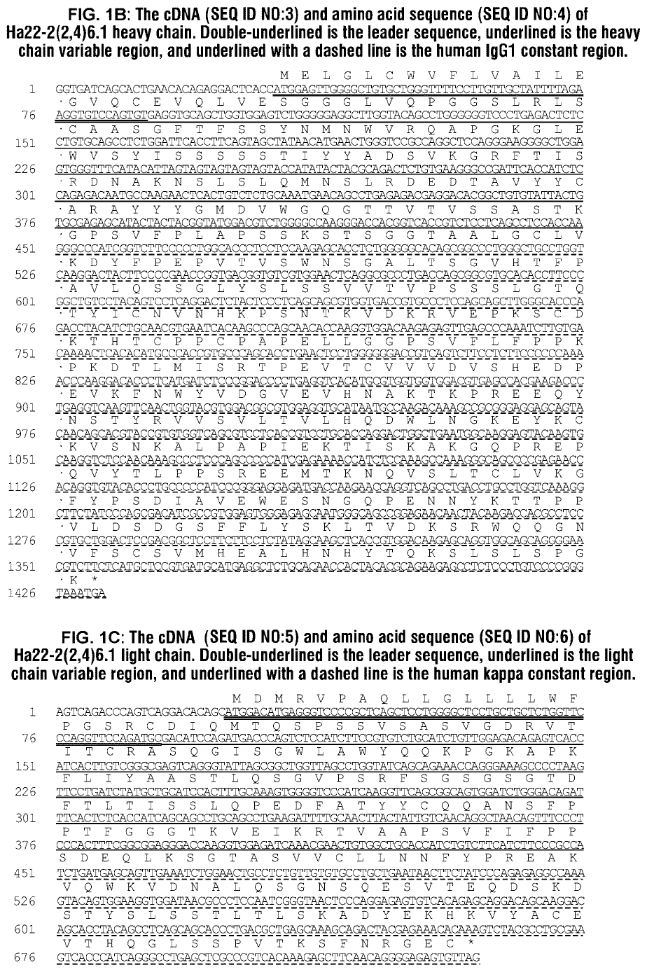

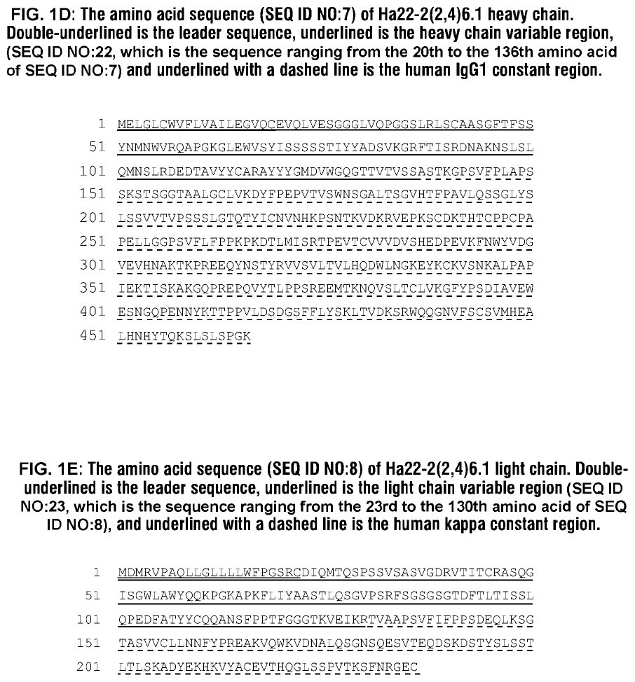

The term “variant” refers to a molecule that exhibits a variation from a described type or norm, such as a protein that has one or more different amino acid residues in the corresponding position(s) of a specifically described protein (e.g. the 191P4D12 protein shown in FIG. 1A.) An analog is an example of a variant protein. Splice isoforms and single nucleotides polymorphisms (SNPs) are further examples of variants.

The “191P4D12 proteins” and/or “191P4D12 related proteins” of the disclosure include those specifically identified herein (see, FIG. 1A), as well as allelic variants, conservative substitution variants, analogs and homologs that can be isolated/generated and characterized without undue experimentation following the methods outlined herein or readily available in the art. Fusion proteins that combine parts of different 191P4D12 proteins or fragments thereof, as well as fusion proteins of a 191P4D12 protein and a heterologous polypeptide are also included. Such 191P4D12 proteins are collectively referred to as the 191P4D12-related proteins, the proteins of the disclosure, or 191P4D12. The term “191P4D12-related protein” refers to a polypeptide fragment or a 191P4D12 protein sequence of 4, 5, 6, 7, 8, 9, 10, 11, 12, 13, 14, 15, 16, 17, 18, 19, 20, 21, 22, 23, 24, 25, or more than 25 amino acids; or, at least 30, 35, 40, 45, 50, 55, 60, 65, 70, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 330, 335, 339 or more amino acids. The term “191P4D12” is used interchangeably with nectin-4.

### 5.2 Methods of Treating Cancer

Urothelial cancer and bladder cancer (including locally advanced urothelial cancer, metastatic urothelial cancer, locally advanced bladder cancer and metastatic bladder cancer), in patients who are ineligible for cisplatin are particularly difficult diseases to treat. Typically, these patients are frail, suffer from multiple comorbidities beyond their urothelial cancer/bladder cancer and are not able to tolerate additional treatment, leading many to discontinue therapy altogether. As such, these patients have a poor prognosis and few treatment options. This disclosure is based in part upon the results of the first clinical trial to demonstrate objective responses, in which a combination of enfortumab vedotin and pembrolizumab was administered as first-line treatment to patients with unresectable locally advanced or metastatic urothelial cancer (la/mUC) who are unable to receive cisplatin-based chemotherapy. The disclosure thus provides demonstrated efficacious methods to treat patients with urothelial cancer and/or bladder cancer (including locally advanced urothelial cancer, metastatic urothelial cancer, locally advanced bladder cancer and metastatic bladder cancer) who are unable to receive cisplatin-based chemotherapy in this setting due to inadequate kidney function or other conditions as provided herein. Prior to the results described herein, there was considerable uncertainty whether the methods which are provided herein would be efficacious given that this patient population has historically proven so difficult to treat. As described further below, the level of efficacy obtained was particularly notable and surprising.

### 5.2.1 Methods of Treating Cancer in General and for Selected Patients

Provided herein are methods for the first-line and second-line treatment of various cancers in subjects, including subjects with unresectable locally advanced or metastatic urothelial cancer (la/mUC) who are unable to receive cisplatin-based chemotherapy, using an antibody drug conjugate (ADC) that binds 191P4D12 in combination with an anti-PD-1 antibody (e.g., pembrolizumab).

In one aspect, provided herein are methods for the treatment of cancer in a subject using an ADC that binds 191P4D12 and an anti-PD-1 antibody (e.g., pembrolizumab). In some embodiments, the human subject treated with the methods provided herein has not received previous cancer treatment other than the ADC that binds 191P4D12. In certain embodiments, the human subject treated with the methods provided herein has not received previous treatment that includes or consists of an immune checkpoint inhibitor (CPI). In some embodiments, the CPI is an anti-PD-1 antibody (e.g., pembrolizumab). In some embodiments, the CPI is a PD-1 inhibitor, PD-L1 inhibitor, or PD-L2 inhibitor (including, but not limited to, atezolizumab, pembrolizumab, nivolumab, durvalumab, or avelumab). In particular embodiments, the CPI is atezolizumab, pembrolizumab, nivolumab, durvalumab, or avelumab. In certain embodiments, the human subject treated with the methods provided herein has not received an agent directed to another stimulatory or co inhibitory T-cell receptor (including but not limited to CD137 agonists, CTLA 4 inhibitors, or OX-40 agonists). In particular embodiments, the agent directed to another stimulatory or co inhibitory T-cell receptor is a CD137 agonist, a CTLA 4 inhibitor, or an OX-40 agonist. In some embodiments, the human subject treated with the methods provided herein is ineligible to receive cisplatin treatment. In other embodiments, the human subject treated with the methods provided herein is ineligible to receive cisplatin treatment and has not received previous treatment including or consisting of a CPI. In certain embodiments, the human subject treated with the methods provided herein is ineligible to receive cisplatin treatment, has not received previous treatment including or consisting of a CPI, and has not received adjuvant/neoadjuvant platinum-based therapy within 12 months prior to randomization. In still further embodiments, the human subject treated with the methods provided herein is ineligible to receive cisplatin treatment, has not received previous treatment including or consisting of a CPI, has not received adjuvant/neoadjuvant platinum-based therapy within 12 months prior to randomization, and has not received prior systemic treatment for locally advanced or metastatic disease. In some embodiments, the cancer is urothelial cancer. In certain embodiments, the cancer is bladder cancer. In certain embodiments, the cancer is cancer of the renal pelvis. In certain embodiments, the cancer is cancer of the ureter. In certain embodiments, the cancer is cancer of the urethra. In one embodiment, the cancer is locally advanced cancer. In another embodiment, the cancer is metastatic cancer. In a further embodiment, the cancer is locally advanced urothelial cancer. In a further embodiment, the cancer is unresectable locally advanced urothelial cancer. In yet another embodiment, the cancer is metastatic urothelial cancer. In one embodiment, the cancer is locally advanced bladder cancer. In another embodiment, the cancer is metastatic bladder cancer.

### 5.2.1.1 Cisplatin Ineligible Patients

Various conditions can be used to determine the cisplatin ineligibility for the human subjects for the methods provided herein, including but not limited to the methods of the preceding paragraphs. In one embodiment, the conditions for determining the cisplatin ineligibility comprise or consist of ECOG performance status score of 2. In some embodiments, the conditions for determining the cisplatin ineligibility comprise or consist of impaired renal function (e.g., glomerular filtration rate (GFR) or creatinine clearance <60 mL/min but ≥30 mL/min (estimated by the Cockcroft-Gault formula, modification of diet in renal disease [MDRD] or 24 hour urine)). In certain embodiments, the conditions for determining the cisplatin ineligibility comprise or consist of no less than Grade 2 hearing loss (e.g., CI CTCAE Version 4.03 Grade ≥2 hearing loss). In certain embodiments, the conditions for determining the cisplatin ineligibility comprise or consist of NYHA Class III heart failure. In one embodiment, the conditions for determining the cisplatin ineligibility comprise or consist of ECOG performance status score of 2 and impaired renal function. In some embodiments, the conditions for determining the cisplatin ineligibility comprise or consist of ECOG performance status score of 2 and no less than Grade 2 hearing loss. In further embodiments, the conditions for determining the cisplatin ineligibility comprise or consist of impaired renal function and no less than Grade 2 hearing loss. In further embodiments, the conditions for determining the cisplatin ineligibility comprise or consist of impaired renal function and NYHA Class III heart failure. In yet other embodiments, the conditions for determining the cisplatin ineligibility comprise or consist of ECOG performance status score of 2, impaired renal function, and no less than Grade 2 hearing loss. In some embodiments, the conditions for determining the cisplatin ineligibility comprise or consist of impaired renal function, no less than Grade 2 hearing loss, and NYHA Class III heart failure. In some embodiments, the conditions for determining the cisplatin ineligibility comprise or consist of any one of ECOG performance status score of 2, impaired renal function, and no less than Grade 2 hearing loss. In some embodiments, the conditions for determining the cisplatin ineligibility comprise or consist of any one of impaired renal function, no less than Grade 2 hearing loss, and NYHA Class III heart failure. In some embodiments, the conditions for determining the cisplatin ineligibility comprise or consist of any two of ECOG performance status score of 2, impaired renal function, and no less than Grade 2 hearing loss, in any combination or permutation. In some embodiments, the conditions for determining the cisplatin ineligibility comprise or consist of any two of impaired renal function, no less than Grade 2 hearing loss, and NYHA Class III heart failure, in any combination or permutation. In some embodiments, the conditions for determining the cisplatin ineligibility comprise or consist of all three of ECOG performance status score of 2, impaired renal function, and no less than Grade 2 hearing loss. In some embodiments, the conditions for determining the cisplatin ineligibility comprise or consist of all three of impaired renal function, no less than Grade 2 hearing loss, and NYHA Class III heart failure. In some embodiments of the methods provided herein, the subject did not receive prior systemic treatment for locally advanced or metastatic disease. In some embodiments of the methods provided herein, the subject did not receive adjuvant/neoadjuvant platinum-based therapy within 12 months prior to randomization. In some embodiments of the methods provided herein, wherein the subject has ECOG performance status score of 2, the subject (i) has Hemoglobin ≥10 g/dL; (ii) has GFR ≥50 mL/min; and (iii) does not have NYHA Class III heart failure.

Impaired renal function can be determined as various means known and available in the art. Various embodiments are provided herein to determine the impaired renal function for the human subjects for the methods provided herein, including but not limited to the methods of the preceding paragraph. In one embodiment, the impaired renal function is determined by glomerular filtration rate (GFR) less than 60 mL/min. In some embodiments, the impaired renal function is determined by GFR less than 60 but no less than 30 mL/min. In certain embodiments, the impaired renal function is determined by GFR less than 30 but no less than 15 mL/min. In some embodiments of the methods provided in this paragraph, the GFR is measured by 24 hour urine collection. In other embodiments of the methods provided in this paragraph, the GFR is estimated by the Cockcroft-Gault criteria. In other embodiments of the methods provided in this paragraph, the GFR is measured by modification of diet in renal disease [MDRD].

In a further embodiment, the impaired renal function is determined by creatinine clearance (CrCl) less than 60 mL/min. In some embodiments, the impaired renal function is determined by CrCl less than 60 but no less than 30 mL/min. In certain embodiments, the impaired renal function is determined by CrCl less than 30 but no less than 15 mL/min. In some embodiments of the methods provided in this paragraph, the CrCl is measured by 24 hour urine collection. In other embodiments of the methods provided in this paragraph, the CrCl is estimated by the Cockcroft-Gault criteria.

As such, some specific conditions based on GFR or creatinine clearance can be used to determine the cisplatin ineligibility for the human subjects for the methods provided herein, including but not limited to the methods of the preceding paragraphs. In some embodiments, the conditions for determining the cisplatin ineligibility comprise or consist of GFR less than 60 mL/min. In one embodiment, the conditions for determining the cisplatin ineligibility comprise or consist of ECOG performance status score of 2 and CrCl less than 60 mL/min. In further embodiments, the conditions for determining the cisplatin ineligibility comprise or consist of GFR less than 60 m/min and no less than Grade 2 hearing loss. In further embodiments, the conditions for determining the cisplatin ineligibility comprise or consist of GFR less than 60 mL/min, and NYHA Class III heart failure. In yet other embodiments, the conditions for determining the cisplatin ineligibility comprise or consist of ECOG performance status score of 2, GFR less than 60 mL/min, and no less than Grade 2 hearing loss. In yet other embodiments, the conditions for determining the cisplatin ineligibility comprise or consist of GFR less than 60 mL/min, and no less than Grade 2 hearing loss, and NYHA Class III heart failure. In some embodiments, the conditions for determining the cisplatin ineligibility comprise or consist of any one of ECOG performance status score of 2, GFR less than 60 mL/min, and no less than Grade 2 hearing loss. In yet other embodiments, the conditions for determining the cisplatin ineligibility comprise or consist of any one of GFR less than 60 mL/min, and no less than Grade 2 hearing loss, and NYHA Class III heart failure. In some embodiments, the conditions for determining the cisplatin ineligibility comprise or consist of any two of ECOG performance status score of 2, GFR less than 60 mL/min, and no less than Grade 2 hearing loss, in any combination or permutation. In yet other embodiments, the conditions for determining the cisplatin ineligibility comprise or consist of any two of GFR 1 less than 60 mL/min, and no less than Grade 2 hearing loss, and NYHA Class III heart failure, in any combination or permutation. In some embodiments, the conditions for determining the cisplatin ineligibility comprise or consist of all three of ECOG performance status score of 2, GFR less than 60 mL/min, and no less than Grade 2 hearing loss. In some embodiments, the conditions for determining the cisplatin ineligibility comprise or consist of all three of GFR less than 60 mL/min, and no less than Grade 2 hearing loss, and NYHA Class III heart failure. In some embodiments of the methods provided in this paragraph, the GFR is measured by 24 hour urine collection. In other embodiments of the methods provided in this paragraph, the GFR is estimated by the Cockcroft-Gault criteria. In other embodiments of the methods provided in this paragraph, the GFR is measured by modification of diet in renal disease [MDRD]. In some embodiments of the methods provided herein, the subject did not receive prior systemic treatment for locally advanced or metastatic disease. In some embodiments of the methods provided herein, the subject did not receive adjuvant/neoadjuvant platinum-based therapy within 12 months prior to randomization. In some embodiments of the methods provided herein, wherein the subject has ECOG performance status score of 2, the subject (i) has Hemoglobin ≥10 g/dL; (ii) has GFR ≥50 mL/min; and (iii) does not have NYHA Class III heart failure.

In further embodiments, the conditions for determining the cisplatin ineligibility comprise or consist of CrCl less than 60 mL/min. In one embodiment, the conditions for determining the cisplatin ineligibility comprise or consist of ECOG performance status score of 2 and CrCl less than 60 mL/min. In further embodiments, the conditions for determining the cisplatin ineligibility comprise or consist of CrCl less than 60 mL/min and no less than Grade 2 hearing loss. In further embodiments, the conditions for determining the cisplatin ineligibility comprise or consist of CrCl less than 60 mL/min, and NYHA Class III heart failure. In yet other embodiments, the conditions for determining the cisplatin ineligibility comprise or consist of ECOG performance status score of 2, CrCl less than 60 mL/min, and no less than Grade 2 hearing loss. In yet other embodiments, the conditions for determining the cisplatin ineligibility comprise or consist of CrCl less than 60 m/min, and no less than Grade 2 hearing loss, and NYHA Class III heart failure. In some embodiments, the conditions for determining the cisplatin ineligibility comprise or consist of any one of ECOG performance status score of 2, CrCl less than 60 mL/min, and no less than Grade 2 hearing loss. In yet other embodiments, the conditions for determining the cisplatin ineligibility comprise or consist of any one of CrCl less than 60 mL/min, and no less than Grade 2 hearing loss, and NYHA Class III heart failure. In some embodiments, the conditions for determining the cisplatin ineligibility comprise or consist of any two of ECOG performance status score of 2, CrCl less than 60 mL/min, and no less than Grade 2 hearing loss, in any combination or permutation. In yet other embodiments, the conditions for determining the cisplatin ineligibility comprise or consist of any two of CrCl less than 60 mL/min, and no less than Grade 2 hearing loss, and NYHA Class III heart failure, in any combination or permutation. In some embodiments, the conditions for determining the cisplatin ineligibility comprise or consist of all three of ECOG performance status score of 2, CrCl less than 60 m/min, and no less than Grade 2 hearing loss. In some embodiments, the conditions for determining the cisplatin ineligibility comprise or consist of all three of CrCl less than 60 m/min, and no less than Grade 2 hearing loss, and NYHA Class III heart failure. In some embodiments of the methods provided in this paragraph, the CrCl is measured by 24 hour urine collection. In other embodiments of the methods provided in this paragraph, the CrCl is estimated by the Cockcroft-Gault criteria. In some embodiments of the methods provided herein, the subject did not receive prior systemic treatment for locally advanced or metastatic disease. In some embodiments of the methods provided herein, the subject did not receive adjuvant/neoadjuvant platinum-based therapy within 12 months prior to randomization. In some embodiments of the methods provided herein, wherein the subject has ECOG performance status score of 2, the subject (i) has Hemoglobin ≥10 g/dL; (ii) has GFR ≥50 mL/min; and (iii) does not have NYHA Class III heart failure.

Alternatively, other specific conditions based on GFR or creatinine clearance can be used to determine the cisplatin ineligibility for the human subjects for the methods provided herein, including but not limited to the methods of the preceding paragraphs. In some embodiments, embodiments, the conditions for determining the cisplatin ineligibility comprise or consist of GFR less than 60 but no less than 30 mL/min. In one embodiment, the conditions for determining the cisplatin ineligibility comprise or consist of ECOG performance status score of 2 and GFR less than 60 but no less than 30 mL/min. In further embodiments, the conditions for determining the cisplatin ineligibility comprise or consist of GFR less than 60 but no less than 30 m/min and no less than Grade 2 hearing loss. In further embodiments, the conditions for determining the cisplatin ineligibility comprise or consist of GFR less than 60 but no less than 30 m/min and NYHA Class III heart failure. In yet other embodiments, the conditions for determining the cisplatin ineligibility comprise or consist of ECOG performance status score of 2, GFR less than 60 but no less than 30 m/min, and no less than Grade 2 hearing loss. In yet other embodiments, the conditions for determining the cisplatin ineligibility comprise or consist of GFR less than 60 but no less than 30 mL/min, no less than Grade 2 hearing loss, and NYHA Class III heart failure. In some embodiments, the conditions for determining the cisplatin ineligibility comprise or consist of any one of ECOG performance status score of 2, GFR less than 60 but no less than 30 mL/min, and no less than Grade 2 hearing loss. In some embodiments, the conditions for determining the cisplatin ineligibility comprise or consist of any one of GFR less than 60 but no less than 30 m/min, no less than Grade 2 hearing loss, and NYHA Class III heart failure. In some embodiments, the conditions for determining the cisplatin ineligibility comprise or consist of any two of ECOG performance status score of 2, GFR less than 60 but no less than 30 m/min, and no less than Grade 2 hearing loss, in any combination or permutation. In some embodiments, the conditions for determining the cisplatin ineligibility comprise or consist of any two of GFR less than 60 but no less than 30 m/min, no less than Grade 2 hearing loss, and NYHA Class III heart failure, in any combination or permutation. In some embodiments, the conditions for determining the cisplatin ineligibility comprise or consist of all three of ECOG performance status score of 2, GFR less than 60 but no less than 30 mL/min, and no less than Grade 2 hearing loss. In some embodiments, the conditions for determining the cisplatin ineligibility comprise or consist of all three of GFR less than 60 but no less than 30 mL/min, no less than Grade 2 hearing loss, and NYHA Class III heart failure. In some embodiments of the methods provided in this paragraph, the GFR is measured by 24 hour urine collection. In other embodiments of the methods provided in this paragraph, the GFR is estimated by the Cockcroft-Gault criteria. In other embodiments of the methods provided in this paragraph, the GFR is measured by modification of diet in renal disease [MDRD]. In some embodiments of the methods provided herein, the subject did not receive prior systemic treatment for locally advanced or metastatic disease. In some embodiments of the methods provided herein, the subject did not receive adjuvant/neoadjuvant platinum-based therapy within 12 months prior to randomization. In some embodiments of the methods provided herein, wherein the subject has ECOG performance status score of 2, the subject (i) has Hemoglobin ≥10 g/dL; (ii) has GFR ≥50 mL/min; and (iii) does not have NYHA Class III heart failure.

In further embodiments, the conditions for determining the cisplatin ineligibility comprise or consist of CrCl less than 60 but no less than 30 mL/min. In one embodiment, the conditions for determining the cisplatin ineligibility comprise or consist of ECOG performance status score of 2 and CrCl less than 60 but no less than 30 mL/min. In further embodiments, the conditions for determining the cisplatin ineligibility comprise or consist of CrCl less than 60 but no less than 30 mL/min and no less than Grade 2 hearing loss. In further embodiments, the conditions for determining the cisplatin ineligibility comprise or consist of CrCl less than 60 but no less than 30 mL/min and NYHA Class III heart failure. In yet other embodiments, the conditions for determining the cisplatin ineligibility comprise or consist of ECOG performance status score of 2, CrCl less than 60 but no less than 30 mL/min, and no less than Grade 2 hearing loss. In yet other embodiments, the conditions for determining the cisplatin ineligibility comprise or consist of CrCl less than 60 but no less than 30 mL/min, no less than Grade 2 hearing loss, and NYHA Class III heart failure. In some embodiments, the conditions for determining the cisplatin ineligibility comprise or consist of any one of ECOG performance status score of 2, CrCl less than 60 but no less than 30 mL/min, and no less than Grade 2 hearing loss. In some embodiments, the conditions for determining the cisplatin ineligibility comprise or consist of any one of CrCl less than 60 but no less than 30 mL/min, no less than Grade 2 hearing loss, and NYHA Class III heart failure. In some embodiments, the conditions for determining the cisplatin ineligibility comprise or consist of any two of ECOG performance status score of 2, CrCl less than 60 but no less than 30 mL/min, and no less than Grade 2 hearing loss, in any combination or permutation. In some embodiments, the conditions for determining the cisplatin ineligibility comprise or consist of any two of CrCl less than 60 but no less than 30 mL/min, no less than Grade 2 hearing loss, and NYHA Class III heart failure, in any combination or permutation. In some embodiments, the conditions for determining the cisplatin ineligibility comprise or consist of all three of ECOG performance status score of 2, CrCl less than 60 but no less than 30 mL/min, and no less than Grade 2 hearing loss. In some embodiments, the conditions for determining the cisplatin ineligibility comprise or consist of all three of CrCl less than 60 but no less than 30 mL/min, no less than Grade 2 hearing loss, and NYHA Class III heart failure. In some embodiments of the methods provided in this paragraph, the CrCl is measured by 24 hour urine collection. In other embodiments of the methods provided in this paragraph, the CrCl is estimated by the Cockcroft-Gault criteria. In some embodiments of the methods provided herein, the subject did not receive prior systemic treatment for locally advanced or metastatic disease. In some embodiments of the methods provided herein, the subject did not receive adjuvant/neoadjuvant platinum-based therapy within 12 months prior to randomization. In some embodiments of the methods provided herein, wherein the subject has ECOG performance status score of 2, the subject (i) has Hemoglobin ≥10 g/dL; (ii) has GFR ≥50 mL/min; and (iii) does not have NYHA Class III heart failure.

Similarly, further specific conditions based on GFR or creatinine clearance can be used to determine the cisplatin ineligibility for the human subjects for the methods provided herein, including but not limited to the methods of the preceding paragraphs. some embodiments, the conditions for determining the cisplatin ineligibility comprise or consist of GFR less than 30 but no less than 15 mL/min. In one embodiment, the conditions for determining the cisplatin ineligibility comprise or consist of ECOG performance status score of 2 and GFR less than 30 but no less than 15 mL/min. In further embodiments, the conditions for determining the cisplatin ineligibility comprise or consist of GFR less than 30 but no less than 15 mL/min and no less than Grade 2 hearing loss. In further embodiments, the conditions for determining the cisplatin ineligibility comprise or consist of GFR less than 30 but no less than 15 mL/min, and NYHA Class III heart failure. In yet other embodiments, the conditions for determining the cisplatin ineligibility comprise or consist of ECOG performance status score of 2, GFR less than 30 but no less than 15 mL/min, and no less than Grade 2 hearing loss. In yet other embodiments, the conditions for determining the cisplatin ineligibility comprise or consist of GFR less than 30 but no less than 15 mL/min, no less than Grade 2 hearing loss, and NYHA Class III heart failure. In some embodiments, the conditions for determining the cisplatin ineligibility comprise or consist of any one of ECOG performance status score of 2, GFR less than 30 but no less than 15 mL/min, and no less than Grade 2 hearing loss. In some embodiments, the conditions for determining the cisplatin ineligibility comprise or consist of any one of GFR less than 30 but no less than 15 mL/min, no less than Grade 2 hearing loss, and NYHA Class III heart failure. In some embodiments, the conditions for determining the cisplatin ineligibility comprise or consist of any two of ECOG performance status score of 2, GFR less than 30 but no less than 15 mL/min, and no less than Grade 2 hearing loss, in any combination or permutation. In some embodiments, the conditions for determining the cisplatin ineligibility comprise or consist of any two of GFR less than 30 but no less than 15 mL/min, no less than Grade 2 hearing loss, and NYHA Class III heart failure, in any combination or permutation. In some embodiments, the conditions for determining the cisplatin ineligibility comprise or consist of all three of ECOG performance status score of 2, GFR less than 30 but no less than 15 mL/min, and no less than Grade 2 hearing loss. In some embodiments, the conditions for determining the cisplatin ineligibility comprise or consist of all three of GFR less than 30 but no less than 15 mL/min, no less than Grade 2 hearing loss, and NYHA Class III heart failure. In some embodiments of the methods provided in this paragraph, the GFR is measured by 24 hour urine collection. In other embodiments of the methods provided in this paragraph, the GFR is estimated by the Cockcroft-Gault criteria. In other embodiments of the methods provided in this paragraph, the GFR is measured by modification of diet in renal disease [MDRD]. In some embodiments of the methods provided herein, the subject did not receive prior systemic treatment for locally advanced or metastatic disease. In some embodiments of the methods provided herein, the subject did not receive adjuvant/neoadjuvant platinum-based therapy within 12 months prior to randomization. In some embodiments of the methods provided herein, wherein the subject has ECOG performance status score of 2, the subject (i) has Hemoglobin ≥10 g/dL; (ii) has GFR ≥50 mL/min; and (iii) does not have NYHA Class III heart failure.

In some embodiments, the conditions for determining the cisplatin ineligibility comprise or consist of CrCl less than 30 but no less than 15 mL/min. In one embodiment, the conditions for determining the cisplatin ineligibility comprise or consist of ECOG performance status score of 2 and CrCl less than 30 but no less than 15 mL/min. In further embodiments, the conditions for determining the cisplatin ineligibility comprise or consist of CrCl less than 30 but no less than 15 mL/min and no less than Grade 2 hearing loss. In further embodiments, the conditions for determining the cisplatin ineligibility comprise or consist of CrCl less than 30 but no less than 15 mL/min, and NYHA Class III heart failure. In yet other embodiments, the conditions for determining the cisplatin ineligibility comprise or consist of ECOG performance status score of 2, CrCl less than 30 but no less than 15 mL/min, and no less than Grade 2 hearing loss. In yet other embodiments, the conditions for determining the cisplatin ineligibility comprise or consist of CrCl less than 30 but no less than 15 mL/min, no less than Grade 2 hearing loss, and NYHA Class III heart failure. In some embodiments, the conditions for determining the cisplatin ineligibility comprise or consist of any one of ECOG performance status score of 2, CrCl less than 30 but no less than 15 mL/min, and no less than Grade 2 hearing loss. In some embodiments, the conditions for determining the cisplatin ineligibility comprise or consist of any one of CrCl less than 30 but no less than 15 mL/min, no less than Grade 2 hearing loss, and NYHA Class III heart failure. In some embodiments, the conditions for determining the cisplatin ineligibility comprise or consist of any two of ECOG performance status score of 2, CrCl less than 30 but no less than 15 mL/min, and no less than Grade 2 hearing loss, in any combination or permutation. In some embodiments, the conditions for determining the cisplatin ineligibility comprise or consist of any two of CrCl less than 30 but no less than 15 mL/min, no less than Grade 2 hearing loss, and NYHA Class III heart failure, in any combination or permutation. In some embodiments, the conditions for determining the cisplatin ineligibility comprise or consist of all three of ECOG performance status score of 2, CrCl less than 30 but no less than 15 mL/min, and no less than Grade 2 hearing loss. In some embodiments, the conditions for determining the cisplatin ineligibility comprise or consist of all three of CrCl less than 30 but no less than 15 mL/min, no less than Grade 2 hearing loss, and NYHA Class III heart failure. In some embodiments of the methods provided in this paragraph, the CrCl is measured by 24 hour urine collection. In other embodiments of the methods provided in this paragraph, the CrCl is estimated by the Cockcroft-Gault criteria. In some embodiments of the methods provided herein, the subject did not receive prior systemic treatment for locally advanced or metastatic disease. In some embodiments of the methods provided herein, the subject did not receive adjuvant/neoadjuvant platinum-based therapy within 12 months prior to randomization. In some embodiments of the methods provided herein, wherein the subject has ECOG performance status score of 2, the subject (i) has Hemoglobin ≥10 g/dL; (ii) has GFR ≥50 mL/min; and (iii) does not have NYHA Class III heart failure.

### 5.2.1.2 Additional Patient Demographics

Additionally, the human subjects for whom the methods provided herein can be used are human subjects having various other conditions. In one embodiment, the human subjects for whom the methods provided herein may have histologically documented locally advanced or metastatic urothelial (previously known as transitional cell) cancer (e.g., cancer of the bladder, renal pelvis, ureter, or urethra). In some embodiments, the human subjects for whom the methods provided herein may be eligible for CPI therapy. In certain embodiments, the human subjects for whom the methods provided herein may measurable disease according to RECIST Version 1.1. In other embodiments, the human subjects for whom the methods provided herein may have lesions in a prior irradiated field that have progressed to be considered measurable. In yet other embodiments, the human subjects for whom the methods provided herein can have ECOG performance status score of 0. In yet other embodiments, the human subjects for whom the methods provided herein can have ECOG performance status score of 1. In yet other embodiments, the human subjects for whom the methods provided herein can have ECOG performance status score of 2. In yet other embodiments, the human subjects for whom the methods provided herein can have ECOG performance status score of 1 to 2. In yet other embodiments, the human subjects for whom the methods provided herein can have ECOG performance status score of 1 or 2. In yet other embodiments, the human subjects for whom the methods provided herein can have ECOG performance status score of 0 to 1. In yet other embodiments, the human subjects for whom the methods provided herein can have ECOG performance status score of 0 or 1. In yet other embodiments, the human subjects for whom the methods provided herein can have ECOG performance status score of 0 to 2. In yet other embodiments, the human subjects for whom the methods provided herein can have ECOG performance status score of 0 or 2. In yet other embodiments, the human subjects for whom the methods provided herein can have ECOG performance status score of 0, 1, or 2. In one embodiment, the human subjects for whom the methods provided herein may have an anticipated life expectancy of ≥3 months. Some embodiments of the methods provided in this paragraph may have any of the preceding embodiments in any combination or permutation.

In further embodiments of the methods provided herein, including the methods of the preceding paragraphs, the human subjects for whom the methods provided herein can be used are human subjects having various other conditions. In one embodiment, the human subjects for whom the methods provided herein can be used also have the conditions of absolute neutrophil count (ANC) no less than 1500/μL. In some embodiments, the human subjects for whom the methods provided herein can be used also have the conditions of platelet count no less than 100,000/μL. In certain embodiments, the human subjects for whom the methods provided herein can be used also have the conditions of hemoglobin no less than 9 g/dL. In certain embodiments, the human subjects for whom the methods provided herein can be used also have the conditions of hemoglobin no less than 9 g/dL, wherein the subject does not have erythropoietin dependency and wherein the subject has not been administered a packed red blood cell (pRBC) transfusion within 2 weeks prior to treatment. In certain embodiments, the human subjects for whom the methods provided herein can be used also have the conditions of hemoglobin no less than 5.6 mmol/L. In certain embodiments, the human subjects for whom the methods provided herein can be used also have the conditions of hemoglobin no less than 5.6 mmol/L, wherein the subject does not have erythropoietin dependency and wherein the subject has not been administered a packed red blood cell (pRBC) transfusion within 2 weeks prior to treatment. In certain embodiments, the human subjects for whom the methods provided herein can be used also have the conditions of hemoglobin no less than 9 g/dL or 5.6 mmol/L. In other embodiments, the human subjects for whom the methods provided herein can be used also have the conditions of serum bilirubin no more than either of 1.5 times of upper limit of normal (ULN), direct bilirubin ≤ULN, wherein the human subjects have the conditions of total bilirubin levels >1.5×ULN, or serum bilirubin 3 times ULN for patients with Gilbert's disease. In yet other embodiments, the human subjects for whom the methods provided herein can be used also have the conditions of CrCl no less than 30 mL/min. In yet other embodiments, the human subjects for whom the methods provided herein can be used also have the conditions of GFR no less than 30 mL/min. In yet other embodiments, the human subjects for whom the methods provided herein can be used also have the conditions of CrCl or GFR no less than 30 mL/min. In yet other embodiments, the human subjects for whom the methods provided herein can be used also have the condition of CrCl no less than 30 mL/min, wherein the subjects have creatinine levels >1.5× institutional ULN and/or ≤1.5 ULN. In yet other embodiments, the human subjects for whom the methods provided herein can be used also have the condition of GFR no less than 30 mL/min, wherein the subjects have creatinine levels >1.5× institutional ULN and/or ≤1.5 ULN. In another embodiment, the human subjects for whom the methods provided herein can be used also have the conditions of alanine aminotransferase (ALT) and aspartate aminotransferase (AST) no more than 3 fold of ULN. In one embodiment, the human subjects for whom the methods provided herein can be used also have the conditions of absolute neutrophil count no less than 1500/μL and platelet count no less than 100,000/μL. In some embodiments, the human subjects for whom the methods provided herein can be used also have the conditions of absolute neutrophil count no less than 1500/μL and hemoglobin no less than 9 g/dL or 5.6 mmol/L. In certain embodiments, the human subjects for whom the methods provided herein can be used also have the conditions of absolute neutrophil count no less than 1500/μL and serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease. In other embodiments, the human subjects for whom the methods provided herein can be used also have the conditions of absolute neutrophil count no less than 1500/μL, and CrCl or GFR no less than 30 mL/min. In other embodiments, the human subjects for whom the methods provided herein can be used also have the conditions of absolute neutrophil count no less than 1500/μL and CrCl no less than 30 mL/min, wherein the subjects have creatinine levels >1.5× institutional ULN and/or ≤1.5 ULN. In some embodiments, the human subjects for whom the methods provided herein can be used also have the conditions of absolute neutrophil count no less than 1500/μL and ALT and AST no more than 3 fold of ULN. In further embodiments, the human subjects for whom the methods provided herein can be used also have the conditions of platelet count no less than 100,000/μL and hemoglobin no less than 9 g/dL or 5.6 mmol/L. In one embodiment, the human subjects for whom the methods provided herein can be used also have the conditions of platelet count no less than 100,000/μL and serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease. In some embodiments, the human subjects for whom the methods provided herein can be used also have the conditions of platelet count no less than 100,000/μL and CrCl or GFR no less than 30 mL/min. In certain embodiments, the human subjects for whom the methods provided herein can be used also have the conditions of platelet count no less than 100,000/μL and ALT and AST no more than 3 fold of ULN. In other embodiments, the human subjects for whom the methods provided herein can be used also have the conditions of hemoglobin no less than 9 g/dL or 5.6 mmol/L and serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease. In yet other embodiments, the human subjects for whom the methods provided herein can be used also have the conditions of hemoglobin no less than 9 g/dL or 5.6 mmol/L and CrCl or GFR no less than 30 mL/min. In some embodiments, the human subjects for whom the methods provided herein can be used also have the conditions of hemoglobin no less than 9 g/dL or 5.6 mmol/L and ALT and AST no more than 3 fold of ULN. In one embodiment, the human subjects for whom the methods provided herein can be used also have the conditions of serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease and CrCl no less than 30 mL/min. In another embodiment, the human subjects for whom the methods provided herein can be used also have the conditions of serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease and ALT and AST no more than 3 fold of ULN. In another embodiment, the human subjects for whom the methods provided herein can be used also have the conditions of CrCl or GFR no less than 30 mL/min and ALT and AST no more than 3 fold of ULN. In other embodiments, the human subjects for whom the methods provided herein can be used also have the conditions of absolute neutrophil count no less than 1500/μL, platelet count no less than 100,000/μL, and hemoglobin no less than 9 g/dL or 5.6 mmol/L. In yet other embodiments, the human subjects for whom the methods provided herein can be used also have the conditions of absolute neutrophil count no less than 1500/μL, platelet count no less than 100,000/μL and serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease. In further embodiments, the human subjects for whom the methods provided herein can be used also have the conditions of absolute neutrophil count no less than 1500/μL, platelet count no less than 100,000/μL, and CrCl or GFR no less than 30 mL/min. In some embodiments, the human subjects for whom the methods provided herein can be used also have the conditions of absolute neutrophil count no less than 1500/μL, platelet count no less than 100,000/μL, and ALT and AST no more than 3 fold of ULN. In some embodiments, the human subjects for whom the methods provided herein can be used also have the conditions of absolute neutrophil count no less than 1500/μL, hemoglobin no less than 9 g/dL or 5.6 mmol/L and serum bilirubin no more than either of 1.5 times of ULN or 3 times ULN for patients with Gilbert's disease. In certain embodiments, the human subjects for whom the methods provided herein can be used also have the conditions of absolute neutrophil count no less than 1500/μL, hemoglobin no less than 9 g/dL or 5.6 mmol/L, and CrCl or GFR no less than 30 mL/min. In some embodiments, the human subjects for whom the methods provided herein can be used also have the conditions of absolute neutrophil count no less than 1500/μL, hemoglobin no less than 9 g/dL or 5.6 mmol/L, and ALT and AST no more than 3 fold of ULN. In yet other embodiments, the human subjects for whom the methods provided herein can be used also have the conditions of absolute neutrophil count no less than 1500/μL, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and CrCl or GFR no less than 30 mL/min. In some embodiments, the human subjects for whom the methods provided herein can be used also have the conditions of absolute neutrophil count no less than 1500/μL, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and ALT and AST no more than 3 fold of ULN. In some embodiments, the human subjects for whom the methods provided herein can be used also have the conditions of absolute neutrophil count no less than 1500/μL, CrCl or GFR no less than 30 mL/min, and ALT and AST no more than 3 fold of ULN. In some embodiments, the human subjects for whom the methods provided herein can be used also have the conditions of platelet count no less than 100,000/μL, hemoglobin no less than 9 g/dL or 5.6 mmol/L, and serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease. In certain embodiments, the human subjects for whom the methods provided herein can be used also have the conditions of platelet count no less than 100,000/μL, hemoglobin no less than 9 g/dL or 5.6 mmol/L, and CrCl or GFR no less than 30 mL/min. In some embodiments, the human subjects for whom the methods provided herein can be used also have the conditions of platelet count no less than 100,000/μL, hemoglobin no less than 9 g/dL or 5.6 mmol/L, and ALT and AST no more than 3 fold of ULN. In other embodiments, the human subjects for whom the methods provided herein can be used also have the conditions of platelet count no less than 100,000/μL, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and CrCl or GFR no less than 30 mL/min. In yet other embodiments, the human subjects for whom the methods provided herein can be used also have the conditions of platelet count no less than 100,000/μL, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and ALT and AST no more than 3 fold of ULN. In some embodiments, the human subjects for whom the methods provided herein can be used also have the conditions of platelet count no less than 100,000/μL, CrCl or GFR no less than 30 mL/min, and ALT and AST no more than 3 fold of ULN. In yet other embodiments, the human subjects for whom the methods provided herein can be used also have the conditions of hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and CrCl or GFR no less than 30 mL/min. In other embodiments, the human subjects for whom the methods provided herein can be used also have the conditions of hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and ALT and AST no more than 3 fold of ULN. In certain embodiments, the human subjects for whom the methods provided herein can be used also have the conditions of hemoglobin no less than 9 g/dL or 5.6 mmol/L, CrCl or GFR no less than 30 mL/min, and ALT and AST no more than 3 fold of ULN. In some embodiments, the human subjects for whom the methods provided herein can be used also have the conditions of serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN. In other embodiments, the human subjects for whom the methods provided herein can be used also have the conditions of absolute neutrophil count no less than 1500/μL, platelet count no less than 100,000/μL, hemoglobin no less than 9 g/dL or 5.6 mmol/L, and serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease. In further embodiments, the human subjects for whom the methods provided herein can be used also have the conditions of absolute neutrophil count no less than 1500/μL, platelet count no less than 100,000/μL, hemoglobin no less than 9 g/dL or 5.6 mmol/L, and CrCl or GFR no less than 30 mL/min. In some embodiments, the human subjects for whom the methods provided herein can be used also have the conditions of absolute neutrophil count no less than 1500/μL, platelet count no less than 100,000/μL, hemoglobin no less than 9 g/dL or 5.6 mmol/L, and ALT and AST no more than 3 fold of ULN. In some embodiments, the human subjects for whom the methods provided herein can be used also have the conditions of absolute neutrophil count no less than 1500/μL, platelet count no less than 100,000/μL, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and CrCl or GFR no less than 30 mL/min. In some embodiments, the human subjects for whom the methods provided herein can be used also have the conditions of absolute neutrophil count no less than 1500/μL, platelet count no less than 100,000/μL, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and ALT and AST no more than 3 fold of ULN. In some embodiments, the human subjects for whom the methods provided herein can be used also have the conditions of absolute neutrophil count no less than 1500/μL, platelet count no less than 100,000/μL, CrCl or GFR no less than 30 mL/min, and ALT and AST no more than 3 fold of ULN. In certain embodiments, the human subjects for whom the methods provided herein can be used also have the conditions of absolute neutrophil count no less than 1500/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and CrCl or GFR no less than 30 mL/min. In certain embodiments, the human subjects for whom the methods provided herein can be used also have the conditions of absolute neutrophil count no less than 1500/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and ALT and AST no more than 3 fold of ULN. In certain embodiments, the human subjects for whom the methods provided herein can be used also have the conditions of absolute neutrophil count no less than 1500/μL, hemoglobin no less than 9 g/dL or 5.6 mmol/L, CrCl or GFR no less than 30 mL/min, and ALT and AST no more than 3 fold of ULN. In yet other embodiments, the human subjects for whom the methods provided herein can be used also have the conditions of absolute neutrophil count no less than 1500/μL, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN. In further embodiments, the human subjects for whom the methods provided herein can be used also have the conditions of platelet count no less than 100,000/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and CrCl or GFR no less than 30 mL/min. In some embodiments, the human subjects for whom the methods provided herein can be used also have the conditions of platelet count no less than 100,000/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and ALT and AST no more than 3 fold of ULN. In further embodiments, the human subjects for whom the methods provided herein can be used also have the conditions of platelet count no less than 100,000/μL, hemoglobin no less than 9 g/dL or 5.6 mmol/L, CrCl or GFR no less than 30 mL/min, and ALT and AST no more than 3 fold of ULN. In some embodiments, the human subjects for whom the methods provided herein can be used also have the conditions of platelet count no less than 100,000/μL, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN. In some embodiments, the human subjects for whom the methods provided herein can be used also have the conditions of hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN. In certain embodiments, the human subjects for whom the methods provided herein can be used also have the conditions of absolute neutrophil count no less than 1500/μL, platelet count no less than 100,000/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and CrCl or GFR no less than 30 mL/min. In some embodiments, the human subjects for whom the methods provided herein can be used also have the conditions of absolute neutrophil count no less than 1500/μL, platelet count no less than 100,000/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, and ALT and AST no more than 3 fold of ULN. In some embodiments, the human subjects for whom the methods provided herein can be used also have the conditions of absolute neutrophil count no less than 1500/μL, platelet count no less than 100,000/μL, hemoglobin no less than 9 g/dL or 5.6 mmol/L, CrCl or GFR no less than 30 mL/min, and ALT and AST no more than 3 fold of ULN. In certain embodiments, the human subjects for whom the methods provided herein can be used also have the conditions of absolute neutrophil count no less than 1500/μL, platelet count no less than 100,000/μL, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN. In certain embodiments, the human subjects for whom the methods provided herein can be used also have the conditions of absolute neutrophil count no less than 1500/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN. In some embodiments, the human subjects for whom the methods provided herein can be used also have the conditions of platelet count no less than 100,000/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN. In some embodiments, the human subjects for whom the methods provided herein can be used also have the conditions of absolute neutrophil count no less than 1500/μL, platelet count no less than 100,000/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN. In some embodiments, the human subjects for whom the methods provided herein can be used also have the conditions of any one of absolute neutrophil count no less than 1500/μL, platelet count no less than 100,000/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN. In some embodiments, the human subjects for whom the methods provided herein can be used also have the conditions of any two of absolute neutrophil count no less than 1500/μL, platelet count no less than 100,000/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN, in any combination or permutation. In some embodiments, the human subjects for whom the methods provided herein can be used also have the conditions of any three of absolute neutrophil count no less than 1500/μL, platelet count no less than 100,000/μL, hemoglobin no less than 9 g/dL or 5.6 mmol/L, serum bilirubin no more than either of 1.5 times of ULN or serum bilirubin 3 times ULN for patients with Gilbert's disease, CrCl or GFR no less than 30 mL/min, and ALT and AST no more than 3 fold of ULN, in any combination or permutation. In some embodiments, the human subjects for whom the methods provided herein can be used also have the conditions of any four of absolute neutrophil count no less than 1500/μL, platelet count no less than 100,000/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN, in any combination or permutation. In some embodiments, the human subjects for whom the methods provided herein can be used also have the conditions of any five of absolute neutrophil count no less than 1500/μL, platelet count no less than 100,000/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN, in any combination or permutation. In some embodiments, the human subjects for whom the methods provided herein can be used also have the conditions of all six of absolute neutrophil count no less than 1500/μL, platelet count no less than 100,000/μL, hemoglobin no less than 9 g/dL or 5.6 mmol/L, serum bilirubin no more than either of 1.5 times of ULN, direct bilirubin ≤ULN, wherein the human subjects have the conditions of total bilirubin levels >1.5×ULN, or serum bilirubin 3 times ULN for patients with Gilbert's disease, CrCl or GFR no less than 30 mL/min, and ALT and AST no more than 3 fold of ULN.

In certain embodiments, the human subjects for whom the methods provided herein may have previously had prostate cancer treated surgically or with radiation therapy at least 1 year prior to treatment with any of the methods provided herein, wherein the subject is prostate cancer-free and wherein (i) if the subject has undergone or received radical prostatectomy, the subject must have undetectable PSA for >1 year prior to treatment with any of the methods provided herein, (ii) if the subject has received radiation, the subject has a PSA doubling time >1 year (based on at least 3 values determined >1 month apart) and a total PSA value that does not meet Phoenix criteria for biochemical recurrence (e.g., <2.0 ng/mL above nadir). In certain embodiments, the human subjects for whom the methods provided herein may have untreated low-risk prostate cancer, wherein the subject has a Gleason score ≤6, on active surveillance with PSA doubling time >1 year (based on at least 3 values determined >1 month apart).

In other embodiments of the methods provided herein, including the methods of the preceding paragraphs, the human subjects for whom the methods provided herein can be used are human subjects free from certain conditions. In one embodiment, the human subjects for whom the methods provided herein have not received any prior treatment with a CPI. A CPI is defined as a PD-1 inhibitor, PD-L1 inhibitor, or PD-L2 inhibitor (including, but not limited to, atezolizumab, pembrolizumab, nivolumab, durvalumab, or avelumab). In some embodiments, the human subjects for whom the methods provided herein have not received a PD-1 inhibitor, PD-L1 inhibitor, or PD-L2 inhibitor. In certain embodiments, the human subjects for whom the methods provided herein have not received atezolizumab, pembrolizumab, nivolumab, durvalumab, or avelumab. In certain embodiments, the human subjects for whom the methods provided herein have not received any prior treatment with an agent directed to another stimulatory or co inhibitory T-cell receptor (including, but not limited to, CD137 agonists, CTLA 4 inhibitors, or OX-40 agonists). In certain embodiments, the human subjects for whom the methods provided herein have not received any prior treatment with CD137 agonists, CTLA 4 inhibitors, or OX-40 agonists. In one embodiment, the human subjects for whom the methods provided herein can have no more than Grade 2 sensory or motor neuropathy. In one embodiment, the human subjects for whom the methods provided herein can have no active central nervous system metastases. In one embodiment, the human subjects for whom the methods provided herein can have no uncontrolled diabetes. In one embodiment, the human subjects for whom the methods provided herein can have no more than Grade 2 sensory or motor neuropathy and no active central nervous system metastases. In some embodiments, the human subjects for whom the methods provided herein can have no more than Grade 2 sensory or motor neuropathy and no uncontrolled diabetes. In yet other embodiments, the human subjects for whom the methods provided herein can have no more than Grade 2 sensory or motor neuropathy no active central nervous system metastases, and no uncontrolled diabetes. In some embodiments, the human subjects for whom the methods provided herein can have any one of no more than Grade 2 sensory or motor neuropathy, no active central nervous system metastases, and no uncontrolled diabetes. In some embodiments, the human subjects for whom the methods provided herein can have any two of no more than Grade 2 sensory or motor neuropathy, no active central nervous system metastases, and no uncontrolled diabetes, in any combination or permutation. In some embodiments, the human subjects for whom the methods provided herein can have all three of no more than Grade 2 sensory or motor neuropathy, no active central nervous system metastases, and no uncontrolled diabetes. In one embodiment of the methods provided in this paragraph, the uncontrolled diabetes is determined by hemoglobin A1c (HbA1c) no less than 8%. In some embodiments of the methods provided in this paragraph, the uncontrolled diabetes is determined by HbA1c between 7 and 8% with associated diabetes symptoms that are not otherwise explained. In further embodiments of the methods provided in this paragraph, the associated diabetes symptoms comprise or consist of polyuria. In some other embodiments of the methods provided in this paragraph, the associated diabetes symptoms comprise or consist of polydipsia. In yet other embodiments of the methods provided in this paragraph, the associated diabetes symptoms comprise or consist of both polyuria and polydipsia.

In certain embodiments, the human subjects for whom the methods provided herein may have no ongoing clinically significant toxicity (Grade 2 or greater) associated with prior treatment. In certain embodiments, the human subjects for whom the methods provided herein may have no ongoing clinically significant toxicity (Grade 2 or greater) associated with prior treatment, wherein the prior treatment is radiotherapy or surgery. In one embodiment, the human subjects for whom the methods provided herein may have no conditions requiring high doses of steroids (e.g., >10 mg/day of prednisone or equivalent) or other immunosuppressive medications. In one embodiment, the human subjects for whom the methods provided herein may have no conditions requiring high doses of steroids or other immunosuppressive medications, wherein the steroids or other immunosuppressive medications are not inhaled or topical steroids. In certain embodiments, the human subjects for whom the methods provided herein may have no prior treatment with enfortumab vedotin or other MMAE-based ADCs for urothelial cancer. In certain embodiments, the human subjects for whom the methods provided herein may have no history of another invasive malignancy within 3 years before treatment with the methods provided herein. In certain embodiments, the human subjects for whom the methods provided herein may have no evidence of residual disease from a previously diagnosed malignancy. In certain embodiments, the human subjects for whom the methods provided herein may not receive systemic antimicrobial treatment for active infection, wherein the infection is a viral, bacterial, or fungal infection, at the time of first dose of enfortumab vedotin. systemic antimicrobial treatment for active infection (viral, bacterial, or fungal) at the time of first dose of enfortumab vedotin. In certain embodiments, the human subjects for whom the methods provided herein may have no positive hepatitis B surface antigen and/or antihepatitis B core antibody. In certain embodiments, the human subjects for whom the methods provided herein may have no active hepatitis C infection or known human immunodeficiency virus (HIV) infection. In certain embodiments, the human subjects for whom the methods provided herein may have no active tuberculosis. In certain embodiments, the human subjects for whom the methods provided herein may have no documented history of a cerebral vascular event (e.g., a stroke or transient ischemic attack), unstable angina, myocardial infarction, or cardiac symptoms (including congestive heart failure) consistent with NYHA Class IV within 6 months prior to the first dose of enfortumab vedotin. In certain embodiments, the human subjects for whom the methods provided herein may have no radiotherapy or major surgery within 2 weeks prior to treatment with the methods provided herein. In certain embodiments, the human subjects for whom the methods provided herein may have no treatment with chemotherapy, biologics, or investigational agents not completed 4 weeks prior to treatment with the methods provided herein. In certain embodiments, the human subjects for whom the methods provided herein may have no known severe (≥Grade 3) hypersensitivity to enfortumab vedotin or to any excipient contained in the drug formulation of enfortumab vedotin (including histidine, trehalose dihydrate, and polysorbate 20). In certain embodiments, the human subjects for whom the methods provided herein may have no known severe (≥Grade 3) hypersensitivity to pembrolizumab or to any excipient contained in the drug formulations of pembrolizumab. In certain embodiments, the human subjects for whom the methods provided herein may have no active keratitis or corneal ulcerations. In certain embodiments, the human subjects for whom the methods provided herein may have no active autoimmune disease that has required systemic treatment in past 2 years (e.g., use of disease modifying agents, corticosteroids, or immunosuppressive drugs). In certain embodiments, the human subjects for whom the methods provided herein may have no active autoimmune disease that has required systemic treatment in past 2 years, wherein the systemic treatment comprises disease modifying agents, corticosteroids, or immunosuppressive drugs. In some embodiments, the systemic treatment is not replacement therapy (e.g., thyroxine, insulin, or physiologic corticosteroid replacement therapy for adrenal or pituitary insufficiency). In some embodiments, the systemic treatment is not thyroxine, insulin, or physiologic corticosteroid replacement therapy for adrenal or pituitary insufficiency. In certain embodiments, the human subjects for whom the methods provided herein may have no history of idiopathic pulmonary fibrosis; organizing pneumonia, drug-induced pneumonitis, idiopathic pneumonitis, or evidence of active pneumonitis on screening chest CT scan. In certain embodiments, the human subjects for whom the methods provided herein may have no prior allogeneic stem cell or solid organ transplant. In certain embodiments, the human subjects for whom the methods provided herein may not have received a live, attenuated vaccine within 30 days prior to treatment with the methods provided herein. In particular, embodiments, the live vaccine is a measles, mumps, rubella, varicella/zoster (chicken pox), yellow fever, rabies, BCG, or typhoid vaccine. In particular, embodiments, the live, attenuated vaccine is an intranasal influenza vaccine. In certain embodiments, the human subjects for whom the methods provided herein may have no underlying medical condition that impairs the ability of the subject to receive or tolerate the methods provided herein.

In some embodiments of the methods provided herein, the CrCl is measured by 24 hour urine collection. In other embodiments of the methods provided herein, the CrCl is estimated by the Cockcroft-Gault criteria.

In some embodiments of the methods provided herein, the GFR is measured by 24 hour urine collection. In other embodiments of the methods provided herein, the GFR is estimated by the Cockcroft-Gault criteria. In other embodiments of the methods provided in this paragraph, the GFR is measured by modification of diet in renal disease [MDRD].

In some embodiments of the methods provided herein, the subject has been treated with one or more other cancer treatments. In certain embodiments of the methods provided herein, the urothelial cancer, including locally advanced or metastatic urothelial cancer, has been treated with one or more other cancer treatments.

In some embodiments, the CPI described herein can comprise or consist of any CPI as described in this Section (Section 5.2.1).

In all the methods provided herein and specifically those described in the preceding paragraphs: the ADCs that can be used are described in Sections 3, 5.2, 5.3, 5.4, 5.5, and 6, selection of patients for treatment is described herein and exemplified in this Section (Section 5.2) and Sections 3 and 6, dosing regimens and pharmaceutical composition for administering the therapeutic agent are described in this Section (Section 5.2), Sections 5.4, 5.6, 5.7 and 6 below, the biomarkers that can be used for identifying the therapeutic agents, selecting the patients, determining the outcome of these methods, and/or serving as criteria in any way for these methods are described herein and exemplified in this Section (Section 5.2, including 5.2.1 and 5.2.2) and Section 6, the biomarkers can be determined as described in Section 5.8 or as known in the art, therapeutic outcomes for the methods provided herein are described in this Section (Section 5.2 including Section 5.2.1.4) and Sections 3 and 6, additional therapeutic outcomes for the methods provided herein can be improvement of the biomarkers described herein, for example, those described and exemplified in this Section (Section 5.2 including 5.2.2) and Sections 3 and 6, and combination therapies including the ADCs and other therapeutic agents are described in this Section (Section 5.2) and in Section 5.5. Therefore, a person skilled in the art would understand that the methods provided herein include all permutations and combinations of the patients, therapeutic agents, dosing regiments, biomarkers, and therapeutic outcomes as described above and below.

In certain embodiments, the methods provided herein are used for treating subjects having urothelial cancers that express 191P4D12 RNA, express 191P4D12 protein, or express both 191P4D12 RNA and 191P4D12 protein. In one embodiment, the methods provided herein are used for treating subjects who have urothelial cancers that express 191P4D12 RNA, express 191P4D12 protein, or express both 191P4D12 RNA and 191P4D12 protein.

In certain embodiments, the methods provided herein are used for treating subjects having locally advanced urothelial cancers that express 191P4D12 RNA, express 191P4D12 protein, or express both 191P4D12 RNA and 191P4D12 protein. In one embodiment, the methods provided herein are used for treating subjects who have locally advanced urothelial cancers that express 191P4D12 RNA, express 191P4D12 protein, or express both 191P4D12 RNA and 191P4D12 protein and who have been previously treated with a CPI.

In certain embodiments, the methods provided herein are used for treating subjects having metastatic cancers that express 191P4D12 RNA, express 191P4D12 protein, or express both 191P4D12 RNA and 191P4D12 protein. In one embodiment, the methods provided herein are used for treating subjects who have metastatic urothelial cancers that express 191P4D12 RNA, express 191P4D12 protein, or express both 191P4D12 RNA and 191P4D12 protein.

In some embodiments, the 191P4D12 RNA expression in the cancers is determined by polynucleotide hybridization, sequencing (assessing the relative abundance of the sequences), and/or PCR (including RT-PCR). In some embodiments, the 191P4D12 protein expression in the cancers is determined by IHC, analysis in fluorescence-activated cell sorting (FACS), and/or western blotting. In some embodiments, the 191P4D12 protein expression in the cancers is determined by more than one method. In some embodiments, the 191P4D12 protein expression in the cancers is determined by two methods of IHC.

In some embodiments, the locally advanced or metastatic urothelial cancers are confirmed histologically, cytologically, or both histologically and cytologically. In some embodiments, the locally advanced or metastatic bladder cancers are confirmed histologically, cytologically, or both histologically and cytologically.

In some embodiments, the subject has visceral metastases. In some embodiments, the subject has lymph nodes only disease. In some embodiments, the disease site of origin is in the upper tract. In some embodiments, the disease site of origin is in the lower tract.

In some embodiments, the subject has a PD-L1 expression combined positive score (CPS) greater than or equal to 10. PD-L1 protein expression is determined by using the Combined Positive Score (CPS), which is the number of PD-L1 positive cells (i.e., tumor cells, lymphocytes, and macrophages) divided by the total number of viable tumor cells, and then multiplied by 100. In some embodiments, the subject has a PD-L1 expression CPS less than 10. Nectin H-scores range from 0-300, based upon the following calculation: H-score=[(0×% negative cells)+(1×% weakly positive cells)+(2×% moderately positive cells)+(3×% strongly positive cells)]. Urothelial cancer generally has a median H-score from 260-270. In some embodiments, the subject has a Nectin-4H-score between 0 and 300. In some embodiments, the subject has a Nectin-4H-score between 0 and 250, between 0 and 200, between 0 and 150, between 0 and 100, or between 0 and 50. In some embodiments, the subject has a Nectin-4H-score between 0 and 200.

5.2.1.3 Checkpoint Inhibitors and Combination Therapy with Pembrolizumab

### Checkpoint Inhibitors

In some embodiments, the subjects that can be treated in the methods provided herein have certain phenotypic or genotypic characteristics. In some embodiments, the subjects have any permutation and combination of the phenotypic or genotypic characteristics described herein.

In some embodiments, the phenotypic or genotypic characteristics are determined histologically, cytologically, or both histologically and cytologically. In some embodiments of methods provided herein, the histological and/or the cytological determination of the phenotypic and/or genotypic characteristics are performed as described in American Society of Clinical Oncology/College of American Pathologists (ASCO/CAP) guidelines based on the most recently analyzed tissue, which is incorporated herein in their entirety by reference. In some embodiments, the phenotypic or genotypic characteristics are determined by sequencing including the next generation sequencing (e.g. NGS from Illumina, Inc), DNA hybridization, and/or RNA hybridization.

In various aspects or embodiments of the methods provided herein, including the methods provided in this Section (Section 5.2) such as the methods provided in this and the preceding paragraphs, the human subjects for whom the methods are provided herein have not received any prior treatment with a CPI. A CPI is defined as a PD-1 inhibitor, PD-L1 inhibitor, or PD-L2 inhibitor (including, but not limited to, atezolizumab, pembrolizumab, nivolumab, durvalumab, or avelumab). In some embodiments, the human subjects for whom the methods provided herein have not received prior treatment with a PD-1 inhibitor, PD-L1 inhibitor, or PD-L2 inhibitor. In certain embodiments, the human subjects for whom the methods provided herein have not received prior treatment with atezolizumab, pembrolizumab, nivolumab, durvalumab, or avelumab As used herein, the term “immune checkpoint inhibitor” or “checkpoint inhibitor” (CPI) refers to molecules that totally or partially reduce, inhibit, interfere with or modulate one or more checkpoint proteins. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). Other exemplary checkpoint proteins include LAG-3, B7, TIM3 (HAVCR2), OX40 (CD134), GITR, CD137, CD40, VTCN1, IDO1, CD276, PVRIG, TIGIT, CD25 (IL2RA), IFNAR2, IFNAR1, CSF1R, VSIR (VISTA), or HLA. These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In certain embodiments, the checkpoint inhibitor for the methods provided herein can be an inhibitors or activators against a checkpoint protein that upregulated in cancer. In some specific embodiments, the checkpoint inhibitor for the methods provided herein can be an inhibitors or activators against a checkpoint protein including LAG-3, B7, TIM3 (HAVCR2), OX40 (CD134), GITR, CD137, CD40, VTCN1, IDO1, CD276, PVRIG, TIGIT, CD25 (IL2RA), IFNAR2, IFNAR1, CSF1R, VSIR (VISTA), or HLA. In some embodiments, the checkpoint inhibitor for the methods provided herein can be an inhibitors or activators selected from the group consisting of a PD-1 inhibitor, a PD-L1 inhibitor, a PD-L2 inhibitor, a CTLA-4 inhibitor, a LAG-3 inhibitor, a B7 inhibitor, a TIM3 (HAVCR2) inhibitor, an OX40 (CD134) inhibitor, a GITR agonist, a CD137 agonist, or a CD40 agonist, a VTCN1 inhibitor, an IDO1 inhibitor, a CD276 inhibitor, a PVRIG inhibitor, a TIGIT inhibitor, a CD25 (IL2RA) inhibitor, an IFNAR2 inhibitor, an IFNAR1 inhibitor, a CSF1R inhibitor, a VSIR (VISTA) inhibitor, or a therapeutic agent targeting HLA. Such inhibitors, activators, or therapeutic agents are further provided below.

In some embodiments, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In certain embodiments, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

“PD-1 antagonist” means any chemical compound or biological molecule that blocks binding of PD-L1 expressed on a cancer cell to PD-1 expressed on an immune cell (T cell, B cell or Natural Killer T cell) and in specific embodiments also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and in specific embodiments blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

In some embodiments, the checkpoint inhibitor is a PD-1 inhibitor or antagonist. In one embodiment, the PD-1 inhibitor or antagonist is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity. In one embodiment, the PD-1 antibody is cemiplimab. In another embodiment, the PD-1 antibody is camrelizumab. In a further embodiment, the PD-1 antibody is sintilimab. In some embodiments, the PD-1 antibody is tislelizumab. In certain embodiments, the PD-1 antibody is TSR-042. In yet another embodiment, the PD-1 antibody is PDR001. In yet another embodiment, the PD-1 antibody is toripalimab.

In certain embodiments, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®). In a further embodiment, the PD-L1 inhibitor is avelumab.

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitors is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitors is a TIM3 (T-cell immunoglobulin domain and mucin domain 3) inhibitor (Fourcade et al., J. Ep. Med., 2010, 207, 2175-86; Sakuishi et al., J. Ep. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhIL-15).

In one embodiment, the checkpoint inhibitor is a VTCN inhibitor. In one embodiment, the VTCN inhibitor is FPA150.

In one embodiment, the checkpoint inhibitor is an IDO inhibitor. In one embodiment, the IDO inhibitor is INCB024360. In another embodiment, the IDO inhibitor is indoximod. In one embodiment, the IDO inhibitor is epacadostat. In another embodiment, the IDO inhibitor is BMS986205. In yet another embodiment, the IDO inhibitor is Navoximod. In one embodiment, the IDO inhibitor is PF-06840003. In another embodiment, the IDO inhibitor is KHK2455. In yet another embodiment, the IDO inhibitor is RG70099. In one embodiment, the IDO inhibitor is IOM-E. In another embodiment, the IDO inhibitor is or IOM-D.

In some embodiments, the checkpoint inhibitor is a TIGIT inhibitor. In certain embodiments, the TIGIT inhibitor is an anti-TIGIT antibody. In one embodiment, the TIGIT inhibitor is MTIG7192A. In another embodiment, the TIGIT inhibitor is BMS-986207. In yet another embodiment, the TIGIT inhibitor is OMP-313M32. In one embodiment, the TIGIT inhibitor is MK-7684. In another embodiment, the TIGIT inhibitor is AB154. In yet another embodiment, the TIGIT inhibitor is CGEN-15137. In one embodiment, the TIGIT inhibitor is SEA-TIGIT. In another embodiment, the TIGIT inhibitor is ASP8374. In yet another embodiment, the TIGIT inhibitor is AJUD008.

In some embodiments, the checkpoint inhibitor is a VSIR inhibitor. In certain embodiments, the VSIR inhibitor is an anti-VSIR antibody. In one embodiment, the VSIR inhibitor is MTIG7192A. In another embodiment, the VSIR inhibitor is CA-170. In yet another embodiment, the VSIR inhibitor is JNJ 61610588. In one embodiment, the VSIR inhibitor is HMBD-002.

In some embodiments, the checkpoint inhibitor is a TIM3 inhibitor. In certain embodiments, the TIM3 inhibitor is an anti-TIM3 antibody. In one embodiment, the TIM3 inhibitor is AJUD009.

In some embodiments, the checkpoint inhibitor is a CD25 (IL2RA) inhibitor. In certain embodiments, the CD25 (IL2RA) inhibitor is an anti-CD25 (IL2RA) antibody. In one embodiment, the CD25 (IL2RA) inhibitor is daclizumab. In another embodiment, the CD25 (IL2RA) inhibitor is basiliximab.

In some embodiments, the checkpoint inhibitor is an IFNAR1 inhibitor. In certain embodiments, the IFNAR1 inhibitor is an anti-IFNAR1 antibody. In one embodiment, the IFNAR1 inhibitor is anifrolumab. In another embodiment, the IFNAR1 inhibitor is sifalimumab.

In some embodiments, the checkpoint inhibitor is a CSF1R inhibitor. In certain embodiments, the CSF1R inhibitor is an anti-CSF1R antibody. In one embodiment, the CSF1R inhibitor is pexidartinib. In another embodiment, the CSF1R inhibitor is emactuzumab. In yet another embodiment, the CSF1R inhibitor is cabiralizumab. In one embodiment, the CSF1R inhibitor is ARRY-382. In another embodiment, the CSF1R inhibitor is BLZ945. In yet another embodiment, the CSF1R inhibitor is AJUD010. In one embodiment, the CSF1R inhibitor is AMG820. In another embodiment, the CSF1R inhibitor is IMC-CS4. In yet another embodiment, the CSF1R inhibitor is JNJ-40346527. In one embodiment, the CSF1R inhibitor is PLX5622. In another embodiment, the CSF1R inhibitor is FPA008.

In some embodiments, the checkpoint inhibitor is a therapeutic agent targeting HLA. In certain embodiments, the therapeutic agent targeting HLA is an anti-HLA antibody. In one embodiment, the therapeutic agent targeting HLA is GSK01. In another embodiment, the therapeutic agent targeting HLA is IMC-C103C. In yet another embodiment, the therapeutic agent targeting HLA is IMC-F106C. In one embodiment, the therapeutic agent targeting HLA is IMC-G107C. In another embodiment, the therapeutic agent targeting HLA is ABBV-184.

The methods described herein can be used in combination with one or more second active agents as described herein where appropriate for treating diseases described herein and understood in the art.

### PD-1 Antagonists and Pembrolizumab

Provided herein are methods for the treatment of various cancers in subjects, including subjects with unresectable locally advanced or metastatic urothelial cancer (la/mUC) who are unable to receive cisplatin-based chemotherapy, using an antibody drug conjugate (ADC) that binds 191P4D12 in combination with pembrolizumab. In certain embodiments, the treatment is a first-line treatment. In other embodiments, the treatment is a second-line treatment.

“Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 4. Pembrolizumab has been approved by the U.S. FDA as described in the Prescribing Information for KEYTRUDA™ (Merck & Co., Inc., Rahway, NJ, USA; initial U.S. approval 2014, updated March 2021).

As used herein, a “pembrolizumab variant” or “a variant thereof” pertaining to a pembrolizumab sequence means a monoclonal antibody that comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

In one embodiment, the PD-1 antagonist useful in the treatment, medicaments and uses of the present invention include a monoclonal antibody (mAb), or antigen binding fragment thereof, that specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1, and useful in the treatment method, medicaments and uses of the present invention, are described in U.S. patent nos. U.S. Pat. Nos. 7,488,802, 7,521,051, 8,008,449, 8,354,509, and 8,168,757, and International application publn. nos. WO2004/004771, WO2004/072286, WO2004/056875, US2011/0271358, and WO 2008/156712. Specific anti-human PD-1 mAbs useful as the PD-1 antagonist in the treatment method, medicaments and uses of the present invention include: pembrolizumab (also known as MK-3475), a humanized IgG4 mAb with the structure described in WHO Drug Information, Vol. 27, No. 2, pages 161-162 (2013) and that comprises the heavy and light chain amino acid sequences shown in Table 2; nivolumab (BMS-936558), a human IgG4 mAb with the structure described in WHO Drug Information, Vol. 27, No. 1, pages 68-69 (2013) and that comprises the heavy and light chain amino acid sequences shown in Table 2; the humanized antibodies h409A11, h409A16 and h409A17, which are described in WO2008/156712, and AMP-514, which is being developed by MedImmune; cemiplimab; camrelizumab; sintilimab; tislelizumab; and toripalimab. Additional anti-PD-1 antibodies contemplated for use herein include MEDI0680 (U.S. Pat. No. 8,609,089), BGB-A317 (U.S. Patent publ. no. 2015/0079109), INCSHR1210 (SHR-1210) (PCT International application publ. no. WO2015/085847), REGN-2810 (PCT International application publ. no. WO2015/112800), PDRO01 (PCT International application publ. no. WO2015/112900), TSR-042 (ANB011) (PCT International application publ. no. WO2014/179664) and STI-1110 (PCT International application publ. no. WO2014/194302).

Examples of mAbs that bind to human PD-L1, and useful in the treatment method, medicaments and uses of the present invention, are described in U.S. Pat. No. 8,383,796. Specific anti-human PD-L1 mAbs useful as the PD-1 antagonist in the treatment method, medicaments and uses of the present invention include BMS-936559, MEDI4736, and MSB0010718C.

In some embodiments, the PD-1 antagonist is pembrolizumab (KEYTRUDA™, Merck & Co., Inc., Rahway, NJ, USA), nivolumab (OPDIVO™, Bristol-Myers Squibb Company, Princeton, NJ, USA), atezolizumab (TECENTRIQ™, Genentech, San Francisco, CA, USA), durvalumab (IMFINZI™, AstraZeneca Pharmaceuticals LP, Wilmington, DE), cemiplimab (LIBTAYO™, Regeneron Pharmaceuticals, Tarrytown, NY, USA) avelumab (BAVENCIO™, Merck KGaA, Darmstadt, Germany) or dostarlimab (JEMPERLI™, GlaxoSmithKline LLC, Philadelphia, PA). In other embodiments, the PD-1 antagonist is pidilizumab (U.S. Pat. No. 7,332,582), AMP-514 (MedImmune LLC, Gaithersburg, MD, USA), PDR001 (U.S. Pat. No. 9,683,048), BGB-A317 (U.S. Pat. No. 8,735,553), or MGA012 (MacroGenics, Rockville, MD).

In one embodiment, the PD-1 antagonist useful in the methods of the invention is an anti-PD-1 antibody that blocks the binding of PD-1 to PD-L1 and PD-L2. In some embodiments of the treatment methods, medicaments and uses of the present invention, the PD-1 antagonist is a monoclonal antibody, or antigen binding fragment thereof, that comprises: (a) a light chain variable region comprising light chain CDR1, CDR2 and CDR3 of SEQ ID NOs: 24, 25 and 26, respectively and (b) a heavy chain variable region comprising heavy chain CDR1, CDR2 and CDR3 of SEQ ID NOs: 29, 30 and 31, respectively.

In other embodiments of the treatment methods, medicaments and uses of the present invention, the PD-1 antagonist is a monoclonal antibody, or antigen binding fragment thereof, that specifically binds to human PD-1 and comprises (a) a heavy chain variable region comprising SEQ ID NO:32 or a variant thereof, and (b) a light chain variable region comprising SEQ ID NO:37 or a variant thereof. A variant of a heavy chain variable region sequence is identical to the reference sequence except having up to six conservative amino acid substitutions in the framework region (i.e., outside of the CDRs). A variant of a light chain variable region sequence is identical to the reference sequence except having up to three conservative amino acid substitutions in the framework region (i.e., outside of the CDRs).

In another embodiment of the treatment methods, medicaments and uses of the present invention, the PD-1 antagonist is a monoclonal antibody that specifically binds to human PD-1 and comprises (a) a heavy chain comprising SEQ ID NO: 33 and (b) a light chain comprising SEQ ID NO:28. In one embodiment, the PD-1 antagonist is an anti-PD-1 antibody that comprises two heavy chains and two light chains, and wherein the heavy and light chains comprise the amino acid sequences in SEQ ID NO:33 and SEQ ID NO:28, respectively.

In all of the above treatment methods, medicaments and uses, the PD-1 antagonist inhibits the binding of PD-L1 to PD-1, and in specific embodiments also inhibits the binding of PD-L2 to PD-1. In some embodiments of the above treatment methods, medicaments and uses, the PD-1 antagonist is a monoclonal antibody, or an antigen binding fragment thereof, that specifically binds to PD-1 or to PD-L1 and blocks the binding of PD-L1 to PD-1.

Table 4 below provides a list of the amino acid sequences of exemplary anti-PD-1 mAbs for use in the treatment method, medicaments and uses of the present invention.

TABLE 4
Exemplary PD-1 Antibody Sequences

| | | |
|---|---|---|
| Antibody | | SEQ |
| Feature | Amino Acid Sequence | ID NO. |

Pembrolizumab Light Chain

| | | |
|---|---|---|
| CDR1 | RASKGVSTSGYSYLH | 24 |
| CDR2 | LASYLES | 25 |
| CDR3 | QHSRDLPLT | 26 |
| Variable | EIVLTQSPATLSLSPGERATLSCRASKGVSTSGYSYLHWY | 27 |
| Region | QQKPGQAPRLLIYLASYLESGVPARFSGSGSGTDFTLTISSL | |
| | EPEDFAVYYCQHSRDLPLTFGGGTKVEIK | |
| Light Chain | EIVLTQSPATLSLSPGERATLSCRASKGVSTSGYSYLHWY | 28 |
| | QQKPGQAPRLLIYLASYLESGVPARFSGSGSGTDFTLTISSL | |
| | EPEDFAVYYCQHSRDLPLTFGGGTKVEIKRTVAAPSVFIFP | |
| | PSDEQLKSGTASVVCLLNNFYPREAKVQWKVDNALQSGN | |
| | SQESVTEQDSKDSTYSLSSTLTLSKADYEKHKVYACEVTH | |
| | QGLSSPVTKSFNRGEC | |

Pembrolizumab Heavy Chain

| | | |
|---|---|---|
| CDR1 | NYYMY | 29 |
| CDR2 | GINPSNGGTNFNEKFKN | 30 |
| CDR3 | RDYRFDMGFDY | 31 |
| Variable | QVQLVQSGVEVKKPGASVKVSCKASGYTFTNYYMYWVR | 32 |
| Region | QAPGQGLEWMGGINPSNGGTNFNEKFKNRVTLTTDSSTT | |
| | TAYMELKSLQFDDTAVYYCARRDYRFDMGFDYWGQGTT | |
| | VTVSS | |
| Heavy | QVQLVQSGVEVKKPGASVKVSCKASGYTFTNYYMYWVR | 33 |
| Chain | QAPGQGLEWMGGINPSNGGTNFNEKFKNRVTLTTDSSTT | |
| | TAYMELKSLQFDDTAVYYCARRDYRFDMGFDYWGQGTT | |
| | VTVSSASTKGPSVFPLAPCSRSTSESTAALGCLVKDYFPEP | |
| | VTVSWNSGALTSGVHTFPAVLQSSGLYSLSSVVTVPSSSL | |
| | GTKTYTCNVDHKPSNTKVDKRVESKYGPPCPPCPAPEFLG | |
| | GPSVFLFPPKPKDTLMISRTPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTT | |
| | PPVLDSDGSFFLYSRLTVDKSRWQEGNVFSCSVMHEALH | |
| | NHYTQKSLSLSLGK | |

TABLE 5
Additional PD-1 Antibodies and Antigen Binding
Fragments Useful in the Formulations, Methods and
Uses Provided Herein

A. Antibodies and antigen binding fragments
comprising light and heavy chain CDRs of
hPD-1.08A in WO 2008/156712

| | | |
|---|---|---|
| CDRL1 | RASKSVSTSGFSYLH | SEQ ID NO: 34 |
| CDRL2 | LASNLES | SEQ ID NO: 35 |
| CDRL3 | QHSWELPLT | SEQ ID NO: 36 |
| CDRH1 | SYYLY | SEQ ID NO: 37 |
| CDRH2 | GVNPSNGGTNFSEKFK | SEQ ID NO: 38 |
| CDRH3 | RDSNYDGGFDY | SEQ ID NO: 39 |

C. Antibodies and antigen binding fragments
comprising the mature h109A heavy chain
variable region and one of the mature K09A light
chain variable regions in WO 2008/156712

| | | |
|---|---|---|
| Heavy chain | MDWTWSILFLVAAPTGAHSQVQ | SEQ ID NO: 40 |
| VR | LVQSGVEVKKPGASVKVSCKAS | |
| | GYTFTNYYMYWVRQAPGOGLEW | |
| | MGGINPSNGGTNFNEKFKNRVT | |
| | LTTDSSTTTAYMELKSLQFDDT | |
| | AVYYCARRDYRFDMGFDYWGQG | |
| | TTVTVSS | |
| Light chain | MAPVQLLGLLVLFLPAMRCEIV | SEQ ID NO: 41 |
| VR | LTQSPATLSLSPGERATLSCRA | or |
| | SKGVSTSGYSYLHWYQQKPGQA | |
| | PRLLIYLASYLESGVPARFSGS | |
| | GSGTDFTLTISSLEPEDFAVYY | |
| | CQHSRDLPLTFGGGTKVEIK | |
| | MAPVQLLGLLVLFLPAMRCEIV | SEQ ID NO: 42 |
| | LTQSPLSLPVTPGEPASISCRA | or |
| | SKGVSTSGYSYLHWYLQKPGQS | |
| | POLLIYLASYLESGVPDRFSGS | |
| | GSGTDFTLKISRVEAEDVGVYY | |
| | CQHSRDLPLTFGQGTKLEIK | |
| | MAPVQLLGLLVLFLPAMRCDIV | SEQ ID NO: 43 |
| | MTQTPLSLPVTPGEPASISCRA | |
| | SKGVSTSGYSYLHWYLQKPGOS | |
| | POLLIYLASYLESGVPDRFSGS | |
| | GSGTAFTLKISRVEAEDVGLYY | |
| | CQHSRDLPLTFGQGTKLEIK | |

D. Antibodies and antigen binding fragments
comprising the mature 409 heavy chain and one of
the mature K09A light chains in WO 2008/156712

| | | |
|---|---|---|
| Heavy chain | MAVLGLLFCLVTFPSCVLSQVQ | SEQ ID NO: 44 |
| | LVQSGVEVKKPGASVKVSCKAS | |
| | GYTFTNYYMYWVRQAPGOGLEW | |
| | MGGINPSNGGTNFNEKFKNRVT | |
| | LTTDSSTTTAYMELKSLQFDDT | |
| | AVYYCARRDYRFDMGFDYWGQG | |
| | TTVTVSSASTKGPSVFPLAPCS | |
| | RSTSESTAALGCLVKDYFPEPV | |
| | TVSWNSGALTSGVHTFPAVLOS | |
| | SGLYSLSSVVTVPSSSLGTKTY | |
| | TCNVDHKPSNTKVDKRVESKYG | |
| | PPCPPCPAPEFLGGPSVFLFPP | |
| | KPKDTLMISRTPEVTCVVVDVS | |
| | QEDPEVQFNWYVDGVEVHNAKT | |
| | KPREEQFNSTYRVVSVLTVLHQ | |
| | DWLNGKEYKCKVSNKGLPSSIE | |
| | KTISKAKGQPREPQVYTLPPSQ | |
| | EEMTKNQVSLTCLVKGFYPSDI | |
| | AVEWESNGQPENNYKTTPPVLD | |
| | SDGSFFLYSRLTVDKSRWQEGN | |
| | VFSCSVMHEALHNHYTQKSLSL | |
| | SLGK | |
| Light chain | MAPVQLLGLLVLFLPAMRCEIV | SEQ ID NO: 45 |
| | LTQSPATLSLSPGERATLSCRA | or |
| | SKGVSTSGYSYLHWYQQKPGQA | |
| | PRLLIYLASYLESGVPARFSGS | |
| | GSGTDFTLTISSLEPEDFAVYY | |
| | CQHSRDLPLTFGGGTKVEIKRT | |
| | VAAPSVFIFPPSDEQLKSGTAS | |
| | VVCLLNNFYPREAKVQWKVDNA | |
| | LOSGNSQESVTEQDSKDSTYSL | |
| | SSTLTLSKADYEKHKVYACEVT | |
| | HQGLSSPVTKSFNRGEC | |
| | MAPVOLLGLLVLFLPAMRCEIV | SEQ ID NO: 46 |
| | LTQSPLSLPVTPGEPASISCRA | or |
| | SKGVSTSGYSYLHWYLOKPGQS | |
| | POLLIYLASYLESGVPDRFSGS | |
| | GSGTDFTLKISRVEAEDVGVYY | |
| | CQHSRDLPLTFGQGTKLEIKRT | |
| | VAAPSVFIFPPSDEQLKSGTAS | |
| | VVCLLNNFYPREAKVQWKVDNA | |
| | LQSGNSQESVTEQDSKDSTYSL | |
| | SSTLTLSKADYEKHKVYACEVT | |
| | HQGLSSPVTKSFNRGEC | |
| | MAPVQLLGLLVLFLPAMRCDIV | SEQ ID NO: 47 |
| | MTQTPLSLPVTPGEPASISCRA | |
| | SKGVSTSGYSYLHWYLQKPGOS | |
| | PQLLIYLASYLESGVPDRFSGS | |
| | GSGTAFTLKISRVEAEDVGLYY | |
| | CQHSRDLPLTFGQGTKLEIKRT | |
| | VAAPSVFIFPPSDEQLKSGTAS | |
| | VVCLLNNFYPREAKVQWKVDNA | |
| | LQSGNSQESVTEQDSKDSTYSL | |
| | SSTLTLSKADYEKHKVYACEVT | |
| | HQGLSSPVTKSFNRGEC | |

In one embodiment, the anti-PD-1 antibody or antigen-binding fragment thereof comprises a heavy chain constant region, e.g. a human constant region, such as g1, g2, g3, or g4 human heavy chain constant region or a variant thereof. In another embodiment, the anti-PD-1 antibody or antigen-binding fragment thereof comprises a light chain constant region, e.g. a human light chain constant region, such as lambda or kappa human light chain region or a variant thereof. By way of example, and not limitation, the human heavy chain constant region can be g4 and the human light chain constant region can be kappa. In an alternative embodiment, the Fc region of the antibody is g4 with a Ser228Pro mutation (Schuurman, J et.al., Mol. Immunol. 38: 1-8, 2001). In some embodiments, different constant domains may be appended to humanized VL and VH regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized. Although human IgG1 antibodies provide for long half-life and for effector functions, such as complement activation and antibody-dependent cellular cytotoxicity, such activities may not be desirable for all uses of the antibody. In such instances a human IgG4 constant domain, for example, may be used. The present invention includes the use of anti-PD-1 antibodies or antigen-binding fragments thereof which comprise an IgG4 constant domain. In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, in order to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Imunol. 30:105. In other instances, a modified IgG1 constant domain which has been modified to increase half-life or reduce effector function can be used.

In another embodiment, the PD-1 antagonist is an antibody or antigen binding protein that has a variable light domain and/or a variable heavy domain with at least 95%, 90%, 85%, 80%, 75% or 50% sequence identity to one of the variable light domains or variable heavy domains described above, and exhibits specific binding to PD-1. In another embodiment of the methods of treatment of the invention, the PD-1 antagonist is an antibody or antigen binding protein comprising variable light and variable heavy domains having up to 1, 2, 3, 4, or 5 or more amino acid substitutions, and exhibits specific binding to PD-1.

In some embodiments, the checkpoint inhibitor is administered after the administration of the ADCs provided herein. In other embodiments, the checkpoint inhibitor is administered simultaneously (e.g., in the same dosing period) with the ADCs provided herein. In yet other embodiments, the checkpoint inhibitor is administered after the administration of the ADCs provided herein.

In some embodiments, the amount of the checkpoint inhibitor for the various methods provided herein can be determined by standard clinical techniques. In certain embodiments, the amount of the PD-1 antagonist, e.g., pembrolizumab, for the various methods are provided in Section 5.6.

In some embodiments, the subjects that can be treated in the methods provided herein is a mammal. In some embodiments, the subjects that can be treated in the methods provided herein is a human.

### 5.2.1.4 Therapeutic Outcome of the Methods Provided Herein

Despite the poor prognosis for cisplatin ineligible human subjects who as described above are frail, suffer from multiple comorbidities beyond their urothelial cancer/bladder cancer, the methods provided herein, including in methods described in this Section (Section 5.2) and Sections 3 and 6, can provide beneficial therapeutic outcomes for these cisplatin ineligible human subjects. In one embodiment, the human subject has a complete response following the treatment by a method provided herein. In another embodiment, the human subject has a partial response following the treatment by a method provided herein. In another embodiment, the human subject has complete response or a partial response following the treatment by a method provided herein.

In some embodiments, the response (complete or partial response) is determined by evaluating the tumor or cancer site (lesions). The criteria for determining complete response (CR), partial response (PR), progressive disease (PD), and stable disease (SD) are described in Table 10.

In some embodiments, the human subjects and patients are used interchangeably. Therefore, a person skilled in the art would understand that the human subjects can be interchangeable with patients in any of the methods provided herein.

The therapeutic outcome of the methods provided herein thus can be evaluated based on any one or more of the response criteria described above.

In one embodiment, the human subject has a partial response following the treatment by a method provided herein. In another embodiment, the human subject has a partial response following the treatment by a method provided herein, wherein the partial response is defined by an at least or about 30% decrease in the sum of the diameters of target lesions, taking as reference the baseline sum diameters. In another embodiment, the human subject has a partial response following the treatment by a method provided herein, wherein the partial response is defined by an at least or about 35% decrease in the sum of the diameters of target lesions, taking as reference the baseline sum diameters. In a further embodiment, the human subject has a partial response following the treatment by a method provided herein, wherein the partial response is defined by an at least or about 40% decrease in the sum of the diameters of target lesions, taking as reference the baseline sum diameters. In yet another embodiment, the human subject has a partial response following the treatment by a method provided herein, wherein the partial response is defined by an at least or about 45% decrease in the sum of the diameters of target lesions, taking as reference the baseline sum diameters. In one embodiment, the human subject has a partial response following the treatment by a method provided herein, wherein the partial response is defined by an at least or about 50% decrease in the sum of the diameters of target lesions, taking as reference the baseline sum diameters. In another embodiment, the human subject has a partial response following the treatment by a method provided herein, wherein the partial response is defined by an at least or about 55% decrease in the sum of the diameters of target lesions, taking as reference the baseline sum diameters. In a further embodiment, the human subject has a partial response following the treatment by a method provided herein, wherein the partial response is defined by an at least or about 60% decrease in the sum of the diameters of target lesions, taking as reference the baseline sum diameters. In yet another embodiment, the human subject has a partial response following the treatment by a method provided herein, wherein the partial response is defined by an at least or about 65% decrease in the sum of the diameters of target lesions, taking as reference the baseline sum diameters. In one embodiment, the human subject has a partial response following the treatment by a method provided herein, wherein the partial response is defined by an at least or about 70% decrease in the sum of the diameters of target lesions, taking as reference the baseline sum diameters. In another embodiment, the human subject has a partial response following the treatment by a method provided herein, wherein the partial response is defined by an at least or about 75% decrease in the sum of the diameters of target lesions, taking as reference the baseline sum diameters. In a further embodiment, the human subject has a partial response following the treatment by a method provided herein, wherein the partial response is defined by an at least or about 80% decrease in the sum of the diameters of target lesions, taking as reference the baseline sum diameters. In yet another embodiment, the human subject has a partial response following the treatment by a method provided herein, wherein the partial response is defined by an at least or about 85% decrease in the sum of the diameters of target lesions, taking as reference the baseline sum diameters. In one embodiment, the human subject has a partial response following the treatment by a method provided herein, wherein the partial response is defined by an at least or about 90% decrease in the sum of the diameters of target lesions, taking as reference the baseline sum diameters. In another embodiment, the human subject has a partial response following the treatment by a method provided herein, wherein the partial response is defined by an at least or about 95% decrease in the sum of the diameters of target lesions, taking as reference the baseline sum diameters. In some embodiments, the diameter is determined according to the longest diameter of a lesion. In certain embodiments, the diameter is determined according to the longest diameter of a lesion in the plane of measurement. In some embodiments, the diameter is determined according to the longest diameter of a lesion in the plane of measurement with a minimal size of 10 mm by CT scan. In certain embodiments, the diameter is determined according to the longest diameter of a lesion in the plane of measurement with a minimal size of 10 mm by CT scan and CT slice thickness no greater than 5 mm.

The therapeutic outcomes of the methods provided herein can also be evaluated based on whether the disease is stable following the treatment. In one embodiment, the human subject has a stable disease following the treatment by a method provided herein. In another embodiment, the human subject does not have a progressive disease following the treatment by a method provided herein.

Alternatively, therapeutic outcomes based on the complete response, partial response, or stable disease can be evaluated with respect to a population of human subjects treated by a method provided herein by evaluating the percentage of the subjects having complete response, partial response, or stable disease in the treated population. As such, in some embodiments, the therapeutic outcome or efficacy measure applies to outcomes achieved by actually treating a population of subjects. In other embodiments, the therapeutic outcome or efficacy measure refers to the outcome or efficacy that is capable of being achieved if a population of human subjects was treated with a method as disclosed herein. While the following sections discuss the treatment of an actual population of human subjects, is should be understood that corresponding methods in which the outcome or efficacy measure is capable of being achieved in a patient population are also encompassed herein. In short, both scenarios described above apply to the following sections; only one scenario is described below in the interest of simplicity and to avoid redundancy.

In some embodiments of the methods provided herein, including, but not limited to, in Sections 3, 5.3, 5.8, and 6 and this Section (Section 5.2), the ADC is enfortumab vedotin. In certain embodiments of the methods provided herein, including, but not limited to, in Sections 3, 5.3, 5.8, and 6 and this Section (Section 5.2), the ADC is a biosimilar of enfortumab vedotin. In some embodiments of the methods provided herein, including, but not limited to, in Sections 3, 5.2.1.3, 5.8, and 6 and this Section (Section 5.2), the PD-1 antagonist or anti-PD-1 antibody is pembrolizumab.

In one embodiment, a population of the human subjects is treated by a method provided herein, wherein the percentage of the subjects having complete response in the treated population is at least or about 2%. In another embodiment, a population of the human subjects is treated by a method provided herein, wherein the percentage of the subjects having complete response in the treated population is at least or about 5%. In a further embodiment, a population of the human subjects is treated by a method provided herein, wherein the percentage of the subjects having complete response in the treated population is at least or about 10%. In a further embodiment, a population of the human subjects is treated by a method provided herein, wherein the percentage of the subjects having complete response in the treated population is at least or about 10.5%. In yet another embodiment, a population of the human subjects is treated by a method provided herein, wherein the percentage of the subjects having complete response in the treated population is at least or about 15%. In one embodiment, a population of the human subjects is treated by a method provided herein, wherein the percentage of the subjects having complete response in the treated population is at least or about 20%.

In one embodiment, the complete response rate is at least or about 10% for a population of subjects treated with the method. In another embodiment, the complete response rate is at least or about 2% for a population of subjects treated with the method. In another embodiment, the complete response rate is at least or about 5% for a population of subjects treated with the method. In another embodiment, the complete response rate is at least or about 10% for a population of subjects treated with the method. In another embodiment, the complete response rate is at least or about 10.5% for a population of subjects treated with the method. In another embodiment, the complete response rate is at least or about 15% for a population of subjects treated with the method. In another embodiment, the complete response rate is at least or about 20% for a population of subjects treated with the method.

Similarly, using percentage of partial response as the criteria, in one embodiment, a population of the human subjects is treated by a method provided herein, wherein the percentage of the subjects having partial response in the treated population is at least or about 25%. In another embodiment, a population of the human subjects is treated by a method provided herein, wherein the percentage of the subjects having partial response in the treated population is at least or about 30%. In a further embodiment, a population of the human subjects is treated by a method provided herein, wherein the percentage of the subjects having partial response in the treated population is at least or about 35%. In yet another embodiment, a population of the human subjects is treated by a method provided herein, wherein the percentage of the subjects having partial response in the treated population is at least or about 40%. In one embodiment, a population of the human subjects is treated by a method provided herein, wherein the percentage of the subjects having partial response in the treated population is at least or about 45%. In one embodiment, a population of the human subjects is treated by a method provided herein, wherein the percentage of the subjects having partial response in the treated population is at least or about 50%. In one embodiment, a population of the human subjects is treated by a method provided herein, wherein the percentage of the subjects having partial response in the treated population is at least or about 53.9%. In one embodiment, a population of the human subjects is treated by a method provided herein, wherein the percentage of the subjects having partial response in the treated population is at least or about 54%. In another embodiment, a population of the human subjects is treated by a method provided herein, wherein the percentage of the subjects having partial response in the treated population is at least or about 55%. In another embodiment, a population of the human subjects is treated by a method provided herein, wherein the percentage of the subjects having partial response in the treated population is at least or about 60%. In another embodiment, a population of the human subjects is treated by a method provided herein, wherein the percentage of the subjects having partial response in the treated population is at least or about 65%. In a further embodiment, a population of the human subjects is treated by a method provided herein, wherein the percentage of the subjects having partial response in the treated population is at least or about 70%. In yet another embodiment, a population of the human subjects is treated by a method provided herein, wherein the percentage of the subjects having partial response in the treated population is at least or about 75%.

In one embodiment, the partial response rate is at least or about 25% for a population of subjects treated with the method. In another embodiment, the partial response rate is at least or about 30% for a population of subjects treated with the method. In another embodiment, the partial response rate is at least or about 35% for a population of subjects treated with the method. In another embodiment, the partial response rate is at least or about 40% for a population of subjects treated with the method. In another embodiment, the partial response rate is at least or about 45% for a population of subjects treated with the method. In another embodiment, the partial response rate is at least or about 50% for a population of subjects treated with the method. In another embodiment, the partial response rate is at least or about 53.9% for a population of subjects treated with the method. In another embodiment, the partial response rate is at least or about 54% for a population of subjects treated with the method. In another embodiment, the partial response rate is at least or about 55% for a population of subjects treated with the method. In another embodiment, the partial response rate is at least or about 60% for a population of subjects treated with the method. In another embodiment, the partial response rate is at least or about 65% for a population of subjects treated with the method. In another embodiment, the partial response rate is at least or about 70% for a population of subjects treated with the method. In another embodiment, the partial response rate is at least or about 75% for a population of subjects treated with the method.

Moreover, percentage of the subjects having stable disease can be used as the evaluation criteria for the therapeutic outcome in the human subjects treated by a method provided herein. In one embodiment, a population of the human subjects is treated by a method provided herein, wherein the percentage of the subjects having stable disease in the treated population is at least or about 10%. In one embodiment, a population of the human subjects is treated by a method provided herein, wherein the percentage of the subjects having stable disease in the treated population is at least or about 15%. In one embodiment, a population of the human subjects is treated by a method provided herein, wherein the percentage of the subjects having stable disease in the treated population is at least or about 20%. In one embodiment, a population of the human subjects is treated by a method provided herein, wherein the percentage of the subjects having stable disease in the treated population is at least or about 22%. In one embodiment, a population of the human subjects is treated by a method provided herein, wherein the percentage of the subjects having stable disease in the treated population is at least or about 22.4%. In one embodiment, a population of the human subjects is treated by a method provided herein, wherein the percentage of the subjects having stable disease in the treated population is at least or about 25%. In one embodiment, a population of the human subjects is treated by a method provided herein, wherein the percentage of the subjects having stable disease in the treated population is at least or about 30%. In one embodiment, a population of the human subjects is treated by a method provided herein, wherein the percentage of the subjects having stable disease in the treated population is at least or about 35%. In one embodiment, a population of the human subjects is treated by a method provided herein, wherein the percentage of the subjects having stable disease in the treated population is at least or about 40%. In one embodiment, a population of the human subjects is treated by a method provided herein, wherein the percentage of the subjects having stable disease in the treated population is at least or about 45%. In one embodiment, a population of the human subjects is treated by a method provided herein, wherein the percentage of the subjects having stable disease in the treated population is at least or about 50%.

In one embodiment, the stable disease rate is at least or about 10% for a population of subjects treated with the method. In one embodiment, the stable disease rate is at least or about 15% for a population of subjects treated with the method. In one embodiment, the stable disease rate is at least or about 20% for a population of subjects treated with the method. In one embodiment, the stable disease rate is at least or about 22% for a population of subjects treated with the method. In one embodiment, the stable disease rate is at least or about 22.4% for a population of subjects treated with the method. In one embodiment, the stable disease rate is at least or about 25% for a population of subjects treated with the method. In one embodiment, the stable disease rate is at least or about 30% for a population of subjects treated with the method. In one embodiment, the stable disease rate is at least or about 35% for a population of subjects treated with the method. In one embodiment, the stable disease rate is at least or about 40% for a population of subjects treated with the method. In one embodiment, the stable disease rate is at least or about 45% for a population of subjects treated with the method. In one embodiment, the stable disease rate is at least or about 50% for a population of subjects treated with the method.

Likewise, objective response rate, which is the sum of percentage of subjects having completed response and those having partial response, can be used as the evaluation criteria for the therapeutic outcome in the human subjects treated by a method provided herein. In yet another embodiment, a population of the human subjects is treated by a method provided herein, wherein objective response rate in the treated population is at least or about 30%. In one embodiment, a population of the human subjects is treated by a method provided herein, wherein objective response rate in the treated population is at least or about 35%. In another embodiment, a population of the human subjects is treated by a method provided herein, wherein objective response rate in the treated population is at least or about 40%. In one embodiment, a population of the human subjects is treated by a method provided herein, wherein objective response rate in the treated population is at least or about 45%. In a further embodiment, a population of the human subjects is treated by a method provided herein, wherein objective response rate in the treated population is at least or about 50%. In yet another embodiment, a population of the human subjects is treated by a method provided herein, wherein objective response rate in the treated population is at least or about 52.7%. In yet another embodiment, a population of the human subjects is treated by a method provided herein, wherein objective response rate in the treated population is at least or about 53%. In yet another embodiment, a population of the human subjects is treated by a method provided herein, wherein objective response rate in the treated population is at least or about 55%. In one embodiment, a population of the human subjects is treated by a method provided herein, wherein objective response rate in the treated population is at least or about 60%. In another embodiment, a population of the human subjects is treated by a method provided herein, wherein objective response rate in the treated population is at least or about 64.5%. In a further embodiment, a population of the human subjects is treated by a method provided herein, wherein objective response rate in the treated population is at least or about 65%. In yet another embodiment, a population of the human subjects is treated by a method provided herein, wherein objective response rate in the treated population is at least or about 70%. In one embodiment, a population of the human subjects is treated by a method provided herein, wherein objective response rate in the treated population is at least or about 75%. In one embodiment, a population of the human subjects is treated by a method provided herein, wherein objective response rate in the treated population is at least or about 75.1%. In another embodiment, a population of the human subjects is treated by a method provided herein, wherein objective response rate in the treated population is at least or about 80%. In a further embodiment, a population of the human subjects is treated by a method provided herein, wherein objective response rate in the treated population is at least or about 85%. In yet another embodiment, a population of the human subjects is treated by a method provided herein, wherein objective response rate in the treated population is at least or about 90%.

In one embodiment, a population of the human subjects is treated by a method provided herein, wherein objective response rate in the treated population ranges from 40% to 80%. In another embodiment, a population of the human subjects is treated by a method provided herein, wherein objective response rate in the treated population ranges from 40% to 75%. In another embodiment, a population of the human subjects is treated by a method provided herein, wherein objective response rate in the treated population ranges from 40% to 70%. In another embodiment, a population of the human subjects is treated by a method provided herein, wherein objective response rate in the treated population ranges from 45% to 80%. In another embodiment, a population of the human subjects is treated by a method provided herein, wherein objective response rate in the treated population ranges from 45% to 75%. In another embodiment, a population of the human subjects is treated by a method provided herein, wherein objective response rate in the treated population ranges from 45% to 70%. In a further embodiment, a population of the human subjects is treated by a method provided herein, wherein objective response rate in the treated population ranges from 50% to 80%. In a further embodiment, a population of the human subjects is treated by a method provided herein, wherein objective response rate in the treated population ranges from 50% to 75%. In another embodiment, a population of the human subjects is treated by a method provided herein, wherein objective response rate in the treated population ranges from 52.7% to 75.1%. In yet another embodiment, a population of the human subjects is treated by a method provided herein, wherein objective response rate in the treated population ranges from 55% to 80%. In yet another embodiment, a population of the human subjects is treated by a method provided herein, wherein objective response rate in the treated population ranges from 55% to 75%. In yet another embodiment, a population of the human subjects is treated by a method provided herein, wherein objective response rate in the treated population ranges from 55% to 70%. In yet another embodiment, a population of the human subjects is treated by a method provided herein, wherein objective response rate in the treated population ranges from 55% to 65%. In one embodiment, a population of the human subjects is treated by a method provided herein, wherein objective response rate in the treated population ranges from 60% to 80%. In one embodiment, a population of the human subjects is treated by a method provided herein, wherein objective response rate in the treated population ranges from 60% to 75%. In one embodiment, a population of the human subjects is treated by a method provided herein, wherein objective response rate in the treated population ranges from 60% to 70%. In one embodiment, a population of the human subjects is treated by a method provided herein, wherein objective response rate in the treated population ranges from 60% to 65%.

In one embodiment, the objective response rate is at least or about 30% for a population of subjects treated with the method. In another embodiment, the objective response rate is at least or about 30% for a population of subjects treated with the method. In another embodiment, the objective response rate is at least or about 35% for a population of subjects treated with the method. In another embodiment, the objective response rate is at least or about 40% for a population of subjects treated with the method. In another embodiment, the objective response rate is at least or about 45% for a population of subjects treated with the method. In another embodiment, the objective response rate is at least or about 50% for a population of subjects treated with the method. In another embodiment, the objective response rate is at least or about 55% for a population of subjects treated with the method. In another embodiment, the objective response rate is at least or about 60% for a population of subjects treated with the method. In another embodiment, the objective response rate is at least or about 64.5% for a population of subjects treated with the method. In another embodiment, the objective response rate is at least or about 65% for a population of subjects treated with the method. In another embodiment, the objective response rate is at least or about 70% for a population of subjects treated with the method. In another embodiment, the objective response rate is at least or about 75% for a population of subjects treated with the method. In another embodiment, the objective response rate is at least or about 80% for a population of subjects treated with the method. In another embodiment, the objective response rate is at least or about 85% for a population of subjects treated with the method. In another embodiment, the objective response rate is at least or about 90% for a population of subjects treated with the method.

In one embodiment, the objective response rate is from 40% to 80% for a population of subjects treated with the method. In another embodiment, the objective response rate is from 40% to 75% for a population of subjects treated with the method. In another embodiment, the objective response rate is from 40% to 70% for a population of subjects treated with the method. In another embodiment, the objective response rate is from 45% to 80% for a population of subjects treated with the method. In another embodiment, the objective response rate is from 45% to 75% for a population of subjects treated with the method. In another embodiment, the objective response rate is from 45% to 70% for a population of subjects treated with the method. In another embodiment, the objective response rate is from 50% to 80% for a population of subjects treated with the method. In another embodiment, the objective response rate is from 50% to 75% for a population of subjects treated with the method. In another embodiment, the objective response rate is from 52.7% to 75.1% for a population of subjects treated with the method. In another embodiment, the objective response rate is from 53% to 75% for a population of subjects treated with the method. In another embodiment, the objective response rate is from 55% to 80% for a population of subjects treated with the method. In another embodiment, the objective response rate is from 55% to 75% for a population of subjects treated with the method. In another embodiment, the objective response rate is from 55% to 70% for a population of subjects treated with the method. In another embodiment, the objective response rate is from 55% to 65% for a population of subjects treated with the method. In another embodiment, the objective response rate is from 60% to 80% for a population of subjects treated with the method. In another embodiment, the objective response rate is from 60% to 75% for a population of subjects treated with the method. In another embodiment, the objective response rate is from 60% to 70% for a population of subjects treated with the method. In another embodiment, the objective response rate is from 60% to 65% for a population of subjects treated with the method.

Additionally, the therapeutic outcome of the methods provided herein can be evaluated based on the duration of response as set forth in Section 6.1.8.2(ii). In one embodiment, the human subject has a duration of response of at least or about 5 months following the treatment. In another embodiment, the human subject has a duration of response of at least or about 6 months following the treatment. In a further embodiment, the human subject has a duration of response of at least or about 7 months following the treatment. In yet another embodiment, the human subject has a duration of response of at least or about 8 months following the treatment. In one embodiment, the human subject has a duration of response of at least or about 9 months following the treatment. In another embodiment, the human subject has a duration of response of at least or about 10 months following the treatment. In yet another embodiment, the human subject has a duration of response of at least or about 11 months following the treatment. In one embodiment, the human subject has a duration of response of at least or about 12 months following the treatment. In another embodiment, the human subject has a duration of response of at least or about 13 months following the treatment. In a further embodiment, the human subject has a duration of response of at least or about 14 months following the treatment. In yet another embodiment, the human subject has a duration of response of at least or about 15 months following the treatment. In one embodiment, the human subject has a duration of response of at least or about 16 months following the treatment. In another embodiment, the human subject has a duration of response of at least or about 17 months following the treatment. In a further embodiment, the human subject has a duration of response of at least or about 18 months following the treatment. In yet another embodiment, the human subject has a duration of response of at least or about 19 months following the treatment. In a further embodiment, the human subject has a duration of response of at least or about 20 months following the treatment. In a further embodiment, the human subject has a duration of response of at least or about 21 months following the treatment. In a further embodiment, the human subject has a duration of response of at least or about 22 months following the treatment. In a further embodiment, the human subject has a duration of response of at least or about 23 months following the treatment. In a further embodiment, the human subject has a duration of response of at least or about 24 months following the treatment. In a further embodiment, the human subject has a duration of response of at least or about 25 months following the treatment. In a further embodiment, the human subject has a duration of response of at least or about 26 months following the treatment. In a further embodiment, the human subject has a duration of response of at least or about 27 months following the treatment. In a further embodiment, the human subject has a duration of response of at least or about 28 months following the treatment. In a further embodiment, the human subject has a duration of response of at least or about 29 months following the treatment. In a further embodiment, the human subject has a duration of response of at least or about 30 months following the treatment.

In another embodiment, the human subject has a duration of response ranging from 5 to 30 months following the treatment. In another embodiment, the human subject has a duration of response ranging from 5 to 29 months following the treatment. In another embodiment, the human subject has a duration of response ranging from 5 to 28 months following the treatment. In another embodiment, the human subject has a duration of response ranging from 5 to 27 months following the treatment. In another embodiment, the human subject has a duration of response ranging from 5 to 26 months following the treatment. In another embodiment, the human subject has a duration of response ranging from 5 to 25 months following the treatment. In another embodiment, the human subject has a duration of response ranging from 5 to 24 months following the treatment. In another embodiment, the human subject has a duration of response ranging from 5 to 23 months following the treatment. In some embodiments, the human subject has a duration of response ranging from 5 to 22 months following the treatment. In another embodiment, the human subject has a duration of response ranging from 5 to 21 months following the treatment. In a further embodiment, the human subject has a duration of response ranging from 5 to 20 months following the treatment. In yet another embodiment, the human subject has a duration of response ranging from 5 to 19 months following the treatment. In one embodiment, the human subject has a duration of response ranging from 5 to 18 months following the treatment. In another embodiment, the human subject has a duration of response ranging from 5 to 17 months following the treatment. In a further embodiment, the human subject has a duration of response ranging from 5 to 16 months following the treatment. In yet another embodiment, the human subject has a duration of response ranging from 5 to 15 months following the treatment. In one embodiment, the human subject has a duration of response ranging from 5 to 14 months following the treatment. In another embodiment, the human subject has a duration of response ranging from 5 to 13 months following the treatment. In a further embodiment, the human subject has a duration of response ranging from 5 to 12 months following the treatment. In another embodiment, the human subject has a duration of response ranging from 6 to 30 months following the treatment. In another embodiment, the human subject has a duration of response ranging from 6 to 29 months following the treatment. In another embodiment, the human subject has a duration of response ranging from 6 to 28 months following the treatment. In another embodiment, the human subject has a duration of response ranging from 6 to 27 months following the treatment. In another embodiment, the human subject has a duration of response ranging from 6 to 26 months following the treatment. In another embodiment, the human subject has a duration of response ranging from 6 to 25 months following the treatment. In another embodiment, the human subject has a duration of response ranging from 6 to 24 months following the treatment. In another embodiment, the human subject has a duration of response ranging from 6 to 23 months following the treatment. In yet another embodiment, the human subject has a duration of response ranging from 6 to 22 months following the treatment. In another embodiment, the human subject has a duration of response ranging from 6 to 21 months following the treatment. In a further embodiment, the human subject has a duration of response ranging from 6 to 20 months following the treatment. In yet another embodiment, the human subject has a duration of response ranging from 6 to 19 months following the treatment. In one embodiment, the human subject has a duration of response ranging from 6 to 18 months following the treatment. In another embodiment, the human subject has a duration of response ranging from 6 to 17 months following the treatment. In a further embodiment, the human subject has a duration of response ranging from 6 to 16 months following the treatment. In yet another embodiment, the human subject has a duration of response ranging from 6 to 15 months following the treatment. In one embodiment, the human subject has a duration of response ranging from 6 to 14 months following the treatment. In another embodiment, the human subject has a duration of response ranging from 6 to 13 months following the treatment. In a further embodiment, the human subject has a duration of response ranging from 6 to 12 months following the treatment. In one embodiment, the human subject has a duration of response ranging from 7 to 22 months following the treatment. In another embodiment, the human subject has a duration of response ranging from 7 to 21 months following the treatment. In a further embodiment, the human subject has a duration of response ranging from 7 to 20 months following the treatment. In yet another embodiment, the human subject has a duration of response ranging from 7 to 19 months following the treatment. In one embodiment, the human subject has a duration of response ranging from 7 to 18 months following the treatment. In another embodiment, the human subject has a duration of response ranging from 7 to 17 months following the treatment. In a further embodiment, the human subject has a duration of response ranging from 7 to 16 months following the treatment. In yet another embodiment, the human subject has a duration of response ranging from 7 to 15 months following the treatment. In one embodiment, the human subject has a duration of response ranging from 7 to 14 months following the treatment. In another embodiment, the human subject has a duration of response ranging from 7 to 13 months following the treatment. In a further embodiment, the human subject has a duration of response ranging from 7 to 12 months following the treatment. In another embodiment, the human subject has a duration of response ranging from 8 to 30 months following the treatment. In a further embodiment, the human subject has a duration of response ranging from 9 to 30 months following the treatment. In yet another embodiment, the human subject has a duration of response ranging from 10 to 30 months following the treatment. In a further embodiment, the human subject has a duration of response ranging from 11 to 30 months following the treatment. In one embodiment, the human subject has a duration of response ranging from 12 to 30 months following the treatment. In another embodiment, the human subject has a duration of response ranging from 13 to 30 months following the treatment. In a further embodiment, the human subject has a duration of response ranging from 14 to 30 months following the treatment. In yet another embodiment, the human subject has a duration of response ranging from 15 to 30 months following the treatment. In a further embodiment, the human subject has a duration of response ranging from 16 to 30 months following the treatment. In one embodiment, the human subject has a duration of response ranging from 17 to 30 months following the treatment. In another embodiment, the human subject has a duration of response ranging from 18 to 30 months following the treatment. In another embodiment, the human subject has a duration of response ranging from 8 to 27 months following the treatment. In a further embodiment, the human subject has a duration of response ranging from 9 to 27 months following the treatment. In yet another embodiment, the human subject has a duration of response ranging from 10 to 27 months following the treatment. In a further embodiment, the human subject has a duration of response ranging from 11 to 27 months following the treatment. In one embodiment, the human subject has a duration of response ranging from 12 to 27 months following the treatment. In another embodiment, the human subject has a duration of response ranging from 13 to 27 months following the treatment. In a further embodiment, the human subject has a duration of response ranging from 14 to 27 months following the treatment. In yet another embodiment, the human subject has a duration of response ranging from 15 to 27 months following the treatment. In a further embodiment, the human subject has a duration of response ranging from 16 to 27 months following the treatment. In one embodiment, the human subject has a duration of response ranging from 17 to 27 months following the treatment. In another embodiment, the human subject has a duration of response ranging from 18 to 27 months following the treatment. In another embodiment, the human subject has a duration of response ranging from 8 to 22 months following the treatment. In a further embodiment, the human subject has a duration of response ranging from 9 to 22 months following the treatment. In yet another embodiment, the human subject has a duration of response ranging from 10 to 22 months following the treatment. In a further embodiment, the human subject has a duration of response ranging from 11 to 22 months following the treatment. In one embodiment, the human subject has a duration of response ranging from 12 to 22 months following the treatment. In another embodiment, the human subject has a duration of response ranging from 13 to 22 months following the treatment. In a further embodiment, the human subject has a duration of response ranging from 14 to 22 months following the treatment. In yet another embodiment, the human subject has a duration of response ranging from 15 to 22 months following the treatment. In a further embodiment, the human subject has a duration of response ranging from 16 to 22 months following the treatment. In one embodiment, the human subject has a duration of response ranging from 17 to 22 months following the treatment. In another embodiment, the human subject has a duration of response ranging from 18 to 22 months following the treatment. In a further embodiment, the human subject has a duration of response ranging from 6 to 21 months following the treatment. In yet another embodiment, the human subject has a duration of response ranging from 7 to 20 months following the treatment. In a further embodiment, the human subject has a duration of response ranging from 8 to 19 months following the treatment. In one embodiment, the human subject has a duration of response ranging from 9 to 18 months following the treatment. In another embodiment, the human subject has a duration of response ranging from 10 to 17 months following the treatment. In a further embodiment, the human subject has a duration of response ranging from 11 to 16 months following the treatment. In yet another embodiment, the human subject has a duration of response ranging from 12 to 15 months following the treatment. In a further embodiment, the human subject has a duration of response ranging from 13 to 14 months following the treatment.

In certain embodiments, a population of the human subjects is treated by a method provided herein, wherein the duration of response in the treated population is at least or about 5 months following the treatment. In certain embodiments, a population of the human subjects is treated by a method provided herein, wherein the duration of response in the treated population is at least or about 6 months following the treatment. In certain embodiments, a population of the human subjects is treated by a method provided herein, wherein the duration of response in the treated population is at least or about 7 months following the treatment. In certain embodiments, a population of the human subjects is treated by a method provided herein, wherein the duration of response in the treated population is at least or about 8 months following the treatment. In certain embodiments, a population of the human subjects is treated by a method provided herein, wherein the duration of response in the treated population is at least or about 9 months following the treatment. In certain embodiments, a population of the human subjects is treated by a method provided herein, wherein the duration of response in the treated population is at least or about 10 months following the treatment. In certain embodiments, a population of the human subjects is treated by a method provided herein, wherein the duration of response in the treated population is at least or about 11 months following the treatment. In certain embodiments, a population of the human subjects is treated by a method provided herein, wherein the duration of response in the treated population is at least or about 12 months following the treatment. In certain embodiments, a population of the human subjects is treated by a method provided herein, wherein the duration of response in the treated population is at least or about 13 months following the treatment. In certain embodiments, a population of the human subjects is treated by a method provided herein, wherein the duration of response in the treated population is at least or about 14 months following the treatment. In certain embodiments, a population of the human subjects is treated by a method provided herein, wherein the duration of response in the treated population is at least or about 15 months following the treatment. In certain embodiments, a population of the human subjects is treated by a method provided herein, wherein the duration of response in the treated population is at least or about 16 months following the treatment. In certain embodiments, a population of the human subjects is treated by a method provided herein, wherein the duration of response in the treated population is at least or about 17 months following the treatment. In certain embodiments, a population of the human subjects is treated by a method provided herein, wherein the duration of response in the treated population is at least or about 18 months following the treatment. In certain embodiments, a population of the human subjects is treated by a method provided herein, wherein the duration of response in the treated population is at least or about 19 months following the treatment. In certain embodiments, a population of the human subjects is treated by a method provided herein, wherein the duration of response in the treated population is at least or about 20 months following the treatment. In certain embodiments, a population of the human subjects is treated by a method provided herein, wherein the duration of response in the treated population is at least or about 21 months following the treatment. In certain embodiments, a population of the human subjects is treated by a method provided herein, wherein the duration of response in the treated population is at least or about 22 months following the treatment. In certain embodiments, a population of the human subjects is treated by a method provided herein, wherein the duration of response in the treated population is at least or about 23 months following the treatment. In certain embodiments, a population of the human subjects is treated by a method provided herein, wherein the duration of response in the treated population is at least or about 24 months following the treatment. In certain embodiments, a population of the human subjects is treated by a method provided herein, wherein the duration of response in the treated population is at least or about 25 months following the treatment. In certain embodiments, a population of the human subjects is treated by a method provided herein, wherein the duration of response in the treated population is at least or about 26 months following the treatment. In certain embodiments, a population of the human subjects is treated by a method provided herein, wherein the duration of response in the treated population is at least or about 27 months following the treatment. In certain embodiments, a population of the human subjects is treated by a method provided herein, wherein the duration of response in the treated population is at least or about 28 months following the treatment. In certain embodiments, a population of the human subjects is treated by a method provided herein, wherein the duration of response in the treated population is at least or about 29 months following the treatment. In certain embodiments, a population of the human subjects is treated by a method provided herein, wherein the duration of response in the treated population is at least or about 30 months following the treatment.

In certain embodiments, a population of the human subjects is treated by a method provided herein, wherein the duration of response of in the treated population ranges from 5 to 30 months. In some embodiments, a population of the human subjects is treated by a method provided herein, wherein the duration of response of in the treated population ranges from 5 to 29 months. In some embodiments, a population of the human subjects is treated by a method provided herein, wherein the duration of response of in the treated population ranges from 5 to 28 months. In some embodiments, a population of the human subjects is treated by a method provided herein, wherein the duration of response of in the treated population ranges from 5 to 27 months. In some embodiments, a population of the human subjects is treated by a method provided herein, wherein the duration of response of in the treated population ranges from 5 to 26 months. In some embodiments, a population of the human subjects is treated by a method provided herein, wherein the duration of response of in the treated population ranges from 5 to 25 months. In some embodiments, a population of the human subjects is treated by a method provided herein, wherein the duration of response of in the treated population ranges from 5 to 24 months. In some embodiments, a population of the human subjects is treated by a method provided herein, wherein the duration of response of in the treated population ranges from 5 to 23 months. In certain embodiments, a population of the human subjects is treated by a method provided herein, wherein the duration of response of in the treated population ranges from 5 to 22 months. In some embodiments, a population of the human subjects is treated by a method provided herein, wherein the duration of response of in the treated population ranges from 5 to 21 months. In another embodiment, a population of the human subjects is treated by a method provided herein, wherein the duration of response of in the treated population ranges from 5 to 20 months. In a further embodiment, a population of the human subjects is treated by a method provided herein, wherein the duration of response of in the treated population ranges from 5 to 19 months. In yet another embodiment, a population of the human subjects is treated by a method provided herein, wherein the duration of response of in the treated population ranges from 5 to 18 months. In one embodiment, a population of the human subjects is treated by a method provided herein, wherein the duration of response of in the treated population ranges from 5 to 17 months. In another embodiment, a population of the human subjects is treated by a method provided herein, wherein the duration of response of in the treated population ranges from 5 to 16 months. In a further embodiment, a population of the human subjects is treated by a method provided herein, wherein the duration of response of in the treated population ranges from 5 to 15 months. In yet another embodiment, a population of the human subjects is treated by a method provided herein, wherein the duration of response of in the treated population ranges from 5 to 14 months. In one embodiment, a population of the human subjects is treated by a method provided herein, wherein the duration of response of in the treated population ranges from 5 to 13 months. In another embodiment, a population of the human subjects is treated by a method provided herein, wherein the duration of response of in the treated population ranges from 5 to 12 months. In yet another embodiment, a population of the human subjects is treated by a method provided herein, wherein the duration of response of in the treated population ranges from 6 to 30 months. In some embodiments, a population of the human subjects is treated by a method provided herein, wherein the duration of response of in the treated population ranges from 6 to 29 months. In some embodiments, a population of the human subjects is treated by a method provided herein, wherein the duration of response of in the treated population ranges from 6 to 28 months. In some embodiments, a population of the human subjects is treated by a method provided herein, wherein the duration of response of in the treated population ranges from 6 to 27 months. In some embodiments, a population of the human subjects is treated by a method provided herein, wherein the duration of response of in the treated population ranges from 6 to 26 months. In some embodiments, a population of the human subjects is treated by a method provided herein, wherein the duration of response of in the treated population ranges from 6 to 25 months. In some embodiments, a population of the human subjects is treated by a method provided herein, wherein the duration of response of in the treated population ranges from 6 to 24 months. In some embodiments, a population of the human subjects is treated by a method provided herein, wherein the duration of response of in the treated population ranges from 6 to 23 months. In a further embodiment, a population of the human subjects is treated by a method provided herein, wherein the duration of response of in the treated population ranges from 6 to 22 months. In some embodiments, a population of the human subjects is treated by a method provided herein, wherein the duration of response of in the treated population ranges from 6 to 21 months. In another embodiment, a population of the human subjects is treated by a method provided herein, wherein the duration of response of in the treated population ranges from 6 to 20 months. In a further embodiment, a population of the human subjects is treated by a method provided herein, wherein the duration of response of in the treated population ranges from 6 to 19 months. In yet another embodiment, a population of the human subjects is treated by a method provided herein, wherein the duration of response of in the treated population ranges from 6 to 18 months. In one embodiment, a population of the human subjects is treated by a method provided herein, wherein the duration of response of in the treated population ranges from 6 to 17 months. In another embodiment, a population of the human subjects is treated by a method provided herein, wherein the duration of response of in the treated population ranges from 6 to 16 months. In a further embodiment, a population of the human subjects is treated by a method provided herein, wherein the duration of response of in the treated population ranges from 6 to 15 months. In yet another embodiment, a population of the human subjects is treated by a method provided herein, wherein the duration of response of in the treated population ranges from 6 to 14 months. In one embodiment, a population of the human subjects is treated by a method provided herein, wherein the duration of response of in the treated population ranges from 6 to 13 months. In another embodiment, a population of the human subjects is treated by a method provided herein, wherein the duration of response of in the treated population ranges from 6 to 12 months. In yet another embodiment, a population of the human subjects is treated by a method provided herein, wherein the duration of response of in the treated population ranges from 7 to 30 months. In some embodiments, a population of the human subjects is treated by a method provided herein, wherein the duration of response of in the treated population ranges from 7 to 29 months. In some embodiments, a population of the human subjects is treated by a method provided herein, wherein the duration of response of in the treated population ranges from 7 to 28 months. In some embodiments, a population of the human subjects is treated by a method provided herein, wherein the duration of response of in the treated population ranges from 7 to 27 months. In some embodiments, a population of the human subjects is treated by a method provided herein, wherein the duration of response of in the treated population ranges from 7 to 26 months. In some embodiments, a population of the human subjects is treated by a method provided herein, wherein the duration of response of in the treated population ranges from 7 to 25 months. In some embodiments, a population of the human subjects is treated by a method provided herein, wherein the duration of response of in the treated population ranges from 7 to 24 months. In some embodiments, a population of the human subjects is treated by a method provided herein, wherein the duration of response of in the treated population ranges from 7 to 23 months. In yet another embodiment, a population of the human subjects is treated by a method provided herein, wherein the duration of response of in the treated population ranges from 7 to 22 months. In some embodiments, a population of the human subjects is treated by a method provided herein, wherein the duration of response of in the treated population ranges from 7 to 21 months. In another embodiment, a population of the human subjects is treated by a method provided herein, wherein the duration of response of in the treated population ranges from 7 to 20 months. In a further embodiment, a population of the human subjects is treated by a method provided herein, wherein the duration of response of in the treated population ranges from 7 to 19 months. In yet another embodiment, a population of the human subjects is treated by a method provided herein, wherein the duration of response of in the treated population ranges from 7 to 18 months. In one embodiment, a population of the human subjects is treated by a method provided herein, wherein the duration of response of in the treated population ranges from 7 to 17 months. In another embodiment, a population of the human subjects is treated by a method provided herein, wherein the duration of response of in the treated population ranges from 7 to 16 months. In a further embodiment, a population of the human subjects is treated by a method provided herein, wherein the duration of response of in the treated population ranges from 7 to 15 months. In yet another embodiment, a population of the human subjects is treated by a method provided herein, wherein the duration of response of in the treated population ranges from 7 to 14 months. In one embodiment, a population of the human subjects is treated by a method provided herein, wherein the duration of response of in the treated population ranges from 7 to 13 months. In another embodiment, a population of the human subjects is treated by a method provided herein, wherein the duration of response of in the treated population ranges from 7 to 12 months. In one embodiment, a population of the human subjects is treated by a method provided herein, wherein the duration of response of in the treated population ranges from 6.41 to 22 months. In another embodiment, a population of the human subjects is treated by a method provided herein, wherein the duration of response of in the treated population ranges from 8 to 22 months. In a further embodiment, a population of the human subjects is treated by a method provided herein, wherein the duration of response of in the treated population ranges from 9 to 22 months. In yet another embodiment, a population of the human subjects is treated by a method provided herein, wherein the duration of response of in the treated population ranges from 10 to 22 months. In one embodiment, a population of the human subjects is treated by a method provided herein, wherein the duration of response of in the treated population ranges from 11 to 22 months. In another embodiment, a population of the human subjects is treated by a method provided herein, wherein the duration of response of in the treated population ranges from 12 to 12 months. In a further embodiment, a population of the human subjects is treated by a method provided herein, wherein the duration of response of in the treated population ranges from 13 to 22 months. In yet another embodiment, a population of the human subjects is treated by a method provided herein, wherein the duration of response of in the treated population ranges from 14 to 22 months. In one embodiment, a population of the human subjects is treated by a method provided herein, wherein the duration of response of in the treated population ranges from 15 to 22 months. In another embodiment, a population of the human subjects is treated by a method provided herein, wherein the duration of response of in the treated population ranges from 16 to 22 months. In a further embodiment, a population of the human subjects is treated by a method provided herein, wherein the duration of response of in the treated population ranges from 17 to 22 months. In yet another embodiment, a population of the human subjects is treated by a method provided herein, wherein the duration of response of in the treated population ranges from 18 to 22 months. In another embodiment, a population of the human subjects is treated by a method provided herein, wherein the duration of response of in the treated population ranges from 7 to 27 months. In another embodiment, a population of the human subjects is treated by a method provided herein, wherein the duration of response of in the treated population ranges from 8 to 27 months. In a further embodiment, a population of the human subjects is treated by a method provided herein, wherein the duration of response of in the treated population ranges from 9 to 27 months. In yet another embodiment, a population of the human subjects is treated by a method provided herein, wherein the duration of response of in the treated population ranges from 10 to 27 months. In one embodiment, a population of the human subjects is treated by a method provided herein, wherein the duration of response of in the treated population ranges from 11 to 27 months. In another embodiment, a population of the human subjects is treated by a method provided herein, wherein the duration of response of in the treated population ranges from 12 to 27 months. In a further embodiment, a population of the human subjects is treated by a method provided herein, wherein the duration of response of in the treated population ranges from 13 to 27 months. In yet another embodiment, a population of the human subjects is treated by a method provided herein, wherein the duration of response of in the treated population ranges from 14 to 27 months. In one embodiment, a population of the human subjects is treated by a method provided herein, wherein the duration of response of in the treated population ranges from 15 to 27 months. In another embodiment, a population of the human subjects is treated by a method provided herein, wherein the duration of response of in the treated population ranges from 16 to 27 months. In a further embodiment, a population of the human subjects is treated by a method provided herein, wherein the duration of response of in the treated population ranges from 17 to 27 months. In yet another embodiment, a population of the human subjects is treated by a method provided herein, wherein the duration of response of in the treated population ranges from 18 to 27 months. In yet another embodiment, a population of the human subjects is treated by a method provided herein, wherein the duration of response of in the treated population ranges from 19 to 27 months. In yet another embodiment, a population of the human subjects is treated by a method provided herein, wherein the duration of response of in the treated population ranges from 20 to 27 months. In yet another embodiment, a population of the human subjects is treated by a method provided herein, wherein the duration of response of in the treated population ranges from 21 to 27 months. In yet another embodiment, a population of the human subjects is treated by a method provided herein, wherein the duration of response of in the treated population ranges from 22 to 27 months. In yet another embodiment, a population of the human subjects is treated by a method provided herein, wherein the duration of response of in the treated population ranges from 23 to 27 months. In yet another embodiment, a population of the human subjects is treated by a method provided herein, wherein the duration of response of in the treated population ranges from 24 to 27 months. In another embodiment, a population of the human subjects is treated by a method provided herein, wherein the duration of response of in the treated population ranges from 7 to 30 months. In another embodiment, a population of the human subjects is treated by a method provided herein, wherein the duration of response of in the treated population ranges from 8 to 30 months. In a further embodiment, a population of the human subjects is treated by a method provided herein, wherein the duration of response of in the treated population ranges from 9 to 30 months. In yet another embodiment, a population of the human subjects is treated by a method provided herein, wherein the duration of response of in the treated population ranges from 10 to 30 months. In one embodiment, a population of the human subjects is treated by a method provided herein, wherein the duration of response of in the treated population ranges from 11 to 30 months. In another embodiment, a population of the human subjects is treated by a method provided herein, wherein the duration of response of in the treated population ranges from 12 to 30 months. In a further embodiment, a population of the human subjects is treated by a method provided herein, wherein the duration of response of in the treated population ranges from 13 to 30 months. In yet another embodiment, a population of the human subjects is treated by a method provided herein, wherein the duration of response of in the treated population ranges from 14 to 30 months. In one embodiment, a population of the human subjects is treated by a method provided herein, wherein the duration of response of in the treated population ranges from 15 to 30 months. In another embodiment, a population of the human subjects is treated by a method provided herein, wherein the duration of response of in the treated population ranges from 16 to 30 months. In a further embodiment, a population of the human subjects is treated by a method provided herein, wherein the duration of response of in the treated population ranges from 17 to 30 months. In yet another embodiment, a population of the human subjects is treated by a method provided herein, wherein the duration of response of in the treated population ranges from 18 to 30 months. In yet another embodiment, a population of the human subjects is treated by a method provided herein, wherein the duration of response of in the treated population ranges from 19 to 30 months. In yet another embodiment, a population of the human subjects is treated by a method provided herein, wherein the duration of response of in the treated population ranges from 20 to 30 months. In yet another embodiment, a population of the human subjects is treated by a method provided herein, wherein the duration of response of in the treated population ranges from 21 to 30 months. In yet another embodiment, a population of the human subjects is treated by a method provided herein, wherein the duration of response of in the treated population ranges from 22 to 30 months. In yet another embodiment, a population of the human subjects is treated by a method provided herein, wherein the duration of response of in the treated population ranges from 23 to 30 months. In yet another embodiment, a population of the human subjects is treated by a method provided herein, wherein the duration of response of in the treated population ranges from 24 to 30 months. In yet another embodiment, a population of the human subjects is treated by a method provided herein, wherein the duration of response of in the treated population ranges from 25 to 30 months. In yet another embodiment, a population of the human subjects is treated by a method provided herein, wherein the duration of response of in the treated population ranges from 26 to 30 months. In yet another embodiment, a population of the human subjects is treated by a method provided herein, wherein the duration of response of in the treated population ranges from 27 to 30 months. In one embodiment, a population of the human subjects is treated by a method provided herein, wherein the duration of response of in the treated population ranges from 6 to 21 months. In another embodiment, a population of the human subjects is treated by a method provided herein, wherein the duration of response of in the treated population ranges from 7 to 20 months. In a further embodiment, a population of the human subjects is treated by a method provided herein, wherein the duration of response of in the treated population ranges from 8 to 19 months. In yet another embodiment, a population of the human subjects is treated by a method provided herein, wherein the duration of response of in the treated population ranges from 9 to 18 months. In one embodiment, a population of the human subjects is treated by a method provided herein, wherein the duration of response of in the treated population ranges from 10 to 17 months. In another embodiment, a population of the human subjects is treated by a method provided herein, wherein the duration of response of in the treated population ranges from 11 to 16 months. In a further embodiment, a population of the human subjects is treated by a method provided herein, wherein the duration of response of in the treated population ranges from 12 to 15 months. In yet another embodiment, a population of the human subjects is treated by a method provided herein, wherein the duration of response of in the treated population ranges from 13 to 24 months.

In certain embodiments, the duration of response is at least or about 5 months for a population of subjects treated with the method. In certain embodiments, the duration of response is at least or about 6 months for a population of subjects treated with the method. In certain embodiments, the duration of response is at least or about 7 months for a population of subjects treated with the method. In certain embodiments, the duration of response is at least or about 8 months for a population of subjects treated with the method. In certain embodiments, the duration of response is at least or about 9 months for a population of subjects treated with the method. In certain embodiments, the duration of response is at least or about 10 months for a population of subjects treated with the method. In certain embodiments, the duration of response is at least or about 11 months for a population of subjects treated with the method. In certain embodiments, the duration of response is at least or about 12 months for a population of subjects treated with the method. In certain embodiments, the duration of response is at least or about 13 months for a population of subjects treated with the method. In certain embodiments, the duration of response is at least or about 14 months for a population of subjects treated with the method. In certain embodiments, the duration of response is at least or about 15 months for a population of subjects treated with the method. In certain embodiments, the duration of response is at least or about 16 months for a population of subjects treated with the method. In certain embodiments, the duration of response is at least or about 17 months for a population of subjects treated with the method. In certain embodiments, the duration of response is at least or about 18 months for a population of subjects treated with the method. In certain embodiments, the duration of response is at least or about 19 months for a population of subjects treated with the method. In certain embodiments, the duration of response is at least or about 20 months for a population of subjects treated with the method. In certain embodiments, the duration of response is at least or about 21 months for a population of subjects treated with the method. In certain embodiments, the duration of response is at least or about 22 months for a population of subjects treated with the method. In certain embodiments, the duration of response is at least or about 23 months for a population of subjects treated with the method. In certain embodiments, the duration of response is at least or about 24 months for a population of subjects treated with the method. In certain embodiments, the duration of response is at least or about 25 months for a population of subjects treated with the method. In certain embodiments, the duration of response is at least or about 26 months for a population of subjects treated with the method. In certain embodiments, the duration of response is at least or about 27 months for a population of subjects treated with the method. In certain embodiments, the duration of response is at least or about 28 months for a population of subjects treated with the method. In certain embodiments, the duration of response is at least or about 29 months for a population of subjects treated with the method. In certain embodiments, the duration of response is at least or about 30 months for a population of subjects treated with the method.

Alternatively, the therapeutic outcome of the methods provided herein can be evaluated based on the progression free survival as set forth in Section 6.1.8.2(iv). In one embodiment, the human subject has a progression free survival of at least or about 5 months following the treatment. In another embodiment, the human subject has a progression free survival of at least or about 6 months following the treatment. In a further embodiment, the human subject has a progression free survival of at least or about 7 months following the treatment. In yet another embodiment, the human subject has a progression free survival of at least or about 8 months following the treatment. In one embodiment, the human subject has a progression free survival of at least or about 9 months following the treatment. In another embodiment, the human subject has a progression free survival of at least or about 10 months following the treatment. In a further embodiment, the human subject has a progression free survival of at least or about 11 months following the treatment. In yet another embodiment, the human subject has a progression free survival of at least or about 12 months following the treatment. In one embodiment, the human subject has a progression free survival of at least or about 13 months following the treatment. In another embodiment, the human subject has a progression free survival of at least or about 14 months following the treatment. In a further embodiment, the human subject has a progression free survival of at least or about 15 months following the treatment. In yet another embodiment, the human subject has a progression free survival of at least or about 16 months following the treatment. In one embodiment, the human subject has a progression free survival of at least or about 17 months following the treatment. In another embodiment, the human subject has a progression free survival of at least or about 18 months following the treatment. In a further embodiment, the human subject has a progression free survival of at least or about 19 months following the treatment. In yet another embodiment, the human subject has a progression free survival of at least or about 20 months following the treatment. In yet another embodiment, the human subject has a progression free survival of at least or about 21 months following the treatment. In yet another embodiment, the human subject has a progression free survival of at least or about 22 months following the treatment. In yet another embodiment, the human subject has a progression free survival of at least or about 23 months following the treatment. In yet another embodiment, the human subject has a progression free survival of at least or about 24 months following the treatment. In yet another embodiment, the human subject has a progression free survival of at least or about 25 months following the treatment. In yet another embodiment, the human subject has a progression free survival of at least or about 26 months following the treatment. In yet another embodiment, the human subject has a progression free survival of at least or about 27 months following the treatment. In yet another embodiment, the human subject has a progression free survival of at least or about 28 months following the treatment. In yet another embodiment, the human subject has a progression free survival of at least or about 29 months following the treatment. In yet another embodiment, the human subject has a progression free survival of at least or about 30 months following the treatment. In yet another embodiment, the human subject has a progression free survival of at least or about 31 months following the treatment. In yet another embodiment, the human subject has a progression free survival of at least or about 32 months following the treatment. In yet another embodiment, the human subject has a progression free survival of at least or about 33 months following the treatment.

In another embodiment, the human subject has a progression free survival ranging from 5 to 33 months following the treatment. In another embodiment, the human subject has a progression free survival ranging from 5 to 32 months following the treatment. In another embodiment, the human subject has a progression free survival ranging from 5 to 31 months following the treatment. In another embodiment, the human subject has a progression free survival ranging from 5 to 30 months following the treatment. In another embodiment, the human subject has a progression free survival ranging from 5 to 29 months following the treatment. In another embodiment, the human subject has a progression free survival ranging from 5 to 28 months following the treatment. In another embodiment, the human subject has a progression free survival ranging from 5 to 27 months following the treatment. In another embodiment, the human subject has a progression free survival ranging from 5 to 26 months following the treatment. In another embodiment, the human subject has a progression free survival ranging from 5 to 25 months following the treatment. In another embodiment, the human subject has a progression free survival ranging from 5 to 24 months following the treatment. In another embodiment, the human subject has a progression free survival ranging from 5 to 23 months following the treatment. In some embodiments, the human subject has a progression free survival ranging from 5 to 22 months following the treatment. In another embodiment, the human subject has a progression free survival ranging from 5 to 21 months following the treatment. In a further embodiment, the human subject has a progression free survival ranging from 5 to 20 months following the treatment. In yet another embodiment, the human subject has a progression free survival ranging from 5 to 19 months following the treatment. In one embodiment, the human subject has a progression free survival ranging from 5 to 18 months following the treatment. In another embodiment, the human subject has a progression free survival ranging from 5 to 17 months following the treatment. In a further embodiment, the human subject has a progression free survival ranging from 5 to 16 months following the treatment. In yet another embodiment, the human subject has a progression free survival ranging from 5 to 15 months following the treatment. In one embodiment, the human subject has a progression free survival ranging from 5 to 14 months following the treatment. In another embodiment, the human subject has a progression free survival ranging from 5 to 13 months following the treatment. In a further embodiment, the human subject has a progression free survival ranging from 5 to 12 months following the treatment. In yet another embodiment, the human subject has a progression free survival ranging from 6 to 33 months following the treatment. In yet another embodiment, the human subject has a progression free survival ranging from 6 to 32 months following the treatment. In yet another embodiment, the human subject has a progression free survival ranging from 6 to 31 months following the treatment. In yet another embodiment, the human subject has a progression free survival ranging from 6 to 30 months following the treatment. In yet another embodiment, the human subject has a progression free survival ranging from 6 to 29 months following the treatment. In yet another embodiment, the human subject has a progression free survival ranging from 6 to 28 months following the treatment. In yet another embodiment, the human subject has a progression free survival ranging from 6 to 27 months following the treatment. In yet another embodiment, the human subject has a progression free survival ranging from 6 to 26 months following the treatment. In yet another embodiment, the human subject has a progression free survival ranging from 6 to 25 months following the treatment. In yet another embodiment, the human subject has a progression free survival ranging from 6 to 22 months following the treatment. In yet another embodiment, the human subject has a progression free survival ranging from 6 to 24 months following the treatment. In yet another embodiment, the human subject has a progression free survival ranging from 6 to 23 months following the treatment. In yet another embodiment, the human subject has a progression free survival ranging from 6 to 22 months following the treatment. In another embodiment, the human subject has a progression free survival ranging from 6 to 21 months following the treatment. In a further embodiment, the human subject has a progression free survival ranging from 6 to 20 months following the treatment. In yet another embodiment, the human subject has a progression free survival ranging from 6 to 19 months following the treatment. In one embodiment, the human subject has a progression free survival ranging from 6 to 18 months following the treatment. In another embodiment, the human subject has a progression free survival ranging from 6 to 17 months following the treatment. In a further embodiment, the human subject has a progression free survival ranging from 6 to 16 months following the treatment. In yet another embodiment, the human subject has a progression free survival ranging from 6 to 15 months following the treatment. In one embodiment, the human subject has a progression free survival ranging from 6 to 14 months following the treatment. In another embodiment, the human subject has a progression free survival ranging from 6 to 13 months following the treatment. In a further embodiment, the human subject has a progression free survival ranging from 6 to 12 months following the treatment. In another embodiment, the human subject has a progression free survival ranging from 7 to 33 months following the treatment. In another embodiment, the human subject has a progression free survival ranging from 7 to 32 months following the treatment. In another embodiment, the human subject has a progression free survival ranging from 7 to 31 months following the treatment. In another embodiment, the human subject has a progression free survival ranging from 7 to 30 months following the treatment. In another embodiment, the human subject has a progression free survival ranging from 7 to 29 months following the treatment. In another embodiment, the human subject has a progression free survival ranging from 7 to 28 months following the treatment. In another embodiment, the human subject has a progression free survival ranging from 7 to 27 months following the treatment. In another embodiment, the human subject has a progression free survival ranging from 7 to 26 months following the treatment. In another embodiment, the human subject has a progression free survival ranging from 7 to 25 months following the treatment. In another embodiment, the human subject has a progression free survival ranging from 7 to 24 months following the treatment. In another embodiment, the human subject has a progression free survival ranging from 7 to 23 months following the treatment. In one embodiment, the human subject has a progression free survival ranging from 7 to 22 months following the treatment. In another embodiment, the human subject has a progression free survival ranging from 7 to 21 months following the treatment. In a further embodiment, the human subject has a progression free survival ranging from 7 to 20 months following the treatment. In yet another embodiment, the human subject has a progression free survival ranging from 7 to 19 months following the treatment. In one embodiment, the human subject has a progression free survival ranging from 7 to 18 months following the treatment. In another embodiment, the human subject has a progression free survival ranging from 7 to 17 months following the treatment. In a further embodiment, the human subject has a progression free survival ranging from 7 to 16 months following the treatment. In yet another embodiment, the human subject has a progression free survival ranging from 7 to 15 months following the treatment. In one embodiment, the human subject has a progression free survival ranging from 7 to 14 months following the treatment. In another embodiment, the human subject has a progression free survival ranging from 7 to 13 months following the treatment. In a further embodiment, the human subject has a progression free survival ranging from 7 to 12 months following the treatment. In another embodiment, the human subject has a progression free survival ranging from 8 to 22 months following the treatment. In a further embodiment, the human subject has a progression free survival ranging from 9 to 22 months following the treatment. In yet another embodiment, the human subject has a progression free survival ranging from 10 to 22 months following the treatment. In a further embodiment, the human subject has a progression free survival ranging from 11 to 22 months following the treatment. In one embodiment, the human subject has a progression free survival ranging from 12 to 22 months following the treatment. In another embodiment, the human subject has a progression free survival ranging from 13 to 22 months following the treatment. In a further embodiment, the human subject has a progression free survival ranging from 14 to 22 months following the treatment. In yet another embodiment, the human subject has a progression free survival progression free survival ranging from 15 to 22 months following the treatment. In a further embodiment, the human subject has a progression free survival ranging from 16 to 22 months following the treatment. In one embodiment, the human subject has a progression free survival ranging from 17 to 22 months following the treatment. In another embodiment, the human subject has a progression free survival ranging from 18 to 22 months following the treatment. In another embodiment, the human subject has a progression free survival ranging from 8 to 33 months following the treatment. In a further embodiment, the human subject has a progression free survival ranging from 9 to 33 months following the treatment. In yet another embodiment, the human subject has a progression free survival ranging from 10 to 33 months following the treatment. In a further embodiment, the human subject has a progression free survival ranging from 11 to 33 months following the treatment. In one embodiment, the human subject has a progression free survival ranging from 12 to 33 months following the treatment. In another embodiment, the human subject has a progression free survival ranging from 13 to 33 months following the treatment. In a further embodiment, the human subject has a progression free survival ranging from 14 to 33 months following the treatment. In yet another embodiment, the human subject has a progression free survival progression free survival ranging from 15 to 33 months following the treatment. In a further embodiment, the human subject has a progression free survival ranging from 16 to 33 months following the treatment. In one embodiment, the human subject has a progression free survival ranging from 17 to 33 months following the treatment. In another embodiment, the human subject has a progression free survival ranging from 18 to 33 months following the treatment. In another embodiment, the human subject has a progression free survival ranging from 19 to 33 months following the treatment. In another embodiment, the human subject has a progression free survival ranging from 20 to 33 months following the treatment. In another embodiment, the human subject has a progression free survival ranging from 21 to 33 months following the treatment. In another embodiment, the human subject has a progression free survival ranging from 22 to 33 months following the treatment. In another embodiment, the human subject has a progression free survival ranging from 23 to 33 months following the treatment. In another embodiment, the human subject has a progression free survival ranging from 24 to 33 months following the treatment. In another embodiment, the human subject has a progression free survival ranging from 25 to 33 months following the treatment. In another embodiment, the human subject has a progression free survival ranging from 8 to 29 months following the treatment. In a further embodiment, the human subject has a progression free survival ranging from 9 to 29 months following the treatment. In yet another embodiment, the human subject has a progression free survival ranging from 10 to 29 months following the treatment. In a further embodiment, the human subject has a progression free survival ranging from 11 to 29 months following the treatment. In one embodiment, the human subject has a progression free survival ranging from 12 to 29 months following the treatment. In another embodiment, the human subject has a progression free survival ranging from 13 to 29 months following the treatment. In a further embodiment, the human subject has a progression free survival ranging from 14 to 29 months following the treatment. In yet another embodiment, the human subject has a progression free survival progression free survival ranging from 15 to 29 months following the treatment. In a further embodiment, the human subject has a progression free survival ranging from 16 to 29 months following the treatment. In one embodiment, the human subject has a progression free survival ranging from 17 to 29 months following the treatment. In another embodiment, the human subject has a progression free survival ranging from 18 to 29 months following the treatment. In another embodiment, the human subject has a progression free survival ranging from 19 to 29 months following the treatment. In another embodiment, the human subject has a progression free survival ranging from 20 to 29 months following the treatment. In another embodiment, the human subject has a progression free survival ranging from 21 to 29 months following the treatment. In another embodiment, the human subject has a progression free survival ranging from 22 to 29 months following the treatment. In another embodiment, the human subject has a progression free survival ranging from 23 to 29 months following the treatment. In another embodiment, the human subject has a progression free survival ranging from 24 to 29 months following the treatment. In another embodiment, the human subject has a progression free survival ranging from 25 to 29 months following the treatment. In a further embodiment, the human subject has a progression free survival ranging from 6 to 21 months following the treatment. In yet another embodiment, the human subject has a progression free survival ranging from 7 to 20 months following the treatment. In a further embodiment, the human subject has a progression free survival ranging from 8 to 19 months following the treatment. In one embodiment, the human subject has a progression free survival ranging from 9 to 18 months following the treatment. In another embodiment, the human subject has a progression free survival ranging from 10 to 17 months following the treatment. In a further embodiment, the human subject has a progression free survival ranging from 11 to 16 months following the treatment. In yet another embodiment, the human subject has a progression free survival ranging from 12 to 15 months following the treatment. In a further embodiment, the human subject has a progression free survival ranging from 13 to 14 months following the treatment.

In certain embodiments, a population of the human subjects is treated by a method provided herein, wherein the progression free survival in the treated population is at least or about 5 months following the treatment. In certain embodiments, a population of the human subjects is treated by a method provided herein, wherein the progression free survival in the treated population is at least or about 6 months following the treatment. In certain embodiments, a population of the human subjects is treated by a method provided herein, wherein the progression free survival in the treated population is at least or about 7 months following the treatment. In certain embodiments, a population of the human subjects is treated by a method provided herein, wherein the progression free survival in the treated population is at least or about 8 months following the treatment. In certain embodiments, a population of the human subjects is treated by a method provided herein, wherein the progression free survival in the treated population is at least or about 9 months following the treatment. In certain embodiments, a population of the human subjects is treated by a method provided herein, wherein the progression free survival in the treated population is at least or about 10 months following the treatment. In certain embodiments, a population of the human subjects is treated by a method provided herein, wherein the progression free survival in the treated population is at least or about 11 months following the treatment. In certain embodiments, a population of the human subjects is treated by a method provided herein, wherein the progression free survival in the treated population is at least or about 5 months following the treatment. In certain embodiments, a population of the human subjects is treated by a method provided herein, wherein the progression free survival in the treated population is at least or about 12 months following the treatment. In certain embodiments, a population of the human subjects is treated by a method provided herein, wherein the progression free survival in the treated population is at least or about 13 months following the treatment. In certain embodiments, a population of the human subjects is treated by a method provided herein, wherein the progression free survival in the treated population is at least or about 14 months following the treatment. In certain embodiments, a population of the human subjects is treated by a method provided herein, wherein the progression free survival in the treated population is at least or about 15 months following the treatment. In certain embodiments, a population of the human subjects is treated by a method provided herein, wherein the progression free survival in the treated population is at least or about 16 months following the treatment. In certain embodiments, a population of the human subjects is treated by a method provided herein, wherein the progression free survival in the treated population is at least or about 17 months following the treatment. In certain embodiments, a population of the human subjects is treated by a method provided herein, wherein the progression free survival in the treated population is at least or about 18 months following the treatment. In certain embodiments, a population of the human subjects is treated by a method provided herein, wherein the progression free survival in the treated population is at least or about 19 months following the treatment. In certain embodiments, a population of the human subjects is treated by a method provided herein, wherein the progression free survival in the treated population is at least or about 20 months following the treatment. In certain embodiments, a population of the human subjects is treated by a method provided herein, wherein the progression free survival in the treated population is at least or about 21 months following the treatment. In certain embodiments, a population of the human subjects is treated by a method provided herein, wherein the progression free survival in the treated population is at least or about 22 months following the treatment. In certain embodiments, a population of the human subjects is treated by a method provided herein, wherein the progression free survival in the treated population is at least or about 23 months following the treatment. In certain embodiments, a population of the human subjects is treated by a method provided herein, wherein the progression free survival in the treated population is at least or about 24 months following the treatment. In certain embodiments, a population of the human subjects is treated by a method provided herein, wherein the progression free survival in the treated population is at least or about 25 months following the treatment. In certain embodiments, a population of the human subjects is treated by a method provided herein, wherein the progression free survival in the treated population is at least or about 26 months following the treatment. In certain embodiments, a population of the human subjects is treated by a method provided herein, wherein the progression free survival in the treated population is at least or about 27 months following the treatment. In certain embodiments, a population of the human subjects is treated by a method provided herein, wherein the progression free survival in the treated population is at least or about 28 months following the treatment. In certain embodiments, a population of the human subjects is treated by a method provided herein, wherein the progression free survival in the treated population is at least or about 29 months following the treatment. In certain embodiments, a population of the human subjects is treated by a method provided herein, wherein the progression free survival in the treated population is at least or about 30 months following the treatment. In certain embodiments, a population of the human subjects is treated by a method provided herein, wherein the progression free survival in the treated population is at least or about 31 months following the treatment. In certain embodiments, a population of the human subjects is treated by a method provided herein, wherein the progression free survival in the treated population is at least or about 32 months following the treatment. In certain embodiments, a population of the human subjects is treated by a method provided herein, wherein the progression free survival in the treated population is at least or about 33 months following the treatment.

In some embodiments, a population of the human subjects is treated by a method provided herein, wherein the progression free survival of in the treated population ranges from 5 to 33 months. In some embodiments, a population of the human subjects is treated by a method provided herein, wherein the progression free survival of in the treated population ranges from 5 to 32 months. In some embodiments, a population of the human subjects is treated by a method provided herein, wherein the progression free survival of in the treated population ranges from 5 to 31 months. In some embodiments, a population of the human subjects is treated by a method provided herein, wherein the progression free survival of in the treated population ranges from 5 to 30 months. In some embodiments, a population of the human subjects is treated by a method provided herein, wherein the progression free survival of in the treated population ranges from 5 to 29 months. In some embodiments, a population of the human subjects is treated by a method provided herein, wherein the progression free survival of in the treated population ranges from 5 to 28 months. In some embodiments, a population of the human subjects is treated by a method provided herein, wherein the progression free survival of in the treated population ranges from 5 to 27 months. In some embodiments, a population of the human subjects is treated by a method provided herein, wherein the progression free survival of in the treated population ranges from 5 to 26 months. In some embodiments, a population of the human subjects is treated by a method provided herein, wherein the progression free survival of in the treated population ranges from 5 to 25 months. In some embodiments, a population of the human subjects is treated by a method provided herein, wherein the progression free survival of in the treated population ranges from 5 to 24 months. In some embodiments, a population of the human subjects is treated by a method provided herein, wherein the progression free survival of in the treated population ranges from 5 to 23 months. In one embodiment, a population of the human subjects is treated by a method provided herein, wherein the progression free survival of in the treated population ranges from 5 to 22 months. In some embodiments, a population of the human subjects is treated by a method provided herein, wherein the progression free survival of in the treated population ranges from 5 to 21 months. In another embodiment, a population of the human subjects is treated by a method provided herein, wherein the progression free survival of in the treated population ranges from 5 to 20 months. In a further embodiment, a population of the human subjects is treated by a method provided herein, wherein the progression free survival of in the treated population ranges from 5 to 19 months. In yet another embodiment, a population of the human subjects is treated by a method provided herein, wherein the progression free survival of in the treated population ranges from 5 to 18 months. In one embodiment, a population of the human subjects is treated by a method provided herein, wherein the progression free survival of in the treated population ranges from 5 to 17 months. In another embodiment, a population of the human subjects is treated by a method provided herein, wherein the progression free survival of in the treated population ranges from 5 to 16 months. In a further embodiment, a population of the human subjects is treated by a method provided herein, wherein the progression free survival of in the treated population ranges from 5 to 15 months. In yet another embodiment, a population of the human subjects is treated by a method provided herein, wherein the progression free survival of in the treated population ranges from 5 to 14 months. In one embodiment, a population of the human subjects is treated by a method provided herein, wherein the progression free survival of in the treated population ranges from 5 to 13 months. In another embodiment, a population of the human subjects is treated by a method provided herein, wherein the progression free survival of in the treated population ranges from 5 to 12 months. In some embodiments, a population of the human subjects is treated by a method provided herein, wherein the progression free survival of in the treated population ranges from 6 to 33 months. In some embodiments, a population of the human subjects is treated by a method provided herein, wherein the progression free survival of in the treated population ranges from 6 to 32 months. In some embodiments, a population of the human subjects is treated by a method provided herein, wherein the progression free survival of in the treated population ranges from 6 to 31 months. In some embodiments, a population of the human subjects is treated by a method provided herein, wherein the progression free survival of in the treated population ranges from 6 to 30 months. In some embodiments, a population of the human subjects is treated by a method provided herein, wherein the progression free survival of in the treated population ranges from 6 to 29 months. In some embodiments, a population of the human subjects is treated by a method provided herein, wherein the progression free survival of in the treated population ranges from 6 to 28 months. In some embodiments, a population of the human subjects is treated by a method provided herein, wherein the progression free survival of in the treated population ranges from 6 to 27 months. In some embodiments, a population of the human subjects is treated by a method provided herein, wherein the progression free survival of in the treated population ranges from 6 to 26 months. In some embodiments, a population of the human subjects is treated by a method provided herein, wherein the progression free survival of in the treated population ranges from 6 to 25 months. In some embodiments, a population of the human subjects is treated by a method provided herein, wherein the progression free survival of in the treated population ranges from 6 to 24 months. In some embodiments, a population of the human subjects is treated by a method provided herein, wherein the progression free survival of in the treated population ranges from 6 to 23 months. In a further embodiment, a population of the human subjects is treated by a method provided herein, wherein the progression free survival of in the treated population ranges from 6 to 22 months. In some embodiments, a population of the human subjects is treated by a method provided herein, wherein the progression free survival of in the treated population ranges from 6 to 21 months. In another embodiment, a population of the human subjects is treated by a method provided herein, wherein the progression free survival of in the treated population ranges from 6 to 20 months. In a further embodiment, a population of the human subjects is treated by a method provided herein, wherein the progression free survival of in the treated population ranges from 6 to 19 months. In yet another embodiment, a population of the human subjects is treated by a method provided herein, wherein the progression free survival of in the treated population ranges from 6 to 18 months. In one embodiment, a population of the human subjects is treated by a method provided herein, wherein the progression free survival of in the treated population ranges from 6 to 17 months. In another embodiment, a population of the human subjects is treated by a method provided herein, wherein the progression free survival of in the treated population ranges from 6 to 16 months. In a further embodiment, a population of the human subjects is treated by a method provided herein, wherein the progression free survival of in the treated population ranges from 6 to 15 months. In yet another embodiment, a population of the human subjects is treated by a method provided herein, wherein the progression free survival of in the treated population ranges from 6 to 14 months. In one embodiment, a population of the human subjects is treated by a method provided herein, wherein the progression free survival of in the treated population ranges from 6 to 13 months. In another embodiment, a population of the human subjects is treated by a method provided herein, wherein the progression free survival of in the treated population ranges from 6 to 12 months. In some embodiments, a population of the human subjects is treated by a method provided herein, wherein the progression free survival of in the treated population ranges from 7 to 33 months. In some embodiments, a population of the human subjects is treated by a method provided herein, wherein the progression free survival of in the treated population ranges from 7 to 32 months. In some embodiments, a population of the human subjects is treated by a method provided herein, wherein the progression free survival of in the treated population ranges from 7 to 31 months. In some embodiments, a population of the human subjects is treated by a method provided herein, wherein the progression free survival of in the treated population ranges from 7 to 30 months. In some embodiments, a population of the human subjects is treated by a method provided herein, wherein the progression free survival of in the treated population ranges from 7 to 29 months. In some embodiments, a population of the human subjects is treated by a method provided herein, wherein the progression free survival of in the treated population ranges from 7 to 28 months. In some embodiments, a population of the human subjects is treated by a method provided herein, wherein the progression free survival of in the treated population ranges from 7 to 27 months. In some embodiments, a population of the human subjects is treated by a method provided herein, wherein the progression free survival of in the treated population ranges from 7 to 26 months. In some embodiments, a population of the human subjects is treated by a method provided herein, wherein the progression free survival of in the treated population ranges from 7 to 25 months. In some embodiments, a population of the human subjects is treated by a method provided herein, wherein the progression free survival of in the treated population ranges from 7 to 24 months. In some embodiments, a population of the human subjects is treated by a method provided herein, wherein the progression free survival of in the treated population ranges from 7 to 23 months. In yet another embodiment, a population of the human subjects is treated by a method provided herein, wherein the progression free survival of in the treated population ranges from 7 to 22 months. In some embodiments, a population of the human subjects is treated by a method provided herein, wherein the progression free survival of in the treated population ranges from 7 to 21 months. In another embodiment, a population of the human subjects is treated by a method provided herein, wherein the progression free survival of in the treated population ranges from 7 to 20 months. In a further embodiment, a population of the human subjects is treated by a method provided herein, wherein the progression free survival of in the treated population ranges from 7 to 19 months. In yet another embodiment, a population of the human subjects is treated by a method provided herein, wherein the progression free survival of in the treated population ranges from 7 to 18 months. In one embodiment, a population of the human subjects is treated by a method provided herein, wherein the progression free survival of in the treated population ranges from 7 to 17 months. In another embodiment, a population of the human subjects is treated by a method provided herein, wherein the progression free survival of in the treated population ranges from 7 to 16 months. In a further embodiment, a population of the human subjects is treated by a method provided herein, wherein the progression free survival of in the treated population ranges from 7 to 15 months. In yet another embodiment, a population of the human subjects is treated by a method provided herein, wherein the progression free survival of in the treated population ranges from 7 to 14 months. In one embodiment, a population of the human subjects is treated by a method provided herein, wherein the progression free survival of in the treated population ranges from 7 to 13 months. In another embodiment, a population of the human subjects is treated by a method provided herein, wherein the progression free survival of in the treated population ranges from 7 to 12 months. In one embodiment, a population of the human subjects is treated by a method provided herein, wherein the progression free survival of in the treated population ranges from 6.41 to 22 months. In another embodiment, a population of the human subjects is treated by a method provided herein, wherein the progression free survival of in the treated population ranges from 8 to 33 months. In a further embodiment, a population of the human subjects is treated by a method provided herein, wherein the progression free survival of in the treated population ranges from 9 to 33 months. In yet another embodiment, a population of the human subjects is treated by a method provided herein, wherein the progression free survival of in the treated population ranges from 10 to 33 months. In one embodiment, a population of the human subjects is treated by a method provided herein, wherein the progression free survival of in the treated population ranges from 11 to 33 months. In another embodiment, a population of the human subjects is treated by a method provided herein, wherein the progression free survival of in the treated population ranges from 12 to 33 months. In a further embodiment, a population of the human subjects is treated by a method provided herein, wherein the progression free survival of in the treated population ranges from 13 to 33 months. In yet another embodiment, a population of the human subjects is treated by a method provided herein, wherein the progression free survival of in the treated population ranges from 14 to 33 months. In one embodiment, a population of the human subjects is treated by a method provided herein, wherein the progression free survival of in the treated population ranges from 15 to 33 months. In another embodiment, a population of the human subjects is treated by a method provided herein, wherein the progression free survival of in the treated population ranges from 16 to 33 months. In a further embodiment, a population of the human subjects is treated by a method provided herein, wherein the progression free survival of in the treated population ranges from 17 to 33 months. In yet another embodiment, a population of the human subjects is treated by a method provided herein, wherein the progression free survival of in the treated population ranges from 18 to 33 months. In yet another embodiment, a population of the human subjects is treated by a method provided herein, wherein the progression free survival of in the treated population ranges from 19 to 33 months. In yet another embodiment, a population of the human subjects is treated by a method provided herein, wherein the progression free survival of in the treated population ranges from 20 to 33 months. In yet another embodiment, a population of the human subjects is treated by a method provided herein, wherein the progression free survival of in the treated population ranges from 21 to 33 months. In yet another embodiment, a population of the human subjects is treated by a method provided herein, wherein the progression free survival of in the treated population ranges from 22 to 33 months. In yet another embodiment, a population of the human subjects is treated by a method provided herein, wherein the progression free survival of in the treated population ranges from 23 to 33 months. In yet another embodiment, a population of the human subjects is treated by a method provided herein, wherein the progression free survival of in the treated population ranges from 24 to 33 months. In yet another embodiment, a population of the human subjects is treated by a method provided herein, wherein the progression free survival of in the treated population ranges from 25 to 33 months. In yet another embodiment, a population of the human subjects is treated by a method provided herein, wherein the progression free survival of in the treated population ranges from 26 to 33 months. In yet another embodiment, a population of the human subjects is treated by a method provided herein, wherein the progression free survival of in the treated population ranges from 27 to 33 months. In yet another embodiment, a population of the human subjects is treated by a method provided herein, wherein the progression free survival of in the treated population ranges from 28 to 33 months. In yet another embodiment, a population of the human subjects is treated by a method provided herein, wherein the progression free survival of in the treated population ranges from 29 to 33 months. In another embodiment, a population of the human subjects is treated by a method provided herein, wherein the progression free survival of in the treated population ranges from 8 to 29 months. In a further embodiment, a population of the human subjects is treated by a method provided herein, wherein the progression free survival of in the treated population ranges from 9 to 29 months. In yet another embodiment, a population of the human subjects is treated by a method provided herein, wherein the progression free survival of in the treated population ranges from 10 to 29 months. In one embodiment, a population of the human subjects is treated by a method provided herein, wherein the progression free survival of in the treated population ranges from 11 to 29 months. In another embodiment, a population of the human subjects is treated by a method provided herein, wherein the progression free survival of in the treated population ranges from 12 to 29 months. In a further embodiment, a population of the human subjects is treated by a method provided herein, wherein the progression free survival of in the treated population ranges from 13 to 29 months. In yet another embodiment, a population of the human subjects is treated by a method provided herein, wherein the progression free survival of in the treated population ranges from 14 to 29 months. In one embodiment, a population of the human subjects is treated by a method provided herein, wherein the progression free survival of in the treated population ranges from 15 to 29 months. In another embodiment, a population of the human subjects is treated by a method provided herein, wherein the progression free survival of in the treated population ranges from 16 to 29 months. In a further embodiment, a population of the human subjects is treated by a method provided herein, wherein the progression free survival of in the treated population ranges from 17 to 29 months. In yet another embodiment, a population of the human subjects is treated by a method provided herein, wherein the progression free survival of in the treated population ranges from 18 to 29 months. In yet another embodiment, a population of the human subjects is treated by a method provided herein, wherein the progression free survival of in the treated population ranges from 19 to 29 months. In yet another embodiment, a population of the human subjects is treated by a method provided herein, wherein the progression free survival of in the treated population ranges from 20 to 29 months. In yet another embodiment, a population of the human subjects is treated by a method provided herein, wherein the progression free survival of in the treated population ranges from 21 to 29 months. In yet another embodiment, a population of the human subjects is treated by a method provided herein, wherein the progression free survival of in the treated population ranges from 22 to 29 months. In yet another embodiment, a population of the human subjects is treated by a method provided herein, wherein the progression free survival of in the treated population ranges from 23 to 29 months. In yet another embodiment, a population of the human subjects is treated by a method provided herein, wherein the progression free survival of in the treated population ranges from 24 to 29 months. In yet another embodiment, a population of the human subjects is treated by a method provided herein, wherein the progression free survival of in the treated population ranges from 25 to 29 months. In yet another embodiment, a population of the human subjects is treated by a method provided herein, wherein the progression free survival of in the treated population ranges from 26 to 29 months. In yet another embodiment, a population of the human subjects is treated by a method provided herein, wherein the progression free survival of in the treated population ranges from 27 to 29 months. In yet another embodiment, a population of the human subjects is treated by a method provided herein, wherein the progression free survival of in the treated population ranges from 28 to 29 months. In another embodiment, a population of the human subjects is treated by a method provided herein, wherein the progression free survival of in the treated population ranges from 8 to 22 months. In a further embodiment, a population of the human subjects is treated by a method provided herein, wherein the progression free survival of in the treated population ranges from 9 to 22 months. In yet another embodiment, a population of the human subjects is treated by a method provided herein, wherein the progression free survival of in the treated population ranges from 10 to 22 months. In one embodiment, a population of the human subjects is treated by a method provided herein, wherein the progression free survival of in the treated population ranges from 11 to 22 months. In another embodiment, a population of the human subjects is treated by a method provided herein, wherein the progression free survival of in the treated population ranges from 12 to 12 months. In a further embodiment, a population of the human subjects is treated by a method provided herein, wherein the progression free survival of in the treated population ranges from 13 to 22 months. In yet another embodiment, a population of the human subjects is treated by a method provided herein, wherein the progression free survival of in the treated population ranges from 14 to 22 months. In one embodiment, a population of the human subjects is treated by a method provided herein, wherein the progression free survival of in the treated population ranges from 15 to 22 months. In another embodiment, a population of the human subjects is treated by a method provided herein, wherein the progression free survival of in the treated population ranges from 16 to 22 months. In a further embodiment, a population of the human subjects is treated by a method provided herein, wherein the progression free survival of in the treated population ranges from 17 to 22 months. In yet another embodiment, a population of the human subjects is treated by a method provided herein, wherein the progression free survival of in the treated population ranges from 18 to 22 months. In one embodiment, a population of the human subjects is treated by a method provided herein, wherein the progression free survival of in the treated population ranges from 6 to 21 months. In another embodiment, a population of the human subjects is treated by a method provided herein, wherein the progression free survival of in the treated population ranges from 7 to 20 months. In a further embodiment, a population of the human subjects is treated by a method provided herein, wherein the progression free survival of in the treated population ranges from 8 to 19 months. In yet another embodiment, a population of the human subjects is treated by a method provided herein, wherein the progression free survival of in the treated population ranges from 9 to 18 months. In one embodiment, a population of the human subjects is treated by a method provided herein, wherein the progression free survival of in the treated population ranges from 10 to 17 months. In another embodiment, a population of the human subjects is treated by a method provided herein, wherein the progression free survival of in the treated population ranges from 11 to 16 months. In a further embodiment, a population of the human subjects is treated by a method provided herein, wherein the progression free survival of in the treated population ranges from 12 to 15 months. In yet another embodiment, a population of the human subjects is treated by a method provided herein, wherein the progression free survival of in the treated population ranges from 13 to 24 months.

In some embodiments, the progression free survival is at least or about 6 months for a population of subjects treated with the method. In some embodiments, some embodiments, the progression free survival is at least or about 5 months for a population of subjects treated with the method. In some embodiments, some embodiments, the progression free survival is at least or about 6 months for a population of subjects treated with the method. In some embodiments, some embodiments, the progression free survival is at least or about 7 months for a population of subjects treated with the method. In some embodiments, some embodiments, the progression free survival is at least or about 8 months for a population of subjects treated with the method. In some embodiments, some embodiments, the progression free survival is at least or about 9 months for a population of subjects treated with the method. In some embodiments, some embodiments, the progression free survival is at least or about 10 months for a population of subjects treated with the method. In some embodiments, some embodiments, the progression free survival is at least or about 11 months for a population of subjects treated with the method. In some embodiments, some embodiments, the progression free survival is at least or about 12 months for a population of subjects treated with the method. In some embodiments, some embodiments, the progression free survival is at least or about 13 months for a population of subjects treated with the method. In some embodiments, some embodiments, the progression free survival is at least or about 14 months for a population of subjects treated with the method. In some embodiments, some embodiments, the progression free survival is at least or about 15 months for a population of subjects treated with the method. In some embodiments, some embodiments, the progression free survival is at least or about 16 months for a population of subjects treated with the method. In some embodiments, some embodiments, the progression free survival is at least or about 17 months for a population of subjects treated with the method. In some embodiments, some embodiments, the progression free survival is at least or about 18 months for a population of subjects treated with the method. In some embodiments, some embodiments, the progression free survival is at least or about 19 months for a population of subjects treated with the method. In some embodiments, some embodiments, the progression free survival is at least or about 20 months for a population of subjects treated with the method. In some embodiments, some embodiments, the progression free survival is at least or about 21 months for a population of subjects treated with the method. In some embodiments, some embodiments, the progression free survival is at least or about 22 months for a population of subjects treated with the method. In some embodiments, some embodiments, the progression free survival is at least or about 23 months for a population of subjects treated with the method. In some embodiments, some embodiments, the progression free survival is at least or about 24 months for a population of subjects treated with the method. In some embodiments, some embodiments, the progression free survival is at least or about 25 months for a population of subjects treated with the method. In some embodiments, some embodiments, the progression free survival is at least or about 26 months for a population of subjects treated with the method. In some embodiments, some embodiments, the progression free survival is at least or about 27 months for a population of subjects treated with the method. In some embodiments, some embodiments, the progression free survival is at least or about 28 months for a population of subjects treated with the method. In some embodiments, some embodiments, the progression free survival is at least or about 29 months for a population of subjects treated with the method. In some embodiments, some embodiments, the progression free survival is at least or about 30 months for a population of subjects treated with the method. In some embodiments, some embodiments, the progression free survival is at least or about 31 months for a population of subjects treated with the method. In some embodiments, some embodiments, the progression free survival is at least or about 32 months for a population of subjects treated with the method. In some embodiments, some embodiments, the progression free survival is at least or about 33 months for a population of subjects treated with the method.

Alternatively, the therapeutic outcome of the methods provided herein can be evaluated based on the overall survival as set forth in Section 6.1.8.2(v). In one embodiment, the human subject has an overall survival of at least or about 5 months following the treatment. In another embodiment, the human subject has an overall survival of at least or about 6 months following the treatment. In a further embodiment, the human subject has an overall survival of at least or about 7 months following the treatment. In yet another embodiment, the human subject has an overall survival of at least or about 8 months following the treatment. In one embodiment, the human subject has an overall survival of at least or about 9 months following the treatment. In another embodiment, the human subject has an overall survival of at least or about 10 months following the treatment. In a further embodiment, the human subject has an overall survival of at least or about 11 months following the treatment. In yet another embodiment, the human subject has an overall survival of at least or about 12 months following the treatment. In one embodiment, the human subject has an overall survival of at least or about 13 months following the treatment. In another embodiment, the human subject has an overall survival of at least or about 14 months following the treatment. In a further embodiment, the human subject has an overall survival of at least or about 14.7 months following the treatment. In yet another embodiment, the human subject has an overall survival of at least or about 15 months following the treatment. In one embodiment, the human subject has an overall survival of at least or about 16 months following the treatment. In one embodiment, the human subject has an overall survival of at least or about 16.1 months following the treatment. In another embodiment, the human subject has an overall survival of at least or about 17 months following the treatment. In a further embodiment, the human subject has an overall survival of at least or about 18 months following the treatment. In yet another embodiment, the human subject has an overall survival of at least or about 19 months following the treatment. In one embodiment, the human subject has an overall survival of at least or about 20 months following the treatment. In another embodiment, the human subject has an overall survival of at least or about 21 months following the treatment. In a further embodiment, the human subject has an overall survival of at least or about 22 months following the treatment. In a further embodiment, the human subject has an overall survival of at least or about 22.3 months following the treatment. In a further embodiment, the human subject has an overall survival of at least or about 23 months following the treatment. In yet another embodiment, the human subject has an overall survival of at least or about 24 months following the treatment. In one embodiment, the human subject has an overall survival of at least or about 25 months following the treatment. In another embodiment, the human subject has an overall survival of at least or about 26 months following the treatment. In a further embodiment, the human subject has an overall survival of at least or about 27 months following the treatment. In one embodiment, the human subject has an overall survival of at least or about 28 months following the treatment. In another embodiment, the human subject has an overall survival of at least or about 29 months following the treatment. In a further embodiment, the human subject has an overall survival of at least or about 30 months following the treatment. In a further embodiment, the human subject has an overall survival of at least or about 31 months following the treatment. In a further embodiment, the human subject has an overall survival of at least or about 32 months following the treatment. In a further embodiment, the human subject has an overall survival of at least or about 33 months following the treatment. In a further embodiment, the human subject has an overall survival of at least or about 34 months following the treatment. In a further embodiment, the human subject has an overall survival of at least or about 35 months following the treatment.

In one embodiment, the human subject has an overall survival ranging from 10 to 35 months following the treatment. In another embodiment, the human subject has an overall survival ranging from 10 to 34 months following the treatment. In a further embodiment, the human subject has an overall survival ranging from 10 to 33 months following the treatment. In yet another embodiment, the human subject has an overall survival ranging from 10 to 32 months following the treatment. In one embodiment, the human subject has an overall survival ranging from 10 to 31 months following the treatment. In another embodiment, the human subject has an overall survival ranging from 10 to 30 months following the treatment. In a further embodiment, the human subject has an overall survival ranging from 10 to 29 months following the treatment. In yet another embodiment, the human subject has an overall survival ranging from 10 to 28 months following the treatment. In one embodiment, the human subject has an overall survival ranging from 10 to 27 months following the treatment. In one embodiment, the human subject has an overall survival ranging from 10 to 26 months following the treatment. In one embodiment, the human subject has an overall survival ranging from 10 to 25 months following the treatment. In another embodiment, the human subject has an overall survival ranging from 11 to 35 months following the treatment. In a further embodiment, the human subject has an overall survival ranging from 12 to 35 months following the treatment. In yet another embodiment, the human subject has an overall survival ranging from 13 to 35 months following the treatment. In one embodiment, the human subject has an overall survival ranging from 14 to 35 months following the treatment. In one embodiment, the human subject has an overall survival ranging from 14 to 35 months following the treatment. In one embodiment, the human subject has an overall survival ranging from 15 to 35 months following the treatment. In one embodiment, the human subject has an overall survival ranging from 16 to 35 months following the treatment. In one embodiment, the human subject has an overall survival ranging from 17 to 35 months following the treatment. In one embodiment, the human subject has an overall survival ranging from 18 to 35 months following the treatment. In one embodiment, the human subject has an overall survival ranging from 19 to 35 months following the treatment. In one embodiment, the human subject has an overall survival ranging from 20 to 35 months following the treatment. In one embodiment, the human subject has an overall survival ranging from 21 to 35 months following the treatment. In one embodiment, the human subject has an overall survival ranging from 22 to 35 months following the treatment. In one embodiment, the human subject has an overall survival ranging from 23 to 35 months following the treatment. In one embodiment, the human subject has an overall survival ranging from 24 to 35 months following the treatment. In one embodiment, the human subject has an overall survival ranging from 25 to 35 months following the treatment. In one embodiment, the human subject has an overall survival ranging from 26 to 35 months following the treatment. In one embodiment, the human subject has an overall survival ranging from 27 to 35 months following the treatment. In one embodiment, the human subject has an overall survival ranging from 28 to 35 months following the treatment. In one embodiment, the human subject has an overall survival ranging from 29 to 35 months following the treatment. In one embodiment, the human subject has an overall survival ranging from 30 to 35 months following the treatment. In one embodiment, the human subject has an overall survival ranging from 31 to 35 months following the treatment. In another embodiment, the human subject has an overall survival ranging from 15 to 32 months following the treatment. In a further embodiment, the human subject has an overall survival ranging from 16 to 32 months following the treatment. In a further embodiment, the human subject has an overall survival ranging from 17 to 32 months following the treatment. In a further embodiment, the human subject has an overall survival ranging from 18 to 32 months following the treatment. In a further embodiment, the human subject has an overall survival ranging from 19 to 32 months following the treatment. In a further embodiment, the human subject has an overall survival ranging from 20 to 32 months following the treatment. In a further embodiment, the human subject has an overall survival ranging from 21 to 32 months following the treatment. In a further embodiment, the human subject has an overall survival ranging from 22 to 32 months following the treatment. In a further embodiment, the human subject has an overall survival ranging from 23 to 32 months following the treatment. In a further embodiment, the human subject has an overall survival ranging from 24 to 32 months following the treatment. In a further embodiment, the human subject has an overall survival ranging from 25 to 32 months following the treatment. In a further embodiment, the human subject has an overall survival ranging from 26 to 32 months following the treatment. In a further embodiment, the human subject has an overall survival ranging from 27 to 32 months following the treatment. In a further embodiment, the human subject has an overall survival ranging from 28 to 32 months following the treatment. In a further embodiment, the human subject has an overall survival ranging from 29 to 32 months following the treatment. In a further embodiment, the human subject has an overall survival ranging from 30 to 32 months following the treatment. In a further embodiment, the human subject has an overall survival ranging from 31 to 32 months following the treatment. In another embodiment, the human subject has an overall survival ranging from 15 to 30 months following the treatment. In a further embodiment, the human subject has an overall survival ranging from 16 to 30 months following the treatment. In a further embodiment, the human subject has an overall survival ranging from 17 to 30 months following the treatment. In a further embodiment, the human subject has an overall survival ranging from 18 to 30 months following the treatment. In a further embodiment, the human subject has an overall survival ranging from 19 to 30 months following the treatment. In a further embodiment, the human subject has an overall survival ranging from 20 to 30 months following the treatment. In a further embodiment, the human subject has an overall survival ranging from 21 to 30 months following the treatment. In a further embodiment, the human subject has an overall survival ranging from 22 to 30 months following the treatment. In a further embodiment, the human subject has an overall survival ranging from 23 to 30 months following the treatment. In a further embodiment, the human subject has an overall survival ranging from 24 to 30 months following the treatment. In a further embodiment, the human subject has an overall survival ranging from 25 to 30 months following the treatment. In a further embodiment, the human subject has an overall survival ranging from 26 to 30 months following the treatment. In a further embodiment, the human subject has an overall survival ranging from 27 to 30 months following the treatment. In a further embodiment, the human subject has an overall survival ranging from 28 to 30 months following the treatment. In a further embodiment, the human subject has an overall survival ranging from 29 to 30 months following the treatment. In yet another embodiment, the human subject has an overall survival ranging from 16 to 29 months following the treatment. In yet another embodiment, the human subject has an overall survival ranging from 17 to 29 months following the treatment. In one embodiment, the human subject has an overall survival ranging from 17 to 28 months following the treatment. In another embodiment, the human subject has an overall survival ranging from 18 to 28 months following the treatment. In a further embodiment, the human subject has an overall survival ranging from 18 to 27 months following the treatment. In a further embodiment, the human subject has an overall survival ranging from 17 to 27 months following the treatment. In yet another embodiment, the human subject has an overall survival ranging from 17 to 26 months following the treatment. In one embodiment, the human subject has an overall survival ranging from 18 to 26 months following the treatment. In another embodiment, the human subject has an overall survival ranging from 18 to 25 months following the treatment. In one embodiment, the human subject has an overall survival ranging from 19 to 25 months following the treatment. In one embodiment, the human subject has an overall survival ranging from 19 to 24 months following the treatment. In one embodiment, the human subject has an overall survival ranging from 20 to 24 months following the treatment. In one embodiment, the human subject has an overall survival ranging from 20 to 23 months following the treatment.

Additionally, in some embodiments, the overall survival is evaluated for a population of human subjects treated by a method provided herein by evaluating the median or mean overall survival in the treated population. In one embodiment, a population of the human subjects is treated by a method provided herein, wherein the median or mean overall survival in the treated population is at least or about 5 months. In another embodiment, a population of the human subjects is treated by a method provided herein, wherein the median or mean overall survival in the treated population is at least or about 6 months. In a further embodiment, a population of the human subjects is treated by a method provided herein, wherein the median or mean overall survival in the treated population is at least or about 7 months. In yet another embodiment, a population of the human subjects is treated by a method provided herein, wherein the median or mean overall survival in the treated population is at least or about 8 months. In one embodiment, a population of the human subjects is treated by a method provided herein, wherein the median or mean overall survival in the treated population is at least or about 9 months. In another embodiment, a population of the human subjects is treated by a method provided herein, wherein the median or mean overall survival in the treated population is at least or about 10 months. In another embodiment, a population of the human subjects is treated by a method provided herein, wherein the median or mean overall survival in the treated population is at least or about 10.51 months. In a further embodiment, a population of the human subjects is treated by a method provided herein, wherein the median or mean overall survival in the treated population is at least or about 11 months. In yet another embodiment, a population of the human subjects is treated by a method provided herein, wherein the median or mean overall survival in the treated population is at least or about 12 months. In one embodiment, a population of the human subjects is treated by a method provided herein, wherein the median or mean overall survival in the treated population is at least or about 13 months. In another embodiment, a population of the human subjects is treated by a method provided herein, wherein the median or mean overall survival in the treated population is at least or about 14 months. In a further embodiment, a population of the human subjects is treated by a method provided herein, wherein the median or mean overall survival in the treated population is at least or about 14.7 months. In yet another embodiment, a population of the human subjects is treated by a method provided herein, wherein the median or mean overall survival in the treated population is at least or about 15 months. In one embodiment, a population of the human subjects is treated by a method provided herein, wherein the median or mean overall survival in the treated population is at least or about 16 months. In one embodiment, a population of the human subjects is treated by a method provided herein, wherein the median or mean overall survival in the treated population is at least or about 16.1 months. In another embodiment, a population of the human subjects is treated by a method provided herein, wherein the median or mean overall survival in the treated population is at least or about 17 months. In a further embodiment, a population of the human subjects is treated by a method provided herein, wherein the median or mean overall survival in the treated population is at least or about 18 months. In yet another embodiment, a population of the human subjects is treated by a method provided herein, wherein the median or mean overall survival in the treated population is at least or about 19 months. In one embodiment, a population of the human subjects is treated by a method provided herein, wherein the median or mean overall survival in the treated population is at least or about 20 months. In another embodiment, a population of the human subjects is treated by a method provided herein, wherein the median or mean overall survival in the treated population is at least or about 21 months. In a further embodiment, a population of the human subjects is treated by a method provided herein, wherein the median or mean overall survival in the treated population is at least or about 22 months. In a further embodiment, a population of the human subjects is treated by a method provided herein, wherein the median or mean overall survival in the treated population is at least or about 22.3 months. In yet another embodiment, a population of the human subjects is treated by a method provided herein, wherein the median or mean overall survival in the treated population is at least or about 23 months. In one embodiment, a population of the human subjects is treated by a method provided herein, wherein the median or mean overall survival in the treated population is at least or about 24 months. In another embodiment, a population of the human subjects is treated by a method provided herein, wherein the median or mean overall survival in the treated population is at least or about 25 months. In a further embodiment, a population of the human subjects is treated by a method provided herein, wherein the median or mean overall survival in the treated population is at least or about 26 months. In yet another embodiment, a population of the human subjects is treated by a method provided herein, wherein the median or mean overall survival in the treated population is at least or about 27 months. In one embodiment, a population of the human subjects is treated by a method provided herein, wherein the median or mean overall survival in the treated population is at least or about 28 months. In another embodiment, a population of the human subjects is treated by a method provided herein, wherein the median or mean overall survival in the treated population is at least or about 29 months. In a further embodiment, a population of the human subjects is treated by a method provided herein, wherein the median or mean overall survival in the treated population is at least or about 30 months. In a further embodiment, a population of the human subjects is treated by a method provided herein, wherein the median or mean overall survival in the treated population is at least or about 31 months. In a further embodiment, a population of the human subjects is treated by a method provided herein, wherein the median or mean overall survival in the treated population is at least or about 32 months. In a further embodiment, a population of the human subjects is treated by a method provided herein, wherein the median or mean overall survival in the treated population is at least or about 33 months. In a further embodiment, a population of the human subjects is treated by a method provided herein, wherein the median or mean overall survival in the treated population is at least or about 34 months. In a further embodiment, a population of the human subjects is treated by a method provided herein, wherein the median or mean overall survival in the treated population is at least or about 35 months.

In one embodiment, a population of the human subjects is treated by a method provided herein, wherein the overall survival in the treated population ranges from 10 to 35 months. In another embodiment, a population of the human subjects is treated by a method provided herein, wherein the overall survival in the treated population ranges from 10 to 34 months. In a further embodiment, a population of the human subjects is treated by a method provided herein, wherein the overall survival in the treated population ranges from 10 to 33 months. In yet another embodiment, a population of the human subjects is treated by a method provided herein, wherein the overall survival in the treated population ranges from 10 to 32 months. In one embodiment, a population of the human subjects is treated by a method provided herein, wherein the overall survival in the treated population ranges from 10 to 31 months. In another embodiment, a population of the human subjects is treated by a method provided herein, wherein the overall survival in the treated population ranges from 10 to 30 months. In a further embodiment, a population of the human subjects is treated by a method provided herein, wherein the overall survival in the treated population ranges from 10 to 29 months. In yet another embodiment, a population of the human subjects is treated by a method provided herein, wherein the overall survival in the treated population ranges from 10 to 28 months. In one embodiment, a population of the human subjects is treated by a method provided herein, wherein the overall survival in the treated population ranges from 10 to 27 months. In one embodiment, a population of the human subjects is treated by a method provided herein, wherein the overall survival in the treated population ranges from 10 to 26 months. In one embodiment, a population of the human subjects is treated by a method provided herein, wherein the overall survival in the treated population ranges from 10 to 25 months. In another embodiment, a population of the human subjects is treated by a method provided herein, wherein the overall survival in the treated population ranges from 11 to 35 months. In a further embodiment, a population of the human subjects is treated by a method provided herein, wherein the overall survival in the treated population ranges from 12 to 35 months. In yet another embodiment, a population of the human subjects is treated by a method provided herein, wherein the overall survival in the treated population ranges from 13 to 35 months. In one embodiment, a population of the human subjects is treated by a method provided herein, wherein the overall survival in the treated population ranges from 14 to 35 months. In one embodiment, 14 to 35 months. In one embodiment, a population of the human subjects is treated by a method provided herein, wherein the overall survival in the treated population ranges from 15 to 35 months. In one embodiment, a population of the human subjects is treated by a method provided herein, wherein the overall survival in the treated population ranges from 16 to 35 months. In one embodiment, a population of the human subjects is treated by a method provided herein, wherein the overall survival in the treated population ranges from 17 to 35 months. In one embodiment, a population of the human subjects is treated by a method provided herein, wherein the overall survival in the treated population ranges from 18 to 35 months. In one embodiment, a population of the human subjects is treated by a method provided herein, wherein the overall survival in the treated population ranges from 19 to 35 months. In another embodiment, a population of the human subjects is treated by a method provided herein, wherein the overall survival in the treated population ranges from 15 to 30 months. In a further embodiment, a population of the human subjects is treated by a method provided herein, wherein the overall survival in the treated population ranges from 16 to 30 months. In yet another embodiment, a population of the human subjects is treated by a method provided herein, wherein the overall survival in the treated population ranges from 16 to 29 months. In yet another embodiment, a population of the human subjects is treated by a method provided herein, wherein the overall survival in the treated population ranges from 17 to 29 months. In one embodiment, a population of the human subjects is treated by a method provided herein, wherein the overall survival in the treated population ranges from 17 to 28 months. In another embodiment, a population of the human subjects is treated by a method provided herein, wherein the overall survival in the treated population ranges from 18 to 28 months. In a further embodiment, a population of the human subjects is treated by a method provided herein, wherein the overall survival in the treated population ranges from 18 to 27 months. In a further embodiment, a population of the human subjects is treated by a method provided herein, wherein the overall survival in the treated population ranges from 17 to 27 months. In yet another embodiment, a population of the human subjects is treated by a method provided herein, wherein the overall survival in the treated population ranges from 17 to 26 months. In one embodiment, a population of the human subjects is treated by a method provided herein, wherein the overall survival in the treated population ranges from 18 to 26 months. In another embodiment, a population of the human subjects is treated by a method provided herein, wherein the overall survival in the treated population ranges from 18 to 25 months. In one embodiment, a population of the human subjects is treated by a method provided herein, wherein the overall survival in the treated population ranges from 19 to 25 months. In one embodiment, a population of the human subjects is treated by a method provided herein, wherein the overall survival in the treated population ranges from 19 to 24 months. In one embodiment, a population of the human subjects is treated by a method provided herein, wherein the overall survival in the treated population ranges from 20 to 24 months. In one embodiment, a population of the human subjects is treated by a method provided herein, wherein the overall survival in the treated population ranges from 20 to 23 months.

In one embodiment, the median overall survival is at least or about 5 months for a population of subjects treated with the method. In one embodiment, the median overall survival is at least or about 6 months for a population of subjects treated with the method. In one embodiment, the median overall survival is at least or about 7 months for a population of subjects treated with the method. In one embodiment, the median overall survival is at least or about 8 months for a population of subjects treated with the method. In one embodiment, the median overall survival is at least or about 9 months for a population of subjects treated with the method. In one embodiment, the median overall survival is at least or about 10 months for a population of subjects treated with the method. In one embodiment, the median overall survival is at least or about 11 months for a population of subjects treated with the method. In one embodiment, the median overall survival is at least or about 12 months for a population of subjects treated with the method. In one embodiment, the median overall survival is at least or about 13 months for a population of subjects treated with the method. In one embodiment, the median overall survival is at least or about 14 months for a population of subjects treated with the method. In one embodiment, the median overall survival is at least or about 15 months for a population of subjects treated with the method. In one embodiment, the median overall survival is at least or about 16 months for a population of subjects treated with the method. In one embodiment, the median overall survival is at least or about 17 months for a population of subjects treated with the method. In one embodiment, the median overall survival is at least or about 18 months for a population of subjects treated with the method. In one embodiment, the median overall survival is at least or about 19 months for a population of subjects treated with the method. In one embodiment, the median overall survival is at least or about 20 months for a population of subjects treated with the method. In one embodiment, the median overall survival is at least or about 21 months for a population of subjects treated with the method. In one embodiment, the median overall survival is at least or about 22 months for a population of subjects treated with the method. In one embodiment, the median overall survival is at least or about 22.3 months for a population of subjects treated with the method. In one embodiment, the median overall survival is at least or about 23 months for a population of subjects treated with the method. In one embodiment, the median overall survival is at least or about 24 months for a population of subjects treated with the method. In one embodiment, the median overall survival is at least or about 25 months for a population of subjects treated with the method. In one embodiment, the median overall survival is at least or about 26 months for a population of subjects treated with the method. In one embodiment, the median overall survival is at least or about 27 months for a population of subjects treated with the method. In one embodiment, the median overall survival is at least or about 28 months for a population of subjects treated with the method. In one embodiment, the median overall survival is at least or about 29 months for a population of subjects treated with the method. In one embodiment, the median overall survival is at least or about 30 months for a population of subjects treated with the method. In one embodiment, the median overall survival is at least or about 31 months for a population of subjects treated with the method. In one embodiment, the median overall survival is at least or about 32 months for a population of subjects treated with the method. In one embodiment, the median overall survival is at least or about 33 months for a population of subjects treated with the method. In one embodiment, the median overall survival is at least or about 34 months for a population of subjects treated with the method. In one embodiment, the median overall survival is at least or about 35 months for a population of subjects treated with the method.

In one embodiment, the overall survival is from 10 to 35 months for a population of subjects treated with the method. In one embodiment, the overall survival is from 10 to 34 months for a population of subjects treated with the method. In one embodiment, the overall survival is from 10 to 33 months for a population of subjects treated with the method. In one embodiment, the overall survival is from 10 to 32 months for a population of subjects treated with the method. In one embodiment, the overall survival is from 10 to 31 months for a population of subjects treated with the method. In one embodiment, the overall survival is from 10 to 30 months for a population of subjects treated with the method. In one embodiment, the overall survival is from 10 to 29 months for a population of subjects treated with the method. In one embodiment, the overall survival is from 10 to 28 months for a population of subjects treated with the method. In one embodiment, the overall survival is from 10 to 27 months for a population of subjects treated with the method. In one embodiment, the overall survival is from 10 to 26 months for a population of subjects treated with the method. In one embodiment, the overall survival is from 10 to 25 months for a population of subjects treated with the method. In one embodiment, the overall survival is from 11 to 35 months for a population of subjects treated with the method. In one embodiment, the overall survival is from 12 to 35 months for a population of subjects treated with the method. In one embodiment, the overall survival is from 13 to 35 months for a population of subjects treated with the method. In one embodiment, the overall survival is from 14 to 35 months for a population of subjects treated with the method. In one embodiment, the overall survival is from 15 to 35 months for a population of subjects treated with the method. In one embodiment, the overall survival is from 16 to 35 months for a population of subjects treated with the method. In one embodiment, the overall survival is from 17 to 35 months for a population of subjects treated with the method. In one embodiment, the overall survival is from 18 to 35 months for a population of subjects treated with the method. In one embodiment, the overall survival is from 19 to 35 months for a population of subjects treated with the method. In one embodiment, the overall survival is from 15 to 30 months for a population of subjects treated with the method. In one embodiment, the overall survival is from 16 to 30 months for a population of subjects treated with the method. In one embodiment, the overall survival is from 16 to 29 months for a population of subjects treated with the method. In one embodiment, the overall survival is from 17 to 29 months for a population of subjects treated with the method. In one embodiment, the overall survival is from 17 to 28 months for a population of subjects treated with the method. In one embodiment, the overall survival is from 18 to 28 months for a population of subjects treated with the method. In one embodiment, the overall survival is from 18 to 27 months for a population of subjects treated with the method. In one embodiment, the overall survival is from 17 to 27 months for a population of subjects treated with the method. In one embodiment, the overall survival is from 17 to 26 months for a population of subjects treated with the method. In one embodiment, the overall survival is from 18 to 26 months for a population of subjects treated with the method. In one embodiment, the overall survival is from 18 to 25 months for a population of subjects treated with the method. In one embodiment, the overall survival is from 19 to 25 months for a population of subjects treated with the method. In one embodiment, the overall survival is from 19 to 24 months for a population of subjects treated with the method. In one embodiment, the overall survival is from 20 to 24 months for a population of subjects treated with the method. In one embodiment, the overall survival is from 20 to 23 months for a population of subjects treated with the method.

In one embodiment, the overall survival is from 25 to 30 months for a population of subjects treated with the method. In one embodiment, the overall survival is from 26 to 30 months for a population of subjects treated with the method. In one embodiment, the overall survival is from 27 to 30 months for a population of subjects treated with the method. In one embodiment, the overall survival is from 28 to 30 months for a population of subjects treated with the method. In one embodiment, the overall survival is from 26 to 31 months for a population of subjects treated with the method. In one embodiment, the overall survival is from 27 to 31 months for a population of subjects treated with the method. In one embodiment, the overall survival is from 28 to 31 months for a population of subjects treated with the method. In one embodiment, the overall survival is from 29 to 31 months for a population of subjects treated with the method. In one embodiment, the overall survival is from 27 to 32 months for a population of subjects treated with the method. In one embodiment, the overall survival is from 28 to 32 months for a population of subjects treated with the method. In one embodiment, the overall survival is from 29 to 32 months for a population of subjects treated with the method. In one embodiment, the overall survival is from 30 to 32 months for a population of subjects treated with the method. In one embodiment, the overall survival is from 28 to 33 months for a population of subjects treated with the method. In one embodiment, the overall survival is from 29 to 33 months for a population of subjects treated with the method. In one embodiment, the overall survival is from 30 to 33 months for a population of subjects treated with the method. In one embodiment, the overall survival is from 31 to 33 months for a population of subjects treated with the method. In one embodiment, the overall survival is from 29 to 34 months for a population of subjects treated with the method. In one embodiment, the overall survival is from 30 to 34 months for a population of subjects treated with the method. In one embodiment, the overall survival is from 31 to 34 months for a population of subjects treated with the method. In one embodiment, the overall survival is from 32 to 34 months for a population of subjects treated with the method. In one embodiment, the overall survival is from 30 to 35 months for a population of subjects treated with the method. In one embodiment, the overall survival is from 31 to 35 months for a population of subjects treated with the method. In one embodiment, the overall survival is from 32 to 35 months for a population of subjects treated with the method. In one embodiment, the overall survival is from 33 to 35 months for a population of subjects treated with the method.

### 5.2.2 Methods of Treating Cancer in Patient Populations Based on Additional Selection Criteria

Provided herein are methods for the treatment of various cancers in subjects, wherein the cancers have any of the suitable markers and/or characteristics as provided in Section 6. Also provided herein are methods for the treatment of various cancers in subjects, wherein the subjects have any of the suitable characteristics as provided in Section 6.

In one aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has any of the suitable characteristics as provided in Section 6.

In some aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the cancer has any of the suitable markers and/or characteristics as provided in Section 6.

In another aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), and wherein the subject has any of the suitable characteristics as provided in Section 6. In a further aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), and wherein the cancer has any of the suitable markers and/or characteristics as provided in Section 6.

In some embodiments of the methods provided herein including in Section 5.2 including Sections 5.2.1 and 5.2.2 and Sections 3 and 6, the subject is a human subject.

In all the methods provided herein and specifically those described in the Sections 5.2.1 and 5.2.2: the therapeutic agents including ADCs that can be used are described in Sections 3, 5.2, 5.3, 5.4, 5.5, and 6, selection of patients for treatment is described herein and exemplified in Section 5.2 including Sections 5.2.1 and 5.2.2 and Sections 3 and 6, dosing regimens and pharmaceutical composition for administering the therapeutic agent are described in Section 5.4, 5.6, 5.7, and Section 6 below, the biomarkers that can be used for identifying the therapeutic agents, selecting the patients, determining the outcome of these methods, and/or serving as criteria in any way for these methods are described herein and exemplified in Section 5.2 including Sections 5.2.1 and 5.2.2 and Sections 3 and 6, the biomarkers can be determined as described in Section 5.8 or as known in the art, therapeutic outcomes for the methods provided herein are described in this Section (Section 5.2 including Section 5.2.1.4) and Sections 3 and 6, additional therapeutic outcomes for the methods provided herein can be improvement of the biomarkers described herein, for example, those described and exemplified in in Section 5.2 including Sections 5.2.1 and 5.2.2 and Sections 3 and 6, and combination therapies including the ADCs and other therapeutic agents are described in this Section (Section 5.2) and in Section 5.5. Therefore, a person skilled in the art would understand that the methods provided herein include all permutations and combinations of the patients, therapeutic agents, dosing regiments, biomarkers, and therapeutic outcomes as described above and below.

### 5.3 Antibody Drug Conjugates for the Methods

In various embodiments of the methods provided herein, including the methods provided in Section 5.2, the ADC used in the methods comprises or is an anti-191P4D12 ADC described herein and/or in U.S. Pat. No. 8,637,642, which is herein incorporated in its entirety by reference. In some embodiments, the anti-191P4D12 antibody drug conjugate provided for the methods herein comprises an antibody or antigen binding fragment thereof that binds to 191P4D12 as provided herein, including in Sections 3, 5.3.1, and 6, conjugated to one or more units of cytotoxic agents (drug units, or D) as provided herein, including in Sections 3 and 6 and this Section (Section 5.3) with further disclosures in Sections 5.3.2 and 5.3.4. In certain embodiments, the cytotoxic agents (drug units, or D) can be covalently linked directly or via a linker unit (LU) as provided herein, including in Sections 3 and 6 and this Section (Section 5.3) with further disclosures in Section 5.3.3.

In some embodiments, the antibody drug conjugate compound has the following formula:

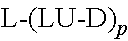

or a pharmaceutically acceptable salt or solvate thereof, wherein:
L is the antibody unit, e.g., the anti-nectin-4 antibody or an antigen binding fragment thereof for example as provided in Sections 3, 5.3.1, and 6, and
(LU-D) is a linker unit-drug unit moiety, wherein:
LU- is a linker unit for example as provided in Sections 3 and 6 and this Section (Section 5.3) with further disclosures in Section 5.3.3, and
D is a drug unit having cytostatic or cytotoxic activity against a target cell for example as provided Sections 3 and 6 and this Section (Section 5.3) with further disclosures in Sections 5.3.2 and 5.3.4; and
p is an integer from 1 to 20 with further examples provided in Sections 3 and 6 and this Section (Section 5.3).

In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20.

In some embodiments, the antibody drug conjugate compound has the following formula:

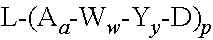

or a pharmaceutically acceptable salt or solvate thereof, wherein:
L is the Antibody unit, e.g., the anti-nectin-4 antibody or an antigen binding fragment thereof for example as provided in Sections 3, 5.3.1, and 6; and
Aₐ-W_{w}—Y_{y}— is a linker unit (LU), wherein:
A- is a stretcher unit,
a is 0 or 1,
each —W— is independently an amino acid unit,
w is an integer ranging from 0 to 12,
—Y— is a self-immolative spacer unit,
y is 0, 1 or 2,
each for example as provided in Sections 3 and 6 and this Section (Section 5.3) with further disclosures in Section 5.3.3;
D is a drug units having cytostatic or cytotoxic activity against the target cell for example as provided Sections 3 and 6 and this Section (Section 5.3) with further disclosures in Sections 5.3.2 and 5.3.4; and
p is an integer from 1 to 20 with further examples provided in Sections 3 and 6 and this Section (Section 5.3).

In some embodiments, a is 0 or 1, w is 0 or 1, and y is 0, 1 or 2. In some embodiments, a is 0 or 1, w is 0 or 1, and y is 0 or 1. In some embodiments, p ranges from 1 to 20, 1 to 19, 1to 18, 1to 17, 1to 16, 1to 15, 1to 14, 1to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2to 11, 2to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20. In some embodiments, when w is not zero, y is 1 or 2. In some embodiments, when w is 1 to 12, y is 1 or 2. In some embodiments, w is 2 to 12 and y is 1 or 2. In some embodiments, a is 1 and w and y are 0.

In some specific embodiments of the methods provided herein, including the methods provided in Section 5.2, the cytotoxic agent as part of any of the ADCs provided herein for the methods comprises, consists of, or is MMAE.

For compositions comprising a plurality antibodies or antigen binding fragments thereof, the drug loading is represented by p, the average number of drug molecules per antibody unit. Drug loading can range from 1 to 20 drugs (D) per antibody. The average number of drugs per antibody in preparation of conjugation reactions can be characterized by conventional means such as mass spectroscopy, ELISA assay, and HPLC. The quantitative distribution of antibody drug conjugates in terms of p can also be determined. In some instances, separation, purification, and characterization of homogeneous antibody drug conjugates where p is a certain value from antibody drug conjugates with other drug loadings can be achieved by means such as reverse phase HPLC or electrophoresis. In certain exemplary embodiments, p is from 2 to 8.

Additional embodiments of the ADC for the methods provided herein have been described in U.S. Pat. No. 8,637,642 and International Application No. PCT/US2019/056214 (Publication No. WO2020/117373), both of which are hereby incorporated in their entireties by reference.

In some embodiments of the methods provided herein, including in Sections 3, 5.2, and 6 and this Section (Section 5.3), the ADC is enfortumab vedotin. In certain embodiments of the methods provided herein, including in Sections 3, 5.2, and 6 and this Section (Section 5.3), the ADC is a biosimilar of enfortumab vedotin.

### 5.3.1 Anti-191P4D12 Antibodies or Antigen Binding Fragments

In one embodiment, the antibody or antigen binding fragment thereof that binds to nectin-4-related proteins is an antibody or antigen binding fragment that specifically binds to nectin-4 protein comprising amino acid sequence of SEQ ID NO:2 (see FIG. 1A). The corresponding cDNA encoding the 191P4D12 protein has a sequence of SEQ ID NO:1 (see FIG. 1A).

The antibody that specifically binds to nectin-4 protein comprising amino acid sequence of SEQ ID NO:2 includes antibodies that can bind to other nectin-4-related proteins. For example, antibodies that bind nectin-4 protein comprising amino acid sequence of SEQ ID NO:2 can bind nectin-4-related proteins such as nectin-4 variants and the homologs or analogs thereof.

In some embodiments, the anti-nectin-4 antibody provided herein is a monoclonal antibody.

In some embodiments, the antibody comprises a heavy chain comprising an amino acid sequence of SEQ ID NO:4 (cDNA sequence of SEQ ID NO:3), and/or a light chain comprising an amino acid sequence of SEQ ID NO:6 (cDNA sequence of SEQ ID NO:5), as shown in FIGS. 1B and 1C.

In some embodiments, the anti-nectin-4 antibody or antigen binding fragment thereof comprises a heavy chain variable region comprising complementarity determining regions (CDRs) comprising the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs comprising the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). In certain embodiments, the anti-nectin-4 antibody or antigen binding fragment thereof comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO:22 (which is the amino acid sequence ranging from the 20th amino acid (glutamic acid) to the 136th amino acid (serine) of SEQ ID NO:7) and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO:23 (which is the amino acid sequence ranging from the 23rd amino acid (aspartic acid) to the 130th amino acid (arginine) of SEQ ID NO:8). In some embodiments, the anti-nectin-4 antibody or antigen binding fragment thereof comprises a heavy chain variable region comprising complementarity determining regions (CDRs) consisting of the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs consisting of the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). In certain embodiments, the anti-nectin-4 antibody or antigen binding fragment thereof comprises a heavy chain variable region comprising complementarity determining region 1 (CDR-H1), CDR-H2, and CDR-H3 consisting of the amino acid sequences of the corresponding CDR-H1, CDR-H2, and CDR-H3 in the heavy chain variable region sequence set forth in SEQ ID NO:22 (which is the amino acid sequence ranging from the 20th amino acid (glutamic acid) to the 136th amino acid (serine) of SEQ ID NO:7) and a light chain variable region comprising CDR-L1, CDR-L2, and CDR-L3 consisting of the amino acid sequences of the corresponding CDR-L1, CDR-L2, and CDR-L3 in the light chain variable region sequence set forth in SEQ ID NO:23 (which is the amino acid sequence ranging from the 23rd amino acid (aspartic acid) to the 130th amino acid (arginine) of SEQ ID NO:8). SEQ ID NO:22, SEQ ID NO:23, SEQ ID NO:7 and SEQ ID NO:8 are as shown in FIGS. 1D and 1E and listed below:

SEQ ID NO: 22
EVQLVESGGGLVQPGGSLRLSCAASGFTFSSYNMNWVRQAPGKGLEWVSY
ISSSSSTIYYADSVKGRFTISRDNAKNSLSLQMNSLRDEDTAVYYCARAY
YYGMDVWGQGTTVTVSS
SEQ ID NO: 23
DIQMTQSPSSVSASVGDRVTITCRASQGISGWLAWYQQKPGKAPKFLIYA
ASTLQSGVPSRFSGSGSGTDFTLTISSLQPEDFATYYCQQANSFPPTFGG
GTKVEIKR
SEQ ID NO: 7
MELGLCWVFLVAILEGVQCEVQLVESGGGLVQPGGSLRLSCAASGFTFSS
YNMNWVRQAPGKGLEWVSYISSSSSTIYYADSVKGRFTISRDNAKNSLSL
QMNSLRDEDTAVYYCARAYYYGMDVWGQGTTVTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEA
LHNHYTQKSLSLSPGK
SEQ ID NO: 8
MDMRVPAQLLGLLLLWFPGSRCDIQMTQSPSSVSASVGDRVTITCRASQG
ISGWLAWYQQKPGKAPKFLIYAASTLQSGVPSRFSGSGSGTDFTLTISSL
QPEDFATYYCQQANSFPPTFGGGTKVEIKRTVAAPSVFIFPPSDEQLKSG
TASVVCLLNNFYPREAKVQWKVDNALQSGNSQESVTEQDSKDSTYSLSST
LTLSKADYEKHKVYACEVTHQGLSSPVTKSFNRGEC

CDR sequences can be determined according to well-known numbering systems. As described above, CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Pluckthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted in Table 1 above.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Kabat numbering and a light chain variable region comprising CDRs comprising the amino acid sequences of the CDRs of the light chain variable region set forth in SEQ ID NO:23 according to Kabat numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to AbM numbering and a light chain variable region comprising CDRs comprising the amino acid sequences of the CDRs of the light chain variable region set forth in SEQ ID NO:23 according to AbM numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Chothia numbering and a light chain variable region comprising CDRs comprising the amino acid sequences of the CDRs of the light chain variable region set forth in SEQ ID NO:23 according to Chothia numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Contact numbering and a light chain variable region comprising CDRs comprising the amino acid sequences of the CDRs of the light chain variable region set forth in SEQ ID NO:23 according to Contact numbering.

In yet other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to IMGT numbering and a light chain variable region comprising CDRs comprising the amino acid sequences of the CDRs of the light chain variable region set forth in SEQ ID NO:23 according to IMGT numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Kabat numbering and a light chain variable region comprising CDRs consisting of the amino acid sequences of the CDRs of the light chain variable region set forth in SEQ ID NO:23 according to Kabat numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to AbM numbering and a light chain variable region comprising CDRs consisting of the amino acid sequences of the CDRs of the light chain variable region set forth in SEQ ID NO:23 according to AbM numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Chothia numbering and a light chain variable region comprising CDRs consisting of the amino acid sequences of the CDRs of the light chain variable region set forth in SEQ ID NO:23 according to Chothia numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Contact numbering and a light chain variable region comprising CDRs consisting of the amino acid sequences of the CDRs of the light chain variable region set forth in SEQ ID NO:23 according to Contact numbering.

In yet other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to IMGT numbering and a light chain variable region comprising CDRs consisting of the amino acid sequences of the CDRs of the light chain variable region set forth in SEQ ID NO:23 according to IMGT numbering.

As described above, the CDR sequences according to different numbering systems can be readily determined, e.g., using online tools such as the one provided by Antigen receptor Numbering And Receptor ClassificatIon (ANARCI). For example, the heavy chain CDR sequences within SEQ ID NO:22, and the light chain CDR sequences within SEQ ID NO:23 according to Kabat numbering as determined by ANARCI are listed in Table 6 below.

TABLE 6

| | VH of SEQ ID NO: 22 | VL of SEQ ID NO: 23 |
|---|---|---|
| CDR1 | SYNMN (SEQ ID NO: | RASQGISGWLA (SEQ ID NO: |
| | 9) | 12) |
| CDR2 | YISSSSSTIYYADSVKG | AASTLQS (SEQ ID NO: 13) |
| | (SEQ ID NO: 10) | |
| CDR3 | AYYYGMDV (SEQ ID | QQANSFPPT (SEQ ID NO: |
| | NO: 11) | 14) |

For another example, the heavy chain CDR sequences within SEQ ID NO:22, and the light chain CDR sequences within SEQ ID NO:23 according to IMGT numbering as determined by ANARCI are listed in Table 7 below.

TABLE 7

| | VH of SEQ ID NO: 22 | VL of SEQ ID NO: 23 |
|---|---|---|
| CDR1 | GFTFSSYN | QGISGW |
| | (SEQ ID NO: 16) | (SEQ ID NO: 19) |
| CDR2 | ISSSSSTI | AAS (SEQ ID NO: 20) |
| | (SEQ ID NO: 17) | |
| CDR3 | ARAYYYGMDV | QQANSFPPT |
| | (SEQ ID NO: 18) | (SEQ ID NO: 21) |

In some embodiments, the antibody or antigen binding fragment thereof comprises CDR-H1 comprising an amino acid sequence of SEQ ID NO:9, CDR-H2 comprising an amino acid sequence of SEQ ID NO:10, CDR-H3 comprising an amino acid sequence of SEQ ID NO:11, CDR-L1 comprising an amino acid sequence of SEQ ID NO:12, CDR-L2 comprising an amino acid sequence of SEQ ID NO:13, and CDR-L3 comprising an amino acid sequence of SEQ ID NO:14.

In some embodiments, the antibody or antigen binding fragment thereof comprises CDR-H1 comprising an amino acid sequence of SEQ ID NO:16, CDR-H2 comprising an amino acid sequence of SEQ ID NO:17, CDR-H3 comprising an amino acid sequence of SEQ ID NO:18, CDR-L1 comprising an amino acid sequence of SEQ ID NO:19, CDR-L2 comprising an amino acid sequence of SEQ ID NO:20, and CDR-L3 comprising an amino acid sequence of SEQ ID NO:21.

In some embodiments, the antibody or antigen binding fragment thereof comprises CDR-H1 consisting of an amino acid sequence of SEQ ID NO:9, CDR-H2 consisting of an amino acid sequence of SEQ ID NO:10, CDR-H3 consisting of an amino acid sequence of SEQ ID NO:11, CDR-L1 consisting of an amino acid sequence of SEQ ID NO:12, CDR-L2 consisting of an amino acid sequence of SEQ ID NO:13, and CDR-L3 consisting of an amino acid sequence of SEQ ID NO:14.

In some embodiments, the antibody or antigen binding fragment thereof comprises CDR-H1 consisting of an amino acid sequence of SEQ ID NO:16, CDR-H2 consisting of an amino acid sequence of SEQ ID NO:17, CDR-H3 consisting of an amino acid sequence of SEQ ID NO:18, CDR-L1 consisting of an amino acid sequence of SEQ ID NO:19, CDR-L2 consisting of an amino acid sequence of SEQ ID NO:20, and CDR-L3 consisting of an amino acid sequence of SEQ ID NO:21.

In some embodiments, the antibody or antigen binding fragment thereof comprises a heavy chain variable region comprising the amino acid sequence of SEQ ID NO:22 and a light chain variable region comprising the amino acid sequence of SEQ ID NO:23.

In some embodiments, the antibody or antigen binding fragment thereof comprises a heavy chain variable region consisting of the amino acid sequence of SEQ ID NO:22 and a light chain variable region consisting of the amino acid sequence of SEQ ID NO:23.

In some embodiments, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

In some embodiments, the antibody comprises a heavy chain consisting of the amino acid sequence ranging from the 20th amino acid (glutamic acid) to the 466th amino acid (lysine) of SEQ ID NO:7 and a light chain consisting of the amino acid sequence ranging from the 23rd amino acid (aspartic acid) to the 236th amino acid (cysteine) of SEQ ID NO:8.

In some embodiments, amino acid sequence modification(s) of antibodies described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art who appreciate that amino acid changes can alter post-translational processes of the antibody, such as changing the number or position of glycosylation sites or altering the membrane anchoring characteristics.

In some embodiments, the antibodies provided herein are chemically modified, for example, by the covalent attachment of any type of molecule to the antibody. The antibody derivatives can include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formulation, metabolic synthesis of tunicamycin, etc. Additionally, the antibody can contain one or more non-classical amino acids.

Variations can be a substitution, deletion, or insertion of one or more codons encoding the single domain antibody or polypeptide that results in a change in the amino acid sequence as compared with the original antibody or polypeptide. Amino acid substitutions can be the result of replacing one amino acid with another amino acid comprising similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions can optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed can be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue comprising a side chain with a similar charge. As described above, families of amino acid residues comprising side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined conservative (e.g., within an amino acid group with similar properties and/or side chains) substitutions can be made, so as to maintain or not significantly change the properties.

Amino acids can be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the antibody also can be substituted, for example, with another amino acid, such as alanine or serine, to improve the oxidative stability of the molecule and to prevent aberrant crosslinking.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the anti-anti-MSLN antibody variant DNA.

Covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

In certain embodiments, the antibody or antigen binding fragment provided herein comprises a heavy chain having certain homology or identity to the heavy chain as set forth in SEQ ID NO:7 and a light chain having certain homology or identity to the light chain as set forth in SEQ ID NO:8. Such embodiments of heavy/light chains with homology or identity are further provided as follows. In some embodiments, the antibody or antigen binding fragment provided herein comprises a heavy chain having more than 70% homology or identity to the heavy chain as set forth in SEQ ID NO:7. In some embodiments, the antibody or antigen binding fragment provided herein comprises a heavy chain having more than 75% homology or identity to the heavy chain as set forth in SEQ ID NO:7. In some embodiments, the antibody or antigen binding fragment provided herein comprises a heavy chain having more than 80% homology or identity to the heavy chain as set forth in SEQ ID NO:7. In some embodiments, the antibody or antigen binding fragment provided herein comprises a heavy chain having more than 85% homology or identity to the heavy chain as set forth in SEQ ID NO:7. In some embodiments, the antibody or antigen binding fragment provided herein comprises a heavy chain having more than 90% homology or identity to the heavy chain as set forth in SEQ ID NO:7. In some embodiments, the antibody or antigen binding fragment provided herein comprises a heavy chain having more than 95% homology or identity to the heavy chain as set forth in SEQ ID NO:7. In certain embodiments, the antibody or antigen binding fragment provided herein comprises a heavy chain having any of the provided homology or identity to the heavy chain as set forth in SEQ ID NO:7, wherein the CDRs (CDR-H1, CDR-H2, and CDR-H3) are identical to the CDRs in the heavy chain as set forth in SEQ ID NO:7. In some embodiments, the antibody or antigen binding fragment provided herein comprises a light chain having more than 70% homology or identity to the light chain as set forth in SEQ ID NO:8. In some embodiments, the antibody or antigen binding fragment provided herein comprises a light chain having more than 75% homology or identity to the light chain as set forth in SEQ ID NO:8. In some embodiments, the antibody or antigen binding fragment provided herein comprises a light chain having more than 80% homology or identity to the light chain as set forth in SEQ ID NO:8. In some embodiments, the antibody or antigen binding fragment provided herein comprises a light chain having more than 85% homology or identity to the light chain as set forth in SEQ ID NO:8. In some embodiments, the antibody or antigen binding fragment provided herein comprises a light chain having more than 90% homology or identity to the light chain as set forth in SEQ ID NO:8. In some embodiments, the antibody or antigen binding fragment provided herein comprises a light chain having more than 95% homology or identity to the light chain as set forth in SEQ ID NO:8. In certain embodiments, the antibody or antigen binding fragment provided herein comprises a light chain having any of the provided homology or identity to the light chain as set forth in SEQ ID NO:8, wherein the CDRs (CDR-L1, CDR-L2, and CDR-L3) are identical to the CDRs in the light chain as set forth in SEQ ID NO:8. In certain embodiments, the antibody or antigen binding fragment provided herein comprises any homologous light chain and any homologous heavy chain as provided in this paragraph in any combination or permutation.

In certain embodiments, the antibody or antigen binding fragment provided herein comprises a heavy chain variable region having certain homology or identity to the heavy chain variable region as set forth in SEQ ID NO:22 and a light chain variable region having certain homology or identity to the light chain variable region as set forth in SEQ ID NO:23. Such embodiments of heavy chain variable regions and light chain variable regions with homology or identity are further provided as follows. In some embodiments, the antibody or antigen binding fragment provided herein comprises a heavy chain variable region having more than 70% homology or identity to the heavy chain variable region as set forth in SEQ ID NO:22. In some embodiments, the antibody or antigen binding fragment provided herein comprises a heavy chain variable region having more than 75% homology or identity to the heavy chain variable region as set forth in SEQ ID NO:22. In some embodiments, the antibody or antigen binding fragment provided herein comprises a heavy chain variable region having more than 80% homology or identity to the heavy chain variable region as set forth in SEQ ID NO:22. In some embodiments, the antibody or antigen binding fragment provided herein comprises a heavy chain variable region having more than 85% homology or identity to the heavy chain variable region as set forth in SEQ ID NO:22. In some embodiments, the antibody or antigen binding fragment provided herein comprises a heavy chain variable region having more than 90% homology or identity to the heavy chain variable region as set forth in SEQ ID NO:22. In some embodiments, the antibody or antigen binding fragment provided herein comprises a heavy chain variable region having more than 95% homology or identity to the heavy chain variable region as set forth in SEQ ID NO:22. In certain embodiments, the antibody or antigen binding fragment provided herein comprises a heavy chain variable region having any of the provided homology or identity to the heavy chain variable region as set forth in SEQ ID NO:22, wherein the CDRs (CDR-H1, CDR-H2, and CDR-H3) are identical to the CDRs in the heavy chain variable region as set forth in SEQ ID NO:22. In some embodiments, the antibody or antigen binding fragment provided herein comprises a light chain variable region having more than 70% homology or identity to the light chain variable region as set forth in SEQ ID NO:23. In some embodiments, the antibody or antigen binding fragment provided herein comprises a light chain variable region having more than 75% homology or identity to the light chain variable region as set forth in SEQ ID NO:23. In some embodiments, the antibody or antigen binding fragment provided herein comprises a light chain variable region having more than 80% homology or identity to the light chain variable region as set forth in SEQ ID NO:23. In some embodiments, the antibody or antigen binding fragment provided herein comprises a light chain variable region having more than 85% homology or identity to the light chain variable region as set forth in SEQ ID NO:23. In some embodiments, the antibody or antigen binding fragment provided herein comprises a light chain variable region having more than 90% homology or identity to the light chain variable region as set forth in SEQ ID NO:23. In some embodiments, the antibody or antigen binding fragment provided herein comprises a light chain variable region having more than 95% homology or identity to the light chain variable region as set forth in SEQ ID NO:23. In certain embodiments, the antibody or antigen binding fragment provided herein comprises a light chain variable region having any of the provided homology or identity to the light chain variable region as set forth in SEQ ID NO:23, wherein the CDRs (CDR-L1, CDR-L2, and CDR-L3) are identical to the CDRs in the light chain variable region as set forth in SEQ ID NO:23. In certain embodiments, the antibody or antigen binding fragment provided herein comprises any homologous light chain variable region and any homologous heavy chain variable region as provided in this paragraph in any combination or permutation.

In some embodiments, the anti-nectin-4 antibody provided herein comprises heavy and light chain CDR regions of an antibody designated Ha22-2(2,4)6.1 produced by a hybridoma deposited under the American Type Culture Collection (ATCC) Accession NO: PTA-11267, or heavy and light chain CDR regions comprising amino acid sequences that are homologous to the amino acid sequences of the heavy and light chain CDR regions of Ha22-2(2,4)6.1, and wherein the antibodies retain the desired functional properties of the anti-nectin-4 antibody designated Ha22-2(2,4)6.1 produced by a hybridoma deposited under the American Type Culture Collection (ATCC) Accession NO: PTA-11267.

In some embodiments, the anti-nectin-4 antibody provided herein comprises heavy and light chain CDR regions (CDR-H1, CDR-H2, CDR-H3, CDR-L1, CDR-L2, and CDR-L3) of an antibody designated Ha22-2(2,4)6.1 produced by a hybridoma deposited under the American Type Culture Collection (ATCC) Accession NO: PTA-11267, or heavy and light chain CDR regions consisting of amino acid sequences that are homologous to the amino acid sequences of the heavy and light chain CDR regions of Ha22-2(2,4)6.1, and wherein the antibodies retain the desired functional properties of the anti-nectin-4 antibody designated Ha22-2(2,4)6.1 produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:
(a) the heavy chain variable region comprises CDRs (CDR-H1, CDR-H2, and CDR-H3) comprising the amino acid sequences of the heavy chain variable region CDRs set forth in the antibody produced by a hybridoma deposited under the American Type Culture Collection (ATCC) Accession NO: PTA-11267;
(b) the light chain variable region comprises CDRs (CDR-L1, CDR-L2, and CDR-L3) comprising the amino acid sequences of the light chain variable region CDRs set forth in the antibody produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:
(a) the heavy chain variable region comprises CDRs (CDR-H1, CDR-H2, and CDR-H3) consisting of the amino acid sequences of the heavy chain variable region CDRs set forth in the antibody produced by a hybridoma deposited under the American Type Culture Collection (ATCC) Accession NO: PTA-11267;
(b) the light chain variable region comprises CDRs (CDR-L1, CDR-L2, and CDR-L3) consisting of the amino acid sequences of the light chain variable region CDRs set forth in the antibody produced by a hybridoma deposited under the American Type Culture Collection (ATCC) Accession NO: PTA-11267.

In some embodiments, the anti-nectin-4 antibody provided herein comprises heavy and light chain variable regions of an antibody designated Ha22-2(2,4)6.1 produced by a hybridoma deposited under the American Type Culture Collection (ATCC) Accession NO: PTA-11267, or heavy and light variable regions comprising amino acid sequences that are homologous to the amino acid sequences of the heavy and light chain variable regions of Ha22-2(2,4)6.1, and wherein the antibodies retain the desired functional properties of the anti-nectin-4 antibody provided herein. In some embodiments, the anti-nectin-4 antibody provided herein comprises heavy and light chain variable regions of an antibody designated Ha22-2(2,4)6.1 produced by a hybridoma deposited under the American Type Culture Collection (ATCC) Accession NO: PTA-11267, or heavy and light variable regions consisting of amino acid sequences that are homologous to the amino acid sequences of the heavy and light chain variable regions of Ha22-2(2,4)6.1, and wherein the antibodies retain the desired functional properties of the anti-nectin-4 antibody provided herein. As the constant region of the antibody of the disclosure, any subclass of constant region can be chosen. In one embodiment, human IgG1 constant region as the heavy chain constant region and human Ig kappa constant region as the light chain constant region can be used.

In some embodiments, the anti-nectin-4 antibody provided herein comprises heavy and light chains of an antibody designated Ha22-2(2,4)6.1 produced by a hybridoma deposited under the American Type Culture Collection (ATCC) Accession NO: PTA-11267, or heavy and light chains comprising amino acid sequences that are homologous to the amino acid sequences of the heavy and light chains of Ha22-2(2,4)6.1, and wherein the antibodies retain the desired functional properties of the anti-nectin-4 antibody provided herein. In some embodiments, the anti-nectin-4 antibody provided herein comprises heavy and light chains of an antibody designated Ha22-2(2,4)6.1 produced by a hybridoma deposited under the American Type Culture Collection (ATCC) Accession NO: PTA-11267, or heavy and light chains consisting of amino acid sequences that are homologous to the amino acid sequences of the heavy and light chains of Ha22-2(2,4)6.1, and wherein the antibodies retain the desired functional properties of the anti-nectin-4 antibody provided herein.

In some embodiments, the antibody or antigen binding fragment thereof provided herein comprises a heavy chain variable region and a light chain variable region, wherein:
(a) the heavy chain variable region comprises an amino acid sequence that is at least 80% homologous or identical to the heavy chain variable region amino acid sequence of the antibody produced by a hybridoma deposited under the American Type Culture Collection (ATCC) Accession NO: PTA-11267; and
(b) the light chain variable region comprises an amino acid sequence that is at least 80% homologous or identical to the light chain variable region amino acid sequence of the antibody produced by a hybridoma deposited under the American Type Culture Collection (ATCC) Accession NO: PTA-11267.

In certain embodiments, the antibody or antigen binding fragment provided herein comprises a heavy chain variable region having certain homology or identity to the heavy chain variable region amino acid sequence of the antibody produced by a hybridoma deposited under the American Type Culture Collection (ATCC) Accession NO: PTA-11267 and a light chain variable region having certain homology or identity to the light chain variable region amino acid sequence of the antibody produced by a hybridoma deposited under the American Type Culture Collection (ATCC) Accession NO: PTA-11267. Such embodiments of heavy chain variable regions and light chain variable regions with homology or identity are further provided as follows. In some embodiments, the heavy chain variable region comprises an amino acid sequence that is at least 85% homologous or identical to the heavy chain variable region amino acid sequence of the antibody produced by a hybridoma deposited under the American Type Culture Collection (ATCC) Accession NO: PTA-11267. In other embodiments, the heavy chain variable region comprises an amino acid sequence that is at least 90% homologous or identical to the heavy chain variable region amino acid sequence of the antibody produced by a hybridoma deposited under the American Type Culture Collection (ATCC) Accession NO: PTA-11267. In yet other embodiments, the heavy chain variable region comprises an amino acid sequence that is at least 95% homologous or identical to the heavy chain variable region amino acid sequence of the antibody produced by a hybridoma deposited under the American Type Culture Collection (ATCC) Accession NO: PTA-11267. In other embodiments, the heavy chain variable region can be 85%, 86%, 87%, 88%, 89%, 90%, 91%, 92%, 93%, 94%, 95%, 96%, 97%, 98% or 99% homologous or identical to the heavy chain variable region amino acid sequence of the antibody produced by a hybridoma deposited under the American Type Culture Collection (ATCC) Accession NO: PTA-11267. In some embodiments, the light chain variable region comprises an amino acid sequence that is at least 85% homologous or identical to the light chain variable region amino acid sequence of the antibody produced by a hybridoma deposited under the American Type Culture Collection (ATCC) Accession NO: PTA-11267. In other embodiments, the light chain variable region comprises an amino acid sequence that is at least 90% homologous or identical to the light chain variable region amino acid sequence of the antibody produced by a hybridoma deposited under the American Type Culture Collection (ATCC) Accession NO: PTA-11267. In yet other embodiments, the light chain variable region comprises an amino acid sequence that is at least 95% homologous or identical to the light chain variable region amino acid sequence of the antibody produced by a hybridoma deposited under the American Type Culture Collection (ATCC) Accession NO: PTA-11267. In other embodiments, the light chain variable region can be 85%, 86%, 87%, 88%, 89%, 90%, 91%, 92%, 93%, 94%, 95%, 96%, 97%, 98% or 99% homologous or identical to the light chain variable region amino acid sequence of the antibody produced by a hybridoma deposited under the American Type Culture Collection (ATCC) Accession NO: PTA-11267. In certain embodiments, the antibody or antigen binding fragment provided herein comprises any homologous light chain variable region and any homologous heavy chain variable region as provided in this paragraph in any combination or permutation.

In other embodiments, the antibody or antigen binding fragment thereof provided herein comprises a heavy chain and a light chain, wherein:
(a) the heavy chain comprises an amino acid sequence that is at least 80% homologous or identical to the heavy chain amino acid sequence of the antibody produced by a hybridoma deposited under the American Type Culture Collection (ATCC) Accession NO: PTA-11267; and
(b) the light chain comprises an amino acid sequence that is at least 80% homologous or identical to the light chain amino acid sequence of the antibody produced by a hybridoma deposited under the American Type Culture Collection (ATCC) Accession NO: PTA-11267.

In certain embodiments, the antibody or antigen binding fragment provided herein comprises a heavy chain having certain homology or identity to the heavy chain amino acid sequence of the antibody produced by a hybridoma deposited under the American Type Culture Collection (ATCC) Accession NO: PTA-11267 and a light chain having certain homology or identity to the light chain amino acid sequence of the antibody produced by a hybridoma deposited under the American Type Culture Collection (ATCC) Accession NO: PTA-11267. Such embodiments of heavy chains and light chains with homology or identity are further provided as follows. In some embodiments, the heavy chain comprises an amino acid sequence that is at least 85% homologous or identical to the heavy chain amino acid sequence of the antibody produced by a hybridoma deposited under the American Type Culture Collection (ATCC) Accession NO: PTA-11267. In other embodiments, the heavy chain comprises an amino acid sequence that is at least 90% homologous or identical to the heavy chain amino acid sequence of the antibody produced by a hybridoma deposited under the American Type Culture Collection (ATCC) Accession NO: PTA-11267. In yet other embodiments, the heavy chain comprises an amino acid sequence that is at least 95% homologous or identical to the heavy chain amino acid sequence of the antibody produced by a hybridoma deposited under the American Type Culture Collection (ATCC) Accession NO: PTA-11267. In other embodiments, the heavy chain can be 85%, 86%, 87%, 88%, 89%, 90°/, 91%, 92%, 93%, 94%, 95%, 96%, 97%, 98% or 99% homologous or identical to the heavy chain amino acid sequence of the antibody produced by a hybridoma deposited under the American Type Culture Collection (ATCC) Accession NO: PTA-11267. In some embodiments, the light chain comprises an amino acid sequence that is at least 85% homologous or identical to the light chain amino acid sequence of the antibody produced by a hybridoma deposited under the American Type Culture Collection (ATCC) Accession NO: PTA-11267. In other embodiments, the light chain comprises an amino acid sequence that is at least 90% homologous or identical to the light chain amino acid sequence of the antibody produced by a hybridoma deposited under the American Type Culture Collection (ATCC) Accession NO: PTA-11267. In yet other embodiments, the light chain comprises an amino acid sequence that is at least 95% homologous or identical to the light chain amino acid sequence of the antibody produced by a hybridoma deposited under the American Type Culture Collection (ATCC) Accession NO: PTA-11267. In other embodiments, the light chain can be 85%, 86%, 87%, 88%, 89%, 90%, 91%, 92%, 93%, 94%, 95%, 96%, 97%, 98% or 99% homologous or identical to the light chain amino acid sequence of the antibody produced by a hybridoma deposited under the American Type Culture Collection (ATCC) Accession NO: PTA-11267. In certain embodiments, the antibody or antigen binding fragment provided herein comprises any homologous light chain and any homologous heavy chain as provided in this paragraph in any combination or permutation.

In some embodiments, the antibody or antigen binding fragment thereof provided herein binds to a specific epitope in 191P4D12. In some embodiments, the antibody or antigen binding fragment thereof provided herein binds to VC1 domain of 191P4D12. In some embodiments, the antibody or antigen binding fragment thereof provided herein binds to VC1 domain but not to C1C2 domain of 191P4D12. In some embodiments, the antibody or antigen binding fragment thereof provided herein binds to the 1st to 147th amino acid residues of 191P4D12. In some embodiments, the antibody or antigen binding fragment thereof provided herein binds to an epitope located in the 1st to 147th amino acid residues of 191P4D12. In some embodiments, the antibody or antigen binding fragment thereof provided herein binds to the 1st to 10th amino acid residues of 191P4D12. In some embodiments, the antibody or antigen binding fragment thereof provided herein binds to the 11th to 20th amino acid residues of 191P4D12. In some embodiments, the antibody or antigen binding fragment thereof provided herein binds to the 21st to 30th amino acid residues of 191P4D12. In some embodiments, the antibody or antigen binding fragment thereof provided herein binds to the 31st to 40th amino acid residues of 191P4D12. In some embodiments, the antibody or antigen binding fragment thereof provided herein binds to the 41st to 50th amino acid residues of 191P4D12. In some embodiments, the antibody or antigen binding fragment thereof provided herein binds to the 51st to 60th amino acid residues of 191P4D12. In some embodiments, the antibody or antigen binding fragment thereof provided herein binds to the 61st to 70th amino acid residues of 191P4D12. In some embodiments, the antibody or antigen binding fragment thereof provided herein binds to the 71st to 80th amino acid residues of 191P4D12. In some embodiments, the antibody or antigen binding fragment thereof provided herein binds to the 81st to 90th amino acid residues of 191P4D12. In some embodiments, the antibody or antigen binding fragment thereof provided herein binds to the 91st to 100th amino acid residues of 191P4D12. In some embodiments, the antibody or antigen binding fragment thereof provided herein binds to the 101st to 110th amino acid residues of 191P4D12. In some embodiments, the antibody or antigen binding fragment thereof provided herein binds to the 111th to 120th amino acid residues of 191P4D12. In some embodiments, the antibody or antigen binding fragment thereof provided herein binds to the 121st to 130th amino acid residues of 191P4D12. In some embodiments, the antibody or antigen binding fragment thereof provided herein binds to the 131st to 140th amino acid residues of 191P4D12. In some embodiments, the antibody or antigen binding fragment thereof provided herein binds to the 141st to 147th amino acid residues of 191P4D12. The binding epitopes of certain embodiments the antibodies or antigen binding fragments thereof provided herein have been determined and described in WO 2012/047724, which is incorporated herein in its entirety by reference.

In some embodiments, the antibody or antigen binding fragment thereof provided herein binds to epitopes in 191P4D12 that are common between the 191P4D12 variants observed in human. In some embodiments, the antibody or antigen binding fragment thereof provided herein binds to epitopes in 191P4D12 that are common between the 191P4D12 polymorphism observed in human. In some embodiments, the antibody or antigen binding fragment thereof provided herein binds to epitopes in 191P4D12 that are common between the 191P4D12 polymorphism observed in human cancers. In some embodiments, the antibody or antigen binding fragment thereof provided herein binds to epitopes in 191P4D12 that would bind, internalize, disrupt or modulate the biological function of 191P4D12 or 191P4D12 variants. In some embodiments, the antibody or antigen binding fragment thereof provided herein binds to epitopes in 191P4D12 that would disrupt the interaction between 191P4D12 with ligands, substrates, and binding partners.

Engineered antibodies provided herein include those in which modifications have been made to framework residues within VH and/or VL (e.g. to improve the properties of the antibody). Typically, such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis (e.g., “backmutated” from leucine to methionine). Such “backmutated” antibodies are also intended to be encompassed by the disclosure.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr et al.

In addition or alternative to modifications made within the framework or CDR regions, antibodies of the disclosure can be engineered to include modifications within the Fc region, typically to alter one or more functional properties of the antibody, such as serum half-life, complement fixation, Fc receptor binding, and/or antigen-dependent cellular cytotoxicity. Furthermore, an anti-191P4D12 antibody provided herein can be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CHI is modified such that the number of cysteine residues in the hinge region is altered, e.g., increased or decreased. This approach is described further in U.S. Pat. No. 5,677,425 by Bodmer et al. The number of cysteine residues in the hinge region of CHI is altered to, for example, facilitate assembly of the light and heavy chains or to increase or decrease the stability of the anti-191P4D12 antibody.

In another embodiment, the Fc hinge region of an antibody is mutated to decrease the biological half-life of the anti-191P4D12 antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the anti-191P4D12 antibody is modified to increase its biological half-life. Various approaches are possible. For example, mutations can be introduced as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid specific residues can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

Reactivity of the anti-191P4D12 antibodies with a 191P4D12-related protein can be established by a number of well-known means, including Western blot, immunoprecipitation, ELISA, and FACS analyses using, as appropriate, 191P4D12-related proteins, 191P4D12-expressing cells or extracts thereof. A 191P4D12 antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more 191P4D12 epitopes are generated using methods generally known in the art. Homodimeric antibodies can also be generated by cross-linking techniques known in the art (e.g., Wolff et al., Cancer Res. 53: 2560-2565).

In yet another specific embodiment, the anti-191P4D12 antibody provided herein is an antibody comprising heavy and light chain of an antibody designated Ha22-2(2,4)6.1. The heavy chain of Ha22-2(2,4)6.1 consists of the amino acid sequence ranging from 20m E residue to the 466^{th} K residue of SEQ ID NO:7 and the light chain of Ha22-2(2,4)6.1 consists of amino acid sequence ranging from 23^{rd} D residue to the 236^{th} residue of SEQ ID NO:8 sequence.

The hybridoma producing the antibody designated Ha22-2(2,4)6.1 was sent (via Federal Express) to the American Type Culture Collection (ATCC), P.O. Box 1549, Manassas, VA 20108 on 18 Aug. 2010 and assigned Accession number PTA-11267.

Additional embodiments of anti-nectin-4 antibody have been described in U.S. Pat. No. 8,637,642 and International Application No. PCT/US2019/056214 (Publication No. WO2020/117373), both of which are hereby incorporated in their entireties by reference.

### 5.3.2 Cytotoxic Agents (Drug Units)

As the ADC used in the methods provided herein comprises an antibody or antigen binding fragment thereof conjugated to a cytotoxic agent, the disclosure further provides various embodiments for the cytotoxic agent as part of the ADC for use in the methods. In various embodiments of the methods provided herein, including the methods provided in Section 5.2, the cytotoxic agent as part of any of the ADCs provided herein for the methods comprises, consists of, or is a tubulin disrupting agent. In one embodiment, the cytotoxic agent is a tubulindisrupting agent. In some embodiments, the tubulin disrupting agent is selected from the group consisting of a dolastatin, an auristatin, a hemiasterlin, a vinca alkaloid, a maytansinoid, an eribulin, a colchicine, a plocabulin, a phomopsin, an epothilone, a cryptophycin, and a taxane. In one specific embodiment, the tubulin disrupting agent is an auristatin. In a further specific embodiment, the auristatin is monomethyl auristatin E (MMAE), monomethyl auristatin F (MMAF), AFP, or auristain T. In yet another specific embodiment, the auristatin is monomethyl auristatin E (MMAE).

In various embodiments of the methods provided herein, including the methods provided in Section 5.2, the cytotoxic agent as part of any of the ADCs provided herein for the methods comprises, consists of, or is any agent selected from the cytotoxic agents described in U.S. Pat. No. 8,637,642 and International Application No. PCT/US2019/056214 (Publication No. WO2020/117373), both of which are hereby incorporated in their entireties by reference

In some embodiments, the auristatin is MMAE (wherein the wavy line indicates the covalent attachment to a linker of an antibody drug conjugate).

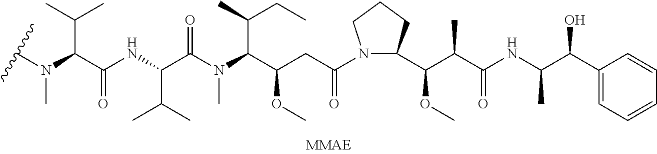 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, an exemplary embodiment comprising MMAE and a linker component (described further herein) has the following structure (wherein L presents the antibody (e.g. anti-nectin-4 antibody or antigen binding fragment thereof) and p ranges from 1 to 12):

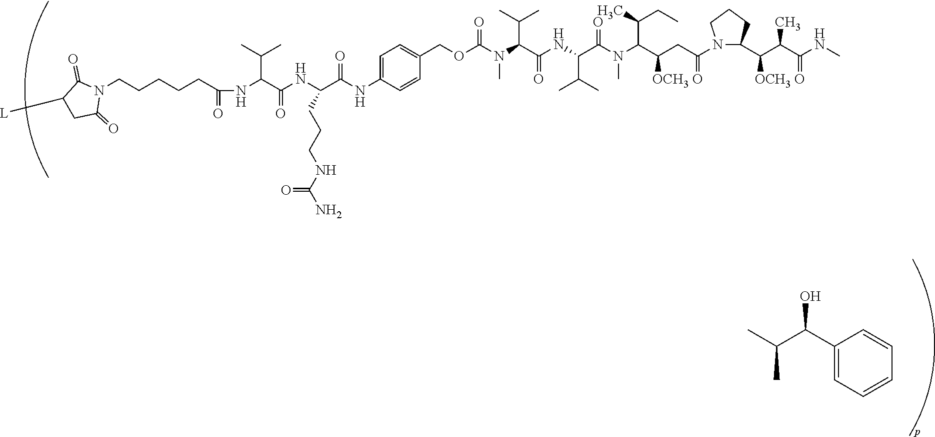

In some embodiments of the formula described in the preceding paragraph, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments of the formula described in the preceding paragraph, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments of the formula described in the preceding paragraph, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments of the formula described in the preceding paragraph, p is about 1. In some embodiments of the formula described in the preceding paragraph, p is about 2. In some embodiments of the formula described in the preceding paragraph, p is about 3. In some embodiments of the formula described in the preceding paragraph, p is about 4. In some embodiments of the formula described in the preceding paragraph, p is about 3.8. In some embodiments of the formula described in the preceding paragraph, p is about 5. In some embodiments of the formula described in the preceding paragraph, p is about 6. In some embodiments of the formula described in the preceding paragraph, p is about 7. In some embodiments of the formula described in the preceding paragraph, p is about 8. In some embodiments of the formula described in the preceding paragraph, p is about 9. In some embodiments of the formula described in the preceding paragraph, p is about 10. In some embodiments of the formula described in the preceding paragraph, p is about 11. In some embodiments of the formula described in the preceding paragraph, p is about 12. In some embodiments of the formula described in the preceding paragraph, p is about 13. In some embodiments of the formula described in the preceding paragraph, p is about 14. In some embodiments of the formula described in the preceding paragraph, p is about 15. In some embodiments of the formula described in the preceding paragraph, p is about 16. In some embodiments of the formula described in the preceding paragraph, p is about 17. In some embodiments of the formula described in the preceding paragraph, p is about 18. In some embodiments of the formula described in the preceding paragraph, p is about 19. In some embodiments of the formula described in the preceding paragraph, p is about 20.

Typically, peptide-based drug units can be prepared by forming a peptide bond between two or more amino acids and/or peptide fragments. Such peptide bonds can be prepared, for example, according to the liquid phase synthesis method (see E. Schroder and K. Lubke, “The Peptides”, volume 1, pp 76-136, 1965, Academic Press) that is well-known in the field of peptide chemistry. The auristatin/dolastatin drug units can be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat Biotechnol 21(7):778-784.

Additional embodiments of cytotoxic agent have been described in U.S. Pat. No. 8,637,642 and International Application No. PCT/US2019/056214 (Publication No. WO2020/117373), both of which are hereby incorporated in their entireties by reference.

### 5.3.3 Linkers

Typically, the antibody drug conjugates comprise a linker unit between the drug unit (e.g., MMAE) and the antibody unit (e.g., the anti-191P4D12 antibody or antigen binding fragment thereof). In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO:15)). In some embodiments, the peptidyl linker is at least two amino acids long or at least three amino acids long. In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT.

A “linker unit” (LU) is a bifunctional compound that can be used to link a drug unit and an antibody unit to form an antibody drug conjugate. In some embodiments, the linker unit has the formula:

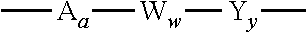

wherein: -A- is a stretcher unit,
a is 0 or 1,
each —W— is independently an amino acid unit,
w is an integer ranging from 0 to 12,
—Y— is a self-immolative spacer unit, and
y is 0, 1 or 2.

In some embodiments, a is 0 or 1, w is 0 or 1, and y is 0, 1 or 2. In some embodiments, a is 0 or 1, w is 0 or 1, and y is 0 or 1. In some embodiments, when w is 1 to 12, y is 1 or 2. In some embodiments, w is 2 to 12 and y is 1 or 2. In some embodiments, a is 1 and w and y are 0. The linker and each of the stretcher unit, the amino acid unit, and the spacer unit have been described in U.S. Pat. No. 8,637,642 and International Application No. PCT/US2019/056214 (Publication No. WO2020/117373), both of which are hereby incorporated in their entireties by reference.

Embodiments of the antibody-drug conjugates can include:

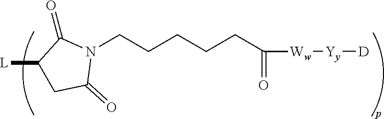

wherein w and y are each 0, 1 or 2, and,

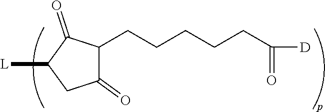

wherein w and y are each 0,

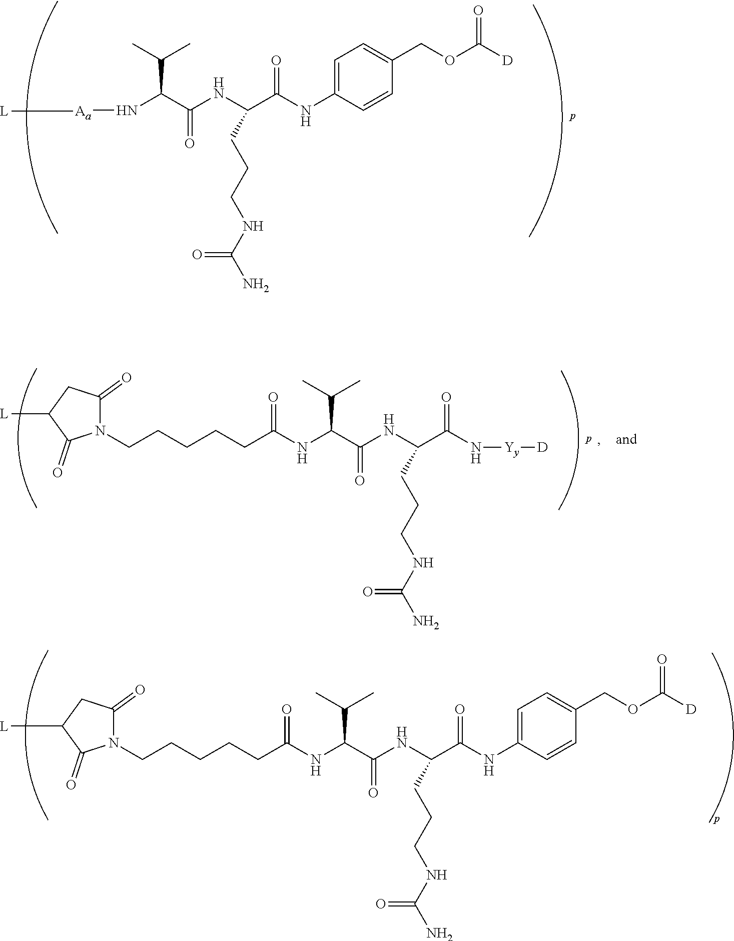

### 5.3.4 Drue Loading

Drug loading is represented by p and is the average number of drug units per antibody in a molecule. Drug loading can range from 1 to 20 drug units (D) per antibody. The ADCs provided herein include collections of antibodies or antigen binding fragments conjugated with a range of drug units, e.g., from 1 to 20. The average number of drug units per antibody in preparations of ADC from conjugation reactions can be characterized by conventional means such as mass spectroscopy and, ELISA assay. The quantitative distribution of ADC in terms of p can also be determined. In some instances, separation, purification, and characterization of homogeneous ADC where p is a certain value from ADC with other drug loadings can be achieved by means such as electrophoresis.

In certain embodiments, the drug loading for an ADC provided herein ranges from 1 to 20. In certain embodiments, the drug loading for an ADC provided herein ranges from 1 to 18. In certain embodiments, the drug loading for an ADC provided herein ranges from 1 to 15. In certain embodiments, the drug loading for an ADC provided herein ranges from 1 to 12. In certain embodiments, the drug loading for an ADC provided herein ranges from 1 to 10. In certain embodiments, the drug loading for an ADC provided herein ranges from 1 to 9. In certain embodiments, the drug loading for an ADC provided herein ranges from 1 to 8. In certain embodiments, the drug loading for an ADC provided herein ranges from 1 to 7. In certain embodiments, the drug loading for an ADC provided herein ranges from 1 to 6. In certain embodiments, the drug loading for an ADC provided herein ranges from 1 to 5. In certain embodiments, the drug loading for an ADC provided herein ranges from 1 to 4. In certain embodiments, the drug loading for an ADC provided herein ranges from 1 to 3. In certain embodiments, the drug loading for an ADC provided herein ranges from 2 to 12. In certain embodiments, the drug loading for an ADC provided herein ranges from 2 to 10. In certain embodiments, the drug loading for an ADC provided herein ranges from 2 to 9. In certain embodiments, the drug loading for an ADC provided herein ranges from 2 to 8. In certain embodiments, the drug loading for an ADC provided herein ranges from 2 to 7. In certain embodiments, the drug loading for an ADC provided herein ranges from 2 to 6. In certain embodiments, the drug loading for an ADC provided herein ranges from 2 to 5. In certain embodiments, the drug loading for an ADC provided herein ranges from 2 to 4. In certain embodiments, the drug loading for an ADC provided herein ranges from 3 to 12. In certain embodiments, the drug loading for an ADC provided herein ranges from 3 to 10. In certain embodiments, the drug loading for an ADC provided herein ranges from 3 to 9. In certain embodiments, the drug loading for an ADC provided herein ranges from 3 to 8. In certain embodiments, the drug loading for an ADC provided herein ranges from 3 to 7. In certain embodiments, the drug loading for an ADC provided herein ranges from 3 to 6. In certain embodiments, the drug loading for an ADC provided herein ranges from 3 to 5. In certain embodiments, the drug loading for an ADC provided herein ranges from 3 to 4.

In certain embodiments, the drug loading for an ADC provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, the drug loading for an ADC provided herein is about 1, about 2, about 3, about 4, about 5, about 6, about 7, about 8, about 9, about 10, about 11, about 12, or more. In some embodiments, the drug loading for an ADC provided herein is about 3.1, about 3.2, about 3.3, about 3.4, about 3.5, about 3.6, about 3.7, about 3.8, or about 3.9.

In some embodiments, the drug loading for an ADC provided herein ranges from 2 to 20, 2 to 19, 2 to 18, 2 to 17, 2 to 16, 2 to 15, 2 to 14, or 2 to 13. In some embodiments, the drug loading for an ADC provided herein ranges from 3 to 20, 3 to 19, 3 to 18, 3 to 17, 3 to 16, 3 to 15, 3 to 14, or 3 to 13. In some embodiments, the drug loading for an ADC provided herein is about 1. In some embodiments, the drug loading for an ADC provided herein is about 2. In some embodiments, the drug loading for an ADC provided herein is about 3. In some embodiments, the drug loading for an ADC provided herein is about 4. In some embodiments, the drug loading for an ADC provided herein is about 3.8. In some embodiments, the drug loading for an ADC provided herein is about 5. In some embodiments, the drug loading for an ADC provided herein is about 6. In some embodiments, the drug loading for an ADC provided herein is about 7. In some embodiments, the drug loading for an ADC provided herein is about 8. In some embodiments, the drug loading for an ADC provided herein is about 9. In some embodiments, the drug loading for an ADC provided herein is about 10. In some embodiments, the drug loading for an ADC provided herein is about 11. In some embodiments, the drug loading for an ADC provided herein is about 12. In some embodiments, the drug loading for an ADC provided herein is about 13. In some embodiments, the drug loading for an ADC provided herein is about 14. In some embodiments, the drug loading for an ADC provided herein is about 15. In some embodiments, the drug loading for an ADC provided herein is about 16. In some embodiments, the drug loading for an ADC provided herein is about 17. In some embodiments, the drug loading for an ADC provided herein is about 18. In some embodiments, the drug loading for an ADC provided herein is about 19. In some embodiments, the drug loading for an ADC provided herein is about 20.

In certain embodiments, fewer than the theoretical maximum of drug units are conjugated to an antibody during a conjugation reaction. An antibody can contain, for example, lysine residues that do not react with the drug-linker intermediate or linker reagent. Generally, antibodies do not contain many free and reactive cysteine thiol groups which can be linked to a drug unit; indeed most cysteine thiol residues in antibodies exist as disulfide bridges. In certain embodiments, an antibody can be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the linker unit or a drug unit is conjugated via a lysine residue on the antibody unit. In some embodiments, the linker unit or a drug unit is conjugated via a cysteine residue on the antibody unit.

In some embodiments, the amino acid that attaches to a linker unit or a drug unit is in the heavy chain of an antibody or antigen binding fragment thereof. In some embodiments, the amino acid that attaches to a linker unit or a drug unit is in the light chain of an antibody or antigen binding fragment thereof. In some embodiments, the amino acid that attaches to a linker unit or a drug unit is in the hinge region of an antibody or antigen binding fragment thereof. In some embodiments, the amino acid that attaches to a linker unit or a drug unit is in the Fc region of an antibody or antigen binding fragment thereof. In other embodiments, the amino acid that attaches to a linker unit or a drug unit is in the constant region (e.g., CH1, CH2, or CH3 of a heavy chain, or CH1 of a light chain) of an antibody or antigen binding fragment thereof. In yet other embodiments, the amino acid that attaches to a linker unit or a drug unit is in the VH framework regions of an antibody or antigen binding fragment thereof. In yet other embodiments, the amino acid that attaches to a linker unit or a drug unit is in the VL framework regions of an antibody or antigen binding fragment thereof.

The loading (drug/antibody ratio) of an ADC can be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug unit reagent, then the resulting product is a mixture of ADC compounds with a distribution of one or more drug unit attached to an antibody unit. The average number of drugs per antibody can be calculated from the mixture by a dual ELISA antibody assay, which is specific for antibody and specific for the drug. Individual ADC molecules can be identified in the mixture by mass spectroscopy and separated by HPLC, e.g. hydrophobic interaction chromatography (see, e.g., Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S.C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value can be isolated from the conjugation mixture by electrophoresis or chromatography.

Methods for preparing, screening, and characterizing the antibody drug conjugates are known to a person of ordinary skill in the art, for example, as described in U.S. Pat. No. 8,637,642, which is herein incorporated in its entirety by reference.

In some embodiments, the antibody drug conjugate for the methods provided herein is AGS-22M6E, which is prepared according to the methods described in U.S. Pat. No. 8,637,642 and has the following formula:

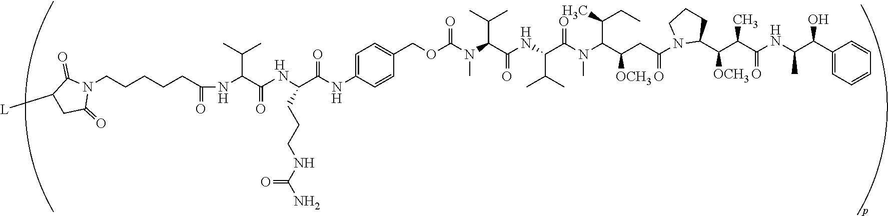

wherein L is Ha22-2(2,4)6.1 and p is from 1 to 20.

In some embodiments, p ranges from 1 to 20, 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is about 1. In other embodiments, p is about 2. In other embodiments, p is about 3. In other embodiments, p is about 4. In other embodiments, p is about 5. In other embodiments, p is about 6. In other embodiments, p is about 7. In other embodiments, p is about 8. In other embodiments, p is about 9. In other embodiments, p is about 10. In some embodiments, p is about 3.1. In some embodiments, p is about 3.2. In some embodiments, p is about 3.3. In some embodiments, p is about 3.4. In some embodiments, p is about 3.5. In other embodiments, p is about 3.6. In some embodiments, p is about 3.7. In some embodiments, p is about 3.8. In some embodiments, p is about 3.9. In some embodiments, p is about 4.0. In some embodiments, p is about 4.1. In some embodiments, p is about 4.2. In some embodiments, p is about 4.3. In some embodiments, p is about 4.4. In some embodiments, p is about 4.5. In other embodiments, p is about 4.6. In some embodiments, p is about 4.7. In some embodiments, p is about 4.8. In some embodiments, p is about 4.9. In some embodiments, p is about 5.0.

In some embodiments, the ADC used in the methods provided herein is enfortumab vedotin. Enfortumab vedotin is an ADC comprised of a fully human immunoglobulin G1 kappa (IgG1κ) antibody conjugated to the microtubule-disrupting agent (MMAE) via a protease-cleavable linker (Challita-Eid P M et al, Cancer Res. 2016; 76(10):3003-13]. Enfortumab vedotin induces antitumor activity by binding to 191P4D12 protein on the cell surface leading to internalization of the ADC-191P4D12 complex, which then traffics to the lysosomal compartment where MMAE is released via proteolytic cleavage of the linker. Intracellular release of MMAE subsequently disrupts tubulin polymerization resulting in G2/M phase cell cycle arrest and apoptotic cell death (Francisco J A et al, Blood. 2003 Aug. 15; 102(4):1458-65).

As described above and in in U.S. Pat. No. 8,637,642, AGS-22M6E is an ADC derived from a murine hybridoma cell line. Enfortumab vedotin is a Chinese hamster ovary (CHO) cell line-derived equivalent of AGS-22M6E ADC and is an exemplary product used for human treatment. Enfortumab vedotin has the same amino acid sequence, linker and cytotoxic drug as AGS-22M6E. The comparability between enfortumab vedotin and AGS-22M6E was confirmed through extensive analytical and biological characterization studies, such as binding affinity to 191P4D12, in vitro cytotoxicity, and in vivo antitumor activity.

In one embodiment, the ADC provided herein is enfortumab vedotin, also known as EV, PADCEV, AGS-22M6E, AGS-22C3E, ASG-22C3E. The enfortumab vedotin includes an anti-191P4D12 antibody, wherein the antibody or antigen binding fragment thereof comprises a heavy chain comprising amino acid residue 20 to amino acid residue 466 of SEQ ID NO:7 and a light chain comprising amino acid residue 23 to amino acid residue 236 of SEQ ID NO:8.

Enfortumab vedotin is a Nectin-4 directed antibody-drug conjugate (ADC) comprised of a fully human anti-nectin-4 IgG1 kappa monoclonal antibody (AGS-22C3) conjugated to the small molecule microtubule disrupting agent, monomethyl auristatin E (MMAE) via a protease-cleavable maleimidocaproyl valine-citrulline (vc) linker (SGD-1006). Conjugation takes place on cysteine residues that comprise the interchain disulfide bonds of the antibody to yield a product with a drug-to-antibody ratio of approximately 3.8:1. The molecular weight is approximately 152 kDa.

Enfortumab vedotin has the following structural formula:

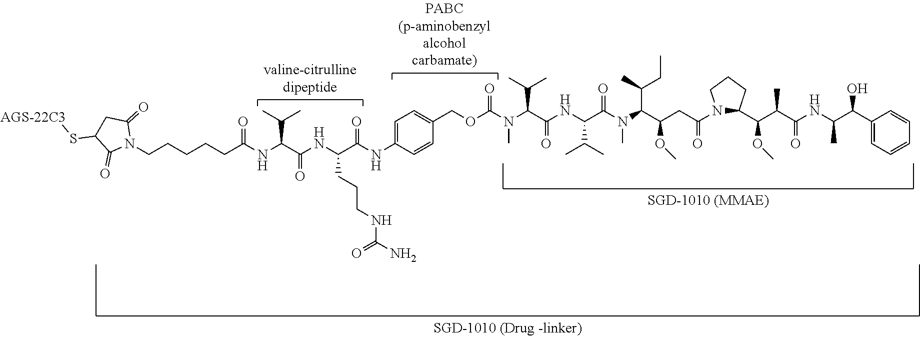

Approximately 4 molecules of MMAE are attached to each antibody molecule. Enfortumab vedotin is produced by chemical conjugation of the antibody and small molecule components. The antibody is produced by mammalian (Chinese hamster ovary) cells and the small molecule components are produced by chemical synthesis.

Enfortumab vedotin injection is provided as a sterile, preservative-free, white to off-white lyophilized powder in single-dose vials for intravenous use. Enfortumab vedotin is supplied as a 20 mg per vial and a 30 mg per vial and requires reconstitution with Sterile Water for Injection, USP, (2.3 mL and 3.3 mL, respectively) resulting in a clear to slightly opalescent, colorless to slightly yellow solution with a final concentration of 10 mg/mL. After reconstitution, each vial allows the withdrawal of 2 mL (20 mg) and 3 mL (30 mg). Each mL of reconstituted solution contains 10 mg of enfortumab vedotin, histidine (1.4 mg), histidine hydrochloride monohydrate (2.31 mg), polysorbate 20 (0.2 mg) and trehalose dihydrate (55 mg) with a pH of 6.0.

### 5.4 Pharmaceutical Compositions

In certain embodiments of the methods provided herein, the active ingredient such as the ADC and pembrolizumab used in the methods is provided in “pharmaceutical compositions.” Such pharmaceutical compositions include an antibody drug conjugate provided herein, and one or more pharmaceutically acceptable or physiologically acceptable excipients. In some embodiments, the antibody drug conjugate are provided in combination with, or separate from, a checkpoint inhibitor (such as a PD-1 inhibitor or a PD-L1 inhibitor, e.g., pembrolizumab). In some embodiments, the antibody drug conjugate are provided in combination with, or separate from pembrolizumab. Also provided is a composition comprising the antibody drug conjugate and pembrolizumab and one or more pharmaceutically acceptable or physiologically acceptable excipients. In particular embodiments, the antibody drug conjugate and pembrolizumab are present in a therapeutically acceptable amount. In certain embodiments, the antibody drug conjugate and pembrolizumab are provided in combination with, or separate from, one or more additional agents. Also provided is a composition comprising such one or more additional agents and one or more pharmaceutically acceptable or physiologically acceptable excipients. In particular embodiments, the antibody drug conjugate and an additional agent(s) are present in a therapeutically acceptable amount. The pharmaceutical compositions can be used in accordance with the methods and uses provided herein. Thus, for example, the pharmaceutical compositions can be administered ex vivo or in vivo to a subject in order to practice treatment methods and uses provided herein. Pharmaceutical compositions provided herein can be formulated to be compatible with the intended method or route of administration; exemplary routes of administration are set forth herein.

In some embodiments, provided are pharmaceutical compositions of antibody drug conjugates and pembrolizumab that modulate a cancer or tumor.

In certain embodiments of the methods provided herein, the pharmaceutical compositions comprising the ADCs and pembrolizumab can further comprise other therapeutically active agents or compounds disclosed herein or known to the skilled artisan which can be used in the treatment or prevention of various diseases and disorders as set forth herein (e.g., a cancer). As set forth above, the additional therapeutically active agents or compounds can be present in a separate pharmaceutical composition(s).

Pharmaceutical compositions typically comprise a therapeutically effective amount of at least one of the antibody drug conjugates provided herein and pembrolizumab and one or more pharmaceutically acceptable formulation agents. In certain embodiments, the pharmaceutical composition further comprises one or more additional agents described herein.

In one embodiment, a pharmaceutical composition comprises an antibody drug conjugate provided herein and pembrolizumab. In some embodiments, a pharmaceutical composition comprises a therapeutically effective amount of an antibody drug conjugate provided herein and pembrolizumab. In certain embodiments, the pharmaceutical composition comprises a pharmaceutically acceptable excipient.

In some embodiments, the antibody drug conjugate in the pharmaceutical composition provided herein is selected from the antibody drug conjugates described in Section 5.3 above.

In certain embodiments, the pharmaceutical composition comprises the antibody drug conjugate provided herein in an amount such that the dosage of antibody drug conjugate administered to a subject is about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg.

In certain embodiments, the pharmaceutical composition comprises the antibody drug conjugate provided herein in an amount such that the dosage of antibody drug conjugate administered to a subject is 0.1 mg/kg, 0.5 mg/kg, 0.75 mg/kg, 1 mg/kg, 1.25 mg/kg, 1.5 mg/kg, 2 mg/kg, 2.5 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg.

In certain embodiments, the pharmaceutical composition comprises the antibody drug conjugate provided herein in an amount such that the dosage of the antibody drug conjugate administered to a subject is about 0.1 mg/kg to about 100 mg/kg of the subject's body weight. In some embodiments, the dosage administered to the patient is about 1 mg/kg to about 75 mg/kg of the subject's body weight. In some embodiments, the dosage administered to a patient is between about 1 mg/kg and about 20 mg/kg of the subject's body weight, such as about 1 mg/kg to about 5 mg/kg of the subject's body weight.

In certain embodiments, the pharmaceutical composition comprises the antibody drug conjugate provided herein in an amount such that the dosage of the antibody drug conjugate administered to a subject is 0.1 mg/kg to 100 mg/kg of the subject's body weight. In some embodiments, the dosage administered to the patient is 1 mg/kg to 75 mg/kg of the subject's body weight. In some embodiments, the dosage administered to a patient is between 1 mg/kg and 20 mg/kg of the subject's body weight, such as 1 mg/kg to 5 mg/kg of the subject's body weight.

In certain embodiments, the pharmaceutical composition comprises the antibody drug conjugate at a concentration of from 0.1-100 mg/mL. In some embodiments, the pharmaceutical composition comprises the antibody drug conjugate at a concentration of from 1 to 20 mg/mL. In other embodiments, the pharmaceutical composition comprises the antibody drug conjugate at a concentration of from 5 to 15 mg/mL. In other embodiments, the pharmaceutical composition comprises the antibody drug conjugate at a concentration of from 8 to 12 mg/mL. In other embodiments, the pharmaceutical composition comprises the antibody drug conjugate at a concentration of from 9 to 11 mg/mL. In some embodiments, the pharmaceutical composition comprises the antibody drug conjugate at a concentration of about 9.5 mg/mL. In some embodiments, the pharmaceutical composition comprises the antibody drug conjugate at a concentration of about 9.6 mg/mL. In some embodiments, the pharmaceutical composition comprises the antibody drug conjugate at a concentration of about 9.7 mg/mL. In some embodiments, the pharmaceutical composition comprises the antibody drug conjugate at a concentration of about 9.8 mg/mL. In some embodiments, the pharmaceutical composition comprises the antibody drug conjugate at a concentration of about 9.9 mg/mL. In yet other embodiments, the pharmaceutical composition comprises the antibody drug conjugate at a concentration of about 10 mg/mL. In yet other embodiments, the pharmaceutical composition comprises the antibody drug conjugate at a concentration of about 10.1 mg/mL. In some embodiments, the pharmaceutical composition comprises the antibody drug conjugate at a concentration of about 10.2 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.4 mg/mL. In some embodiments, the pharmaceutical composition comprises the antibody drug conjugate at a concentration of about 10.5 mg/mL.

In some embodiments, the pharmaceutical composition provided herein comprises L-histidine, TWEEN-20, and at least one of trehalose dihydrate or sucrose. In some embodiments, the pharmaceutical composition provided herein further comprises hydrochloric acid (HCl) or succinic acid.

In certain embodiments, the pharmaceutical composition comprises pembrolizumab in an amount of about 100 mg, about 150 mg, about 200 mg, about 250 mg, about 300 mg, about 350 mg, about 400 mg, about 450 mg or about 500 mg. In particular embodiments, the pharmaceutical composition comprises pembrolizumab in an amount of about 200 mg.

In certain embodiments, the pharmaceutical composition comprises pembrolizumab in an amount of 100 mg, 150 mg, 200 mg, 250 mg, 300 mg, 350 mg, about 400 mg, 450 mg or 500 mg. In particular embodiments, the pharmaceutical composition comprises pembrolizumab in an amount of 200 mg.

In certain embodiments, the pharmaceutical composition comprises pembrolizumab in an amount of about 100 mg to about 500 mg, about 150 mg to about 500 mg, about 200 mg to about 500 mg, about 250 mg to about 500 mg, about 300 mg to about 500 mg, about 350 mg to about 500 mg, about 400 mg to about 500 mg, or about 450 mg to about 500 mg. In certain embodiments, the pharmaceutical composition comprises pembrolizumab in an amount of about 100 mg to about 450 mg, about 100 mg to about 400 mg, about 100 mg to about 350 mg, about 100 mg to about 300 mg, about 100 mg to about 250 mg, about 100 mg to about 200 mg, or about 100 mg to about 150 mg. In certain embodiments, the pharmaceutical composition comprises pembrolizumab in an amount of about 150 mg to about 450 mg, about 150 to about 400 mg, about 150 to about 350 mg, about 200 mg to about 450 mg, about 200 mg to about 400 mg, about 200 mg to about 300 mg, or about 200 mg to about 250 mg.

In certain embodiments, the pharmaceutical composition comprises pembrolizumab in an amount of 100 mg to 500 mg, 150 mg to 500 mg, 200 mg to 500 mg, 250 mg to 500 mg, 300 mg to 500 mg, 350 mg to 500 mg, 400 mg to 500 mg, or 450 mg to 500 mg. In certain embodiments, the pharmaceutical composition comprises pembrolizumab in an amount of 100 mg to 450 mg, 100 mg to 400 mg, 100 mg to 350 mg, 100 mg to 300 mg, 100 mg to 250 mg, 100 mg to 200 mg, or 100 mg to 150 mg. In certain embodiments, the pharmaceutical composition comprises pembrolizumab in an amount of 150 mg to 450 mg, 150 to 400 mg, 150 to 350 mg, 200 mg to 450 mg, 200 mg to 400 mg, 200 mg to 300 mg, or 200 mg to 250 mg.

In some embodiments, the concentration of L-histidine useful in the pharmaceutical compositions provided herein is in the range of between 5 and 50 mM. In some embodiments, the concentration of L-histidine in the pharmaceutical compositions provided herein is in the range of between 10 and 40 mM. In some embodiments, the concentration of L-histidine in the pharmaceutical compositions provided herein is in the range of between 15 and 35 mM.

In some embodiments, the concentration of L-histidine in the pharmaceutical compositions provided herein is in the range of between 15 and 30 mM. In some embodiments, the concentration of L-histidine in the pharmaceutical compositions provided herein is in the range of between 15 and 25 mM. In some embodiments, the concentration of L-histidine in the pharmaceutical compositions provided herein is in the range of between 15 and 35 mM. In some embodiments, the concentration of L-histidine in the pharmaceutical compositions provided herein is about 16 mM. In some embodiments, the concentration of L-histidine in the pharmaceutical compositions provided herein is about 17 mM. In some embodiments, the concentration of L-histidine in the pharmaceutical compositions provided herein is about 18 mM. In some embodiments, the concentration of L-histidine in the pharmaceutical compositions provided herein is about 19 mM. In some embodiments, the concentration of L-histidine in the pharmaceutical compositions provided herein is about 20 mM. In some embodiments, the concentration of L-histidine in the pharmaceutical compositions provided herein is about 21 mM. In some embodiments, the concentration of L-histidine in the pharmaceutical compositions provided herein is about 22 mM. In some embodiments, the concentration of L-histidine in the pharmaceutical compositions provided herein is about 23 mM. In some embodiments, the concentration of L-histidine in the pharmaceutical compositions provided herein is about 24 mM. In some embodiments, the concentration of L-histidine in the pharmaceutical compositions provided herein is about 25 mM.

In some embodiments, the concentration of TWEEN-20 useful in the pharmaceutical compositions provided herein is in the range of from 0.001 to 0.1% (v/v). In another embodiment, the concentration of TWEEN-20 is in the range of from 0.0025 to 0.075% (v/v). In one embodiment, the concentration of TWEEN-20 is in the range of from 0.005 to 0.05% (v/v). In another embodiment, the concentration of TWEEN-20 is in the range of from 0.0075 to 0.025% (v/v). In another embodiment, the concentration of TWEEN-20 is in the range of from 0.0075 to 0.05% (v/v). In another embodiment, the concentration of TWEEN-20 is in the range of from 0.01 to 0.03% (v/v). In one particular embodiment, the concentration of TWEEN-20 is about 0.01% (v/v). In one particular embodiment, the concentration of TWEEN-20 is about 0.015% (v/v). In one particular embodiment, the concentration of TWEEN-20 is about 0.016% (v/v). In one particular embodiment, the concentration of TWEEN-20 is about 0.017% (v/v). In one particular embodiment, the concentration of TWEEN-20 is about 0.018% (v/v). In one particular embodiment, the concentration of TWEEN-20 is about 0.019% (v/v). In one particular embodiment, the concentration of TWEEN-20 is about 0.02% (v/v). In one particular embodiment, the concentration of TWEEN-20 is about 0.021% (v/v). In one particular embodiment, the concentration of TWEEN-20 is about 0.022% (v/v). In one particular embodiment, the concentration of TWEEN-20 is about 0.023% (v/v). In one particular embodiment, the concentration of TWEEN-20 is about 0.024% (v/v). In one particular embodiment, the concentration of TWEEN-20 is about 0.025% (v/v).

In one embodiment, the concentration of trehalose dihydrate useful in the pharmaceutical compositions provided herein is in the range of between 1% and 20% (w/v). In another embodiment, the concentration of trehalose dihydrate is in the range of 2% and 15% (w/v). In one embodiment, the concentration of trehalose dihydrate is in the range of 3% and 10% (w/v). In another embodiment, the concentration of trehalose dihydrate is in the range of 4% and 9% (w/v). In another embodiment, the concentration of trehalose dihydrate is in the range of 4% and 8% (w/v). In another embodiment, the concentration of trehalose dihydrate is in the range of 4% and 7% (w/v). In another embodiment, the concentration of trehalose dihydrate is in the range of 4% and 6% (w/v). In another embodiment, the concentration of trehalose dihydrate is in the range of 4.5% and 6% (w/v). In another embodiment, the concentration of trehalose dihydrate is about 4.6% (w/v). In another embodiment, the concentration of trehalose dihydrate is about 4.7% (w/v). In another embodiment, the concentration of trehalose dihydrate is about 4.8% (w/v). In another embodiment, the concentration of trehalose dihydrate is about 4.9% (w/v). In another embodiment, the concentration of trehalose dihydrate is about 5.0% (w/v). In another embodiment, the concentration of trehalose dihydrate is about 5.1% (w/v). In another embodiment, the concentration of trehalose dihydrate is about 5.2% (w/v). In another embodiment, the concentration of trehalose dihydrate is about 5.3% (w/v). In another embodiment, the concentration of trehalose dihydrate is about 5.4% (w/v). In another embodiment, the concentration of trehalose dihydrate is about 5.5% (w/v). In another embodiment, the concentration of trehalose dihydrate is about 5.6% (w/v). In another embodiment, the concentration of trehalose dihydrate is about 5.7% (w/v). In another embodiment, the concentration of trehalose dihydrate is about 5.8% (w/v). In another embodiment, the concentration of trehalose dihydrate is about 5.9% (w/v). In another embodiment, the concentration of trehalose dihydrate is about 6.0% (w/v). In another embodiment, the concentration of trehalose dihydrate is about 6.1% (w/v). In another embodiment, the concentration of trehalose dihydrate is about 6.2% (w/v). In another embodiment, the concentration of trehalose dihydrate is about 6.3% (w/v). In another embodiment, the concentration of trehalose dihydrate is about 6.4% (w/v). In another embodiment, the concentration of trehalose dihydrate is about 6.5% (w/v).

In certain embodiments, the molarity of the trehalose dihydrate is from 50 to 300 mM. In other embodiments, the molarity of the trehalose dihydrate is from 75 to 250 mM. In some embodiments, the molarity of the trehalose dihydrate is from 100 to 200 mM. In other embodiments, the molarity of the trehalose dihydrate is from 130 to 150 mM. In some embodiments, the molarity of the trehalose dihydrate is from 135 to 150 mM. In certain embodiments, the molarity of the trehalose dihydrate is about 135 mM. In certain embodiments, the molarity of the trehalose dihydrate is about 136 mM. In certain embodiments, the molarity of the trehalose dihydrate is about 137 mM. In certain embodiments, the molarity of the trehalose dihydrate is about 138 mM. In certain embodiments, the molarity of the trehalose dihydrate is about 139 mM. In certain embodiments, the molarity of the trehalose dihydrate is about 140 mM. In certain embodiments, the molarity of the trehalose dihydrate is about 141 mM. In certain embodiments, the molarity of the trehalose dihydrate is about 142 mM. In certain embodiments, the molarity of the trehalose dihydrate is about 143 mM. In certain embodiments, the molarity of the trehalose dihydrate is about 144 mM. In certain embodiments, the molarity of the trehalose dihydrate is about 145 mM. In certain embodiments, the molarity of the trehalose dihydrate is about 146 mM. In certain embodiments, the molarity of the trehalose dihydrate is about 150 mM. In certain embodiments, the molarity of the trehalose dihydrate is about 151 mM. In certain embodiments, the molarity of the trehalose dihydrate is about 151 mM. In certain embodiments, the molarity of the trehalose dihydrate is about 152 mM. In certain embodiments, the molarity of the trehalose dihydrate is about 153 mM. In certain embodiments, the molarity of the trehalose dihydrate is about 154 mM. In certain embodiments, the molarity of the trehalose dihydrate is about 155 mM.

In one embodiment, the concentration of sucrose useful in the pharmaceutical compositions provided herein is in the range of between 1% and 20% (w/v). In another embodiment, the concentration of sucrose is in the range of 2% and 15% (w/v). In one embodiment, the concentration of sucrose is in the range of 3% and 10% (w/v). In another embodiment, the concentration of sucrose is in the range of 4% and 9% (w/v). In another embodiment, the concentration of sucrose is in the range of 4% and 8% (w/v). In another embodiment, the concentration of sucrose is in the range of 4% and 7% (w/v). In another embodiment, the concentration of sucrose is in the range of 4% and 6% (w/v). In another embodiment, the concentration of sucrose is in the range of 4.5% and 6% (w/v). In another embodiment, the concentration of sucrose is about 4.6% (w/v). In another embodiment, the concentration of sucrose is about 4.7% (w/v). In another embodiment, the concentration of sucrose is about 4.8% (w/v). In another embodiment, the concentration of sucrose is about 4.9% (w/v). In another embodiment, the concentration of sucrose is about 5.0% (w/v). In another embodiment, the concentration of sucrose is about 5.1% (w/v). In another embodiment, the concentration of sucrose is about 5.2% (w/v). In another embodiment, the concentration of sucrose is about 5.3% (w/v). In another embodiment, the concentration of sucrose is about 5.4% (w/v). In another embodiment, the concentration of sucrose is about 5.5% (w/v). In another embodiment, the concentration of sucrose is about 5.6% (w/v). In another embodiment, the concentration of sucrose is about 5.7% (w/v). In another embodiment, the concentration of sucrose is about 5.8% (w/v). In another embodiment, the concentration of sucrose is about 5.9% (w/v). In another embodiment, the concentration of sucrose is about 6.0% (w/v). In another embodiment, the concentration of sucrose is about 6.1% (w/v). In another embodiment, the concentration of sucrose is about 6.2% (w/v). In another embodiment, the concentration of sucrose is about 6.3% (w/v). In another embodiment, the concentration of sucrose is about 6.4% (w/v). In another embodiment, the concentration of sucrose is about 6.5% (w/v).

In certain embodiments, the molarity of the sucrose is from 50 to 300 mM. In other embodiments, the molarity of the sucrose is from 75 to 250 mM. In some embodiments, the molarity of the sucrose is from 100 to 200 mM. In other embodiments, the molarity of the sucrose is from 130 to 150 mM. In some embodiments, the molarity of the sucrose is from 135 to 150 mM. In certain embodiments, the molarity of the sucrose is about 135 mM. In certain embodiments, the molarity of the sucrose is about 136 mM. In certain embodiments, the molarity of the sucrose is about 137 mM. In certain embodiments, the molarity of the sucrose is about 138 mM. In certain embodiments, the molarity of the sucrose is about 139 mM. In certain embodiments, the molarity of the sucrose is about 140 mM. In certain embodiments, the molarity of the sucrose is about 141 mM. In certain embodiments, the molarity of the sucrose is about 142 mM. In certain embodiments, the molarity of the sucrose is about 143 mM. In certain embodiments, the molarity of the sucrose is about 144 mM. In certain embodiments, the molarity of the sucrose is about 145 mM. In certain embodiments, the molarity of the sucrose is about 146 mM. In certain embodiments, the molarity of the sucrose is about 150 mM. In certain embodiments, the molarity of the sucrose is about 151 mM. In certain embodiments, the molarity of the sucrose is about 151 mM. In certain embodiments, the molarity of the sucrose is about 152 mM. In certain embodiments, the molarity of the sucrose is about 153 mM. In certain embodiments, the molarity of the sucrose is about 154 mM. In certain embodiments, the molarity of the sucrose is about 155 mM.

In some embodiments, the pharmaceutical composition provided herein comprises HCl. In other embodiments, the pharmaceutical composition provided herein comprises succinic acid.

In some embodiments, the pharmaceutical composition provided herein has a pH in a range of 5.5 to 6.5. In other embodiments, the pharmaceutical composition provided herein has a pH in a range of 5.7 to 6.3. In some embodiments, the pharmaceutical composition provided herein has a pH of about 5.7. In some embodiments, the pharmaceutical composition provided herein has a pH of about 5.8. In some embodiments, the pharmaceutical composition provided herein has a pH of about 5.9. In some embodiments, the pharmaceutical composition provided herein has a pH of about 6.0. In some embodiments, the pharmaceutical composition provided herein has a pH of about 6.1. In some embodiments, the pharmaceutical composition provided herein has a pH of about 6.2. In some embodiments, the pharmaceutical composition provided herein has a pH of about 6.3.

In some embodiments, the pH is taken at room temperature. In other embodiments, the pH is taken at 15° C. to 27° C. In yet other embodiments, the pH is taken at 4° C. In yet other embodiments, the pH is taken at 25° C.

In some embodiments, the pH is adjusted by HCl. In some embodiments, the pharmaceutical composition comprises HCl, and the pharmaceutical composition has a pH in a range of 5.5 to 6.5 at room temperature. In some embodiments, the pharmaceutical composition comprises HCl, and the pharmaceutical composition has a pH in a range of 5.7 to 6.3 at room temperature. In some more specific embodiments, the pharmaceutical composition comprises HCl, and the pharmaceutical composition has a pH of about of 5.7 at room temperature. In some more specific embodiments, the pharmaceutical composition comprises HCl, and the pharmaceutical composition has a pH of about of 5.8 at room temperature. In some more specific embodiments, the pharmaceutical composition comprises HCl, and the pharmaceutical composition has a pH of about of 5.9 at room temperature. In some more specific embodiments, the pharmaceutical composition comprises HCl, and the pharmaceutical composition has a pH of about of 6.0 at room temperature. In some more specific embodiments, the pharmaceutical composition comprises HCl, and the pharmaceutical composition has a pH of about of 6.1 at room temperature. In some more specific embodiments, the pharmaceutical composition comprises HCl, and the pharmaceutical composition has a pH of about of 6.2 at room temperature. In some more specific embodiments, the pharmaceutical composition comprises HCl, and the pharmaceutical composition has a pH of about of 6.3 at room temperature.

In some embodiments, the pharmaceutical composition comprises HCl, and the pharmaceutical composition has a pH in a range of 5.5 to 6.5 at 15° C. to 27° C. In some embodiments, the pharmaceutical composition comprises HCl, and the pharmaceutical composition has a pH in a range of 5.7 to 6.3 at 15° C. to 27° C. In some more specific embodiments, the pharmaceutical composition comprises HCl, and the pharmaceutical composition has a pH of about of 5.7 at 15° C. to 27° C. In some more specific embodiments, the pharmaceutical composition comprises HCl, and the pharmaceutical composition has a pH of about of 5.8 at 15° C. to 27° C. In some more specific embodiments, the pharmaceutical composition comprises HCl, and the pharmaceutical composition has a pH of about of 5.9 at 15° C. to 27° C. In some more specific embodiments, the pharmaceutical composition comprises HCl, and the pharmaceutical composition has a pH of about of 6.0 at 15° C. to 27° C. In some more specific embodiments, the pharmaceutical composition comprises HCl, and the pharmaceutical composition has a pH of about of 6.1 at 15° C. to 27° C. In some more specific embodiments, the pharmaceutical composition comprises HCl, and the pharmaceutical composition has a pH of about of 6.2 at 15° C. to 27° C. In some more specific embodiments, the pharmaceutical composition comprises HCl, and the pharmaceutical composition has a pH of about of 6.3 at 15° C. to 27° C.

In some embodiments, the pH is adjusted by succinic acid. In some embodiments, the pharmaceutical composition comprises succinic acid, and the pharmaceutical composition has a pH in a range of 5.5 to 6.5 at room temperature. In some embodiments, the pharmaceutical composition comprises succinic acid, and the pharmaceutical composition has a pH in a range of 5.7 to 6.3 at room temperature. In some more specific embodiments, the pharmaceutical composition comprises succinic acid, and the pharmaceutical composition has a pH of about of 5.7 at room temperature. In some more specific embodiments, the pharmaceutical composition comprises succinic acid, and the pharmaceutical composition has a pH of about of 5.8 at room temperature. In some more specific embodiments, the pharmaceutical composition comprises succinic acid, and the pharmaceutical composition has a pH of about of 5.9 at room temperature. In some more specific embodiments, the pharmaceutical composition comprises succinic acid, and the pharmaceutical composition has a pH of about of 6.0 at room temperature. In some more specific embodiments, the pharmaceutical composition comprises succinic acid, and the pharmaceutical composition has a pH of about of 6.1 at room temperature. In some more specific embodiments, the pharmaceutical composition comprises succinic acid, and the pharmaceutical composition has a pH of about of 6.2 at room temperature. In some more specific embodiments, the pharmaceutical composition comprises succinic acid, and the pharmaceutical composition has a pH of about of 6.3 at room temperature.

In some embodiments, the pharmaceutical composition comprises succinic acid, and the pharmaceutical composition has a pH in a range of 5.5 to 6.5 at 15° C. to 27° C. In some embodiments, the pharmaceutical composition comprises succinic acid, and the pharmaceutical composition has a pH in a range of 5.7 to 6.3 at 15° C. to 27° C. In some more specific embodiments, the pharmaceutical composition comprises succinic acid, and the pharmaceutical composition has a pH of about of 5.7 at 15° C. to 27° C. In some more specific embodiments, the pharmaceutical composition comprises succinic acid, and the pharmaceutical composition has a pH of about of 5.8 at 15° C. to 27° C. In some more specific embodiments, the pharmaceutical composition comprises succinic acid, and the pharmaceutical composition has a pH of about of 5.9 at 15° C. to 27° C. In some more specific embodiments, the pharmaceutical composition comprises succinic acid, and the pharmaceutical composition has a pH of about of 6.0 at 15° C. to 27° C. In some more specific embodiments, the pharmaceutical composition comprises succinic acid, and the pharmaceutical composition has a pH of about of 6.1 at 15° C. to 27° C. In some more specific embodiments, the pharmaceutical composition comprises succinic acid, and the pharmaceutical composition has a pH of about of 6.2 at 15° C. to 27° C. In some more specific embodiments, the pharmaceutical composition comprises succinic acid, and the pharmaceutical composition has a pH of about of 6.3 at 15° C. to 27° C.

In some specific embodiments, the pharmaceutical composition provided herein comprises about 20 mM L-histidine, about 0.02% (w/v) TWEEN-20, and at least one of about 5.5% (w/v) trehalose dihydrate or about 5% (w/v) sucrose. In some embodiments, the pharmaceutical composition provided herein further comprises HCl or succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5.5% (w/v) trehalose dihydrate and HCl. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and HCl. In some embodiments, the pH is about 6.0 at room temperature. In some embodiments, the pH is about 6.0 at 25° C.

In other specific embodiments, the pharmaceutical composition provided herein comprises about 20 mM L-histidine, about 0.02% (w/v) TWEEN-20, about 5.5% (w/v) trehalose dihydrate and succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and succinic acid. In some embodiments, the pH is about 6.0 at room temperature. In some embodiments, the pH is about 6.0 at 25° C.

In a specific embodiment, provided herein comprises
(a) an antibody drug conjugate comprising the following structure:

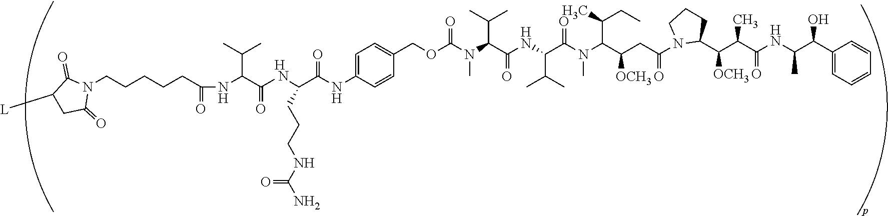

wherein L- represents the antibody or antigen binding fragment (e.g. anti-nectin-4 antibody or antigen binding fragment thereof) thereof and p is from 1 to 10; and
(b) a pharmaceutically acceptable excipient comprising about 20 mM L-histidine, about 0.02% (w/v) TWEEN-20, about 5.5% (w/v) trehalose dihydrate, and HCl, wherein the antibody drug conjugate is at the concentration of about 10 mg/mL, and wherein the pH is about 6.0 at 25° C.

In another specific embodiment, the pharmaceutical composition provided herein comprises:
(a) an antibody drug conjugate comprising the following structure:

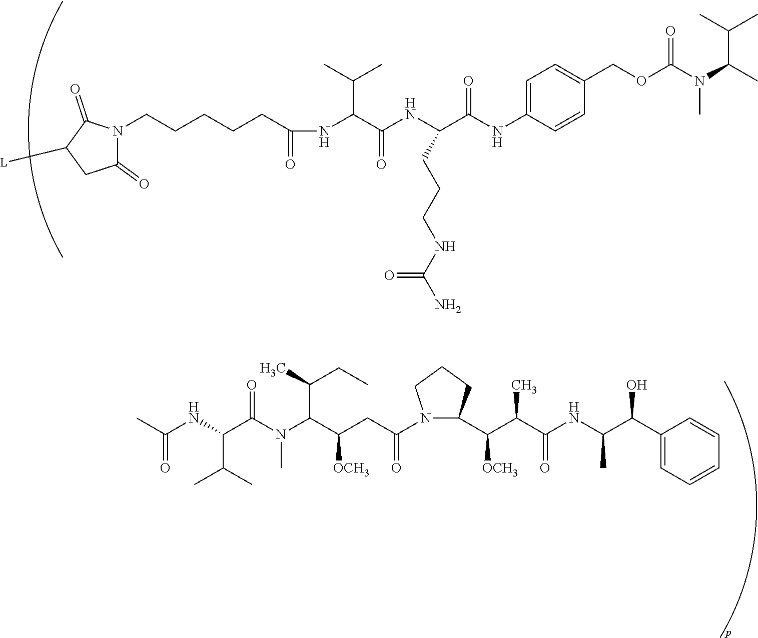

wherein L- represents the antibody or antigen binding fragment thereof (e.g. anti-nectin-4 antibody or antigen binding fragment thereof) and p is from 1 to 10; and
(b) a pharmaceutically acceptable excipient comprising about 20 mM L-histidine, about 0.02% (w/v) TWEEN-20, about 5.5% (w/v) trehalose dihydrate, and succinic acid, wherein the antibody drug conjugate is at the concentration of about 10 mg/mL, and wherein the pH is about 6.0 at 25° C.

In yet another specific embodiment, the pharmaceutical composition provided herein comprises:
(a) an antibody drug conjugate comprising the following structure:

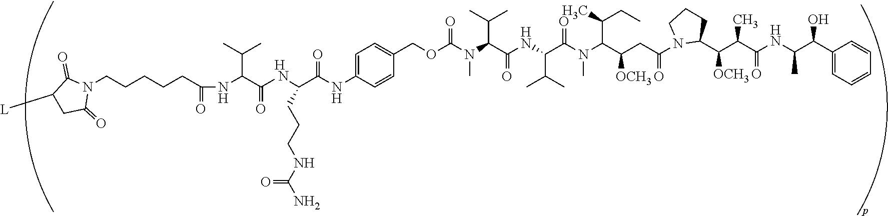

wherein L- represents the antibody or antigen binding fragment thereof (e.g. anti-nectin-4 antibody or antigen binding fragment thereof) and p is from 1 to 10; and
(b) a pharmaceutically acceptable excipient comprising about 20 mM L-histidine, about 0.02% (w/v) TWEEN-20, about 5.0% (w/v) sucrose, and HCl, wherein the antibody drug conjugate is at the concentration of about 10 mg/mL, and wherein the pH is about 6.0 at 25° C.

Although certain numbers (and numerical ranges thereof) are provided, it is understood that, in certain embodiments, numerical values within, e.g., 2%, 5%, 10%, 15% or 20% of said numbers (or numerical ranges) are also contemplated.

A primary solvent in a vehicle can be either aqueous or non-aqueous in nature. In addition, the vehicle can contain other pharmaceutically acceptable excipients for modifying or maintaining the pH, osmolarity, viscosity, sterility or stability of the pharmaceutical composition. In certain embodiments, the pharmaceutically acceptable vehicle is an aqueous buffer. In other embodiments, a vehicle comprises, for example, sodium chloride and/or sodium citrate.

Pharmaceutical compositions provided herein can contain still other pharmaceutically acceptable formulation agents for modifying or maintaining the rate of release of an antibody drug conjugate and/or an additional agent, as described herein. Such formulation agents include those substances known to artisans skilled in preparing sustained-release formulations. For further reference pertaining to pharmaceutically and physiologically acceptable formulation agents, see, for example, Remington's Pharmaceutical Sciences, 18th Ed. (1990, Mack Publishing Co., Easton, Pa. 18042) pages 1435-1712, The Merck Index, 12th Ed. (1996, Merck Publishing Group, Whitehouse, NJ); and Pharmaceutical Principles of Solid Dosage Forms (1993, Technonic Publishing Co., Inc., Lancaster, Pa.). Additional pharmaceutical compositions appropriate for administration are known in the art and are applicable in the methods and compositions provided herein.

In some embodiments, the pharmaceutical composition provided herein is in a liquid form. In other embodiments, the pharmaceutical composition provided herein is lyophilized.

A pharmaceutical composition can be formulated to be compatible with its intended route of administration. Thus, pharmaceutical compositions include excipients suitable for administration by routes including parenteral (e.g., subcutaneous (s.c.), intravenous, intramuscular, or intraperitoneal), intradermal, oral (e.g., ingestion), inhalation, intracavity, intracranial, and transdermal (topical). Other exemplary routes of administration are set forth herein.

Pharmaceutical compositions can be in the form of a sterile injectable aqueous or oleagenous suspension. This suspension can be formulated using suitable dispersing or wetting agents and suspending agents disclosed herein or known to the skilled artisan. The sterile injectable preparation can also be a sterile injectable solution or suspension in a non-toxic parenterally-acceptable diluent or solvent, for example, as a solution in 1,3-butane diol. Acceptable diluents, solvents and dispersion media that can be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil can be employed, including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

In one embodiment, the pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In one embodiment, the pharmaceutical compositions provided herein can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, e.g., Remington, The Science and Practice of Pharmacy, supra).

In one embodiment, the pharmaceutical compositions intended for parenteral administration can include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

In one embodiment, suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

In one embodiment, suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, KS).

In one embodiment, the pharmaceutical compositions provided herein can be formulated for single or multiple dosage administration. The single dosage formulations are packaged in an ampoule, a vial, or a syringe. The multiple dosage parenteral formulations can contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

In one embodiment, the pharmaceutical compositions provided herein can be formulated as immediate or modified release dosage forms, including delayed-, sustained, pulsed-, controlled, targeted-, and programmed-release forms.

Dispersible powders and granules suitable for preparation of an aqueous suspension by addition of water provide the active ingredient in admixture with a dispersing or wetting agent, suspending agent and one or more preservatives. Suitable dispersing or wetting agents and suspending agents are exemplified herein.

Pharmaceutical compositions can also include excipients to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, can be employed. Prolonged absorption of injectable pharmaceutical compositions can be achieved by including an agent that delays absorption, for example, aluminum monostearate or gelatin. Prevention of the action of microorganisms can be achieved by various antibacterial and antifungal agents, for example, parabens, chlorobutanol, phenol, ascorbic acid, thimerosal, and the like.

The pharmaceutical composition provided herein can be stored at −80° C., 4° C., 25° C. or 37° C.

A lyophilized composition can be made by freeze-drying the liquid pharmaceutical composition provided herein. In a specific embodiment, the pharmaceutical composition provided here is a lyophilized pharmaceutical composition. In some embodiments, the pharmaceutical formulations are lyophilized powders, which can be reconstituted for administration as solutions, emulsions and other mixtures. They can also be reconstituted and formulated as solids or gels.

In some embodiments, preparation of the lyophilized formulation provided herein involves batching of the formulated bulk solution for lyophilization, aseptic filtration, filling in vials, freezing vials in a freeze-dryer chamber, followed by lyophilization, stoppering and capping.

A lyophilizer can be used in preparing the lyophilized formulation. For example, a VirTis Genesis Model EL pilot unit can be employed. The unit incorporates a chamber with three working shelves (to a total usable shelf area of ca 0.4 square meters), an external condenser, and a mechanical vacuum pumping system. Cascaded mechanical refrigeration allows the shelves to be cooled to −70° C. or lower, and the external condenser to −90° C. or lower. Shelf temperature and chamber pressure were controlled automatically to +/−0.5° C. and +/−2 microns (milliTorr), respectively. The unit was equipped with a capacitance manometer vacuum gauge, a Pirani vacuum gauge, a pressure transducer (to measure from 0 to 1 atmosphere), and a relative humidity sensor.

The lyophilized powder can be prepared by dissolving an antibody drug conjugate provided herein, or a pharmaceutically acceptable derivative thereof, in a suitable solvent. In some embodiments, the lyophilized powder is sterile. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the antibody drug conjugate. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable excipient. Such amount can be empirically determined and adjusted according to specific needs.

An exemplary reconstitution procedure is illustrated as follows: (1) fit the 5 mL or 3 mL syringe with a with a 18 or 20 Gauge needle and filled the syringe with water of the grade Water for Injection (WFI); (2) measure appropriate amount of WFI using the syringe graduations, ensuring that the syringe was free of air bubbles; (3) inserted the needle through the rubber stopper; (4) dispense the entire contents of the syringe into the container down the vial wall, removed the syringe and needle and put into the sharp container; (4) swirl the vial continuously to carefully solubilize the entire vial contents until fully reconstituted (e.g., about 20-40 seconds) and minimize excessive agitation of the protein solution that could result in foaming.

In some embodiments, the pharmaceutical composition provided herein is supplied as a dry sterilized lyophilized powder or water free concentrate in a hermetically sealed container and can be reconstituted, e.g., with water or saline to the appropriate concentration for administration to a subject. In certain embodiments, the antibody drug conjugate is supplied as a dry sterile lyophilized powder in a hermetically sealed container at a unit dosage of at least 0.1 mg, at least 0.5 mg, at least 1 mg, at least 2 mg, at least 3 mg, at least 5 mg, at least 10 mg, at least 15 mg, at least 25 mg, at least 30 mg, at least 35 mg, at least 45 mg, at least 50 mg, at least 60 mg, at least 75 mg, at least 80 mg, at least 85 mg, at least 90 mg, at least 95 mg, or at least 100 mg. The lyophilized antibody drug conjugate can be stored at between 2 and 8° C. in its original container and the antibody drug conjugate can be administered within 12 hours, such as within 6 hours, within 5 hours, within 3 hours, or within 1 hour after being reconstituted. In an alternative embodiment, the pharmaceutical composition comprising the antibody drug conjugate provided herein is supplied in liquid form in a hermetically sealed container indicating the quantity and concentration of the antibody drug conjugate. In certain embodiments, the liquid form of the antibody drug conjugate is supplied in a hermetically sealed container at least 0.1 mg/ml, at least 0.5 mg/ml, at least 1 mg/ml, at least 5 mg/ml, at least 10 mg/ml, at least 15 mg/ml, at least 25 mg/ml, at least 30 mg/ml, at least 40 mg/ml, at least 50 mg/ml, at least 60 mg/ml, at least 70 mg/ml, at least 80 mg/ml, at least 90 mg/ml, or at least 100 mg/ml.

Additional embodiments for the pharmaceutical compositions have been described in U.S. Pat. No. 8,637,642 and International Application No. PCT/US2019/056214 (Publication No. WO2020/117373), both of which are hereby incorporated in their entireties by reference.

### 5.5 Methods for a Combination Therapy

The method for inhibiting growth of tumor cells using the pharmaceutical composition provided herein in combination with chemotherapy or radiation or both comprises administering the present pharmaceutical composition before, during, or after commencing chemotherapy or radiation therapy, as well as any combination thereof (i.e. before and during, before and after, during and after, or before, during, and after commencing the chemotherapy and/or radiation therapy). Depending on the treatment protocol and the specific patient needs, the method is performed in a manner that will provide the most efficacious treatment and ultimately prolong the life of the patient. Additional embodiments for such combination therapy have been described in U.S. Pat. No. 8,637,642 and International Application No. PCT/US2019/056214 (Publication No. WO2020/117373), both of which are hereby incorporated in their entireties by reference.

### 5.6 Doses of the Immune Checkpoint Inhibitors (e.g. Pembrolizumab)

In some embodiments, the amount of the checkpoint inhibitor (such as a PD-1 inhibitor or a PD-L1 inhibitor, e.g., an anti-PD-1 antibody, e.g., pembrolizumab), for the various methods provided herein be determined by standard clinical techniques.

A dosage of the checkpoint inhibitor results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose of the checkpoint inhibitor to be employed will also depend on the route of administration, and the seriousness of a cancer in a subject, and should be decided according to the judgment of the practitioner and each patient's circumstances.

In some embodiments, the dosage of the checkpoint inhibitor (e.g., a PD-1 inhibitor or a PD-L1 inhibitor, e.g., an anti-PD-1 antibody, e.g., pembrolizumab) administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 1 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight. In some embodiments, dosage administered to a patient is about 9.0 mg/kg of the subject's body weight. In some embodiments, dosage administered to a patient is about 10.0 mg/kg of the subject's body weight. In some embodiments, dosage administered to a patient is about 15.0 mg/kg of the subject's body weight. In some embodiments, dosage administered to a patient is about 20.0 mg/kg of the subject's body weight.

In certain embodiments, the checkpoint inhibitor (such as a PD-1 inhibitor or a PD-L1 inhibitor, e.g., an anti-PD-1 antibody, e.g., pembrolizumab) is administered at a dose of about 100 mg, about 150 mg, about 200 mg, about 250 mg, about 300 mg, about 350 mg, about 400 mg, about 450 mg or about 500 mg. In particular embodiments, the checkpoint inhibitor (such as a PD-1 inhibitor or a PD-L1 inhibitor, e.g., an anti-PD-1 antibody, e.g., pembrolizumab) is administered at a dose of about 200 mg.

In certain embodiments, the checkpoint inhibitor (such as a PD-1 inhibitor or a PD-L1 inhibitor, e.g., an anti-PD-1 antibody, e.g., pembrolizumab) is administered at a dose of about 100 mg, 150 mg, 200 mg, 250 mg, 300 mg, 350 mg, 400 mg, 450 mg or 500 mg. In particular embodiments, the checkpoint inhibitor (such as a PD-1 inhibitor or a PD-L1 inhibitor, e.g., an anti-PD-1 antibody, e.g., pembrolizumab) is administered at a dose of about 200 mg. In particular embodiments, the pembrolizumab is administered at a dose of about 200 mg.

In certain embodiments, the checkpoint inhibitor (such as a PD-1 inhibitor or a PD-L1 inhibitor, e.g., an anti-PD-1 antibody, e.g., pembrolizumab) is administered at a dose of about 100 mg to about 500 mg, about 150 mg to about 500 mg, about 200 mg to about 500 mg, about 250 mg to about 500 mg, about 300 mg to about 500 mg, about 350 mg to about 500 mg, about 400 mg to about 500 mg, or about 450 mg to about 500 mg. In certain embodiments, the checkpoint inhibitor (such as a PD-1 inhibitor or a PD-L1 inhibitor, e.g., an anti-PD-1 antibody, e.g., pembrolizumab) is administered at a dose of about 100 mg to about 450 mg, about 100 mg to about 400 mg, about 100 mg to about 350 mg, about 100 mg to about 300 mg, about 100 mg to about 250 mg, about 100 mg to about 200 mg, or about 100 mg to about 150 mg. In certain embodiments, the checkpoint inhibitor (such as a PD-1 inhibitor or a PD-L1 inhibitor, e.g., an anti-PD-1 antibody, e.g., pembrolizumab) is administered at a dose of about 150 mg to about 450 mg, about 150 to about 400 mg, about 150 to about 350 mg, about 200 mg to about 450 mg, about 200 mg to about 400 mg, about 200 mg to about 300 mg, or about 200 mg to about 250 mg.

In certain embodiments, the checkpoint inhibitor (such as a PD-1 inhibitor or a PD-L1 inhibitor, e.g., an anti-PD-1 antibody, e.g., pembrolizumab) is administered at a dose of 100 mg to 500 mg, 150 mg to 500 mg, 200 mg to 500 mg, 250 mg to 500 mg, 300 mg to 500 mg, 350 mg to 500 mg, 400 mg to 500 mg, or 450 mg to 500 mg. In certain embodiments, the checkpoint inhibitor (such as a PD-1 inhibitor or a PD-L1 inhibitor, e.g., an anti-PD-1 antibody, e.g., pembrolizumab) is administered at a dose of 100 mg to 450 mg, 100 mg to 400 mg, 100 mg to 350 mg, 100 mg to 300 mg, 100 mg to 250 mg, 100 mg to 200 mg, or 100 mg to 150 mg. In certain embodiments, the checkpoint inhibitor (such as a PD-1 inhibitor or a PD-L1 inhibitor, e.g., an anti-PD-1 antibody, e.g., pembrolizumab) is administered at a dose of 150 mg to 450 mg, 150 to 400 mg, 150 to 350 mg, 200 mg to 450 mg, 200 mg to 400 mg, 200 mg to 300 mg, or 200 mg to 250 mg.

In some embodiments, (a) the checkpoint inhibitor (such as a PD-1 inhibitor or a PD-L1 inhibitor, e.g., an anti-PD-1 antibody, e.g., pembrolizumab) is administered at a dose of about 100 mg to about 400 mg; and (b) after step (a), the anti-PD-1 antibody is administered to the subject at a dose of about 300 mg to about 500 mg mg every 40 to 45 days. In some embodiments, (a) the checkpoint inhibitor (such as a PD-1 inhibitor or a PD-L1 inhibitor, e.g., an anti-PD-1 antibody, e.g., pembrolizumab) is administered at a dose of about 100 mg to about 300 mg; and (b) after step (a), the anti-PD-1 antibody is administered to the subject at a dose of about 350 mg to about 450 mg mg every 40 to 45 days. In some embodiments, (a) the checkpoint inhibitor (such as a PD-1 inhibitor or a PD-L1 inhibitor, e.g., an anti-PD-1 antibody, e.g., pembrolizumab) is administered at a dose of about 150 mg to about 250 mg; and (b) after step (a), the anti-PD-1 antibody is administered to the subject at a dose of about 350 mg to about 450 mg mg every 40 to 45 days. In certain embodiments, (a) the checkpoint inhibitor (such as a PD-1 inhibitor or a PD-L1 inhibitor, e.g., an anti-PD-1 antibody, e.g., pembrolizumab) is administered at a dose of about 200 mg; and (b) after step (a), the anti-PD-1 antibody is administered to the subject at a dose of about 400 mg every 42 days.

In some embodiments, (a) the checkpoint inhibitor (such as a PD-1 inhibitor or a PD-L1 inhibitor, e.g., an anti-PD-1 antibody, e.g., pembrolizumab) is administered at a dose of 100 mg to 400 mg; and (b) after step (a), the anti-PD-1 antibody is administered to the subject at a dose of 300 mg to 500 mg mg every 40 to 45 days. In some embodiments, (a) the checkpoint inhibitor (such as a PD-1 inhibitor or a PD-L1 inhibitor, e.g., an anti-PD-1 antibody, e.g., pembrolizumab) is administered at a dose of 100 mg to 300 mg; and (b) after step (a), the anti-PD-1 antibody is administered to the subject at a dose of 350 mg to 450 mg mg every 40 to 45 days. In some embodiments, (a) the checkpoint inhibitor (such as a PD-1 inhibitor or a PD-L1 inhibitor, e.g., an anti-PD-1 antibody, e.g., pembrolizumab) is administered at a dose of 150 mg to 250 mg; and (b) after step (a), the anti-PD-1 antibody is administered to the subject at a dose of 350 mg to 450 mg mg every 40 to 45 days. In certain embodiments, (a) the checkpoint inhibitor (such as a PD-1 inhibitor or a PD-L1 inhibitor, e.g., an anti-PD-1 antibody, e.g., pembrolizumab) is administered at a dose of 200 mg; and (b) after step (a), the anti-PD-1 antibody is administered to the subject at a dose of 400 mg every 42 days.

In some embodiments, pembrolizumab is administered at a dose of about 200 mg. In some embodiments, pembrolizumab is administered at a dose of about 200 mg every 3 weeks. In some embodiments, pembrolizumab is administered at a dose of about 200 mg on day 1 of each 3 week cycle.

In some embodiments, pembrolizumab is administered at a dose of about 400 mg. In some embodiments, pembrolizumab is administered at a dose of about 400 mg every 6 weeks. In some embodiments, pembrolizumab is administered at a dose of about 400 mg on day 1 of each 6 week cycle.

In some embodiment, pembrolizumab is administered at a dose of about 2 mg/kg. In some embodiment, pembrolizumab is administered at a dose of about 2 mg/kg every three weeks. In some embodiment, pembrolizumab is administered at a dose of about 2 mg/kg on day 1 of each 3 week cycle. In particular embodiments, the patient is a pediatric patient.

In some embodiment, pembrolizumab is administered as a 30 minute (−5 minutes/+10 minutes) intravenous infusion. In one embodiment, the selected dose of pembrolizumab is administered by IV infusion over a time period of between 25 and 40 minutes, or about 30 minutes.

In one aspect, pembrolizumab in included in a pharmaceutical composition with a pharmaceutically acceptable carrier or diluent and may include additional pharmaceutically acceptable excipients.

In particular embodiments, the patient is an adult patient. In particular embodiments, the patient is an adult patient, wherein the adult patient is 18 years or older. 5.7 Dosage of the ADCs for the Methods

In some embodiments, the amount of a prophylactic or therapeutic agent (e.g., an antibody drug conjugate provided herein), or a pharmaceutical composition provided herein that will be effective in the prevention and/or treatment of a cancer can be determined by standard clinical techniques.

In some embodiments, the ADC of the methods for which the various dosages are described in this Section (Section 5.7) is enfortumab vedotin (EV).

Accordingly, a dosage of an antibody drug conjugate in the pharmaceutical composition that results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose to be employed in the formulation will also depend on the route of administration, and the seriousness of a cancer in a subject, and should be decided according to the judgment of the practitioner and each patient's circumstances.

Effective doses can be extrapolated from dose-response curves derived from in vitro or animal model test systems.

For the pharmaceutical composition comprising the antibody drug conjugate provided herein, the dosage of the antibody drug conjugate administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 0.5 mg/kg of the subject's body weight. In some embodiments, dosage administered to a patient is about 0.75 mg/kg of the subject's body weight. In some embodiments, dosage administered to a patient is about 1 mg/kg of the subject's body weight. In some embodiments, dosage administered to a patient is about 1.25 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight.

In some embodiments, the antibody drug conjugate formulated in the pharmaceutical composition provided herein is administered based on the patient's actual body weight at baseline and doses will not change unless the patient's weight changes by ≥10% from baseline of the previous cycle, or the dose adjustment criteria is met. In some embodiments, actual weight will be used except for patients weighing greater than 100 kg, in such cases, the dose will be calculated based on a weight of 100 kg. In some embodiments, the maximum doses are 100 mg for patients receiving the 1.00 mg/kg dose level and 125 mg for patients receiving the 1.25 mg/kg dose level.

In one embodiment, approximately 100 mg/kg or less, approximately 75 mg/kg or less, approximately 50 mg/kg or less, approximately 25 mg/kg or less, approximately 10 mg/kg or less, approximately 5 mg/kg or less, approximately 1.5 mg/kg or less, approximately 1.25 mg/kg or less, approximately 1 mg/kg or less, approximately 0.75 mg/kg or less, approximately 0.5 mg/kg or less, or approximately 0.1 mg/kg or less of an antibody drug conjugate formulated in the present pharmaceutical composition is administered 5 times, 4 times, 3 times, 2 times or 1 time to treat a cancer. In some embodiments, the pharmaceutical composition comprising the antibody drug conjugate provided herein is administered about 1-12 times, wherein the doses can be administered as necessary, e.g., weekly, biweekly, monthly, bimonthly, trimonthly, etc., as determined by a physician. In some embodiments, a lower dose (e.g., 0.1-15 mg/kg) can be administered more frequently (e.g., 3-6 times). In other embodiments, a higher dose (e.g., 25-100 mg/kg) can be administered less frequently (e.g., 1-3 times).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every two-week cycle (e.g., about 14 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every three-week cycle (e.g., about 21 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every four-week cycle (e.g., about 28 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, 6, 7, 8, 9, 10, 11, or 12 times at about monthly (e.g., about 30 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, or 6 times at about bi-monthly (e.g., about 60 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In yet another embodiment, a single dose of an antibody drug conjugate formulated in the pharmaceutical composition provided herein is administered to patient to prevent and/or treat a cancer 1, 2, 3 or 4 times at about tri-monthly (e.g., about 120 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In certain embodiments, the route of administration for a dose of an antibody drug conjugate formulated in the pharmaceutical composition provided herein to a patient is intranasal, intramuscular, intravenous, or a combination thereof, but other routes described herein are also acceptable. Each dose may or may not be administered by an identical route of administration. In some embodiments, an antibody drug conjugate formulated in the pharmaceutical composition provided herein can be administered via multiple routes of administration simultaneously or subsequently to other doses of one or more additional therapeutic agents.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion over about 30 minutes twice every three-week cycle. In some embodiments, the antibody drug conjugate formulated in the pharmaceutical composition is administered by an intravenous (IV) injection or infusion over about 30 minutes on Days 1 and 8 of every three-week cycle. In some embodiments, the method further comprises administering an immune checkpoint inhibitor by an intravenous (IV) injection or infusion one or more times in each three-week cycle. In some embodiments, the method further comprises administering an immune checkpoint inhibitor by an intravenous (IV) injection or infusion on Day 1 of every three-week cycle. In some embodiments, the immune checkpoint inhibitor is pembrolizumab, and wherein pembrolizumab is administered at amount of about 200 mg over about 30 minutes. In some embodiments, the antibody drug conjugate is administered to patients with locally advanced urothelial or bladder cancer who have shown disease progression or relapse during or after treatment with another cancer treatment. In some embodiments, the ADC of the methods for which the various dosages are described in this paragraph is enfortumab vedotin (EV).

In other more specific embodiments, the antibody drug conjugate formulated in the pharmaceutical composition provided herein is administered at a dose of about 0.5 mg/kg, about 0.75 mg/kg, 1 mg/kg, about 1.25 mg/kg, or about 1.5 mg/kg of the subject's body weight by an intravenous (IV) injection or infusion over about 30 minutes three times every four-week cycle. In some embodiments, the antibody drug conjugate formulated in the pharmaceutical composition is administered on Days 1, 8 and 15 of every 28-day (four-week) cycle. In some embodiments, the antibody drug conjugate formulated in the pharmaceutical composition is administered by an intravenous (IV) injection or infusion over about 30 minutes on Days 1, 8 and 15 of every 28-day (four-week) cycle. In some embodiments, the method further comprises administering an immune checkpoint inhibitor by an intravenous (IV) injection or infusion one or more times in each four-week cycle. In some embodiments, the immune checkpoint inhibitor is pembrolizumab. In other embodiments, the immune checkpoint inhibitor is atezolizumab. In some embodiments, the antibody drug conjugate is administered to patients with urothelial or bladder cancer who have shown disease progression or relapse during or after treatment with another cancer treatment. In some embodiments, the antibody drug conjugate is administered to patients with metastatic urothelial or bladder cancer who have shown disease progression or relapse during or after treatment with another cancer treatment. In some embodiments, the antibody drug conjugate is administered to patients with locally advanced urothelial or bladder cancer who have shown disease progression or relapse during or after treatment with another cancer treatment. In some embodiments, the ADC of the methods for which the various dosages are described in this paragraph is enfortumab vedotin (EV).

In some embodiments of the various methods provided herein, the ADC is administered at a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments, the ADC is administered at a dose of about 1 to about 10 mg/kg of the subject's body weight. In certain embodiments, the ADC is administered at a dose of about 1 to about 5 mg/kg of the subject's body weight. In other embodiments, the ADC is administered at a dose of about 1 to about 2.5 mg/kg of the subject's body weight. In further embodiments, the ADC is administered at a dose of about 1 to about 1.25 mg/kg of the subject's body weight. In some embodiments, the ADC is administered at a dose of about 0.25 mg/kg of the subject's body weight. In some embodiments, the ADC is administered at a dose of about 0.5 mg/kg of the subject's body weight. In some embodiments, the ADC is administered at a dose of about 0.75 mg/kg of the subject's body weight. In some embodiments, the ADC is administered at a dose of about 1.0 mg/kg of the subject's body weight. In some embodiments, the ADC is administered at a dose of about 1.25 mg/kg of the subject's body weight. In some embodiments, the ADC is administered at a dose of about 1.5 mg/kg of the subject's body weight. In some embodiments, the ADC is administered at a dose of about 1.75 mg/kg of the subject's body weight. In some embodiments, the ADC is administered at a dose of about 2.0 mg/kg of the subject's body weight. In some embodiments, the ADC is administered at a dose of about 2.25 mg/kg of the subject's body weight. In some embodiments, the ADC is administered at a dose of about 2.5 mg/kg of the subject's body weight.

In certain embodiments of the various methods provided herein, the ADC is administered at a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In some embodiments, the ADC is administered at a dose of 1 to 10 mg/kg of the subject's body weight. In certain embodiments, the ADC is administered at a dose of 1 to 5 mg/kg of the subject's body weight. In other embodiments, the ADC is administered at a dose of 1 to 2.5 mg/kg of the subject's body weight. In further embodiments, the ADC is administered at a dose of 1 to 1.25 mg/kg of the subject's body weight. In some embodiments, the ADC is administered at a dose of 0.25 mg/kg of the subject's body weight. In some embodiments, the ADC is administered at a dose of 0.5 mg/kg of the subject's body weight. In some embodiments, the ADC is administered at a dose of 0.75 mg/kg of the subject's body weight. In some embodiments, the ADC is administered at a dose of 1.0 mg/kg of the subject's body weight. In some embodiments, the ADC is administered at a dose of 1.25 mg/kg of the subject's body weight. In some embodiments, the ADC is administered at a dose of 1.5 mg/kg of the subject's body weight. In some embodiments, the ADC is administered at a dose of 1.75 mg/kg of the subject's body weight. In some embodiments, the ADC is administered at a dose of 2.0 mg/kg of the subject's body weight. In some embodiments, the ADC is administered at a dose of 2.25 mg/kg of the subject's body weight. In some embodiments, the ADC is administered at a dose of 2.5 mg/kg of the subject's body weight.

In some embodiments of the various methods provided herein, including those methods requiring a first and a second dose, the first dose of the ADC is a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments of the various methods provided herein, including those methods requiring a first and a second dose, the first dose of the ADC is a dose of about 1 to about 10 mg/kg of the subject's body weight. In some embodiments of the various methods provided herein, including those methods requiring a first and a second dose, the first dose of the ADC is a dose of about 1 to about 5 mg/kg of the subject's body weight. In some embodiments of the various methods provided herein, including those methods requiring a first and a second dose, the first dose of the ADC is a dose of about 1 to about 2.5 mg/kg of the subject's body weight. In some embodiments of the various methods provided herein, including those methods requiring a first and a second dose, the first dose of the ADC is a dose of about 1 to about 1.25 mg/kg of the subject's body weight. In some embodiments of the various methods provided herein, including those methods requiring a first and a second dose, the first dose of the ADC is a dose of about 0.25 mg/kg of the subject's body weight. In some embodiments of the various methods provided herein, including those methods requiring a first and a second dose, the first dose of the ADC is a dose of about 0.5 mg/kg of the subject's body weight. In some embodiments, the first dose of the ADC is a dose of about 0.75 mg/kg of the subject's body weight. In some embodiments, the first dose of the ADC is a dose of about 1.0 mg/kg of the subject's body weight. In some embodiments, the first dose of the ADC is a dose of about 1.25 mg/kg of the subject's body weight. In some embodiments, the first dose of the ADC is a dose of about 1.5 mg/kg of the subject's body weight. In some embodiments, the first dose of the ADC is a dose of about 1.75 mg/kg of the subject's body weight. In some embodiments, the first dose of the ADC is a dose of about 2.0 mg/kg of the subject's body weight. In some embodiments, the first dose of the ADC is a dose of about 2.25 mg/kg of the subject's body weight. In some embodiments, the first dose of the ADC is a dose of or about 2.5 mg/kg of the subject's body weight.

In certain embodiments of the various methods provided herein, including those methods requiring a first and a second dose, the first dose of the ADC is a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In certain embodiments of the various methods provided herein, including those methods requiring a first and a second dose, the first dose of the ADC is a dose of 1 to 10 mg/kg of the subject's body weight. In some embodiments of the various methods provided herein, including those methods requiring a first and a second dose, the first dose of the ADC is a dose of 1 to 5 mg/kg of the subject's body weight. In some embodiments of the various methods provided herein, including those methods requiring a first and a second dose, the first dose of the ADC is a dose of 1 to 2.5 mg/kg of the subject's body weight. In some embodiments of the various methods provided herein, including those methods requiring a first and a second dose, the first dose of the ADC is a dose of 1 to 1.25 mg/kg of the subject's body weight. In some embodiments of the various methods provided herein, including those methods requiring a first and a second dose, the first dose of the ADC is a dose of 0.25 mg/kg of the subject's body weight. In some embodiments of the various methods provided herein, including those methods requiring a first and a second dose, the first dose of the ADC is a dose of 0.5 mg/kg of the subject's body weight. In some embodiments, the first dose of the ADC is a dose of 0.75 mg/kg of the subject's body weight. In some embodiments, the first dose of the ADC is a dose of 1.0 mg/kg of the subject's body weight. In some embodiments, the first dose of the ADC is a dose of 1.25 mg/kg of the subject's body weight. In some embodiments, the first dose of the ADC is a dose of 1.5 mg/kg of the subject's body weight. In some embodiments, the first dose of the ADC is a dose of 1.75 mg/kg of the subject's body weight. In some embodiments, the first dose of the ADC is a dose of 2.0 mg/kg of the subject's body weight. In some embodiments, the first dose of the ADC is a dose of 2.25 mg/kg of the subject's body weight. In some embodiments, the first dose of the ADC is a dose of 2.5 mg/kg of the subject's body weight.

In some embodiments of the various methods provided herein, including those methods requiring a first and a second dose, the second dose of the ADC is lower than the first dose by about 0.1 mg/kg to about 2 mg/kg of the subject's body weight. In some embodiments of the various methods provided herein, including those methods requiring a first and a second dose, the second dose of the ADC is lower than the first dose by about 0.1 mg/kg of the subject's body weight. In some embodiments of the various methods provided herein, including those methods requiring a first and a second dose, the second dose of the ADC is lower than the first dose by about 0.2 mg/kg of the subject's body weight. In some embodiments of the various methods provided herein, including those methods requiring a first and a second dose, the second dose of the ADC is lower than the first dose by about 0.25 mg/kg of the subject's body weight. In some embodiments of the various methods provided herein, including those methods requiring a first and a second dose, the second dose of the ADC is lower than the first dose by about 0.3 mg/kg of the subject's body weight. In some embodiments of the various methods provided herein, including those methods requiring a first and a second dose, the second dose of the ADC is lower than the first dose by about 0.4 mg/kg of the subject's body weight. In some embodiments of the various methods provided herein, including those methods requiring a first and a second dose, the second dose of the ADC is lower than the first dose by about 0.5 mg/kg of the subject's body weight. In some embodiments of the various methods provided herein, including those methods requiring a first and a second dose, the second dose of the ADC is lower than the first dose by about 0.6 mg/kg of the subject's body weight. In some embodiments of the various methods provided herein, including those methods requiring a first and a second dose, the second dose of the ADC is lower than the first dose by about 0.7 mg/kg of the subject's body weight. In some embodiments of the various methods provided herein, including those methods requiring a first and a second dose, the second dose of the ADC is lower than the first dose by about 0.75 mg/kg of the subject's body weight. In some embodiments of the various methods provided herein, including those methods requiring a first and a second dose, the second dose of the ADC is lower than the first dose by about 0.8 mg/kg of the subject's body weight. In some embodiments of the various methods provided herein, including those methods requiring a first and a second dose, the second dose of the ADC is lower than the first dose by about 0.9 mg/kg of the subject's body weight. In some embodiments of the various methods provided herein, including those methods requiring a first and a second dose, the second dose of the ADC is lower than the first dose by about 1 mg/kg of the subject's body weight. In some embodiments of the various methods provided herein, including those methods requiring a first and a second dose, the second dose of the ADC is lower than the first dose by about 1.1 mg/kg of the subject's body weight. In some embodiments of the various methods provided herein, including those methods requiring a first and a second dose, the second dose of the ADC is lower than the first dose by about 1.2 mg/kg of the subject's body weight. In some embodiments of the various methods provided herein, including those methods requiring a first and a second dose, the second dose of the ADC is lower than the first dose by about 1.25 mg/kg of the subject's body weight. In some embodiments of the various methods provided herein, including those methods requiring a first and a second dose, the second dose of the ADC is lower than the first dose by about 1.3 mg/kg of the subject's body weight. In some embodiments of the various methods provided herein, including those methods requiring a first and a second dose, the second dose of the ADC is lower than the first dose by about 1.4 mg/kg of the subject's body weight. In some embodiments of the various methods provided herein, including those methods requiring a first and a second dose, the second dose of the ADC is lower than the first dose by about 1.5 mg/kg of the subject's body weight. In some embodiments of the various methods provided herein, including those methods requiring a first and a second dose, the second dose of the ADC is lower than the first dose by about 1.6 mg/kg of the subject's body weight. In some embodiments of the various methods provided herein, including those methods requiring a first and a second dose, the second dose of the ADC is lower than the first dose by about 1.7 mg/kg of the subject's body weight. In some embodiments of the various methods provided herein, including those methods requiring a first and a second dose, the second dose of the ADC is lower than the first dose by about 1.75 mg/kg of the subject's body weight. In some embodiments of the various methods provided herein, including those methods requiring a first and a second dose, the second dose of the ADC is lower than the first dose by about 1.8 mg/kg of the subject's body weight. In some embodiments of the various methods provided herein, including those methods requiring a first and a second dose, the second dose of the ADC is lower than the first dose by about 1.9 mg/kg of the subject's body weight. In some embodiments of the various methods provided herein, including those methods requiring a first and a second dose, the second dose of the ADC is lower than the first dose by about 2 mg/kg of the subject's body weight.

In certain embodiments of the various methods provided herein, including those methods requiring a first and a second dose, the second dose of the ADC is lower than the first dose by 0.1 mg/kg to 2 mg/kg of the subject's body weight. In some embodiments of the various methods provided herein, including those methods requiring a first and a second dose, the second dose of the ADC is lower than the first dose by 0.1 mg/kg of the subject's body weight. In some embodiments of the various methods provided herein, including those methods requiring a first and a second dose, the second dose of the ADC is lower than the first dose by 0.2 mg/kg of the subject's body weight. In some embodiments of the various methods provided herein, including those methods requiring a first and a second dose, the second dose of the ADC is lower than the first dose by 0.25 mg/kg of the subject's body weight. In some embodiments of the various methods provided herein, including those methods requiring a first and a second dose, the second dose of the ADC is lower than the first dose by 0.3 mg/kg of the subject's body weight. In some embodiments of the various methods provided herein, including those methods requiring a first and a second dose, the second dose of the ADC is lower than the first dose by 0.4 mg/kg of the subject's body weight. In some embodiments of the various methods provided herein, including those methods requiring a first and a second dose, the second dose of the ADC is lower than the first dose by 0.5 mg/kg of the subject's body weight. In some embodiments of the various methods provided herein, including those methods requiring a first and a second dose, the second dose of the ADC is lower than the first dose by 0.6 mg/kg of the subject's body weight. In some embodiments of the various methods provided herein, including those methods requiring a first and a second dose, the second dose of the ADC is lower than the first dose by 0.7 mg/kg of the subject's body weight. In some embodiments of the various methods provided herein, including those methods requiring a first and a second dose, the second dose of the ADC is lower than the first dose by 0.75 mg/kg of the subject's body weight. In some embodiments of the various methods provided herein, including those methods requiring a first and a second dose, the second dose of the ADC is lower than the first dose by 0.8 mg/kg of the subject's body weight. In some embodiments of the various methods provided herein, including those methods requiring a first and a second dose, the second dose of the ADC is lower than the first dose by 0.9 mg/kg of the subject's body weight. In some embodiments of the various methods provided herein, including those methods requiring a first and a second dose, the second dose of the ADC is lower than the first dose by 1 mg/kg of the subject's body weight. In some embodiments of the various methods provided herein, including those methods requiring a first and a second dose, the second dose of the ADC is lower than the first dose by 1.1 mg/kg of the subject's body weight. In some embodiments of the various methods provided herein, including those methods requiring a first and a second dose, the second dose of the ADC is lower than the first dose by 1.2 mg/kg of the subject's body weight. In some embodiments of the various methods provided herein, including those methods requiring a first and a second dose, the second dose of the ADC is lower than the first dose by 1.25 mg/kg of the subject's body weight. In some embodiments of the various methods provided herein, including those methods requiring a first and a second dose, the second dose of the ADC is lower than the first dose by 1.3 mg/kg of the subject's body weight. In some embodiments of the various methods provided herein, including those methods requiring a first and a second dose, the second dose of the ADC is lower than the first dose by 1.4 mg/kg of the subject's body weight. In some embodiments of the various methods provided herein, including those methods requiring a first and a second dose, the second dose of the ADC is lower than the first dose by 1.5 mg/kg of the subject's body weight. In some embodiments of the various methods provided herein, including those methods requiring a first and a second dose, the second dose of the ADC is lower than the first dose by 1.6 mg/kg of the subject's body weight. In some embodiments of the various methods provided herein, including those methods requiring a first and a second dose, the second dose of the ADC is lower than the first dose by 1.7 mg/kg of the subject's body weight. In some embodiments of the various methods provided herein, including those methods requiring a first and a second dose, the second dose of the ADC is lower than the first dose by 1.75 mg/kg of the subject's body weight. In some embodiments of the various methods provided herein, including those methods requiring a first and a second dose, the second dose of the ADC is lower than the first dose by 1.8 mg/kg of the subject's body weight. In some embodiments of the various methods provided herein, including those methods requiring a first and a second dose, the second dose of the ADC is lower than the first dose by 1.9 mg/kg of the subject's body weight. In some embodiments of the various methods provided herein, including those methods requiring a first and a second dose, the second dose of the ADC is lower than the first dose by 2 mg/kg of the subject's body weight.

In some embodiments of the various methods provided herein, including those methods requiring a first and a second dose, the second dose of the ADC is a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments of the various methods provided herein, including those methods requiring a first and a second dose, the second dose of the ADC is a dose of about 1 to about 10 mg/kg of the subject's body weight. In some embodiments of the various methods provided herein, including those methods requiring a first and a second dose, the second dose of the ADC is a dose of about 1 to about 5 mg/kg of the subject's body weight. In some embodiments of the various methods provided herein, including those methods requiring a first and a second dose, the second dose of the ADC is a dose of about 1 to about 2.5 mg/kg of the subject's body weight. In some embodiments of the various methods provided herein, including those methods requiring a first and a second dose, the second dose of the ADC is a dose of about 1 to about 1.25 mg/kg of the subject's body weight. In some embodiments of the various methods provided herein, including those methods requiring a first and a second dose, the second dose of the ADC is a dose of about 0.25 mg/kg of the subject's body weight. In some embodiments of the various methods provided herein, including those methods requiring a first and a second dose, the second dose of the ADC is a dose of about 0.5 mg/kg of the subject's body weight. In some embodiments of the various methods provided herein, including those methods requiring a first and a second dose, the second dose of the ADC is a dose of about 0.75 mg/kg of the subject's body weight. In some embodiments of the various methods provided herein, including those methods requiring a first and a second dose, the second dose of the ADC is a dose of about 1.0 mg/kg of the subject's body weight. In some embodiments of the various methods provided herein, including those methods requiring a first and a second dose, the second dose of the ADC is a dose of about 1.25 mg/kg of the subject's body weight. In some embodiments of the various methods provided herein, including those methods requiring a first and a second dose, the second dose of the ADC is a dose of about 1.5 mg/kg of the subject's body weight. In some embodiments of the various methods provided herein, including those methods requiring a first and a second dose, the second dose of the ADC is a dose of about 1.75 mg/kg of the subject's body weight. In some embodiments of the various methods provided herein, including those methods requiring a first and a second dose, the second dose of the ADC is a dose of about 2.0 mg/kg of the subject's body weight. In some embodiments of the various methods provided herein, including those methods requiring a first and a second dose, the second dose of the ADC is a dose of about 2.25 mg/kg of the subject's body weight. In some embodiments of the various methods provided herein, including those methods requiring a first and a second dose, the second dose of the ADC is a dose of about 2.5 mg/kg of the subject's body weight.

In certain embodiments of the various methods provided herein, including those methods requiring a first and a second dose, the second dose of the ADC is a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In certain embodiments of the various methods provided herein, including those methods requiring a first and a second dose, the second dose of the ADC is a dose of 1 to 10 mg/kg of the subject's body weight. In some embodiments of the various methods provided herein, including those methods requiring a first and a second dose, the second dose of the ADC is a dose of 1 to 5 mg/kg of the subject's body weight. In some embodiments of the various methods provided herein, including those methods requiring a first and a second dose, the second dose of the ADC is a dose of 1 to 2.5 mg/kg of the subject's body weight. In some embodiments of the various methods provided herein, including those methods requiring a first and a second dose, the second dose of the ADC is a dose of 1 to 1.25 mg/kg of the subject's body weight. In some embodiments of the various methods provided herein, including those methods requiring a first and a second dose, the second dose of the ADC is a dose of 0.25 mg/kg of the subject's body weight. In some embodiments of the various methods provided herein, including those methods requiring a first and a second dose, the second dose of the ADC is a dose of 0.5 mg/kg of the subject's body weight. In some embodiments of the various methods provided herein, including those methods requiring a first and a second dose, the second dose of the ADC is a dose of 0.75 mg/kg of the subject's body weight. In some embodiments of the various methods provided herein, including those methods requiring a first and a second dose, the second dose of the ADC is a dose of 1.0 mg/kg of the subject's body weight. In some embodiments of the various methods provided herein, including those methods requiring a first and a second dose, the second dose of the ADC is a dose of 1.25 mg/kg of the subject's body weight. In some embodiments of the various methods provided herein, including those methods requiring a first and a second dose, the second dose of the ADC is a dose of 1.5 mg/kg of the subject's body weight. In some embodiments of the various methods provided herein, including those methods requiring a first and a second dose, the second dose of the ADC is a dose of 1.75 mg/kg of the subject's body weight. In some embodiments of the various methods provided herein, including those methods requiring a first and a second dose, the second dose of the ADC is a dose of 2.0 mg/kg of the subject's body weight. In some embodiments of the various methods provided herein, including those methods requiring a first and a second dose, the second dose of the ADC is a dose of 2.25 mg/kg of the subject's body weight. In some embodiments of the various methods provided herein, including those methods requiring a first and a second dose, the second dose of the ADC is a dose of 2.5 mg/kg of the subject's body weight.

In certain embodiments of the various methods provided herein, including those methods requiring a first and a second dose, the second dose of the ADC is identical to the first dose of the ADC.

In some embodiments of the methods provided herein, the ADC is administered by an intravenous (IV) injection or infusion. In one embodiment, the first dose of the ADC is administered by an IV injection. In another embodiment, the first dose of the ADC is administered by an IV infusion. In yet another embodiment, the second dose of the ADC is administered by an IV injection. In yet another embodiment, the second dose of the ADC is administered by an IV injection infusion. In one embodiment, the first dose of the ADC is administered by an IV injection and the second dose of the ADC is administered by an IV injection. In another embodiment, the first dose of the ADC is administered by an IV infusion and the second dose of the ADC is administered by an IV injection. In yet another embodiment, the second dose of the ADC is administered by an IV injection and the second dose of the ADC is administered by an IV injection infusion. In yet another embodiment, the second dose of the ADC is administered by an IV injection infusion and the second dose of the ADC is administered by an IV injection infusion. In some embodiments, the ADC of the methods for which the various dosages are described in this paragraph is enfortumab vedotin (EV).

In certain embodiments of the methods provided herein, the ADC is administered by an IV injection or infusion three times every four-week cycle. In some embodiments of the methods provided herein, the first dose of the ADC is administered by an IV injection or infusion three times every four-week cycle. In some embodiments of the methods provided herein, the second dose of the ADC is administered by an IV injection or infusion three times every four-week cycle. In some embodiments of the methods provided herein, the first dose of the ADC is administered by an IV injection or infusion three times every four-week cycle and the second dose of the ADC is administered by an IV injection or infusion three times every four-week cycle. In some embodiments, the ADC of the methods for which the various dosages are described in this paragraph is enfortumab vedotin (EV).

In some embodiments of the methods provided herein, the ADC is administered by an IV injection or infusion on Days 1, 8 and 15 of every four-week cycle. In some embodiments, the first dose of ADC is administered by an IV injection or infusion on Days 1, 8 and 15 of every four-week cycle. In some embodiments, the second dose of ADC is administered by an IV injection or infusion on Days 1, 8 and 15 of every four-week cycle. In some embodiments, the first dose of ADC is administered by an IV injection or infusion on Days 1, 8 and 15 of every four-week cycle and the second dose of ADC is administered by an IV injection or infusion on Days 1, 8 and 15 of every four-week cycle. In some embodiments, the ADC of the methods for which the various dosages are described in this paragraph is enfortumab vedotin (EV).

In certain embodiments of the methods provided herein, the ADC is administered by an IV injection or infusion over about 30 minutes three times every four-week cycle. In some embodiments, the first dose of the ADC is administered by an IV injection or infusion over about 30 minutes three times every four-week cycle. In some embodiments, the second dose of the ADC is administered by an IV injection or infusion over about 30 minutes three times every four-week cycle. In some embodiments, the first dose of the ADC is administered by an IV injection or infusion over about 30 minutes three times every four-week cycle and the second dose of the ADC is administered by an IV injection or infusion over about 30 minutes three times every four-week cycle. In some embodiments, the ADC of the methods for which the various dosages are described in this paragraph is enfortumab vedotin (EV).

In some embodiments of the methods provided herein, the ADC is administered by an IV injection or infusion over about 30 minutes on Days 1, 8 and 15 of every four-week cycle. In some embodiments of the methods provided herein, the first dose of the ADC is administered by an IV injection or infusion over about 30 minutes on Days 1, 8 and 15 of every four-week cycle. In some embodiments of the methods provided herein, the second dose of the ADC is administered by an IV injection or infusion over about 30 minutes on Days 1, 8 and 15 of every four-week cycle. In some embodiments of the methods provided herein, the first dose of the ADC is administered by an IV injection or infusion over about 30 minutes on Days 1, 8 and 15 of every four-week cycle and the second dose of the ADC is administered by an IV injection or infusion over about 30 minutes on Days 1, 8 and 15 of every four-week cycle. In some embodiments, the ADC of the methods for which the various dosages are described in this paragraph is enfortumab vedotin (EV).

In other more specific embodiments, the antibody drug conjugate formulated in the pharmaceutical composition provided herein is administered at a dose of about 1 mg/kg, 1.25 mg/kg, or about 1.5 mg/kg of the subject's body weight by an intravenous (IV) injection or infusion over about 30 minutes three times every 28-day cycle. In some embodiments, the antibody drug conjugate formulated in the pharmaceutical composition is administered by an intravenous (IV) injection or infusion over about 30 minutes on Days 1, 8 and 15 of every 28-day cycle. In some embodiments, the method further comprises administering an immune checkpoint inhibitor by an intravenous (IV) injection or infusion one or more times in each four-week cycle. In some embodiments of the methods provided herein, the ADC is administered three times within a 28 day cycle. In some embodiments of the methods provided herein, the ADC is administered on Days 1, 8 and 15 of a 28 day cycle. In some embodiments, the ADC of the methods for which the various dosages are described in this paragraph is enfortumab vedotin (EV).

In one specific embodiment of the methods provided herein, the ADC has the following structure:

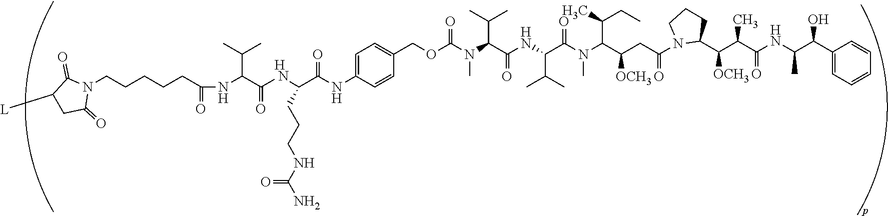

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered at a dose of about 1.25 mg/kg of the subject's body weight, and wherein the dose is administered by an IV injection or infusion over about 30 minutes on Days 1, 8 and 15 of every four-week cycle. 5.8 Combination Therapies

Provided herein are combination therapies using an antibody drug conjugate, wherein the antibody drug conjugate comprises an antibody or antigen binding fragment thereof that binds to 191P4D12 conjugated to one or more units of monomethyl auristatin E (MMAE) in combination with a checkpoint inhibitor (such as a PD-1 inhibitor or a PD-L1 inhibitor, e.g., pembrolizumab) for treating cancer in a subject. In any of the embodiments disclosed herein, the checkpoint inhibitor may be pembrolizumab.

In some embodiments, a pharmaceutically effective amount of the antibody drug conjugate is administered. In some embodiments, a pharmaceutically effective amount of the checkpoint inhibitor (such as a PD-1 inhibitor or a PD-L1 inhibitor, e.g., pembrolizumab) is administered. In some embodiments, a pharmaceutically effective amount of the antibody drug conjugate is administered and a pharmaceutically effective amount of the checkpoint inhibitor (such as a PD-1 inhibitor or a PD-L1 inhibitor, e.g., pembrolizumab) is administered. In some embodiments, a pharmaceutically effective amount of the antibody drug conjugate is administered and a pharmaceutically effective amount of pembrolizumab is administered. In some embodiments, an effective amount of the antibody drug conjugate is administered and an effective amount of the checkpoint inhibitor (such as a PD-1 inhibitor or a PD-L1 inhibitor, e.g., pembrolizumab) is administered. In some embodiments, an effective amount of the antibody drug conjugate is administered and an effective amount of pembrolizumab is administered.

In some embodiments, the subject is a human. In some embodiments, the subject is a subject diagnosed with cancer, e.g., urothelial cancer, bladder cancer, cancer of the renal pelvis, cancer of the ureter, cancer of the urethra, locally advanced cancer, metastatic cancer, locally advanced urothelial cancer, unresectable locally advanced urothelial cancer, metastatic urothelial cancer, locally advanced bladder cancer, or metastatic bladder cancer.

The amount of the antibody drug conjugate provided herein or pembrolizumab, or a pharmaceutical composition that will be effective in the prevention and/or treatment of cancer can be determined by standard clinical techniques. In addition, in vitro assays may optionally be employed to help identify optimal dosage ranges. The precise dose to be employed in the formulation will also depend on the route of administration, and the seriousness of the cancer, and in some embodiments, should be decided according to the judgment of the practitioner and each patient's circumstances.

In some embodiments, the antibody drug conjugate provided herein is administered to the subject as part a composition. In some embodiments, the composition is a pharmaceutical composition described in Section 5.4

In some embodiments, the antibody drug conjugate and pembrolizumab may be formulated in different pharmaceutical compositions and administered separately to the subject in need thereof. In other embodiments, the antibody drug conjugate and pembrolizumab are administered together in the same pharmaceutical composition.

In other embodiments, the antibody drug conjugate and pembrolizumab are administered simultaneously. The term “simultaneously” means at the same time or within a short period of time, for example, less than 1 hour, less than 2 hours, less than 3 hours, less than 4 hours, or less than 12 hours.

In some embodiments, the antibody drug conjugate and pembrolizumab are not administered simultaneously, and instead the two compounds are administered at different times.

In some embodiments, pembrolizumab is administered before the administration of the antibody drug conjugate.

In some embodiments, pembrolizumab is administered after the administration of the antibody drug conjugate.

In certain embodiments, the subject has been previously treated with pembrolizumab prior to the administration of the antibody drug conjugate.

In certain embodiments, the subject has been previously treated with the antibody drug conjugate prior to the administration of pembrolizumab.

In certain embodiments, the subject has not been previously treated with pembrolizumab prior to the co-administration of the antibody drug conjugate and pembrolizumab.

In certain embodiments, the co-administration of the antibody drug conjugate and pembrolizumab is concomitant administration.

In certain embodiments, the co-administration of the antibody drug conjugate and pembrolizumab is pharmaceutically effective to treat a cancer.

Any amount or dose of the antibody drug conjugate disclosed herein may be administered in combination with any amount or dose of pembrolizumab disclosed herein. In some embodiments, the antibody drug conjugate and pembrolizumab are administered at least once during a dosing period. A dosing period as used herein is meant a period of time, during which each therapeutic agent has been administered at least once. A dosing cycle can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, a dosing cycle is 1, 2, 3, 4, 5, 6, 7, 8, 9, or 10 weeks. In certain embodiments, a dosing period is a dosing cycle.

The therapeutic agent (the antibody drug conjugate provided herein and/or pembrolizumab) can be delivered as a single dose (e.g., a single bolus injection), or over time (e.g., continuous infusion over time or divided bolus doses over time). The agent can be administered repeatedly if necessary, for example, until the patient experiences stable disease or regression, or until the patient experiences disease progression or unacceptable toxicity. Stable disease or lack is determined by methods known in the art such as evaluation of patient symptoms, physical examination, and visualization of the tumor that has been imaged using X-ray, CAT, PET, MRI scan, or other commonly accepted evaluation modalities.

The therapeutic agent (the antibody drug conjugate provided herein and/or pembrolizumab) can be administered once daily (QD) or divided into multiple daily doses such as twice daily (BID), three times daily (TID), and four times daily (QID). In addition, the administration can be continuous (i.e., daily for consecutive days or every day) or intermittent, e.g., in cycles (i.e., including days, weeks, or months of rest without drug). As used herein, the term “daily” is intended to mean that a therapeutic compound is administered once or more than once each day, for example, for a period of time. The term “continuous” is intended to mean that a therapeutic compound is administered daily for an uninterrupted period of, e.g., at least 10 days. The term “intermittent” or “intermittently” as used herein is intended to mean stopping and starting at either regular or irregular intervals. For example, intermittent administration of the compound is administration for one to six days per week, administration in cycles (e.g., daily administration for two to eight consecutive weeks, then a rest period with no administration for up to one week), or administration on alternate days.

In some embodiments, the frequency of administration is in the range of about a daily dose to about a monthly dose. In certain embodiments, administration is once a day, twice a day, three times a day, four times a day, once every other day, twice every other day, three times every other day, four times every other day, twice a week, three times a week, four times a week, five times a week, once a week, once every two weeks, once every three weeks, or once every four weeks.

In certain embodiments, the compound is administered once per day, twice a day, three times a day, or four times a day from one day to six months, from one week to three months, from one week to four weeks, from one week to three weeks, or from one week to two weeks.

In some embodiments, the antibody drug conjugate provided herein is administered in a therapeutically effective amount and pembrolizumab is administered in a therapeutically effective amount. A therapeutically effective amount of the antibody drug conjugate may be any amount or dose of the antibody drug conjugate disclosed herein. A therapeutically effective amount of pembrolizumab may be any amount or dose of pembrolizumab disclosed herein. In certain embodiments, any amount or dose of the antibody drug conjugate disclosed herein may be administered in combination with any amount or dose of pembrolizumab disclosed herein (see, e.g., Sections 5.2.1.3, 5.4, and 5.7).

The antibody drug conjugate and/or pembrolizumab may be administered or dosed according to body weight (mg/kg) or body surface area (BSA) (mg/m²). As an example, which is intended to be illustrative, and not limiting, the average body mass globally has been calculated to be 62 kg. See Walpole et al., 2012, BMC Public Health, 12:439 (doi: 10.1186/1471-2458-12-439).

In certain embodiments, the antibody drug conjugate provided herein is administered at a dose of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg.

In certain embodiments, the antibody drug conjugate provided herein is administered at a dose of about 0.1 mg/kg to about 100 mg/kg of the subject's body weight. In some embodiments, the antibody drug conjugate provided herein is administered at a dose of about 1 mg/kg to about 75 mg/kg of the subject's body weight. In some embodiments, the antibody drug conjugate provided herein is administered at a dose of between about 1 mg/kg and about 20 mg/kg of the subject's body weight, such as about 1 mg/kg to about 5 mg/kg of the subject's body weight, about 1 mg/kg to about 4 mg/kg of the subject's body weight, about 1 mg/kg to about 3 mg/kg of the subject's body weight, or about 1 mg/kg to about 2 mg/kg of the subject's body weight.

In certain embodiments, the pembrolizumab is administered at a dose of about 100 mg, about 150 mg, about 200 mg, about 250 mg, about 300 mg, about 350 mg, about 400 mg, about 450 mg or about 500 mg. In particular embodiments, the pembrolizumab is administered at a dose of about 200 mg.

In certain embodiments, the pembrolizumab is administered at a dose of about 100 mg to about about 500 mg, about 150 mg to about about 500 mg, about 200 mg to about 500 mg, about 250 mg to about 500 mg, about 300 mg to about 500 mg, about 350 mg to about 500 mg, about 400 mg to about 500 mg, or about 450 mg to about 500 mg. In certain embodiments, the pembrolizumab is administered at a dose of about 100 mg to about about 450 mg, about 100 mg to about about 400 mg, about 100 mg to about about 350 mg, about 100 mg to about 300 mg, about 100 mg to about 250 mg, about 100 mg to about 200 mg, or about 100 mg to about 150 mg. In certain embodiments, the pembrolizumab is administered at a dose of about 150 mg to about 450 mg, about 150 to about 400 mg, about 150 to about 350 mg, about 200 mg to about 450 mg, about 200 mg to about 400 mg, about 200 mg to about 300 mg, or about 200 mg to about 250 mg.

In some embodiments, the antibody drug conjugate provided herein is administered daily during each treatment cycle of 14 days, 21 days, 28 days, 35 days, or 42 days. In some embodiments, the antibody drug conjugate is administered for 1, 2, 3, 4, 5, 6, 7, 8, 9, 10, 11, 12, 13, or 14 days of a treatment cycle of 14 days. In some embodiments, the antibody drug conjugate is administered for 1, 2, 3, 4, 5, 6, 7, 8, 9, 10, 11, 12, 13, 14, 15, 16, 17, 18, 19, 20, or 21 days of a treatment cycle of 21 days (i.e., 3 weeks). In some embodiments, the antibody drug conjugate is administered for 1, 2, 3, 4, 5, 6, 7, 8, 9, 10, 11, 12, 13, 14, 15, 16, 17, 18, 19, 20, 21, 22, 23, 24, 25, 26, 27, or 28 days of a treatment cycle of 28 days. In some embodiments, the antibody drug conjugate is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days of a treatment cycle of 35 days. In some embodiments, the antibody drug conjugate is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or42 days of a treatment cycle of 42 days. In some embodiments, the antibody drug conjugate is administered on 1, 2, 3, 4, 5, 6, 7, 8, 9, 10, 11, 12, 13, 14, 15, 16, 17, 18, 19, 20, or 21 days of a treatment cycle of 21 days. In certain embodiments, the antibody drug conjugate is administered on days 1, 2, 3, 4, 5, 6, 7, 8, 9, 10, 11, 12, 13, 14, 15, 16, 17, 18, 19, 20, and/or 21 of a treatment cycle of 21 days. In particular embodiments, the antibody drug conjugate is administered on days 1 and 8 of a treatment cycle of 21 days. In some embodiments, the antibody drug conjugate is administered for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 treatment cycles.

In some embodiments, the antibody drug conjugate is administered once, two times, three times, or four times daily during each treatment cycle of 14 days, 21 days, 28 days, 35 days, or 42 days. In some embodiments, the antibody drug conjugate is administered once, two times, three times, or four times daily for 1, 2, 3, 4, 5, 6, 7, 8, 9, 10, 11, 12, 13, or 14 days of each treatment cycle of 14 days. In some embodiments, the antibody drug conjugate is administered once, two times, three times, or four times daily for 1, 2, 3, 4, 5, 6, 7, 8, 9, 10, 11, 12, 13, 14, 15, 16, 17, 18, 19, 20, or 21 days of each treatment cycle of 21 days. In some embodiments, the antibody drug conjugate is administered once, two times, three times, or four times daily for 1, 2, 3, 4, 5, 6, 7, 8, 9, 10, 11, 12, 13, 14, 15, 16, 17, 18, 19, 20, 21, 22, 23, 24, 25, 26, 27, or 28 days of each treatment cycle of 28 days. In some embodiments, the antibody drug conjugate is administered once, two times, three times, or four times daily for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days of each treatment cycle of 35 days. In some embodiments, the antibody drug conjugate is administered once, two times, three times, or four times daily for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or42 days of each treatment cycle of 42 days. In certain embodiments, the antibody drug conjugate is administered once, two times, three times, or four times daily for 2 days of each treatment cycle of 21 days.

In certain embodiments, the antibody drug conjugate is administered at a daily dose of about 1.25 mg/kg for 2 days in each treatment cycle of 21 days. In certain embodiments, the antibody drug conjugate is administered at a daily dose of about 1.25 mg/kg on days 1 and 8 in each treatment cycle of 21 days. In certain embodiments, the antibody drug conjugate is administered at a daily dose of about 1.25 mg/kg on days 1 and 8 in each treatment cycle of 21 days by an intravenous injection or infusion. In certain embodiments, the antibody drug conjugate is administered at a daily dose of about 1.25 mg/kg on days 1 and 8 in each treatment cycle of 21 days by intravenous infusion.

In some embodiments, the antibody drug conjugate is administered intravenously. In some embodiments, the antibody drug conjugate is administered by an intravenous injection or infusion. In some embodiments, the antibody drug conjugate is administered by intravenous infusion. In some embodiments, the antibody drug conjugate is administered as a 30 minute (−5 minutes/+10 minutes) intravenous infusion. In one embodiment, the selected dose of the antibody drug conjugate is administered by intravenous infusion over a time period of between 25 and 40 minutes, or about 30 minutes (−5 minutes/+10 minutes).

In some embodiments, pembrolizumab is administered daily during each treatment cycle of 14 days, 21 days, 28 days, 35 days, or 42 days. In some embodiments, pembrolizumab is administered for 1, 2, 3, 4, 5, 6, 7, 8, 9, 10, 11, 12, 13, or 14 days of a treatment cycle of 14 days. In some embodiments, pembrolizumab is administered for 1, 2, 3, 4, 5, 6, 7, 8, 9, 10, 11, 12, 13, 14, 15, 16, 17, 18, 19, 20, or 21 days of a treatment cycle of 21 days (i.e., 3 weeks). In some embodiments, pembrolizumab is administered for 1, 2, 3, 4, 5, 6, 7, 8, 9, 10, 11, 12, 13, 14, 15, 16, 17, 18, 19, 20, 21, 22, 23, 24, 25, 26, 27, or 28 days of a treatment cycle of 28 days. In some embodiments, pembrolizumab is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days of a treatment cycle of 35 days. In some embodiments, pembrolizumab is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or42 days of a treatment cycle of 42 days. In some embodiments, pembrolizumab is administered on 1, 2, 3, 4, 5, 6, 7, 8, 9, 10, 11, 12, 13, 14, 15, 16, 17, 18, 19, 20, or 21 days of a treatment cycle of 21 days. In certain embodiments, pembrolizumab is administered on days 1, 2, 3, 4, 5, 6, 7, 8, 9, 10, 11, 12, 13, 14, 15, 16, 17, 18, 19, 20, and/or 21 of a treatment cycle of 21 days. In particular embodiments, pembrolizumab is administered on day 1 of a treatment cycle of 21 days. In some embodiments, pembrolizumab is administered for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 treatment cycles.

In some embodiments, pembrolizumab is administered once, two times, three times, or four times daily during each treatment cycle of 14 days, 21 days, 28 days, 35 days, or 42 days. In some embodiments, pembrolizumab is administered once, two times, three times, or four times daily for 1, 2, 3, 4, 5, 6, 7, 8, 9, 10, 11, 12, 13, or 14 days of each treatment cycle of 14 days. In some embodiments, pembrolizumab is administered once, two times, three times, or four times daily for 1, 2, 3, 4, 5, 6, 7, 8, 9, 10, 11, 12, 13, 14, 15, 16, 17, 18, 19, 20, or 21 days of each treatment cycle of 21 days. In some embodiments, pembrolizumab is administered once, two times, three times, or four times daily for 1, 2, 3, 4, 5, 6, 7, 8, 9, 10, 11, 12, 13, 14, 15, 16, 17, 18, 19, 20, 21, 22, 23, 24, 25, 26, 27, or 28 days of each treatment cycle of 28 days. In some embodiments, pembrolizumab is administered once, two times, three times, or four times daily for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days of each treatment cycle of 35 days. In some embodiments, pembrolizumab is administered once, two times, three times, or four times daily for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 days of each treatment cycle of 42 days. In certain embodiments, pembrolizumab is administered once, two times, three times, or four times daily for 1 day of each treatment cycle of 21 days.

In certain embodiments, pembrolizumab is administered at a daily dose of about 200 mg for 1 day in each treatment cycle of 21 days. In certain embodiments, pembrolizumab is administered at a daily dose of about 200 mg on day 1 in each treatment cycle of 21 days. In certain embodiments, pembrolizumab is administered at a daily dose of about 200 mg on day 1 in each treatment cycle of 21 days as an intravenous infusion. In certain embodiments, pembrolizumab is administered at a daily dose of about 200 mg on day 1 in each treatment cycle of 21 days by intravenous infusion.

In some embodiments, pembrolizumab is administered intravenously. In some embodiments, pembrolizumab is administered as an intravenous infusion. In some embodiments, pembrolizumab is administered by intravenous infusion. In some embodiments, pembrolizumab is administered as a 30 minute (−5 minutes/+10 minutes) intravenous infusion.

In some embodiments, the antibody drug conjugate is administered to the subject up to 2 days of a 21-day treatment cycle, and pembrolizumab is administered to the subject on 1 day of the 21-day treatment cycle. In some embodiments, the antibody drug conjugate is administered to the subject on days 1 and 8 of a 21-day treatment cycle, and pembrolizumab is administered to the subject on day 1 of the 21-day treatment cycle.

In some embodiments, the antibody drug conjugate is administered to the subject at a dose of about 1.0 mg/kg to about 5 mg/kg up to 2 days of a 21-day treatment cycle as an intravenous infusion, and pembrolizumab is administered to the subject at a dose of about 100 mg to about 300 mg on 1 day of the 21-day treatment cycle as an intravenous infusion. In some embodiments, the antibody drug conjugate is administered to the subject at a dose of about 1.0 mg/kg to about 5 mg/kg up to 2 days of a 21-day treatment cycle as an intravenous infusion, and pembrolizumab is administered to the subject at a dose of about 100 mg to about 300 mg on 1 day of the 21-day treatment cycle as an intravenous infusion 30 minutes after administration of the antibody drug conjugate. In some embodiments, the antibody drug conjugate is administered to the subject at a dose of about 1.0 mg/kg to about 5 mg/kg up to 2 days of a 21-day treatment cycle as an intravenous infusion, and pembrolizumab is administered to the subject at a dose of about 100 mg to about 300 mg on 1 day of the 21-day treatment cycle as an intravenous infusion 30 minutes after completion of the administration of the antibody drug conjugate. In some embodiments, the antibody drug conjugate is administered to the subject at a dose of about 1.0 mg/kg to about 5 mg/kg on days 1 and 8 of a 21-day treatment cycle by intravenous infusion, and pembrolizumab is administered to the subject at a dose of about 100 mg to about 300 mg on day 1 of the 21-day treatment cycle by intravenous infusion. In some embodiments, the antibody drug conjugate is administered to the subject at a dose of about 1.0 mg/kg to about 5 mg/kg on days 1 and 8 of a 21-day treatment cycle by intravenous infusion, and pembrolizumab is administered to the subject at a dose of about 100 mg to about 300 mg on day 1 of the 21-day treatment cycle by intravenous infusion 30 minutes after administration of the antibody drug conjugate. In some embodiments, the antibody drug conjugate is administered to the subject at a dose of about 1.0 mg/kg to about 5 mg/kg on days 1 and 8 of a 21-day treatment cycle by intravenous infusion, and pembrolizumab is administered at a dose of about 100 mg to about 300 mg to the subject on day 1 of the 21-day treatment cycle by intravenous infusion 30 minutes after completion of the administration of the antibody drug conjugate. In some embodiments, the antibody drug conjugate is administered to the subject at a dose of about 1.0 mg/kg to about 5 mg/kg up to 2 days of a 21-day treatment cycle as a 30 minute intravenous infusion, and pembrolizumab is administered to the subject at a dose of about 100 mg to about 300 mg on 1 day of the 21-day treatment cycle as a 30 minute intravenous infusion 30 minutes after administration of the antibody drug conjugate. In some embodiments, the antibody drug conjugate is administered to the subject at a dose of about 1.0 mg/kg to about 5 mg/kg up to 2 days of a 21-day treatment cycle as a 30 minute intravenous infusion, and pembrolizumab is administered to the subject at a dose of about 100 mg to about 300 mg on 1 day of the 21-day treatment cycle as a 30 minute intravenous infusion 30 minutes after completion of the administration of the antibody drug conjugate. In some embodiments, the antibody drug conjugate is administered to the subject at a dose of about 1.0 mg/kg to about 5 mg/kg on days 1 and 8 of a 21-day treatment cycle as a 30 minute intravenous infusion, and pembrolizumab is administered to the subject at a dose of about 100 mg to about 300 mg on day 1 of the 21-day treatment cycle as a 30 minute intravenous infusion. In some embodiments, the antibody drug conjugate is administered to the subject at a dose of about 1.0 mg/kg to about 5 mg/kg on days 1 and 8 of a 21-day treatment cycle as a 30 minute intravenous infusion, and pembrolizumab is administered to the subject at a dose of about 100 mg to about 300 mg on day 1 of the 21-day treatment cycle as a 30 minute intravenous infusion 30 minutes after administration of the antibody drug conjugate. In some embodiments, the antibody drug conjugate is administered to the subject at a dose of about 1.0 mg/kg to about 5 mg/kg on days 1 and 8 of a 21-day treatment cycle as a 30 minute intravenous infusion, and pembrolizumab is administered at a dose of about 100 mg to about 300 mg to the subject on day 1 of the 21-day treatment cycle as a 30 minute intravenous infusion 30 minutes after completion of the administration of the antibody drug conjugate.

In some embodiments, the antibody drug conjugate is administered to the subject at a dose of 1.0 mg/kg to 5 mg/kg up to 2 days of a 21-day treatment cycle as an intravenous infusion, and pembrolizumab is administered to the subject at a dose of 100 mg to 300 mg on 1 day of the 21-day treatment cycle as an intravenous infusion. In some embodiments, the antibody drug conjugate is administered to the subject at a dose of 1.0 mg/kg to 5 mg/kg up to 2 days of a 21-day treatment cycle as an intravenous infusion, and pembrolizumab is administered to the subject at a dose of 100 mg to 300 mg on 1 day of the 21-day treatment cycle as an intravenous infusion 30 minutes after administration of the antibody drug conjugate. In some embodiments, the antibody drug conjugate is administered to the subject at a dose of 1.0 mg/kg to 5 mg/kg up to 2 days of a 21-day treatment cycle as an intravenous infusion, and pembrolizumab is administered to the subject at a dose of 100 mg to 300 mg on 1 day of the 21-day treatment cycle as an intravenous infusion 30 minutes after completion of the administration of the antibody drug conjugate. In some embodiments, the antibody drug conjugate is administered to the subject at a dose of 1.0 mg/kg to 5 mg/kg on days 1 and 8 of a 21-day treatment cycle by intravenous infusion, and pembrolizumab is administered to the subject at a dose of 100 mg to 300 mg on day 1 of the 21-day treatment cycle by intravenous infusion. In some embodiments, the antibody drug conjugate is administered to the subject at a dose of 1.0 mg/kg to 5 mg/kg on days 1 and 8 of a 21-day treatment cycle by intravenous infusion, and pembrolizumab is administered to the subject at a dose of 100 mg to 300 mg on day 1 of the 21-day treatment cycle by intravenous infusion 30 minutes after administration of the antibody drug conjugate. In some embodiments, the antibody drug conjugate is administered to the subject at a dose of 1.0 mg/kg to 5 mg/kg on days 1 and 8 of a 21-day treatment cycle by intravenous infusion, and pembrolizumab is administered at a dose of 100 mg to 300 mg to the subject on day 1 of the 21-day treatment cycle by intravenous infusion 30 minutes after completion of the administration of the antibody drug conjugate. In some embodiments, the antibody drug conjugate is administered to the subject at a dose of 1.0 mg/kg to 5 mg/kg up to 2 days of a 21-day treatment cycle as a 30 minute intravenous infusion, and pembrolizumab is administered to the subject at a dose of 100 mg to 300 mg on 1 day of the 21-day treatment cycle as a 30 minute intravenous infusion 30 minutes after administration of the antibody drug conjugate. In some embodiments, the antibody drug conjugate is administered to the subject at a dose of 1.0 mg/kg to 5 mg/kg up to 2 days of a 21-day treatment cycle as a 30 minute intravenous infusion, and pembrolizumab is administered to the subject at a dose of 100 mg to 300 mg on 1 day of the 21-day treatment cycle as a 30 minute intravenous infusion 30 minutes after completion of the administration of the antibody drug conjugate. In some embodiments, the antibody drug conjugate is administered to the subject at a dose of 1.0 mg/kg to 5 mg/kg on days 1 and 8 of a 21-day treatment cycle as a 30 minute intravenous infusion, and pembrolizumab is administered to the subject at a dose of 100 mg to 300 mg on day 1 of the 21-day treatment cycle as a 30 minute intravenous infusion. In some embodiments, the antibody drug conjugate is administered to the subject at a dose of 1.0 mg/kg to 5 mg/kg on days 1 and 8 of a 21-day treatment cycle as a 30 minute intravenous infusion, and pembrolizumab is administered to the subject at a dose of 100 mg to 300 mg on day 1 of the 21-day treatment cycle as a 30 minute intravenous infusion 30 minutes after administration of the antibody drug conjugate. In some embodiments, the antibody drug conjugate is administered to the subject at a dose of 1.0 mg/kg to 5 mg/kg on days 1 and 8 of a 21-day treatment cycle as a 30 minute intravenous infusion, and pembrolizumab is administered at a dose of 100 mg to 300 mg to the subject on day 1 of the 21-day treatment cycle as a 30 minute intravenous infusion 30 minutes after completion of the administration of the antibody drug conjugate.

In some embodiments, the antibody drug conjugate is administered to the subject at a dose of about 1.25 mg/kg up to 2 days of a 21-day treatment cycle as an intravenous infusion, and pembrolizumab is administered to the subject at a dose of about 200 mg on 1 day of the 21-day treatment cycle as an intravenous infusion. In some embodiments, the antibody drug conjugate is administered to the subject at a dose of about 1.25 mg/kg up to 2 days of a 21-day treatment cycle as an intravenous infusion, and pembrolizumab is administered to the subject at a dose of about 200 mg on 1 day of the 21-day treatment cycle as an intravenous infusion 30 minutes after administration of the antibody drug conjugate. In some embodiments, the antibody drug conjugate is administered to the subject at a dose of about 1.25 mg/kg up to 2 days of a 21-day treatment cycle as an intravenous infusion, and pembrolizumab is administered to the subject at a dose of about 200 mg on 1 day of the 21-day treatment cycle as an intravenous infusion 30 minutes after completion of the administration of the antibody drug conjugate. In some embodiments, the antibody drug conjugate is administered to the subject at a dose of about 1.25 mg/kg on days 1 and 8 of a 21-day treatment cycle by intravenous infusion, and pembrolizumab is administered to the subject at a dose of about 200 mg on day 1 of the 21-day treatment cycle by intravenous infusion. In some embodiments, the antibody drug conjugate is administered to the subject at a dose of about 1.25 mg/kg on days 1 and 8 of a 21-day treatment cycle by intravenous infusion, and pembrolizumab is administered to the subject at a dose of about 200 mg on day 1 of the 21-day treatment cycle by intravenous infusion 30 minutes after administration of the antibody drug conjugate. In some embodiments, the antibody drug conjugate is administered to the subject at a dose of about 1.25 mg/kg on days 1 and 8 of a 21-day treatment cycle by intravenous infusion, and pembrolizumab is administered at a dose of about 200 mg to the subject on day 1 of the 21-day treatment cycle by intravenous infusion 30 minutes after completion of the administration of the antibody drug conjugate. In some embodiments, the antibody drug conjugate is administered to the subject at a dose of about 1.25 mg/kg up to 2 days of a 21-day treatment cycle as a 30 minute intravenous infusion, and pembrolizumab is administered to the subject at a dose of about 200 mg on 1 day of the 21-day treatment cycle as a 30 minute intravenous infusion 30 minutes after administration of the antibody drug conjugate. In some embodiments, the antibody drug conjugate is administered to the subject at a dose of about 1.25 mg/kg up to 2 days of a 21-day treatment cycle as a 30 minute intravenous infusion, and pembrolizumab is administered to the subject at a dose of about 200 mg on 1 day of the 21-day treatment cycle as a 30 minute intravenous infusion 30 minutes after completion of the administration of the antibody drug conjugate. In some embodiments, the antibody drug conjugate is administered to the subject at a dose of about 1.25 mg/kg on days 1 and 8 of a 21-day treatment cycle as a 30 minute intravenous infusion, and pembrolizumab is administered to the subject at a dose of about 200 mg on day 1 of the 21-day treatment cycle as a 30 minute intravenous infusion. In some embodiments, the antibody drug conjugate is administered to the subject at a dose of about 1.25 mg/kg on days 1 and 8 of a 21-day treatment cycle as a 30 minute intravenous infusion, and pembrolizumab is administered to the subject at a dose of about 200 mg on day 1 of the 21-day treatment cycle as a 30 minute intravenous infusion 30 minutes after administration of the antibody drug conjugate. In some embodiments, the antibody drug conjugate is administered to the subject at a dose of about 1.25 mg/kg on days 1 and 8 of a 21-day treatment cycle as a 30 minute intravenous infusion, and pembrolizumab is administered at a dose of about 200 mg to the subject on day 1 of the 21-day treatment cycle as a 30 minute intravenous infusion 30 minutes after completion of the administration of the antibody drug conjugate.

In some embodiments, the antibody drug conjugate is administered to the subject at a dose of 1.25 mg/kg up to 2 days of a 21-day treatment cycle as an intravenous infusion, and pembrolizumab is administered to the subject at a dose of 200 mg on 1 day of the 21-day treatment cycle as an intravenous infusion. In some embodiments, the antibody drug conjugate is administered to the subject at a dose of 1.25 mg/kg up to 2 days of a 21-day treatment cycle as an intravenous infusion, and pembrolizumab is administered to the subject at a dose of 200 mg on 1 day of the 21-day treatment cycle as an intravenous infusion 30 minutes after administration of the antibody drug conjugate. In some embodiments, the antibody drug conjugate is administered to the subject at a dose of 1.25 mg/kg up to 2 days of a 21-day treatment cycle as an intravenous infusion, and pembrolizumab is administered to the subject at a dose of 200 mg on 1 day of the 21-day treatment cycle as an intravenous infusion 30 minutes after completion of the administration of the antibody drug conjugate. In some embodiments, the antibody drug conjugate is administered to the subject at a dose of 1.25 mg/kg on days 1 and 8 of a 21-day treatment cycle by intravenous infusion, and pembrolizumab is administered to the subject at a dose of 200 mg on day 1 of the 21-day treatment cycle by intravenous infusion. In some embodiments, the antibody drug conjugate is administered to the subject at a dose of 1.25 mg/kg on days 1 and 8 of a 21-day treatment cycle by intravenous infusion, and pembrolizumab is administered to the subject at a dose of 200 mg on day 1 of the 21-day treatment cycle by intravenous infusion 30 minutes after administration of the antibody drug conjugate. In some embodiments, the antibody drug conjugate is administered to the subject at a dose of 1.25 mg/kg on days 1 and 8 of a 21-day treatment cycle by intravenous infusion, and pembrolizumab is administered at a dose of 200 mg to the subject on day 1 of the 21-day treatment cycle by intravenous infusion 30 minutes after completion of the administration of the antibody drug conjugate. In some embodiments, the antibody drug conjugate is administered to the subject at a dose of 1.25 mg/kg up to 2 days of a 21-day treatment cycle as a 30 minute intravenous infusion, and pembrolizumab is administered to the subject at a dose of 200 mg on 1 day of the 21-day treatment cycle as a 30 minute intravenous infusion 30 minutes after administration of the antibody drug conjugate. In some embodiments, the antibody drug conjugate is administered to the subject at a dose of 1.25 mg/kg up to 2 days of a 21-day treatment cycle as a 30 minute intravenous infusion, and pembrolizumab is administered to the subject at a dose of 200 mg on 1 day of the 21-day treatment cycle as a 30 minute intravenous infusion 30 minutes after completion of the administration of the antibody drug conjugate. In some embodiments, the antibody drug conjugate is administered to the subject at a dose of 1.25 mg/kg on days 1 and 8 of a 21-day treatment cycle as a 30 minute intravenous infusion, and pembrolizumab is administered to the subject at a dose of 200 mg on day 1 of the 21-day treatment cycle as a 30 minute intravenous infusion. In some embodiments, the antibody drug conjugate is administered to the subject at a dose of 1.25 mg/kg on days 1 and 8 of a 21-day treatment cycle as a 30 minute intravenous infusion, and pembrolizumab is administered to the subject at a dose of 200 mg on day 1 of the 21-day treatment cycle as a 30 minute intravenous infusion 30 minutes after administration of the antibody drug conjugate. In some embodiments, the antibody drug conjugate is administered to the subject at a dose of 1.25 mg/kg on days 1 and 8 of a 21-day treatment cycle as a 30 minute intravenous infusion, and pembrolizumab is administered at a dose of 200 mg to the subject on day 1 of the 21-day treatment cycle as a 30 minute intravenous infusion 30 minutes after completion of the administration of the antibody drug conjugate.

In some embodiments, the antibody drug conjugate is administered to the subject at a dose of about 1.0 mg/kg up to 2 days of a 21-day treatment cycle as an intravenous infusion, and pembrolizumab is administered to the subject at a dose of about 200 mg on 1 day of the 21-day treatment cycle as an intravenous infusion. In some embodiments, the antibody drug conjugate is administered to the subject at a dose of about 1.0 mg/kg up to 2 days of a 21-day treatment cycle as an intravenous infusion, and pembrolizumab is administered to the subject at a dose of about 200 mg on 1 day of the 21-day treatment cycle as an intravenous infusion 30 minutes after administration of the antibody drug conjugate. In some embodiments, the antibody drug conjugate is administered to the subject at a dose of about 1.0 mg/kg up to 2 days of a 21-day treatment cycle as an intravenous infusion, and pembrolizumab is administered to the subject at a dose of about 200 mg on 1 day of the 21-day treatment cycle as an intravenous infusion 30 minutes after completion of the administration of the antibody drug conjugate. In some embodiments, the antibody drug conjugate is administered to the subject at a dose of about 1.0 mg/kg on days 1 and 8 of a 21-day treatment cycle by intravenous infusion, and pembrolizumab is administered to the subject at a dose of about 200 mg on day 1 of the 21-day treatment cycle by intravenous infusion. In some embodiments, the antibody drug conjugate is administered to the subject at a dose of about 1.0 mg/kg on days 1 and 8 of a 21-day treatment cycle by intravenous infusion, and pembrolizumab is administered to the subject at a dose of about 200 mg on day 1 of the 21-day treatment cycle by intravenous infusion 30 minutes after administration of the antibody drug conjugate. In some embodiments, the antibody drug conjugate is administered to the subject at a dose of about 1.0 mg/kg on days 1 and 8 of a 21-day treatment cycle by intravenous infusion, and pembrolizumab is administered at a dose of about 200 mg to the subject on day 1 of the 21-day treatment cycle by intravenous infusion 30 minutes after completion of the administration of the antibody drug conjugate. In some embodiments, the antibody drug conjugate is administered to the subject at a dose of about 1.0 mg/kg up to 2 days of a 21-day treatment cycle as a 30 minute intravenous infusion, and pembrolizumab is administered to the subject at a dose of about 200 mg on 1 day of the 21-day treatment cycle as a 30 minute intravenous infusion 30 minutes after administration of the antibody drug conjugate. In some embodiments, the antibody drug conjugate is administered to the subject at a dose of about 1.0 mg/kg up to 2 days of a 21-day treatment cycle as a 30 minute intravenous infusion, and pembrolizumab is administered to the subject at a dose of about 200 mg on 1 day of the 21-day treatment cycle as a 30 minute intravenous infusion 30 minutes after completion of the administration of the antibody drug conjugate. In some embodiments, the antibody drug conjugate is administered to the subject at a dose of about 1.0 mg/kg on days 1 and 8 of a 21-day treatment cycle as a 30 minute intravenous infusion, and pembrolizumab is administered to the subject at a dose of about 200 mg on day 1 of the 21-day treatment cycle as a 30 minute intravenous infusion. In some embodiments, the antibody drug conjugate is administered to the subject at a dose of about 1.0 mg/kg on days 1 and 8 of a 21-day treatment cycle as a 30 minute intravenous infusion, and pembrolizumab is administered to the subject at a dose of about 200 mg on day 1 of the 21-day treatment cycle as a 30 minute intravenous infusion 30 minutes after administration of the antibody drug conjugate. In some embodiments, the antibody drug conjugate is administered to the subject at a dose of about 1.0 mg/kg on days 1 and 8 of a 21-day treatment cycle as a 30 minute intravenous infusion, and pembrolizumab is administered at a dose of about 200 mg to the subject on day 1 of the 21-day treatment cycle as a 30 minute intravenous infusion 30 minutes after completion of the administration of the antibody drug conjugate.

In some embodiments, the antibody drug conjugate is administered to the subject at a dose of 1.0 mg/kg up to 2 days of a 21-day treatment cycle as an intravenous infusion, and pembrolizumab is administered to the subject at a dose of 200 mg on 1 day of the 21-day treatment cycle as an intravenous infusion. In some embodiments, the antibody drug conjugate is administered to the subject at a dose of 1.0 mg/kg up to 2 days of a 21-day treatment cycle as an intravenous infusion, and pembrolizumab is administered to the subject at a dose of 200 mg on 1 day of the 21-day treatment cycle as an intravenous infusion 30 minutes after administration of the antibody drug conjugate. In some embodiments, the antibody drug conjugate is administered to the subject at a dose of 1.0 mg/kg up to 2 days of a 21-day treatment cycle as an intravenous infusion, and pembrolizumab is administered to the subject at a dose of 200 mg on 1 day of the 21-day treatment cycle as an intravenous infusion 30 minutes after completion of the administration of the antibody drug conjugate. In some embodiments, the antibody drug conjugate is administered to the subject at a dose of 1.0 mg/kg on days 1 and 8 of a 21-day treatment cycle by intravenous infusion, and pembrolizumab is administered to the subject at a dose of 200 mg on day 1 of the 21-day treatment cycle by intravenous infusion. In some embodiments, the antibody drug conjugate is administered to the subject at a dose of 1.0 mg/kg on days 1 and 8 of a 21-day treatment cycle by intravenous infusion, and pembrolizumab is administered to the subject at a dose of 200 mg on day 1 of the 21-day treatment cycle by intravenous infusion 30 minutes after administration of the antibody drug conjugate. In some embodiments, the antibody drug conjugate is administered to the subject at a dose of 1.0 mg/kg on days 1 and 8 of a 21-day treatment cycle by intravenous infusion, and pembrolizumab is administered at a dose of 200 mg to the subject on day 1 of the 21-day treatment cycle by intravenous infusion 30 minutes after completion of the administration of the antibody drug conjugate. In some embodiments, the antibody drug conjugate is administered to the subject at a dose of 1.0 mg/kg up to 2 days of a 21-day treatment cycle as a 30 minute intravenous infusion, and pembrolizumab is administered to the subject at a dose of 200 mg on 1 day of the 21-day treatment cycle as a 30 minute intravenous infusion 30 minutes after administration of the antibody drug conjugate. In some embodiments, the antibody drug conjugate is administered to the subject at a dose of 1.0 mg/kg up to 2 days of a 21-day treatment cycle as a 30 minute intravenous infusion, and pembrolizumab is administered to the subject at a dose of 200 mg on 1 day of the 21-day treatment cycle as a 30 minute intravenous infusion 30 minutes after completion of the administration of the antibody drug conjugate. In some embodiments, the antibody drug conjugate is administered to the subject at a dose of 1.0 mg/kg on days 1 and 8 of a 21-day treatment cycle as a 30 minute intravenous infusion, and pembrolizumab is administered to the subject at a dose of 200 mg on day 1 of the 21-day treatment cycle as a 30 minute intravenous infusion. In some embodiments, the antibody drug conjugate is administered to the subject at a dose of 1.0 mg/kg on days 1 and 8 of a 21-day treatment cycle as a 30 minute intravenous infusion, and pembrolizumab is administered to the subject at a dose of 200 mg on day 1 of the 21-day treatment cycle as a 30 minute intravenous infusion 30 minutes after administration of the antibody drug conjugate. In some embodiments, the antibody drug conjugate is administered to the subject at a dose of 1.0 mg/kg on days 1 and 8 of a 21-day treatment cycle as a 30 minute intravenous infusion, and pembrolizumab is administered at a dose of 200 mg to the subject on day 1 of the 21-day treatment cycle as a 30 minute intravenous infusion 30 minutes after completion of the administration of the antibody drug conjugate.

In any embodiment disclosed herein, the terms “treatment cycle” and “cycle” are interchangeable.

### 5.9 Methods for Determining the Biomarkers

The disclosure provides that the expression of any of the markers provided herein can be determined by various methods known in the field. In some embodiments, the expression of the markers can be determined by the amount or relative amount of mRNA transcribed from the marker genes. In one embodiment, the expression of the marker genes can be determined by the amount or relative amount of the protein products encoded by the marker genes. In another embodiment, the expression of the marker genes can be determined by the level of biological or chemical response induced by the protein products encoded by the marker genes. Additionally, in certain embodiments, the expression of the marker genes can be determined by the expression of one or more genes that correlates with the expression of the marker genes.

As described above, levels or amounts of gene transcripts (e.g. mRNA) of the marker genes can be used as a proxy for the expression levels of markers genes. Numerous different PCR or qPCR protocols are known in the art including those exemplified herein. In some embodiments, the various PCR or qPCR methods are applied or adapted for determining the mRNA level of the various marker genes. Quantitative PCR (qPCR) (also referred as real-time PCR) is applied and adapted in some embodiments as it provides not only a quantitative measurement, but also reduced time and contamination. As used herein, “quantitative PCR (or “qPCR”) refers to the direct monitoring of the progress of PCR amplification as it is occurring without the need for repeated sampling of the reaction products. In quantitative PCR, the reaction products can be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time. When qPCR is applied to determine mRNA expression level, an extra step of reverse-transcription of mRNA to DNA is performed before the qPCR analysis. Examples of PCR methods can be found in the literature (Wong et al., BioTechniques 39:75-85 (2005); D'haene et al., Methods 50:262-270 (2010)), which is incorporated by reference herein in its entirety. Examples of PCR assays can also be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In one specific embodiment, qPCR can be performed to determine or measure the mRNA levels of the marker genes as follows. Briefly, mean Ct (cycle threshold) values (or referred to herein interchangeably as Cq (quantification cycle)) of replicate qPCR reactions for the marker genes and one or more housekeeping genes are determined. Mean Ct values for the marker genes can be then normalized to the Ct values of the housekeeping genes using the following exemplary formula: marker-gene-ΔCt=(mean Ct of marker gene−mean Ct of housekeeping gene A). The relative marker-gene-ΔCt can then be used to determine relative level of marker gene mRNA, for example by using the formula of mRNA expression=2^{-ΔCt}. For a summary of Ct and Cq values, see MIQE guideline (Bustin et al., The MIQE Guidelines: Minimum Information for Publication of Quantitative Real-Time PCR Experiments, Clinical Chemistry 55:4 (2009)).

Other commonly used methods known in the art can also be used for the quantification of RNA transcripts of the marker genes in a sample as the proxy for the expression of the marker genes, including northern blotting and in situ hybridization (Parker & Barnes, Methods in Molecular Biology 106:247-283 (1999)); RNAse protection assays (Hod, Biotechniques 13:852-854 (1992)); microarrays (Hoheisel et al., Nature Reviews Genetics 7:200-210 (2006); Jaluria et al., Microbial Cell Factories 6:4 (2007)); and polymerase chain reaction (PCR) (Weis et al, Trends in Genetics 8:263-264 (1992)). RNA in situ hybridization (ISH) is a molecular biology technique widely used to measure and localize specific RNA sequences, for example, messenger RNAs (mRNAs), long non-coding RNAs (lncRNAs), and microRNAs (miRNAs) within cells, such as circulating tumor cells (CTCs) or tissue sections, while preserving the cellular and tissue context. ISH is a type of hybridization that uses a directly or indirectly labeled complementary DNA or RNA strand, such as a probe, to bind to and localize a specific nucleic acid, such as DNA or RNA, in a sample, in particular a portion or section of tissue or cells (in situ). The probe types can be double stranded DNA (dsDNA), single stranded DNA (ssDNA), single stranded complimentary RNA (sscRNA), messenger RNA (mRNA), micro RNA (miRNA), ribosomal RNA, mitochondrial RNA, and/or synthetic oligonucleotides. The term “fluorescent in situ hybridization” or “FISH” refers to a type of ISH utilizing a fluorescent label. The term “chromogenic in situ hybridization” or “CISH” refers to a type of ISH with a chromogenic label. ISH, FISH and CISH methods are well known to those skilled in the art (see, for example, Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000)). RNA ISH therefore provides for spatial-temporal visualization as well as quantification of gene expression within cells and tissues. It has wide applications in research and in diagnostics (Hu et al., Biomark. Res. 2(1):1-13, doi: 10.1186/2050-7771-2-3 (2014); Ratan et al., Cureus 9(6):e1325. doi: 10.7759/cureus.1325 (2017); Weier et al., Expert Rev. Mol. Diagn. 2(2):109-119 (2002)). Fluorescent RNA ISH utilizes fluorescent dyes and fluorescent microscopes for RNA labeling and detection, respectively. Fluorescent RNA ISH can provides for multiplexing of four to five target sequences.

Alternatively, RNA transcripts of the marker genes in a sample as the proxy for the expression of the marker genes can be determined by sequencing techniques. Representative methods for sequencing-based gene expression analysis include Serial Analysis of Gene Expression (SAGE), and gene expression analysis by massively parallel signature sequencing (MPSS).

In some embodiments, expression of the marker genes can be determined by the relative abundance of the RNA transcripts (including for example mRNA) of the marker genes in a pool of total transcribed RNA. Such relative abundance of the RNA transcripts of the marker genes can be determined by next generation sequencing, which is known as RNA-seq. In one example of the RNA-seq procedure, RNAs from different sources (blood, tissue, cells) are purified, optionally enriched (e.g. with oligo (dT) primers), converted to cDNA, and fragmented. Millions or even billions of short sequence reads are generated from the randomly fragmented cDNA library. See Zhao et al. BMC genomics 16: 97 (2015); Zhao et al. Scientific Reports 8: 4781 (2018); Shanrong Zhao et al., RNA, published in advance Apr. 13, 2020, doi: 10.1261/rna.074922.120, all of which are incorporated herein in their entirety by reference. The expression level of each mRNA transcript of the marker genes is determined by the total number of mapped fragments upon normalization, which is directly proportional to its abundance level. A few normalization schemes are known and used to facilitate the use of the abundance of the RNA transcripts as the parameter for determining gene expression, including RPKM (Reads Per Kilobase Million), FPKM (Fragments Per Kilobase Million), and/or TPM (Transcripts Per Kilobase Million). Briefly, RPKM can be calculated as follows: count up the total reads in a sample and divide that number by 1,000,000—which is the “per million” scaling factor; divide the read counts by the “per million” scaling factor, which normalizes for sequencing depth, giving the reads per million (RPM); and divide the RPM values by the length of the gene, in kilobases, which gives RPKM. FPKM is closely related to RPKM except with fragment replacing read. RPKM was made for single-end RNA-seq, where every read corresponded to a single fragment that was sequenced. FPKM was made for paired-end RNA-seq, in which two reads can correspond to a single fragment, or, if one read in the pair did not map, one read can correspond to a single fragment. TPM is very similar to RPKM and FPKM and is calculated as follows: divide the read counts by the length of each gene in kilobases, which gives the reads per kilobase (RPK); count up all the RPK values in a sample and divide this number by 1,000,000, which gives the “per million” scaling factor; divide the RPK values by the “per million” scaling factor, which gives TPM. See Zhao et al. BMC genomics 16: 97 (2015); Zhao et al. Scientific Reports 8: 4781 (2018); Shanrong Zhao et al., RNA, published in advance Apr. 13, 2020, doi: 10.1261/rna.074922.120, all of which are incorporated herein in their entirety by reference.

In one embodiment, the expression of the marker genes is determined by RNA-seq, for example by TPM, RPKM, and/or FPKM. In some embodiments, the expression of the marker genes is determined by TPM. In some embodiments, the expression of the marker genes is determined by RPKM. In some embodiments, the expression of the marker genes is determined by FPKM.

As described earlier, the expression of the marker genes can be determined in a sample from a subject. In some embodiments, the sample is a blood sample, a serum sample, a plasma sample, bodily fluid (e.g. tissue fluid including cancer tissue fluid), or a tissue (e.g. cancer tissue or the tissue surrounding the cancer). In some embodiments, the sample is a tissue sample. In some embodiments, the tissue sample is tissue fractions isolated or extracted from a mammal, in particular a human. In some embodiments, the tissue sample is a population of cells isolated or extracted from a mammal, in particular a human. In some embodiments, the tissue sample is a sample obtained from a biopsy. In certain embodiments, the samples can be obtained from a variety of organs of a subject, including a human subject. In some embodiments, the samples are obtained from organs of a subject having a cancer. In some embodiments, the samples are obtained from organs having a cancer in a subject having a cancer. In other embodiments, the samples, for example reference samples, are obtained from normal organs from the patient or from a second human subject.

In certain embodiments of the methods provided herein, the tissue includes a tissue from bladder, ureter, breast, lung, colon, rectum, ovary, Fallopian tube, esophagus, cervix, uterine endometrium, skin, larynx, bone marrow, salivary gland, kidney, prostate, brain, spinal cord, placenta, adrenal, pancreas, parathyroid, hypophysis, testis, thyroid, spleen, tonsil, thymus, heart, stomach, small intestine, liver, skeletal muscle, peripheral nerve, mesothelium, or eye.

In further embodiments of the methods provided herein, the expression of the various marker genes can be detected by a variety of immunoassays known in the art, including an immunohistochemistry (IHC) assay, an immunoblotting assay, a FACS assay, and an ELISA.

The expression of the various marker genes can be detected by antibodies against the protein products encoded by the marker genes in a variety of IHC assays. IHC staining of tissue sections has been shown to be a reliable method of assessing or detecting the presence of proteins in a sample. IHC techniques utilize an antibody to probe and visualize cellular antigens in situ, generally by chromogenic or fluorescent methods. Primary antibodies or antisera, such as polyclonal antisera and monoclonal antibodies that specifically target the protein products encoded by the marker genes, can be used to detect expression of the marker genes in an IHC assay. In some embodiments, the tissue sample is contacted with a primary antibody for a specific target for a period of time sufficient for the antibody-target binding to occur. As discussed in detail earlier, the antibodies can be detected by direct labels on the antibodies themselves, for example, radioactive labels, fluorescent labels, hapten labels such as biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. IHC protocols and kits are well known in the art and are commercially available. Automated systems for slide preparation and IHC processing are available commercially. The Leica BOND Autostainer and Leica Bond Refine Detection system is an example of such an automated system.

In some embodiments, an IHC assay is performed with an unlabeled primary antibody in conjunction with a labeled secondary antibody in an indirect assay. The indirect assay utilizes two antibodies for the detection of the protein products encoded by the marker genes in a tissue sample. First, an unconjugated primary antibody was applied to the tissue (first layer), which reacts with the target antigen in the tissue sample. Next, an enzyme-labeled secondary antibody is applied, which specifically recognize the antibody isotype of the primary antibody (second layer). The secondary antibody reacts with the primary antibody, followed by substrate-chromogen application. The second-layer antibody can be labeled with an enzyme such as a peroxidase, which reacts with the chromogen 3, 3′-diaminobenzidine (DAB) to produce brown precipitate at the reaction site. This method is sensitive and versatile due to the potential signal amplification through a signal amplification system.

In certain embodiments to increase the sensitivity of the detection, a signal amplification system can be used. “A signal amplification system”, as used herein, means a system of reagents and methods that can be used to increase the signal from detecting the bound primary or the secondary antibody. A signal amplification system increases the sensitivity of the target protein detection, increases the detected signal, and decreases the lower boundary of the detection limits. There are several types of signal amplification systems including an enzyme labeling system and macrolabeling system. These systems/approaches are not mutually exclusive and can be used in combination for additive effect.

Macrolabels or macrolabeling system are collections of labels numbering in the tens (e.g. phycobiliproteins) to millions (e.g. fluorescent microspheres) attached to or incorporated in a common scaffold. The scaffold can be coupled to a target-specific affinity reagent such as an antibody, and the incorporated labels are thereby collectively associated with the target upon binding. The labels in the macrolabels can be any of the labels described herein such as fluorophores, haptens, enzymes, and/or radioisotopes. In one embodiment of the signal amplification system, a labeled chain polymer-conjugated secondary antibody was used. The polymer technology utilized an HRP enzyme-labeled inert “spine” molecule of dextran to which 1, 2, 3, 4, 5, 6, 7, 8, 9 10, 15, 20, 25, 30, 50 or more molecules of secondary antibodies can be attached, making the system even more sensitive.

Signal amplification system based on an enzyme labeling system utilizes the catalytic activity of enzymes, such as horseradish peroxidase (HRP) or alkaline phosphatase to generate high-density labeling of a target protein or nucleic acid sequence in situ. In one embodiment, tyramide can be used to increase the signal of HRP. In such a system, HRP enzymatically converts the labeled tyramide derivative into highly reactive, short-lived tyramide radicals. The labeled active tyramide radicals then covalently couple to residues (principally the phenol moiety of protein tyrosine residues) in the vicinity of the HRP-antibody-target interaction site, resulting amplification of the number of labels at the site with minimal diffusion-related loss of signal localization. Consequently, the signal can be amplified 1, 2, 3, 4, 5, 6, 7, 8, 9 10, 15, 20, 25, 30, 50, 75, or 100 folds. As known to a person skilled in the art, the labels on the tyramide can be any labels described herein, including fluorophores, enzymes, haptens, radioisotopes, and/or photophores. Other enzyme-based reactions can be utilized to create signal amplification as well. For example, Enzyme-Labeled Fluorescence (ELF) signal amplification is available for alkaline phosphatase, wherein the alkaline phosphatase enzymatically cleaves a weakly blue-fluorescent substrate (ELF 97 phosphate) and converts it into a bright yellow-green-fluorescent precipitate that exhibits an unusually large Stokes shift and excellent photostability. Both tyramide-based signal amplification system and ELF signal amplification are available commercially, for example from ThermoFisher Scientific (Waltham, MA USA 02451).

Thus in some embodiments of the methods provided herein, the expression level of the marker genes is detected with IHC using a signal amplification system. In some embodiments, the specimen is then counterstained to identify cellular and subcellular elements.

In some embodiments, the expression level of the protein products encoded by the marker genes can also be detected with antibodies against the protein products encoded by the marker genes using an immunoblotting assay. In some embodiments of an immunoblotting assay, proteins are often (but do not have to be) separated by electrophoresis and transferred onto membranes (usually nitrocellulose or PVDF membrane). Similar to the IHC assays, primary antibodies or antisera, such as polyclonal antisera and monoclonal antibodies that specifically target the protein products encoded by the marker genes, can be used to detect expression of the marker genes. In some embodiments, the membrane is contacted with a primary antibody for a specific target for a period of time sufficient for the antibody-antigen binding to occur and the bound antibodies can be detected by direct labels on the primary antibodies themselves, e.g. with radioactive labels, fluorescent labels, hapten labels such as biotin, or enzymes such as horseradish peroxidase or alkaline phosphatase. In other embodiments, unlabeled primary antibody is used in an indirect assay as described above in conjunction with a labeled secondary antibody specific for the primary antibody. As described herein, the secondary antibodies can be labeled, for example, with enzymes or other detectable labels such as fluorescent labels, luminescent labels, colorimetric labels, or radioisotopes. Immunoblotting protocols and kits are well known in the art and are commercially available. Automated systems for immunoblotting, e.g. iBind Western Systems for Western blotting (ThermoFisher, Waltham, MA USA 02451), are available commercially. Immunoblotting includes, but is not limited to, Western blot, in-cell Western blot, and dot blot. Dot blot is a simplified procedure in which protein samples are not separated by electrophoresis but are spotted directly onto a membrane. In cell Western blot involves seeding cells in microtiter plates, fixing/permeabilizing the cells, and subsequent detection with a primary labeled primary antibody or unlabelled primary antibody followed by labeled secondary antibody as described herein.

In other embodiments, the expression levels of the protein products encoded by the marker genes can also be detected with the antibodies described herein in a flow cytometry assay, including a fluorescence-activated cell sorting (FACS) assay. Similar to the IHC or immunoblotting assays, primary antibodies or antisera, such as polyclonal antisera and monoclonal antibodies that specifically target the protein products encoded by the marker genes, can be used to detect protein expression in a FACS assay. In some embodiments, cells are stained with primary antibodies against specific target protein for a period of time sufficient for the antibody-antigen binding to occur and the bound antibodies can be detected by direct labels on the primary antibodies, for example, fluorescent labels or hapten labels such as biotin on the primary antibodies. In other embodiments, unlabeled primary antibody is used in an indirect assay as described above in conjunction with a fluorescently labeled secondary antibody specific for the primary antibody. FACS provides a method for sorting or analyzing a mixture of fluorescently labeled biological cells, one cell at a time, based upon the specific light scattering and fluorescent characteristics of each cell. The flow cytometer thus detects and reports the intensity of the fluorichrome-tagged antibody, which indicates the expression level of the target protein. Therefore, the expression level of the protein products encoded by the marker genes can be detected using antibodies against such protein products. Non-fluorescent cytoplasmic proteins can also be observed by staining permeablized cells. Methods for performing FACS staining and analyses are well known to a person skilled in the art and are described by Teresa S. Hawley and Robert G. Hawley in Flow Cytometry Protocols, Humana Press, 2011 (ISBN 1617379506, 9781617379505).

In other embodiments, the expression levels of the protein products encoded by the marker genes can also be detected using immunoassays such as an Enzyme Immune Assay (EIA) or an ELISA. Both EIA and ELISA assays are known in the art, e.g. for assaying a wide variety of tissues and samples, including blood, plasma, serum or bone marrow. A wide range of ELISA assay formats are available, see, e.g., U.S. Pat. Nos. 4,016,043, 4,424,279, and 4,018,653, which are hereby incorporated by reference in their entireties. These include both single-site and two-site or “sandwich” assays of the non-competitive types, as well as in the traditional competitive binding assays. These assays also include direct binding of a labeled antibody to a target protein. Sandwich assays are commonly used assay format. A number of variations of the sandwich assay technique exist. For example, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results can either be qualitative, by simple observation of the visible signal, or can be quantitated by comparing with a control sample containing known amounts of target protein.

In some embodiments of the EIA or ELISA assays, an enzyme is conjugated to the second antibody. In other embodiments, fluorescently labeled secondary antibodies can be used in lieu of the enzyme-labeled secondary antibody to produce a detectable signal an ELISA assay format. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA and ELISA, the fluorescent labeled antibody is allowed to bind to the first antibody-target protein complex. After washing off the unbound reagent, the remaining tertiary complex is then exposed to the light of the appropriate wavelength; the fluorescence observed indicates the presence of the target protein of interest. Immunofluorescence and EIA techniques are both very well established in the art and are disclosed herein.

For the immunoassays described herein, any of a number of enzymes or non-enzyme labels can be utilized so long as the enzymatic activity or non-enzyme label, respectively, can be detected. The enzyme thereby produces a detectable signal, which can be utilized to detect a target protein. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase,6-phospho-β-D-galatoside 6-phosphogalactohydrolase, β-gluosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods of the present disclosure.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl)umbelliferyl phosphate for alkaline phosphatase; 4-Methylumbelliferyl phosphate bis(2-amino-2-methyl-1,3-propanediol), 4-Methylumbelliferyl phosphate bis(cyclohexylammonium) and 4-Methylumbelliferyl phosphate for phosphatases; QuantaBlu™ and QuantaRed™ for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-α- D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

In some embodiments of the immunoassays, a detectable label can be directly coupled to either the primary antibody or the secondary antibody that detects the unlabeled primary antibody can have. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof, napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene Oreg.); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof, CyDye™ fluorescent dyes (Amersham/GE Healthcare Life Sciences; Piscataway NJ), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

Methods well known to a person skilled in the art such as microscopy or spectroscopy can be utilized to visualize chromogenic or fluorescent detectable signals associated with the bound primary or secondary antibodies.

The methods provided in this Section (Section 5.8) can be used with various cancer models known in the art. In one embodiment, mouse xenograft cancer models are used. Briefly, T-24 and UM-UC-3 cells are purchased from ATCC and cultured using the recommended media conditions. The T-24 hNectin-4 (human nectin-4) and the UM-UC-3 Nectin-4 cells are generated by transducing parental cells with lentivirus containing the human Nectin-4 using the pRCDCMEP-CMV-hNectin-4 EF1-Puro construct and selected using puromycin. The T-24 Nectin-4 (clone 1A9) cells are implanted into nude mice and passaged via trocar, allowed to reach approximately 200 mm³ tumor volume, and subsequently treated with a single intraperitoneal (IP) dose of enfortumab vedotin (3 mg/kg) or non-binding ADC (3 mg/kg) with 7 animals per treatment group. Follow-up ICD studies with this model involve collecting tumors 5 days post treatment for downstream analysis by RNA-seq, flow, immunohistochemistry (IHC), and Luminex. Tumors are fixed in formalin and prepared as FFPE tissue blocks. Blocks are cut at 4 μm and immunohistochemistry is performed using F4/80, CD11c. The immunohistochemically stained slides sections are scanned with a Leica AT2 digital whole slide scanner, and the images are analyzed with Visiopharm software by use of custom-made algorithms for Nectin 4, CD11c and F4/80 staining. The algorithms are optimized on the basis of staining intensity and background staining. Percent positive staining is calculated for Nectin 4 and positive cells per mm² is calculated for F480 and CD11c.

Sections of tumor are lysed in Cell Lysis Buffer 2 (R&D Systems®, Catalog #895347). The cytokines and chemokines from the tumor samples are measured using the MILLIPLEX MAP mouse cytokine/chemokine magnetic bead panel (Millipore) and read on the LUMINEX MAGPIX system.

For the RNA-seq analysis RNA from flash frozen tumors is isolated using the TRIZOL Plus RNA Purification Kit (Life Technologies) according to the manufacturer's protocol yielding high quality RNA (average RNA integrity number >8). RNA selection method is using Poly(A) selection and the mRNA Library Prep Kit from Illumina and read on the Hi-Seq 2×150 bp, single index (Illumina). The sequence reads are mapped to the human and mouse transcriptome and total reads per million were determined.

The disclosure is generally provided using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

Particular embodiments of this disclosure are described herein, including the best mode known to the inventors for carrying out the disclosure. Upon reading the foregoing description, variations of the disclosed embodiments can become apparent to individuals working in the art, and it is expected that those skilled artisans can employ such variations as appropriate. Accordingly, it is intended that the disclosure be practiced otherwise than as specifically described herein, and that the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All publications, patent applications, accession numbers, and other references cited in this specification are herein incorporated by reference in their entireties as if each individual publication or patent application were specifically and individually indicated to be incorporated by reference.

A number of embodiments of the disclosure have been described. Nevertheless, it will be understood that various modifications can be made without departing from the spirit and scope of the disclosure.

### 6. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

### 6.1 Example 1—A Phase 1b/2 Study of Enfortumab Vedotin Combined with Pembrolizumab as First-Line Treatment in Patients with unresectable Locally Advanced or Metastatic Urothelial Cancer (la/mUC) Who are Unable to Receive Cisplatin-Based Chemotherapy

### 6.1.1 Drue Used in the Clinical Study

Enfortumab vedotin is a Nectin-4 targeted monoclonal antibody (AGS-22C3) covalently linked to the microtubule-disrupting agent monomethyl auristatin E (MMAE). Enfortumab vedotin consists of three functional subunits:
A fully human IgG1K antibody (AGS-22C3);
The microtubule-disrupting agent MMAE;
A protease-cleavable maleimidocaproyl-valine-citrulline (vc) linker that covalently attaches MMAE to AGS-22C3.

Enfortumab vedotin binds the V domain of Nectin-4 (Challita-Eid et al., Cancer Res (2016); 76(10): 3003-13.). In the presumed mechanism of action, the drug binds Nectin-4 protein on the cell surface and is internalized, causing proteolytic cleavage of the vc linker and intracellular release of MMAE. Free MMAE subsequently disrupts tubulin polymerization and leads to mitotic arrest.

### 6.1.2 Summary of the Study

### 6.1.2.1 Synopsis

(i) Name of Study Drug

### Enfortumab Vedotin (ASG-22CE)

(ii) Phase of Development

### Phase 1b/2

(iii) Title of Study

A phase 1b/2 study of enfortumab vedotin combined with pembrolizumab as first-line treatment in patients with unresectable locally advanced or metastatic urothelial cancer (la/mUC) who are unable to receive cisplatin-based chemotherapy.

### (iv) Study Objective(s)

### Primary

To assess the antitumor activity of enfortumab vedotin monotherapy or enfortumab vedotin in combination with pembrolizumab as measured by ORR per RECIST Version 1.1 by blinded independent central review (BICR).

### Secondary

To assess the objective response rate (ORR) per RECIST Version 1.1 by investigator assessment
To assess the duration of response (DOR) per RECIST Version 1.1 by BICR and investigator assessment
To assess the disease control rate (DCR) per RECIST Version 1.1 by BICR and investigator assessment
To assess the progression-free survival (PFS) on study therapy per RECIST Version 1.1 by BICR and investigator assessment
To assess overall survival (OS)
To assess the safety and tolerability of enfortumab vedotin monotherapy or enfortumab vedotin in combination with pembrolizumab

### Additional

To assess pharmacokinetics (PK) and the incidence of antitherapeutic antibodies (ATA)
To assess Nectin-4 and PD-L1 expression levels
To assess biomarkers of biological activity and disease resistance, and their potential associations with clinical outcome measures
To assess the PFS on subsequent therapy (PFS2) by investigator assessment
To assess the impact on quality of life (QoL) and symptoms, including pain, from the patient perspective
To assess the ORR, DOR, DCR, and PFS on study therapy per iRECIST for enfortumab vedotin in combination with pembrolizumab (EV+Pembro arm) by investigator assessment

### (v) Study Population

The population studied included patients with la/mUC and measurable disease according to RECIST Version 1.1. Additional requirements included the following: Patients must also be ≥18 years of age, must have an anticipated life expectancy of ≥3 months as assessed by the investigator, and an Eastern Cooperative Oncology Group (ECOG) performance status of 0, 1, or 2, with adequate baseline hematologic, hepatic, and renal function.

Patients must not have ongoing sensory or motor neuropathy (Grade 2 or higher), or active central nervous system (CNS) metastases. Patients must not have received previous treatment with enfortumab vedotin or other monomethyl auristatin E (MMAE)-based antibody-drug conjugates (ADCs) for urothelial cancer. Patients must not have a history of another malignancy within 3 years, or any evidence of residual disease from a previously diagnosed malignancy. Patients were also excluded if they are currently receiving systemic antimicrobial treatment for active infection or high dose steroids. Patients with uncontrolled diabetes wee excluded. Uncontrolled diabetes is defined as hemoglobin A1c (HbA1c)≥8% or HbA1c 7% to <8% with associated diabetes symptoms (polyuria or polydipsia) that are not otherwise explained.

Patients must be able to receive pembrolizumab and must be ineligible for cisplatin-based chemotherapy at the time of enrollment due to at least 1 of the following criteria: glomerular filtration rate (GFR) <60 mL/min but ≥30 mL/min, ECOG performance status of 2, National Cancer Institute Common Terminology Criteria for Adverse Events (NCI CTCAE) Version 4.03 Grade ≥2 hearing loss, or New York Heart Association (NYHA) Class III heart failure. Patients must not have received prior systemic treatment for locally advanced or metastatic disease. Patients may not have previously received adjuvant/neoadjuvant platinum-based therapy within 12 months prior to randomization.

### (vi) Number of Planned Patients

Approximately 150 patients were enrolled in this study.

(vii) Study Design

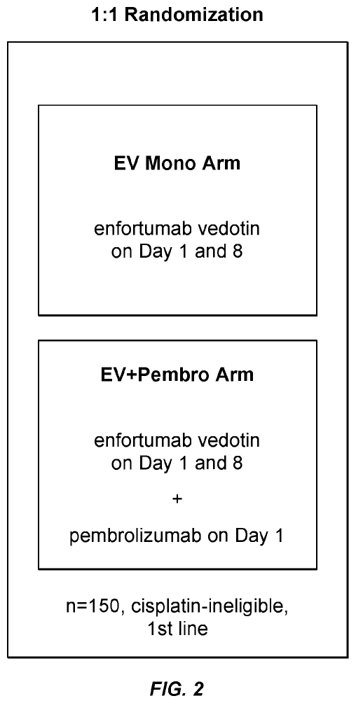

This study is designed to evaluate the safety and antitumor activity of enfortumab vedotin as monotherapy or in combination with pembrolizumab for the treatment of patients with la/mUC. This study included 2 treatment arms randomized in a 1:1 ratio to enfortumab vedotin monotherapy or enfortumab vedotin in combination with pembrolizumab. The study design is depicted in FIG. 2.

All patients received enfortumab vedotin, the investigational agent under study, administered as an IV infusion on Days 1 and 8 of every 3-week cycle. A dose level of 1.25 mg/kg IV enfortumab vedotin on Days 1 and 8 of every 3-week cycle has been determined. Enfortumab vedotin dose de-escalation (up to 2 step-down dose levels) was allowed if recommended by the SMC and approved by the study sponsor.

For patients in the EV Mono arm, the investigator made treatment decisions based on site assessments of scans by RECIST Version 1.1.

Patients were stratified by the absence or presence of liver metastases, and ECOG performance status (0 versus 1/2), and randomized in a 1:1 ratio to receive either enfortumab vedotin monotherapy (EV Mono arm) or enfortumab vedotin in combination with pembrolizumab (EV+Pembro arm):
The EV Mono arm included approximately 75 patients. Patients received enfortumab vedotin on Days 1 and 8 of every 3-week cycle.
The EV+Pembro arm included approximately 75 patients. Patients received enfortumab vedotin on Days 1 and 8, and pembrolizumab on Day 1 of every 3-week cycle.

Patients completed patient reported outcome (PRO) assessments on an electronic device (preferred medium for reporting). Assessments were reported on paper or by clinic staff by telephone only if the use of an electronic device wass not feasible. Safety was monitored by a restricted SMC on an ongoing basis.

After initiation of subsequent anticancer therapy or radiologically confirmed disease progression on or following study treatment (by investigator assessment), patients were followed via imaging per institutional guidelines, but not less frequently than every 12 weeks until PFS2 was documented or the patient started another anticancer treatment, whichever occurs first. All subsequent anticancer therapy including date of progression for PFS2 was recorded on the case report form (CRF). In addition, patients were contacted every 12 weeks (±1-week) after end of treatment (EOT) for survival status and clinical progression status on or following subsequent therapy. Following PFS2, patients entered the survival follow-up period and were followed every 12 weeks (±1 week) for survival status until death, study closure, or withdrawal of consent, whichever occurred first.

(viii) Test Product, Dose, and Mode of Administration

Enfortumab vedotin administered as an IV infusion at 1 or 1.25 mg/kg over approximately 30 minutes on Days 1 and 8 of every 3-week cycle. A dose level of 1.25 mg/kg IV enfortumab vedotin on Days 1 and 8 of every 3-week cycle has been determined. Dose de-escalation at the following step-down dose levels are possible if recommended by the SMC and agreed to by the sponsor.

Table 8 shows the Enfortumab vedotin step-down dose levels for the study.

TABLE 8
Enfortumab vedotin step-down dose levels

| Dose level | Enfortumab Vedotin Dose (mg/kg) |
|---|---|
| 1 | 1.25 |
| −1 | 1 |
| −2 | 0.75 |

Enfortumab vedotin doses were calculated on the basis of a patient's actual body weight at baseline. Doses were recalculated when a patient's body weight changed by ≥10% of baseline or the previous cycle or when dose adjustment criteria were met. Actual weight was used except for patients weighing >100 kg; in such cases, the dose was calculated based on a weight of 100 kg. The maximum dose permitted in this study component was 125 mg.

In the EV+Pembro arm, on Day 1 of each 3-week cycle, pembrolizumab 200 mg was administered as an IV infusion approximately 30 minutes after completion of enfortumab vedotin.

### (ix) Duration of Treatment

Patients were continued on study treatment until radiographically confirmed disease progression, unacceptable toxicity, investigator decision, consent withdrawal, start of a subsequent anticancer therapy, pregnancy, or study termination by the sponsor. Patients in the EV+Pembro arm with a response assessment of iUPD were permitted to continue on treatment until iCPD was confirmed by the investigator with a subsequent scan 4 to 9 weeks after iUPD.

Pembrolizumab was administered for up to a total of 35 cycles.

Patients who experienced unacceptable toxicity that was attributable only to pembrolizumab were continued on enfortumab vedotin monotherapy until radiographically confirmed disease progression, unacceptable toxicity, investigator decision, consent withdrawal, start of a subsequent anticancer therapy, pregnancy, or study termination by the sponsor. Patients who experienced toxicity that was attributable only to enfortumab vedotin were continued on pembrolizumab monotherapy (for up to 35 cycles) until disease progression, unacceptable toxicity, investigator decision, consent withdrawal, start of a subsequent anticancer therapy, pregnancy, or study termination by the sponsor.

### (x) Efficacy Assessments

Measures of antitumor activity were assessed by CT (with IV contrast) of the chest, abdomen, and pelvis at protocol-specified time points. If contrast media were contraindicated, refer to the current Image Acquisition Guidelines (Section 6.1.13) for preferred scanning and contrast options. Patients received the same imaging method throughout the study for response assessments. Brain scans (which are required at screening) were repeated at disease assessment time points if inactive brain metastases were known at baseline, or as clinically indicated throughout the study. Bone scans (which are required at screening) were repeated at response assessment time points if bone metastases were identified at baseline, or if bone metastasis was known or suspected. Other regions should be scanned if the patient has known disease or new symptoms occurred suggestive of disease in that region. Objective responses were confirmed per RECIST Version 1.1 with repeat scans at least 4 weeks and within 5 weeks after the first documentation of response. Treatment decisions by the investigator were based on iRECIST guidelines for EV+Pembro arm. Treatment decisions by the investigator were based on RECIST Version 1.1 for EV Mono arm.

### (xi) Pharmacokinetic and Immunogenicity Assessments

Blood samples for PK and ATA were collected throughout the study at specific time points. Validated or qualified assays were used to measure the concentrations of enfortumab vedotin ADC, total antibody (TAb), and MMAE in serum or plasma. PK samples were collected and archived for possible analysis of concomitant drug levels or other enfortumab vedotin-related species, such as circulating metabolites of MMAE. A validated assay was used to determine the levels of ATA for enfortumab vedotin in plasma. PK and ATA samples for pembrolizumab were collected and archived for potential subsequent analysis the EV+Pembro arm.

(xii) Biomarker Assessments

Peripheral blood, urine, and tumor biopsies were collected at protocol specified time points. Exploratory, predictive, and prognostic biomarkers associated with response, resistance, or safety observations were monitored before and during study treatment. Tumor samples obtained at RC+PLND (for the MIBC component) were used to characterize the clinical mechanisms of action and resistance.

Tumor tissue from diagnostic TURBT specimens and RC+PLND was required (fine needle aspiration is not adequate) to identify novel biomarkers. If additional post-treatment biopsies were done as part of SOC, the samples were also be used to further identify biomarkers of response and mechanism of action and resistance to treatment.

Biomarker assessments in tumor tissue may include, but may not be limited to, measurement of gene expression (GE) and mutation burden, characterization of the tumor microenvironment (TME) and tumor subtype, and drug effects. Assays may include, but may not be limited to, immunohistochemistry (IHC) for Nectin-4 and PD-L1, and Next Generation Sequencing (NGS) of RNA and DNA. Biomarker assessments in blood samples may include, but may not be limited to, measurement of baseline and drug induced changes in circulating blood cell subpopulations, immunoassays, and circulating disease markers. Blood and urine assays may include, but may not be limited to, circulating tumor DNA, proteomic methodologies such as enzyme-linked immunosorbent assay (ELISA), immunoassays as a marker of tumor response or therapy resistance, and markers of immune function, including abundance of immune cell subsets and cytokines.

Other tissue (e.g., skin) from optional tissue sample submissions were analyzed for biomarkers including tissue levels of drug and drug products, nucleic acids, and protein to investigate possible associations with mechanisms of resistance or sensitivity to treatments as well as dynamic changes associated with treatments. Methods of analysis include IHC, NGS of DNA and RNA, T cell receptor beta chain sequencing, polymerase chain reaction, flow cytometry, and immunoassays

(xiii) Other Assessments

PRO assessments included QoL measures, both general and oncology specific (EuroQOL-5 Dimensions [EQ 5D-5L] and European Organisation for Research and Treatment of Cancer [EORTC] Core QoL Assessment [QLQ C30], respectively), as well as outcome-specific PROs around pain (Brief Pain Inventory Short Form [BPI-SF]). A health resource utilization (HRU) questionnaire also aimed to measure clinical burden outside of the clinical trial setting. PRO assessments were completed on an electronic device (preferred medium for reporting) on Day 1 prior to dosing, once weekly for the first 3 cycles, and once every cycle for the remainder of the treatment portion of the study. On dosing visit days, assessments were completed prior to dosing. PRO assessments were completed at each follow-up visit and each long term follow-up contact. PRO assessments were reported on paper or by clinic staff by telephone only if the use of an electronic device is not feasible.

(xiv) Safety Assessments

Safety assessments were based on the information collected through the safety surveillance process and included the data from recorded AEs, including serious adverse events (SAEs), concomitant medications, physical examination findings, cardiac monitoring, and laboratory tests. Safety was monitored over the course of the study by the SMC.

### (xv) Statistical Methods

### (a) Analysis for the Key Safety Endpoint

The safety analysis evaluated the type, incidence, severity, seriousness, and relatedness of AEs, and the type, incidence, and severity of laboratory abnormalities. The incidence, duration, and resolution of AEs of special interest (AESIs) were summarized.

### (b) Analysis for the Key Efficacy Endpoint

Efficacy data was summarized by arm.

The observed ORR per RECIST Version 1.1 and the 95% confidence interval (CI) were provided for the FAS using Clopper-Pearson methodology, by arm.

Randomization: Patients were stratified and randomized in a 1:1 ratio to the EV Mono arm or the EV+Pembro arm. Stratification factors are the absence or presence of liver metastases and ECOG performance status (0 versus 1/2).

### (b) Sample Size

Approximately 150 patients were randomized in a 1:1 ratio to receive either enfortumab vedotin monotherapy (EV Mono arm) or enfortumab vedotin in combination with pembrolizumab (EV+Pembro arm).

The sample size was not based on power calculations for formal hypothesis testing but was selected based on the precision of the estimate for ORR as characterized by 95% CIs. Formal statistical comparisons between the 2 arms of the randomized stdy was not performed.

For a sample size of 75 patients per arm, assuming the ORR is between 50% and 70%, the 2-sided 95% CIs are summarized in Table 9 below.

TABLE 9
Summary of CIs

| ORR | 95% Exact CI (n=75) |
|---|---|
| 50% | (39%, 62%) |
| 60% | (48%, 71%) |
| 70% | (59%, 81%) |

In total, approximately 150 patients were enrolled in the study.

### 6.1.3 Objectives

### Primary Objective

The primary Objective was to assess the antitumor activity of enfortumab vedotin monotherapy or enfortumab vedotin in combination with pembrolizumab as measured by ORR per RECIST Version 1.1 BICR.

### Secondary Objectives Include:

To assess the ORR per RECIST Version 1.1 by investigator assessment,
To assess the DOR per RECIST Version 1.1 by BICR and investigator assessment,
To assess the DCR per RECIST Version 1.1 by BICR and investigator assessment,
To assess PFS on study therapy per RECIST Version 1.1 by BICR and investigator assessment,
To assess OS,
To assess the safety and tolerability of enfortumab vedotin monotherapy or enfortumab vedotin in combination with pembrolizumab.

### Additional Objectives Include:

To assess PK and the incidence of ATA,
To assess Nectin-4 and PD-L1 expression levels,
To assess biomarkers of biological activity and disease resistance, and their potential associations with clinical outcome measures,
To assess PFS on subsequent therapy (PFS2) by investigator assessment,
To assess the impact on QoL and symptoms, including pain, from the patient perspective, and
For the EV+Pembro arm, to assess ORR, DOR, DCR and PFS on study therapy per iRECIST by investigator assessment.

### Endpoints

### Primary Endpoint

ORR (confirmed) per RECIST Version 1.1 by BICR.

### Secondary Endpoints

ORR (confirmed) per RECIST Version 1.1 by investigator assessment,
DOR per RECIST Version 1.1 by BICR,
DOR per RECIST Version 1.1 by investigator assessment,
DCR per RECIST Version 1.1 by BICR,
DCR per RECIST Version 1.1 by investigator assessment,
PFS per RECIST Version 1.1 by BICR,
PFS per RECIST Version 1.1 by investigator,
OS,
Type, incidence, severity, seriousness, and relatedness of AEs, and
Type, incidence, and severity of laboratory abnormalities,

### Additional Endpoints

Selected plasma or serum PK parameters of enfortumab vedotin, MMAE, and Tab,
Incidence of ATA to enfortumab vedotin,
Exploratory biomarkers of clinical activity, including relationship of Nectin-4 expression and PD-L1 expression status to response,
PFS2 by investigator assessment,
Change from baseline in PRO assessments of the EQ-5D-5L, EORTC QLQ-C30, and BPI-SF, and
For the EV+Pembro arm: (1) ORR (confirmed) per iRECIST by investigator assessment; (2) DOR per iRECIST by investigator assessment; (2) DCR per iRECIST by investigator assessment; and (4) PFS per iRECIST by investigator assessment.

### 6.1.4 Study Plan

### 6.1.4.1 Summary of Study Design

This study was designed to evaluate the safety and antitumor activity of enfortumab vedotin as monotherapy or in combination with pembrolizumab for the treatment of patients with la/mUC.

This study included 2 treatment arms randomized in a 1:1 ratio to enfortumab vedotin monotherapy or enfortumab vedotin in combination with pembrolizumab. See FIG. 2.

All patients received enfortumab vedotin, the investigational agent under study, administered as an IV infusion on Days 1 and 8 of every 3-week cycle.

Response were assessed by CT (with IV contrast) of the chest, abdomen, and pelvis every 3 cycles (every 9 weeks [±1 week] timed from Cycle 1 Day 1). If contrast media were contraindicated, please refer to the current Image Acquisition Guidelines (Section 6.1.13) for preferred scanning and contrast options. Objective responses were confirmed per RECIST Version 1.1 (Eisenhauer 2009) (see Table 40) with repeat scans at least 4 weeks after first documentation of response (scans required at 4 to 5 weeks). Subsequent response assessments were performed every 9 weeks (±1 week) according to the original schedule, timed from Cycle 1 Day 1, until 1 year after the first dose, then every 12 weeks (±1 week). Tumor imaging should also be performed whenever disease progression is suspected.

Patients continued on study treatment until radiographically confirmed disease progression, unacceptable toxicity, investigator decision, consent withdrawal, start of a subsequent anticancer therapy, pregnancy, or study termination by the sponsor. Treatment with pembrolizumab was discontinued once the patient received 35 administrations of pembrolizumab (approximately 2 years).

During study treatment, palliative radiotherapy on a nontarget bone lesion that was not progressing was not considered a subsequent anticancer therapy; however, radiotherapy on any target lesion was a subsequent anticancer therapy. Patients with iUPD on therapy per iRECIST guidelines (Seymour 2017) (Section 6.1.15) continued on study treatment until progression was confirmed by the investigator (iCPD) with a subsequent scan 4 to 9 weeks after iUPD.

Patients who discontinued study treatment for reasons other than radiographically confirmed disease progression or consent withdrawal continued to receive response assessments every 9 weeks (±1 week) after the previous response assessment scan, up to 1 year after the first dose, then every 12 weeks (±1 week). Scans were done until the patient had radiologically confirmed PD per RECIST Version 1.1 for EV Mono arm as determined by the investigator, initiated a subsequent anticancer therapy, died, the study closed, or the patient withdrew consent, whichever occurs first.

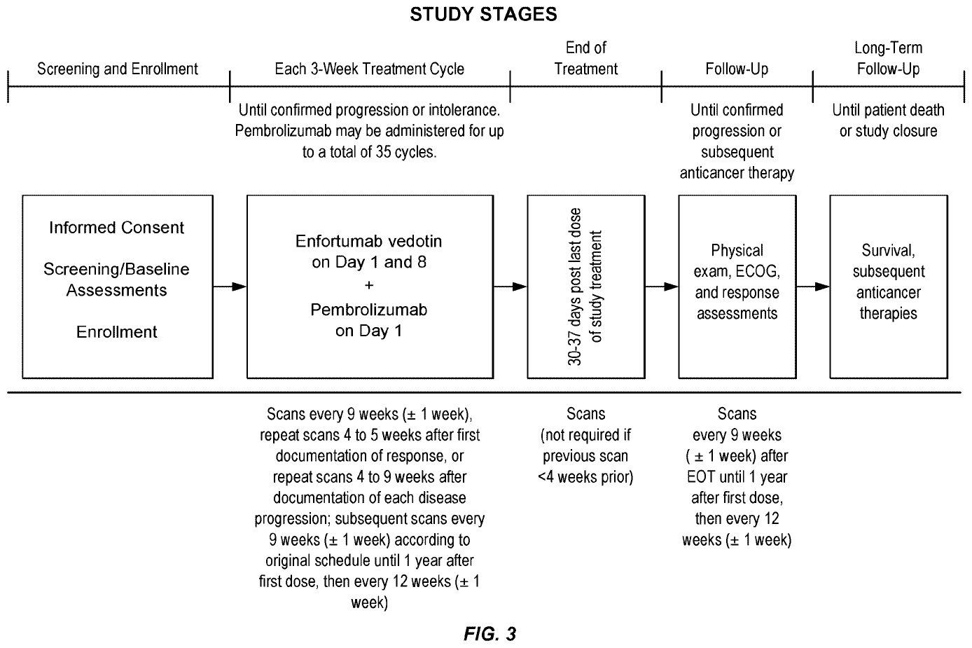

After progression on or following study treatment and discontinuation of study treatment, patients were followed every 12 weeks (±1 week) to obtain information on subsequent anticancer therapy, and to assess survival status until death, study closure, or patient withdraws consent, whichever occurred first. See FIG. 3 for a schema of patient flow through the study.

After initiation of subsequent anticancer therapy or radiologically confirmed disease progression per RECIST Version 1.1 (EV Mono arm) or per iRECIST (EV+Pembro arm) on or following study treatment (by investigator assessment), patients were followed via imaging per institutional guidelines, but not less frequently than every 12 weeks until PFS2 was documented or the patient started another anticancer treatment, whichever occurred first. All subsequent anticancer therapy including date of progression for PFS2 were recorded on the case report form (CRF). In addition, patients were contacted every 12 weeks (±1 week) after end of treatment (EOT) for survival status and clinical progression status on or following subsequent therapy. Following PFS2, patients entered the survival follow-up period and were followed every 12 weeks (±1 week) for survival status until death, study closure, or withdrawal of consent, whichever occurred first. Patient reported outcome assessments (QLQ-C-30, EQ-5D-5L, BPI-SF, HRU) were completed once every cycle.

In the EV+Pembro arm, patients who experienced unacceptable toxicity that was attributable only to pembrolizumab continued on enfortumab vedotin monotherapy until radiographically confirmed disease progression, unacceptable toxicity, investigator decision, consent withdrawal, start of a subsequent anticancer therapy, pregnancy, or study termination by the sponsor. Patients in the EV+Pembro arm who experienced toxicity that was attributable only to enfortumab vedotin continued on pembrolizumab monotherapy (for up to 35 cycles) until disease progression, unacceptable toxicity, investigator decision, consent withdrawal, start of a subsequent anticancer therapy, pregnancy, or study termination by the sponsor.

Safety was monitored over the course of the study by the SMC to determine if any new safety signals emerged to suggest that this was not a tolerable combination or schedule (see Section 6.1.4.1). Safety was monitored by a restricted SMC on an ongoing basis.

There were no dose-escalations or reductions of pembrolizumab.

Approximately 150 patients who were ineligible for cisplatin-based chemotherapy and who have not received prior systemic treatment for locally advanced or metastatic disease were stratified by the absence or presence of liver metastases, and ECOG performance status (0 versus 1/2), and randomized in a 1:1 ratio to receive either enfortumab vedotin monotherapy (EV Mono arm) or enfortumab vedotin in combination with pembrolizumab (EV+Pembro arm).
The EV Mono arm included approximately 75 patients. Patients received enfortumab vedotin (1.25 mg/kg IV) on Days 1 and 8 of each 3-week cycle.
The EV+Pembro arm included approximately 75 patients. Patients received enfortumab vedotin (1.25 mg/kg IV) followed by pembrolizumab (200 mg IV) on Day 1, and enfortumab vedotin alone was administered on Day 8 of each 3-week cycle. Pembrolizumab was administered for up to a total of 35 cycles.

Safetly was monitored by a restricted SMC on an ongoing basis.

### 6.1.4.2 Discussion and Rationale for Study Design

The evaluation of enfortumab vedotin+pembrolizumab versus enfortumab vedotin allowed the generation of additional data on the current combination as well as provided information on the contribution of activity coming from enfortumab vedotin monotherapy in cisplatin ineligible patients with la/mUC in the first-line setting. Randomization ensured that the baseline characteristics of patients in each arm were comparable.

### (i) Method of Assigning Patients to Treatment Groups

Patients were stratified by the absence or presence of liver metastases, and ECOG performance status (0 versus 1/2), and randomized in a 1:1 ratio to the EV Mono arm or EV+Pembro arm based on the stratification factors detailed in Section 6.1.8.3.

### (ii) Blinding

This was an open-label study.

### 6.1.5 Study Population

### 6.1.5.1 Inclusion Criteria

Patients must have had histologically documented locally advanced or metastatic urothelial (previously known as transitional cell) cancer (i.e., cancer of the bladder, renal pelvis, ureter, or urethra). Patients with squamous differentiation or mixed cell types were eligible. Patients with locally advanced disease that is resectable with curative intent were ineligible.

Patient must be eligible for CPI therapy.

Patients must be ineligible for cisplatin-based chemotherapy at the time of enrollment due to at least 1 of the following criteria::
Glomerular filtration rate (GFR) <60 mL/min but ≥30 mL/min (measured by the Cockcroft-Gault formula, modification of diet in renal disease [MDRD] or 24 hour urine)
ECOG performance status of 2 (refer to inclusion criterion #6 for additional criteria for subjects with ECOG 2)
NCI CTCAE Version 4.03 Grade ≥2 hearing loss
NYHA Class III heart failure

Patients must not have received prior systemic treatment for locally advanced or metastatic disease. Patients may not have previously received adjuvant/neoadjuvant platinum-based therapy within 12 months prior to randomization.

Minimum age of 18 years.

Patients must have measurable disease according to RECIST Version 1.1 (Eisenhauer et al., Eur. J. Cancer (2009); 45(2): 228-47). Lesions in a prior irradiated field must have progressed to be considered measurable.

An ECOG performance status of 0, 1, or 2.
Subjects with ECOG performance status of 2 must additionally meet the following criteria:
   i. Hemoglobin ≥10 g/dL
   ii. GFR ≥50 mL/min
   iii. May not have NYHA Class III heart failure

Anticipated life expectancy of ≥3 months as assessed by the investigator.

Have adequate organ function as defined in the Table 10. Specimens must be collected within 7 days prior to the start of study treatment.

TABLE 10
Adequate Organ Function Definitions

| System | Laboratory Value |
|---|---|
| Hematological | |
| ANC | ≥1500/μL |
| Platelets | ≥100,000/μL |
| Hemoglobin | ≥9.0 g/dL or ≥5.6 mmol/L^{a} |
| Renal | |
| Measured or calculated^{b} CrCl | ≥30 mL/min for participants with |
| AND/OR creatinine ≥1.5 × ULN. | creatinine levels (required; see |
| Both creatinine level and creatinine | Inclusion Criterion 3) |
| clearance are applicable when local | >1.5 × institutional ULN, AND/ |
| guidelines require both assessments | OR creatinine levels |
| (GFR can also be used in place of | ≤1.5 × upper limit of normal |
| CrC1) | (ULN) |
| Hepatic | |
| Serum Total bilirubin | ≤1.5 × ULN OR direct bilirubin |
| | ≤ ULN for participants with total |
| | bilirubin levels |
| | >1.5 × ULN |
| | ≤3 × ULN for patients with |
| | Gilbert's disease |
| AST (SGOT) and ALT (SGPT) | ≤3 × ULN |
| Coagulation | |
| International normalized ratio (INR) | ≤1.5 × ULN unless participant is |
| OR prothrombin time (PT) | receiving anticoagulant therapy as |
| Activated partial thromboplastin | long as PT or aPTT is within |
| time (aPTT) OR | therapeutic range of intended use |
| Partial thromboplastin time (PTT) | of anticoagulants |

Note:
Table 10 includes eligibility-defining laboratory value requirements for treatment; laboratory value requirements should be adapted according to local regulations and guidelines for the administration of specific chemotherapies.
ALT (SGPT) = alanine aminotransferase (serum glutamic pyruvic transaminase);
ANC = absolute neutrophil count; AST (SGOT) = aspartate aminotransferase (serum glutamic oxaloacetic transaminase); CrCl = creatinine clearance; GFR = glomerular filtration rate
^{a}Criteria must be met without erythropoietin dependency and without packed red blood cell (pRBC) transfusion within last 2 weeks.
^{b} Creatinine Clearance should be calculated using the Cockcroft-Gault method, Modification of Diet in Renal Disease equations (MDRD), or by 24-hour urine collection.

Agree not to try to become pregnant during the study and for at least 6 months after the final dose of study drug.
Must have a negative urine or serum pregnancy test (minimum sensitivity of 25 mIU/mL or equivalent units of beta human chorionic gonadotropin [β-hCG]) within 3 days prior to Day 1. Female subjects with false positive results and documented verification of negative pregnancy status are eligible for participation.
If heterosexually active, must consistently use highly effective methods of birth control, with a failure rate of less than 1% (as described in Section 6.1.14) starting at screening, throughout the study period, and for at least 6 months after the final dose of study drug.
Female subjects must agree not to breastfeed or donate ova starting at screening and throughout the study period, and for at least 6 months after the final dose of study drug.

A male subject who can father children is anyone born male who has testes and who has not undergone surgical sterilization (eg, vasectomy followed by a clinical test proving that the procedure was effective). Male subjects who can father children, must have met the following conditions:
Must not donate sperm starting at screening and throughout the study period, and for at least 6 months after the final dose of study drug. Male subjects are informed about the negative risk to reproductive function and fertility from the study treatment. Prior to treatment male subjects should be advised to seek information on fertility preservation and sperm cryoconservation.
Must consistently use highly effective methods of birth control, with a failure rate of less than 1% (as described in Section 6.1.14) starting at screening and continue throughout study period and for at least 6 months after the final dose of study drug.
Must subjects with a pregnant or breastfeeding partner(s) must consistently use one of 2 contraception options for preventing secondary exposure to seminal fluid (as described in Section 6.1.14) for the duration of the pregnancy or time partner is breastfeeding throughout the study period and for at least 6 months after the final dose of study drug.

The patient must provide written informed consent.

### 6.1.5.2 Exclusion Criteria

Received any prior treatment with a CPI. A CPI is defined as a PD-1 inhibitor, PD-L1 inhibitor, or PD-L2 inhibitor (including, but not limited to, atezolizumab, pembrolizumab, nivolumab, durvalumab, or avelumab).

Received any prior treatment with an agent directed to another stimulatory or co inhibitory T-cell receptor (including but not limited to CD137 agonists, CTLA 4 inhibitors, or OX-40 agonists).Ongoing sensory or motor neuropathy Grade 2 or higher.

Active central nervous system [CNS] metastases. Patients with treated CNS metastases were permitted on study if all the following were true:
a. CNS metastases have been clinically stable for at least 6 weeks prior to screening and baseline scans show no evidence of new or enlarged metastasis.
b. If requiring steroid treatment for CNS metastases, the patient was on a stable dose <10 mg/day of prednisone or equivalent for at least 2 weeks.
c. Patient does not have leptomeningeal disease.

Ongoing clinically significant toxicity (Grade 2 or greater) associated with prior treatment (including radiotherapy or surgery).

Patients with conditions requiring high doses of steroids (>10 mg/day of prednisone or equivalent) or other immunosuppressive medications were excluded. Inhaled or topical steroids were permitted in the absence of active autoimmune disease.

Prior treatment with enfortumab vedotin or other MMAE-based ADCs for urothelial cancer.

Subjects with a history of another invasive malignancy within 3 years before the first dose of study drug, or any evidence of residual disease from a previously diagnosed malignancy. Subjects with nonmelanoma skin cancer or carcinoma in situ of any type (if complete resection was performed) were allowed.

A history of prostate cancer (T2NXMX or lower with Gleason score ≤7) treated with definitive intent (surgically or with radiation therapy) at least 1 year prior to study entry was acceptable, provided that the subject was considered prostate cancer-free and the following criteria were met:
a. Participants who have undergone radical prostatectomy must have had undetectable PSA for >1 year and at screening.
b. Participants who have had radiation must have had a PSA doubling time >1 year (based on at least 3 values determined >1 month apart) and a total PSA value that does not meet Phoenix criteria for biochemical recurrence (i.e., <2.0 ng/mL above nadir).

Participants with untreated low-risk prostate cancer (Gleason score ≤6) on active surveillance with PSA doubling time >1 year (based on at least 3 values determined >1 month apart) were also eligible.

Currently receiving systemic antimicrobial treatment for active infection (viral, bacterial, or fungal) at the time of first dose of enfortumab vedotin. Routine antimicrobial prophylaxis was permitted.

Patients with a positive hepatitis B surface antigen and/or antihepatitis B core antibody; patients with a negative polymerase chain reaction (PCR) assay were permitted with either universal prophylaxis or the use of a pre-emptive approach. The approach is selected in accordance with regional or national guidelines for patients who receive anticancer therapies.

Active hepatitis C infection or known human immunodeficiency virus (HIV) infection. Patients who have been curatively treated for hepatitis C infection were permitted if they had documented sustained virologic response of 12 weeks. No HIV testing was required unless mandated by local health authority.

Patients with Active Tuberculosis.

Documented history of a cerebral vascular event (stroke or transient ischemic attack), unstable angina, myocardial infarction, or cardiac symptoms (including congestive heart failure) consistent with NYHA Class IV (see Section 6.1.11) within 6 months prior to the first dose of enfortumab vedotin. Patients with NYHA Class III were permitted.

Radiotherapy or major surgery within 2 weeks prior to first dose of study drug. Patient must have recovered adequately from the toxicity and/or complications from the intervention prior to starting study treatment.

Treatment with chemotherapy, biologics, or investigational agents not otherwise prohibited by exclusion criterion No. 2 that was not completed 4 weeks prior to first dose of study drug.

Known severe (≥Grade 3) hypersensitivity to enfortumab vedotin or to any excipient contained in the drug formulation of enfortumab vedotin (including histidine, trehalose dihydrate, and polysorbate 20). Known severe (≥2 Grade 3) hypersensitivity to pembrolizumab or to any excipient contained in the drug formulations of pembrolizumab.

Patients with active keratitis or corneal ulcerations. Patients with superficial punctate keratitis were allowed if the disorder is being adequately treated in the opinion of the investigator.

Had an active autoimmune disease that has required systemic treatment in past 2 years (i.e., use of disease modifying agents, corticosteroids, or immunosuppressive drugs). Replacement therapy (e.g., thyroxine, insulin, or physiologic corticosteroid replacement therapy for adrenal or pituitary insufficiency) is not considered a form of systemic treatment and was allowed.

History of idiopathic pulmonary fibrosis; organizing pneumonia, drug-induced pneumonitis, idiopathic pneumonitis, or evidence of active pneumonitis on screening chest CT scan.

Prior allogeneic stem cell or solid organ transplant.

Administration of a live, attenuated vaccine within 30 days prior to first dose of study drug. Examples of live vaccines include, but are not limited to, the following: measles, mumps, rubella, varicella/zoster (chicken pox), yellow fever, rabies, BCG, and typhoid vaccine. Seasonal influenza vaccines for injection are generally killed virus vaccines and are allowed; however, intranasal influenza vaccines (e.g., FluMist®) are live attenuated vaccines and are not allowed.

Other underlying medical condition that, in the opinion of the investigator, would impair the ability of the patient to receive or tolerate the planned treatment and follow-up; any known psychiatric or substance abuse disorders that would interfere with cooperating with the requirements of the study.

Patients with uncontrolled diabetes. Uncontrolled diabetes is defined as hemoglobin A1c (HbA1c) ≥8% or HbA1c 7% to <8% with associated diabetes symptoms (polyuria or polydipsia) that are not otherwise explained.

### 6.1.6 Treatments

### 6.1.6.1 Treatments Administered

Patients in this study (EV Mono arm or EV+Pembro arm) receive 1.25 mg/kg enfortumab vedotin administered as an IV infusion over 30 minutes on Days 1 and 8 of every 3-week cycle.

On Day 1 of each 3-week cycle, pembrolizumab 200 mg is administered as an IV infusion over 30 minutes approximately 30 minutes after completion of enfortumab vedotin (if randomized to EV+Pembro arm).

### 6.1.6.2 Investigational Study Drugs

### (i) Description—Enfortumab Vedotin

Enfortumab vedotin is generated by conjugation of a chemical intermediate that contains both the MMAE and linker subunits to cysteine residues of the antibody. The resulting ADC contains an average of 3.8 drug molecules per antibody. The enfortumab vedotin drug product is a sterile, preservative free, white to off-white lyophilized powder reconstituted for IV administration. Enfortumab vedotin is supplied in 30 mg single-dose vials.

### (ii) Dose and Administration

Enfortumab vedotin was administered as an IV infusion over approximately 30 minutes on Days 1 and 8 of every 3-week cycle.

In the absence of IRRs, the infusion rate for all patients should have been calculated in order to achieve an approximate 30-minute infusion period. Enfortumab vedotin must not have been administered as an IV push or bolus. Enfortumab vedotin should not have been mixed with other medications. At least 1 week (7 days) must have elapsed between doses of enfortumab vedotin.

Enfortumab vedotin doses were calculated on the basis of a patient's actual body weight at baseline. Doses should have been recalculated when a patient's body weight changes by ≥10% of baseline or the previous cycle or when dose adjustment criteria were met. Actual weight was used except for patients weighing >100 kg; in such cases, the dose was calculated based on a weight of 100 kg. The maximum dose permitted in this study was 125 mg.

Doses must have been adjusted for patients who experience a ≥10% change in weight from baseline or the previous cycle. Patient weight was measured during all relevant assessment windows as described in the schedule of events and additionally per institutional standards, if applicable. Other dose adjustments for changes in body weight <10% were permitted per institutional standards.

The patient should have been observed during enfortumab vedotin administration and for at least 60 minutes following the infusion during the first 3 cycles. All supportive measures consistent with optimal patient care should have been given throughout the study according to institutional standards.

The infusion site should have been monitored closely for redness, swelling, pain, and infection during and at any time after administration. Patients should have been advised to report redness or discomfort promptly at the time of administration or after infusion. Institutional guidelines were followed for the administration of chemotherapy agents and precautions taken to prevent extravasation per institutional standards and as described in “Chemotherapy and Biotherapy Guidelines and Recommendations for Practice” (Polovich 2014) and “Management of Chemotherapy Extravasation: ESMO-EONS Clinical Practice Guidelines” (Perez Fidalgo 2012). In case of enfortumab vedotin extravasation, the combination drug(s) should have been held until consultation and further discussion with the medical monitor/sponsor.

(iii) Dose Modifications for Enfortumab Vedotin

Intrapatient dose reduction by 1 or 2 dose levels (see Table 8) was allowed depending on the type and severity of toxicity. Patients requiring a dose reduction may have been re-escalated by 1 dose level (i.e., patients reduced to 0.75 mg/kg may only have been re-escalated to 1 mg/kg) provided the toxicity does not require study drug discontinuation and has returned to baseline or ≤Grade 1. If the toxicity recurred, re escalation was not permitted. Patients with ≥Grade 2 corneal AEs were not permitted to dose re-escalate.

Dose modification recommendations for enfortumab vedotin associated toxicity are presented in Table 11 and Table 12.

TABLE 11
Recommended dose modifications for enfortumab vedotin-associated
hematologic toxicity

| Grade 1 | Grade 2 | Grade 3 | Grade 4 |
|---|---|---|---|
| Continue at same | Continue at same | Withhold dose until | Withhold dose until |
| dose level. | dose level. | toxicity is ≤ Grade 1 | toxicity is ≤ Grade 1 |
| | For Grade 2 | or has returned to | or has returned to |
| | thrombocytopenia, | baseline, then resume | baseline, then reduce |
| | withhold dose until | treatment at the same | dose by 1 dose level |
| | toxicity is ≤ Grade 1 | dose level or | and resume |
| | or has returned to | consider dose | treatment, or |
| | baseline, then | reduction by 1 dose | discontinue at the |
| | resume treatment at | level. | discretion of the |
| | the same dose level. | Transfusions or | investigator. |
| | | growth factors may | Transfusions or |
| | | be used as indicated | growth factors may |
| | | per institutional | be used as indicated |
| | | guidelines. | per institutional |
| | | | guidelines. |
| | | | For anemia, |
| | | | treatment |
| | | | discontinuation |
| | | | should be strongly |
| | | | considered. |
| . | | | |

Note:
hematologic toxicity refers to anemia, thrombocytopenia, neutropenia, and febrile neutropenia.

TABLE 12
Recommended dose modifications for enfortumab vedotin-associated
nonhematologic toxicity

| Grade 1 | Grade 2 | Grade 3 | Grade 4 |
|---|---|---|---|
| Continue at same | Continue at same | Withhold dose until | For Grade 4 AEs, |
| dose level. | dose level, except in | toxicity is ≤ Grade 1 or | discontinue |
| For Grade 1 rash or | the event of Grade 2 | has returned to | treatment.* |
| skin reactions, may | neuropathy or | baseline, then resume | For confirmed SJS |
| continue at same | corneal AEs. | treatment at the same | or TEN, or Grade 4 |
| dose level. | For worsening rash | dose level or consider | rash, permanently |
| If ocular symptoms | or skin reactions, | dose reduction by 1 | discontinue |
| and/or changes in | consider | dose level .* | treatment. |
| vision are | withholding | For Grade 3 rash or | Grade 4 vomiting |
| identified, the | enfortumab vedotin | skin reactions withhold | and/or diarrhea that |
| patient should be | until toxicity is ≤ | enfortumab vedotin | improves to ≤ |
| evaluated by an | Grade 1 or has | until toxicity is ≤ | Grade 2 within |
| optometrist or | returned to baseline, | Grade 1 or has returned | 72 hours with |
| ophthalmologist. | then resume | to baseline, then | supportive |
| | treatment at the | resume treatment at the | management does |
| | same dose level or | same dose level or | not require |
| | consider dose | consider dose reduction | discontinuation. |
| | reduction by 1 | by 1 level. | For Grade 4 |
| | level. | For suspected | hyperglycemia or |
| | For Grade 2 | Stevens-Johnson | blood glucose >500 |
| | neuropathy or | Syndrome (SJS) or | mg/dL, withhold |
| | corneal AEs, | suspected toxic | enfortumab vedotin |
| | withhold dose until | epidermal necrolysis | treatment and |
| | toxicity is | (TEN) withhold | undertake a full |
| | ≤ Grade 1 or has | enfortumab vedotin. | evaluation of the |
| | returned to baseline, | Consider referral of the | hyperglycemia to |
| | then resume | subject to a | determine the |
| | treatment at the | dermatologist/specialist | underlying |
| | same dose level. | for diagnosis and | diagnosis. Once the |
| | For the second | specialized care. | blood glucose is |
| | occurrence of | Subjects who have | ≤250 mg/dL and |
| | Grade 2 neuropathy | confirmed SJS or | patient is clinically |
| | or corneal AEs, | recurrent Grade 3 rash | and metabolically |
| | withhold dose until | events should have | stable, dosing may |
| | toxicity is | therapy permanently | be resumed with |
| | ≤ Grade 1, then | discontinued. | close monitoring |
| | reduce the dose by | For Grade 3 | after consultation |
| | 1 dose level and | neuropathy or corneal | with medical |
| | resume treatment. | AEs, discontinue | monitor. |
| | If ocular symptoms | treatment at the | |
| | and/or changes in | discretion of the | |
| | vision are | investigator. | |
| | identified, the | For Grade 3 | |
| | patient should be | hyperglycemia or | |
| | evaluated by an | blood glucose >250 | |
| | optometrist or | mg/dL, withhold | |
| | ophthalmologist. | enfortumab vedotin | |
| | | treatment. Resume | |
| | | treatment once | |
| | | hyperglycemia/blood | |
| | | glucose is ≤250 mg/dL | |
| | | and patient is clinically | |
| | | and metabolically | |
| | | stable. | |
| | | If ocular symptoms | |
| | | and/or changes in | |
| | | vision are identified, | |
| | | the patient should be | |
| | | evaluated by an | |
| | | optometrist or | |
| | | ophthalmologist. | |

*Grade 3/4 electrolyte imbalances/laboratory abnormalities, that are not associated with clinical sequelae or are corrected with supplementation/appropriate management within 72 hours of their onset do not require discontinuation (e.g., Grade 4 hyperuricemia). Grade 4 amylase/lipase elevation that is not associated with clinical sequalae does not require discontinuation; withhold dose until toxicity is ≤ Grade 3 or has returned to baseline, then resume treatment at the same dose level. Grade 3 amylase/lipase elevation that is not
associated with clinical sequelae may continue treatment at the same dose level. Hold therapy for ≥ Grade 3 amylase/lipase with clinical sequelae until evaluation is complete.

Intrapatient dose reduction or interruption for other enfortumab vedotin-associated toxicity was permitted at the discretion of the medical monitor and site investigator. On a per-patient basis, dose reductions for toxicity, including DLT, may have been allowed. The DLT-evaluation period was the first treatment cycle. Patients who experienced a DLT in the DLT-evaluation period should not have received further treatment unless clinical benefit was demonstrated with adequately managed toxicity, and there was approval from the medical monitor. The subsequent dose level was defined by the medical monitor in discussion with the site investigator; the type and severity of the AE observed was taken into consideration to inform the decision.

If the medical monitor and site investigator determined that a dose interruption for enfortumab vedotin was needed, an interruption of all Day 1 doses of pembrolizumab must have also occurred and may have lasted for up to 3 weeks (1 cycle).

Dose interruptions for patients without prior dose reductions and who were responding to treatment may have been dose interrupted beyond 3 weeks with approval of the medical monitor, if the patient's toxicity does not otherwise require permanent discontinuation. Patients may not have received other investigational drugs, radiotherapy (except palliative radiotherapy of symptomatic and nonprogressing nontarget bone lesions), or systemic antineoplastic therapy during dose delays. If toxicities warranting a dose delay occurred after Day 1 dosing and were not resolved prior to Day 8 dosing (up to Day 10), Day 8 enfortumab vedotin administration was skipped rather than delayed. If a patient is dose-reduced due to toxicity that subsequently resolved, the patient may have resumed treatment at the original dose at the discretion of the medical monitor and site investigator. If there was a dose interruption, the schedule for response assessments was not adjusted and was still calculated from Cycle 1 Day 1.

Dose interruptions (enfortumab vedotin or pembrolizumab) were allowed for situations other than treatment-related AEs such as medical/surgical events or logistical reasons not related to study therapy. Participants should have been placed back on study therapy within 3 weeks of the scheduled interruption, unless otherwise discussed with the sponsor. The reason for interruption should have been documented in the patient's study record.

See Section 6.1.6.4(i) for recommended management of infusion reactions. See Section 6.1.6.4(iv) for recommended management of hyperglycemia. See Section 6.1.6.4(v) for recommended management of rash.

In the EV+Pembro arm, patients who experienced unacceptable toxicity that was attributable only to pembrolizumab may have permanently discontinued pembrolizumab and continued on enfortumab vedotin monotherapy until radiographically confirmed disease progression, unacceptable toxicity, investigator decision, consent withdrawal, start of a subsequent anticancer therapy, pregnancy, or study termination by the sponsor. Patients in the EV+Pembro arm who experienced toxicity that was attributable only to enfortumab vedotin may have permanently discontinued enfortumab vedotin and continued on pembrolizumab monotherapy (for up to 35 cycles) until disease progression, unacceptable toxicity, investigator decision, consent withdrawal, start of a subsequent anticancer therapy, pregnancy, or study termination by the sponsor.

Patients in the EV Mono arm who experienced unacceptable toxicity that was attributable only to enfortumab vedotin should have been discontinued from study drug.

Patients who discontinued treatment with enfortumab vedotin and continued treatment with pembrolizumab should have returned to the clinic for Day 8 and Day 15 visits (for safety laboratory assessments). Day 8 vital signs are not collected for patients who discontinue treatment with enfortumab vedotin permanently.

### (a) Treatment Discontinuation Recommendations Related to Liver Safety

In the absence of an explanation for increased LFTs, such as viral hepatitis, pre-existing or acute liver disease, or exposure to other agents associated with liver injury, the patient may have been discontinued from the study treatment. The investigator may have determined that it was not in the patient's best interest to continue study treatment.

Discontinuation of treatment should have been considered if:
ALT or aspartate aminotransferase (AST) >8×upper limit of normal (ULN)
ALT or AST >5×ULN for more than 2 weeks
ALT or AST >3×ULN and total bilirubin >2×ULN or international normalized ratio (INR)>1.5 (if INR testing is applicable/evaluated)
ALT or AST>3×ULN with the appearance of symptoms suggestive of liver injury (eg, right upper quadrant pain or tenderness) and/or eosinophilia (>5%)

These treatment discontinuation recommendations are based on the FDA Guidance for Industry (Drug-Induced Liver Injury: Premarketing Clinical Evaluation, July 2009). The recommendations are a basic guide to the investigator based on accumulated clinical experience with drugs in development, and are not specific to clinical experience with enfortumab vedotin.

See Section 6.1.12 for recommended liver safety monitoring and assessment criteria in patients with Grade 2 or greater elevations in ALT, AST, or bilirubin.

### iv) Description—Pembrolizumab

Pembrolizumab is a humanized monoclonal antibody that blocks the interaction between PD-1 and its ligands, PD-L1 and PD-L2. Pembrolizumab is an IgG4 kappa Ig with an approximate molecular weight of 149 kDa.

Pembrolizumab injection is a sterile, preservative-free, clear to slightly opalescent, colorless to slightly yellow solution that requires dilution for IV infusion. Each vial contains 100 mg of pembrolizumab in 4 mL of solution. Each 1 mL of solution contains 25 mg of pembrolizumab and is formulated in L-histidine (1.55 mg), polysorbate 80 (0.2 mg), sucrose (70 mg), and Water for Injection, United States Pharmacopeia (USP).

### v) Dose and Administration

For the EV+Pembro arm, pembrolizumab was administered using IV infusion on Day 1 of each 3-week treatment cycle after all procedures and assessments have been completed, and approximately 30 minutes after completion of enfortumab vedotin administration..

Pembrolizumab was administered as a dose of 200 mg using an approximately 30-minute IV infusion.

Pembrolizumab was administered for up to a total of 35 cycles.

vi) Dose Modification and Toxicity Management for Immune-mediated Adverse Events Associated with Pembrolizumab

AEs associated with pembrolizumab exposure may represent an immunologic etiology. These imAEs may occur shortly after the first dose or several months after the last dose of pembrolizumab treatment and may affect more than 1 body system simultaneously. Therefore, early recognition and initiation of treatment is critical to reduce complications. Based on existing clinical study data, most imAEs were reversible and could be managed with interruptions of pembrolizumab, administration of corticosteroids and/or other supportive care. For suspected imAEs, adequate evaluation was ensured to confirm etiology or exclude other causes. Additional procedures or tests such as bronchoscopy, endoscopy, skin biopsy may have been included as part of the evaluation. Based on the severity of imAEs, pembrolizumab was withheld or permanently discontinued and corticosteroids administered. Dose modification and toxicity management guidelines for imAEs associated with pembrolizumab are provided in Table 13.

TABLE 13
Dose modification and toxicity management guidelines for immune-mediated AEs associated with pembrolizumab.

| | Toxicity Grade or | | imAE Management with | |
|---|---|---|---|---|
| | Conditions (NCI | Action Taken to | Corticosteroid and/or | |
| Immune-mediated AEs | CTCAE Version 4.03) | Pembrolizumab | Other Therapies | Monitor and Follow-up |
| Pneumonitis | Grade 2 | Withhold | Administer corticosteroids | Monitor participants for |
| | Grade 3 or 4, or recurrent | Permanently discontinue | (initial dose of 1-2 mg/kg | signs and symptoms of |
| | Grade 2 | | prednisone or equivalent) | pneumonitis |
| | | | followed by taper | Evaluate participants with |
| | | | | suspected pneumonitis |
| | | | | with radiographic imaging |
| | | | | and initiate corticosteroid |
| | | | | treatment |
| | | | | Add prophylactic |
| | | | | antibiotics for |
| | | | | opportunistic infections |
| Diarrhea/Colitis | Grade 2 or 3 | Withhold | Administer corticosteroids | Monitor participants for |
| | Recurrent Grade 3 or | Permanently discontinue | (initial dose of 1-2 mg/kg | signs and symptoms of |
| | Grade 4 | | prednisone or equivalent) | enterocolitis (i.e., |
| | | | followed by taper | diarrhea, abdominal pain, |
| | | | | blood or mucus in stool |
| | | | | with or without fever) and |
| | | | | of bowel perforation (ie, |
| | | | | peritoneal signs and ileus). |
| | | | | Participants with ≥ Grade |
| | | | | 2 diarrhea suspecting |
| | | | | colitis should consider |
| | | | | gastrointestinal (GI) |
| | | | | consultation and |
| | | | | performing endoscopy to |
| | | | | rule out colitis. |
| | | | | Participants with |
| | | | | diarrhea/colitis should be |
| | | | | advised to drink liberal |
| | | | | quantities of clear fluids. |
| | | | | If sufficient oral fluid |
| | | | | intake is not feasible, fluid |
| | | | | and electrolytes should be |
| | | | | substituted via IV |
| | | | | infusion. |
| AST/ALT elevation or | Grade 2 | Withhold | Administer corticosteroids | Monitor with liver |
| Increased bilirubin | | | (initial dose of 0.5-1 | function tests (consider |
| | | | mg/kg prednisone or | weekly or more frequently |
| | | | equivalent) followed by | until liver enzyme value |
| | | | taper | returned to baseline or is |
| | Grade 3 or 4 | Permanently discontinue | Administer corticosteroids | stable) |
| | | | (initial dose of 1-2 mg/kg | |
| | | | prednisone or equivalent) | |
| | | | followed by taper | |
| Type 1 diabetes mellitus | Newly onset T1DM or | Withhold¹ | Initiate insulin | Monitor participants for |
| (T1DM) or | Grade 3 or 4 | | replacement therapy for | hyperglycemia or other |
| Hyperglycemia | hyperglycemia associated | | participants with T1DM | signs and symptoms of |
| | with evidence of β-cell | | Administer anti- | diabetes. |
| | failure | | hyperglycemic in | See also enfortumab |
| | | | participants with | vedotin dose modification |
| | | | hyperglycemia | recommendations for |
| | | | | hyperglycemia in |
| | | | | Section 6.1.6.2(iii)(a) and |
| | | | | recommended |
| | | | | management of |
| | | | | hyperglycemia in Section |
| | | | | 6.1.6.4(iv) |
| Hypophysitis | Grade 2 | Withhold | Administer corticosteroids | Monitor for signs and |
| | Grade 3 or 4 | Withhold or permanently | and initiate hormonal | symptoms of hypophysitis |
| | | discontinue¹ | replacements as clinically | (including hypopituitarism |
| | | | indicated | and adrenal insufficiency) |
| Hyperthyroidism | Grade 2 | Continue | Treat with non-selective | Monitor for signs and |
| | Grade 3 or 4 | Withhold or | beta-blockers (e.g., | symptoms of thyroid |
| | | permanently discontinue¹ | propranolol) or | disorders |
| | | | thionamides as | |
| | | | appropriate | |
| Hypothyroidism | Grade 2-4 | Continue | Initiate thyroid | Monitor for signs and |
| | | | replacement hormones | symptoms of thyroid |
| | | | (e.g., levothyroxine or | disorders |
| | | | liothyronine) per SOC | |
| Nephritis and Renal | Grade 2 | Withhold | Administer corticosteroids | Monitor changes of renal |
| | Grade 3 or 4 | Permanently discontinue | (prednisone 1-2 mg/kg or | function |
| dysfunction | | | equivalent) followed by | |
| | | | taper. | |
| Myocarditis | Grade 1 | Withhold | Based on severity of AE | Ensure adequate |
| | Grade 2, 3 or 4 | Permanently discontinue | administer corticosteroids | evaluation to confirm |
| | | | | etiology and/or exclude |
| | | | | other causes |
| All other immune- | Persistent Grade 2 | Withhold | Based on severity of AE | Ensure adequate |
| mediated AEs | Grade 3 | Withhold or discontinue² | administer corticosteroids | evaluation to confirm |
| | Grade 4 or recurrent | Permanently discontinue | | etiology or exclude other |
| | Grade 3 | | | causes |

Note:
Non-imAE is managed as appropriate, following clinical practice recommendations.
AE(s)-adverse event(s); ALT = alanine aminotransferase; AST = aspartate aminotransferase; CTCAE = National Cancer Institute Common Terminology Criteria for Adverse Events; DRESS = Drug Rash with Eosinophilia and Systemic Symptom; GI = gastrointestinal; IO = immuno-oncology; ir = immune related; IV = intravenous; SJS = Stevens-Johnson Syndrome; SOC = standard of care; SJS = Stevens-Johnson Syndrome; T1DM = type 1 diabetes mellitus; TEN = Toxic Epidermal Necrolysis; ULN = upper limit of normal.
¹Withhold or permanently discontinue pembrolizumab is at the discretion of the investigator or treating physician.
²Events that require discontinuation include, but are not limited to: Guillain-Barre Syndrome, encephalitis, myelitis, DRESS, SJS, TEN and other clinically important irAEs (e.g., vasculitis and sclerosing cholangitis).
General instructions:
1. Corticosteroid taper should be initiated upon AE improving to Grade 1 or less and continue to taper over at least 4 weeks.
2. For situations where pembrolizumab has been withheld, pembrolizumab can be resumed after AE has been reduced to Grade 1 or 0 and corticosteroid has been tapered. Pembrolizumab should be permanently discontinued if AE does not resolve within 12 weeks of last dose or corticosteroids cannot be reduced to ≤10 mg prednisone or equivalent per day within 12 weeks.
3. For severe and life-threatening imAEs, IV corticosteroid should be initiated first followed by oral steroid. Other immunosuppressive treatment should be initiated if imAEs cannot be controlled by corticosteroids.

### vi) Dose Modification and Toxicity Management of Infusion-Reactions Related to Pembrolizumab

Pembrolizumab may cause severe or life-threatening infusion-reactions including severe hypersensitivity or anaphylaxis. Signs and symptoms usually develop during or shortly after drug infusion and generally resolve completely within 24 hours of completion of infusion. Dose modification and treatment guidelines on pembrolizumab associated infusion reaction are provided in Table 14.

TABLE 14
Pembrolizumab infusion reaction dose modification and treatment guidelines

| | | Premedication at |
|---|---|---|
| NCI CTCAE Grade | Treatment | Subsequent Dosing |
| Grade 1 | Increase monitoring of vital | None |
| Mild reaction; infusion | signs as medically indicated | |
| interruption not indicated; | until the participant is | |
| intervention not indicated | deemed medically stable in | |
| | the opinion of the | |
| | investigator. | |
| Grade 2 | Stop Infusion | Participant may be |
| Requires therapy or infusion | Additional appropriate | premedicated 1.5 h (±30 |
| interruption but responds | medical therapy may include | minutes) prior to infusion of |
| promptly to symptomatic | but is not limited to: | pembrolizumab with: |
| treatment (e.g., | IV fluids | Diphenhydramine 50 mg PO |
| antihistamines, NSAIDs, | Antihistamines | (or equivalent dose of |
| narcotics, IV fluids); | NSAIDs | antihistamine). |
| prophylactic medications | Acetaminophen | Acetaminophen 500-1000 |
| indicated for ≤24 h. | Narcotics | mg PO (or equivalent dose |
| | Increase monitoring of vital | of analgesic). |
| | signs as medically indicated | |
| | until the participant is | |
| | deemed medically stable in | |
| | the opinion of the | |
| | investigator. | |
| | If symptoms resolve within | |
| | 1 hour of stopping drug | |
| | infusion, the infusion may | |
| | be restarted at 50% of the | |
| | original infusion rate (eg, | |
| | from 100 mL/h to 50 mL/h). | |
| | Otherwise dosing is held | |
| | until symptoms resolve and | |
| | the participant should be | |
| | premedicated for the next | |
| | scheduled dose. | |
| | Participants who develop | |
| | Grade 2 toxicity despite | |
| | adequate premedication | |
| | should be permanently | |
| | discontinued from further | |
| | pembrolizumab treatment. | |
| Grade 3 or 4 | Stop Infusion | No subsequent dosing |
| Grade 3 | Additional appropriate | |
| Prolonged (ie, not rapidly | medical therapy may include | |
| responsive to symptomatic | but is not limited to: | |
| medication and/or brief | Epinephrine** | |
| interruption of infusion); | IV fluids | |
| recurrence of symptoms | Antihistamines | |
| following initial | NSAIDS | |
| improvement; | Acetaminophen | |
| hospitalization indicated for | Narcotics | |
| other clinical sequelae (eg, | Oxygen | |
| renal impairment, | Pressors | |
| pulmonary infiltrates) | Corticosteroids | |
| Grade 4 | Increase monitoring of vital | |
| Life-threatening; pressor or | signs as medically indicated | |
| ventilator support indicated | until the participant is | |
| | deemed medically stable in | |
| | the opinion of the | |
| | investigator. | |
| | Hospitalization may be | |
| | indicated. | |
| | **In cases of anaphylaxis, | |
| | epinephrine should be used | |
| | immediately. | |
| | Participant is permanently | |
| | discontinued from further | |
| | pembrolizumab treatment. | |

Appropriate resuscitation equipment should be available at the bedside and a physician readily available during the period of drug administration. For further information, please refer to the National Cancer Institute Common Terminology Criteria for Adverse Events (NCI CTCAE) Version 4.03 at the website accessible at ctep.cancer.gov. NSAID=nonsteroidal anti-inflammatory drug; PO=by mouth

vii. Dose Interruption

If the medical monitor and site investigator determined that a dose interruption for pembrolizumab was needed, an interruption of all Day 1 doses of other drugs (enfortumab vedotin and/or chemotherapy) must also have occurred and may last for up to 3 weeks (1 cycle).

viii. Other Allowed Dose Interruption for Pembrolizumab

Dose interruptions (enfortumab vedotin and pembrolizumab) may have been allowed for situations other than treatment-related AEs such as medical/surgical events or logistical reasons not related to study therapy. Participants should have been placed back on study therapy within 3 weeks of the scheduled interruption, unless otherwise discussed with the sponsor. The reason for interruption should have been documented in the patient's study record.

### 6.1.6.3 Concomitant Therapy

All concomitant medications and blood products administered were recorded from Day 1 (predose) through the safety reporting period (EOT visit or 30 days after the last study treatment, whichever is later). Any concomitant medication given for a study protocol-related AE should have been recorded from the time of informed consent.

### (i) Required Concomitant Therapy

There are no required concomitant therapies.

### (ii) Allowed Concomitant Therapy

During study treatment, palliative radiotherapy on a nontarget bone lesion that was not progressing was allowed in locally advanced/metastatic setting and was not considered a subsequent anticancer therapy (see guidance for target lesions in Section 6.1.6.3(iii)).

The use of anti-emetics was permitted. The use of insulin was permitted as part of SOC. Premedications for IRRs per Section 6.1.6.4 were permitted; however, prophylactic premedication prior to study treatment on Cycle 1 Day 1 for prevention of IRRs may not have been administered.

Patients who were receiving strong CYP3A4 inhibitors or P-glycoprotein (P-gp) inhibitors concomitantly with enfortumab vedotin should have been closely monitored for adverse reactions.

Routine prophylaxis with vaccines was permitted; however, patients may not have been treated with a live, attenuated vaccine during the study.

The following applies to EV+Pembro arm: With approval from the medical monitor, concomitant chronic prednisone (or equivalent) may have been used at a dose of ≤10 mg/day to manage pre-existing conditions. Higher doses of prednisone (or equivalent) were permitted for limited duration to treat acute conditions that arose during the study as medically indicated after discussion with the medical monitor. Chronic use of inhaled or topical steroids were permitted in the absence of active autoimmune disease.

For EV Mono arm, steroids may have been used as clinically indicated.

(iii) Prohibited Concomitant Therapy

Medications or vaccinations specifically prohibited in the exclusion criteria were not allowed during the ongoing trial. If there was a clinical indication for any medication or vaccination specifically prohibited during the trial, discontinuation from trial therapy or vaccination may have been required. The investigator should have discussed any questions regarding this with the medical monitor. The final decision on any supportive therapy or vaccination rested with the investigator and/or the participant's primary physician. However, the decision to continue the participant on study treatment required the mutual agreement of the investigator, the sponsor, and the participant.

Listed below are specific restrictions for concomitant therapy or vaccination during the course of the study:
Antineoplastic systemic chemotherapy or biological therapy.
Immunotherapy not specified in this protocol.
Chemotherapy not specified in this protocol.
Investigational agents other than pembrolizumab or enfortumab vedotin.
Radiation therapy (Note: Radiation therapy to a symptomatic nontarget bone lesion in locally advanced/metastatic setting was allowed at the investigator's discretion.)
Live vaccines within 30 days prior to the first dose of study treatment and while participating in the study. Examples of live vaccines include, but are not limited to, the following: measles, mumps, rubella, varicella/zoster, yellow fever, rabies, BCG, and typhoid vaccine. Seasonal influenza vaccines for injection are generally killed virus vaccines and are allowed; however, intranasal influenza vaccines (eg, FluMist) are live attenuated vaccines and were not allowed.
Pioglitazone.

Participants who, in the assessment of the investigator, required the use of any of the aforementioned treatments for clinical management should have been removed from the study.

### 6.1.6.4 Management of Adverse Reactions

### (i) Management of Infusion Reactions—Enfortumab Vedotin

An IRR may have occurred during the infusion of study treatment. The infusion should have been administered at a site properly equipped and staffed to manage anaphylaxis should it occur. All supportive measures consistent with optimal patient care should have been given throughout the study according to institutional standards. Supportive measures may include administering medications for IRRs.

Premedications for IRRs were permitted as described below; however, prophylactic premedication prior to study treatment on Cycle 1 Day 1 for prevention of IRRs may not have been administered. Patients who have experienced an IRR may have been premedicated for subsequent infusions. Premedication may have included pain medication (e.g., acetaminophen or equivalent), an antihistamine (e.g., diphenhydramine hydrochloride), and a corticosteroid administered approximately 30 to 60 minutes prior to each enfortumab vedotin infusion or according to institutional standards. Should a patient have experienced IRRs in the setting of premedication, continued treatment was discussed with the medical monitor prior to the next planned dose.

If anaphylaxis occurred, study treatment administration should have been immediately and permanently discontinued.

### (ii) Management of Infusion Reactions—Pembrolizumab

For management of infusion reactions, see Table 13, supra.

(iii) Supportive Care Guidelines for Pembrolizumab

Participants should have received appropriate supportive care measures as deemed necessary by the treating investigator. Suggested supportive care measures for the management of AEs with potential immunologic etiology are outlined along with the dose modification guidelines in Section 6.1.6.2, and Table 12. Where appropriate, these guidelines include the use of oral or IV treatment with corticosteroids, as well as additional anti-inflammatory agents if symptoms did not improve with administration of corticosteroids. Note that several courses of steroid tapering may have been necessary as symptoms may worsen when the steroid dose is decreased. For each disorder, attempts should have been made to rule out other causes such as metastatic disease or bacterial or viral infection, which might require additional supportive care. The treatment guidelines were intended to be applied when the investigator determined the events to be related to pembrolizumab.

Note: If after the evaluation of the event, it was determined not to be related to pembrolizumab, the investigator did not need to follow the treatment guidance. Refer to Table 12 in Section 6.1.6.2 for guidelines regarding dose modification and supportive care.

It may have been necessary to perform conditional procedures such as bronchoscopy, endoscopy, or skin photography as part of evaluation of the event.

### (iv) Management of Hyperglycemia

Investigators should monitor blood glucose levels and were advised to perform additional assessments if any symptoms of hyperglycemia were observed, including a thorough evaluation for infection. In addition, if steroids were used to treat any other condition, blood glucose levels may have required additional monitoring. If elevated blood glucose levels were observed, patients should have been treated according to local SOC and referral to endocrinology may be considered.

Patients, especially those with a history of or ongoing diabetes mellitus or hyperglycemia, should have been advised to immediately notify their physician if their glucose level became difficult to control or if they experienced symptoms suggestive of hyperglycemia such as frequent urination, increased thirst, blurred vision, fatigue, and headache.

Patients who entered the study with an elevated HbA1c (≥6.5%) at baseline should have been referred to an appropriate provider during Cycle 1 for glucose management. Blood glucose should have been checked prior to each dosing and Dose should have been withheld for blood glucose >250 mg/dL. Dosing may have continued once the patient's blood glucose improved to ≤250 mg/dL and patient was clinically and metabolically stable. The use of insulin was permitted as part of SOC. Blood glucose >500 mg/dL considered related to enfortumab vedotin required drug interruption and a full evaluation of the hyperglycemia to determine the underlying diagnosis. Once hyperglycemia/elevated blood glucose improved to ≤250 mg/dL, dosing resumed with close monitoring after consultation with medical monitor. If a patient experienced new onset of diabetes mellitus, the patient was evaluated with a metabolic panel, urine ketones, HbA1c, C-peptide, to assess new onset of type 1 diabetes in the setting of combination with CPI.

### (v) Management of Rash Related to Enfortumab Vedotin

Enfortumab vedotin is a Nectin-4 directed antibody drug conjugate. Nectin-4 is a cell adhesion molecule that is highly expressed in urothelial carcinoma. Low to moderate levels of Nectin-4 are also expressed on normal tissues, including skin keratinocytes, sweat glands and hair follicles; thus, skin reactions are anticipated events. As such, skin reactions were AEs of interest in all clinical studies with enfortumab vedotin.

Reports of severe cutaneous adverse reactions were identified in 15 patients receiving enfortumab vedotin, some of whom had fatal outcomes. These reactions occurred predominantly during the first cycle of treatment. AEs reported in these cases included Stevens-Johnson Syndrome (SJS) (5 cases), blister (3 cases), dermatitis bullous (3 cases), symmetrical drug-related intertriginous and flexural exanthema (SDRIFE; 2 cases), and 1 case each of dermatitis exfoliative, exfoliative rash, epidermal necrosis, oropharyngeal blistering, stomatitis, and toxic epidermal necrolysis (TEN).

In enfortumab vedotin monotherapy studies of urothelial carcinoma, SAEs of severe cutaneous adverse reactions were reported in 11 of 749 subjects (1.5%) and included dermatitis bullous (0.4%), drug eruption (0.4%), blister (0.1%), conjunctivitis (0.1%), SJS (0.1%), stomatitis (0.1%), and toxic skin eruption (0.1%).

Subjects should have been advised to contact the Investigator immediately if they had signs and symptoms of skin reactions, oral mucosal and ocular abnormalities including mucositis or conjunctivitis. Starting in the first cycle and throughout treatment, subjects were closely monitored for skin reactions. For mild to moderate skin reactions, appropriate treatment was considered, such as topical corticosteroids and antihistamines as clinically indicated. For worsening Grade 2 rash or skin reactions, withholding of enfortumab vedotin was considered. For severe (Grade 3) rash or skin reactions or suspected SJS or TEN, enfortumab vedotin was withheld and referral for specialized care was considered. Discontinue enfortumab vedotin was permanently discontinued in patients with confirmed SJS or TEN, or Grade 4 or recurrent Grade 3 skin reactions, if any.

For enfortumab vedotin combination therapies, if the rash or skin reaction was determined to be related to study drugs but the investigator was unable to distinguish relatedness to individual study drugs, then management actions (ie, to withhold or discontinue treatment) should have applied to all study drugs.

### 6.1.6.5 Treatment Compliance

Study drug administration was performed by study site staff and documented in source documents and the CRF.

### 6.1.6.6 Treatment Duration

Patients in the EV+Pembro arm may have been treated beyond disease progression per RECIST v1.1 provided they were deriving clinical benefit, as defined by all of the following:
Improvement in clinical symptoms or functional status as assessed by the investigator.
No decline in WHO or ECOG performance status attributable to disease progression.
Absence of symptoms or signs (including worsening laboratory values) of unequivocal disease progression.
Absence of disease progression at critical anatomic sites (e.g., CNS, spinal cord compression).

### 6.1.7 Study Assessments

### 6.1.7.1 Screening/Baseline Assessments

Only patients who met all inclusion and exclusion criteria specified in Section 6.1.5 were enrolled in this study. Enrollment status and date was recorded in CRF.

Patient medical history included a thorough review of significant past medical history, current conditions, any treatment for prior malignancies and response to prior treatment, and any concomitant medications.

### 6.1.7.2 Locally Advanced or Metastatic Urothelial Cancer

Acquisition of a fresh tumor biopsy tissue, collection of an archived tumor specimen, a complete eye examination, brain scan (MRI with gadolinium contrast preferred; see Section 6.1.13 for preferred imaging methods), bone scan, CT (with IV contrast) of the chest, abdomen, and pelvis for baseline tumor imaging (if contrast media are contraindicated, please refer to the current Image Acquisition Guidelines [Section 6.1.13] for preferred scanning and contrast options), serology for hepatitis B surface antigen and antihepatitis B core antibody, serology for antihepatitis C antibody, urinalysis with reflexive microscopic analysis, HbA1c, and thyroid function tests are required for all patients at screening. INR/PT/PTT, a pregnancy test (either urine or serum, for females of childbearing potential), physical examination (including weight), collection of height, vital signs, CBC with differential, serum chemistry panel, CrCl, and ECOG performance status assessment were required at baseline. An ECHO was required at baseline for subjects with NYHA Class III heart failure or a history of coronary heart disease, arrhythmia or other significant heart disease.

### 6.1.7.3 Response/Efficacy Assessments

Disease was assessed by CT (with IV contrast) of the chest, abdomen, and pelvis at protocol-specified time points. Patients must have been evaluated using the same imaging method throughout the study for response assessments. If contrast media were contraindicated, please refer to the current Image Acquisition Guidelines (Section 6.1.13) for preferred scanning and contrast options. Other regions should have been scanned if the patient had known disease or new symptoms suggestive of disease in that region. Brain scans (which are required at screening) should have been repeated at disease assessment time points if inactive brain metastases were known at baseline, or as clinically indicated throughout the study. Bone scans (which are required at screening) should also have been repeated at response assessment time points if bone metastases were identified at baseline, or if bone metastasis was known or suspected.

Lesions in the urinary bladder that are detected on CT/MRI scans may have been selected as target lesions, provided the radiologist considered that the lesion(s) lend themselves to reproducible measurements at post-baseline timepoints and the lesions have been measured with adequate bladder distension. The same imaging modality should have been used throughout the study treatment.

If newly obtained core or excisional biopsy of a tumor lesion was provided, baseline imaging should have been performed (or repeated) after the biopsy to ensure accurate assessment of the tumor response, should that specific lesion have been selected as a target lesion.

Objective responses were confirmed per RECIST Version 1.1 (Eisenhauer 2009) (Table 40) with repeat scans at least 4 weeks after first documentation of response (scans required at 4 to 5 weeks), then subsequent response assessments should have been performed every 9 weeks (±7 days) timed from Cycle 1 Day 1 according to the original schedule until 1 year after first dose, then every 12 weeks (±7 days). Tumor imaging should have also been performed whenever disease progression is suspected.

Patients in the EV+Pembro arm with iUPD on therapy per iRECIST guidelines (Seymour 2017) (Section 6.1.15) may have continued on study treatment until iCPD was confirmed by the investigator with a subsequent scan 4 to 9 weeks after iUPD. For patients in the EV Mono arm, the investigator made treatment decisions based on site assessments of scans by RECIST Version 1.1.

Patients who discontinued study treatment for reasons other than radiographically confirmed disease progression or consent withdrawal continued to receive response assessments, including CT scans with contrast, every 9 weeks (±7 days) according to the original schedule calculated from Cycle 1 Day 1 up to 1 year after first dose, then every 12 weeks (±7 days). Scans were done until the patient had radiologically confirmed disease progression per iRECIST guidelines (or RECIST Version 1.1 guidelines for EV Mono arm) as determined by the investigator, initiated a subsequent anticancer therapy, died, the study closed, or the patient withdrew consent, whichever occurred first. The determination of antitumor activity was based on confirmed objective response assessments as defined by RECIST Version 1.1 as determined by the investigator. The investigator made treatment decisions based on site assessments of scans by iRECIST guidelines (or RECIST Version 1.1 guidelines for EV Mono arm). Clinical response was determined at each assessment per RECIST Version 1.1 or per iRECIST guidelines when applicable. Clinical response of CR, PR, SD, or PD was determined at each assessment.

In addition, images (including via other modalities) that were obtained at an unscheduled time point should have also been submitted to the BICR.

Survival status and information on subsequent anticancer therapy were updated every 12 weeks (±7 days) until death, withdrawal of consent, or study closure, whichever occurred first.

Patients' clinical data must have been available for CRF source verification. Tumor images were submitted to an independent review facility for reading and analysis.

### 6.1.7.4 Pharmacokinetic and Immunogenicity Assessments

Blood samples for PK and ATA were collected throughout the study per the sample collection schedule provided in Table 14. Validated or qualified assays were used to measure the concentrations of enfortumab vedotin ADC, TAb, and MMAE in serum or plasma. PK samples were collected and archived for possible analysis of concomitant drug levels or other enfortumab vedotin-related species, such as circulating metabolites of MMAE. A validated assay was used to determine the levels of ATA for enfortumab vedotin in plasma. PK and ATA samples for pembrolizumab were collected and archived for potential subsequent analysis in the EV+Pembro arm. If enfortumab vedotin or pembrolizumab infusions were discontinued, the corresponding research samples no longer needed to be collected. If at some point prospective PK blood sample collection was no longer required, sites were notified.

### 6.1.7.5 Biomarker Studies

An additional mechanism of action of vedotin ADCs may involve induction of immunogenic cell death, and thus changes in the number and activation state of the tumor-associated and peripheral immune cells (Cao 2018). These changes may potentiate the activity of pembrolizumab.

Samples for exploratory biomarker analyses were collected at protocol-specified time points (see Table 15). Biomarker assessments were not be used for patient selection.

Methods of analysis may have included: immunohistochemistry (IHC), Next Generation Sequencing (NGS) of DNA and RNA, T cell receptor beta chain sequencing, PCR, flow cytometry, and immunoassays.

All patients should have provided tissue for biomarker analysis from an archival tissue sample or newly obtained core or excisional biopsy of a tumor lesion. A formalin-fixed paraffin embedded tumor tissue block was requested. Archival tumor tissue that was collected within 6 months of enrollment after receipt of the most recent treatment was required if available. Core needle and excisional biopsies from tumor tissue (non-bone sites) were preferred. A fresh baseline core needle biopsy of a tumor lesion may have been submitted, if feasible. In the event a biopsy is clinically required as part of SOC, tissue should have been made available for biomarker assessment.

Exploratory, predictive, and prognostic biomarkers associated with response, resistance, or safety observations were monitored before and during treatment with enfortumab vedotin. Biomarker assessments in tumor tissue may have included, but may not have been limited to, measurements of enfortumab vedotin and its metabolites, as well as characterization of the tumor microenvironment (TME) and drug effects. Assays may have included, but may not have been limited to, IHC and next generation sequencing of RNA and DNA.

TABLE 15
Pharmacokinetic, immunogenicity, and biomarker sample collection time points-for locally advanced or metastatic urothelial cancer

| | | | | | | | | | | | Archival | Fresh |
|---|---|---|---|---|---|---|---|---|---|---|---|---|
| | | | | | | | | | | | Tissue | Tissue |
| | | | | | | | | | | | Slides | Blocks^{F} |
| | | | | | Blood | | | | | | Nectin-4, | Nectin-4, |
| | | | | | | | ATA to | | | | TME, | TME, |
| | Study | | | Relative | Enfortumab | Pembro | Enfortumab | ATA to | | | GE | GE |
| | Day | Time | Window | Time | Vedotin PK | PK^{G} | Vedotin | Pembro^{G} | Cytokines | cfDNA | Profiling | Profiling |
| Screening | Days | N/A | N/A | N/A | | | | | | | X^{A} | X^{A} |
| | −28 to | | | | | | | | | | | |
| | 1 | | | | | | | | | | | |
| Cycles 1 | Day 1 | Predose | Within | Start of | X | X | X | X | X | X | | |
| and 2 | | | 24 h | enfortumab | | | | | | | | |
| | | | | vedotin | | | | | | | | |
| | | | | infusion | | | | | | | | |
| | | End of | Within | End of | X | | | | | | | |
| | | enfortumab | 15 min | enfortumab | | | | | | | | |
| | | vedotin IV | | vedotin | | | | | | | | |
| | | infusion | | infusion | | | | | | | | |
| | | End of | Within | End of | | X | | | | | | |
| | | pembro IV | 15 min | pembro | | | | | | | | |
| | | infusion | | infusion | | | | | | | | |
| | Day 8 | Predose | Within | Start of | X | | | | X^{I} | X^{I} | | |
| | | | 4 h | enfortumab | | | | | | | | |
| | | | | vedotin | | | | | | | | |
| | | | | infusion | | | | | | | | |
| | | End of | Within | End of | X | | | | | | | |
| | | enfortumab | 15 min | enfortumab | | | | | | | | |
| | | vedotin IV | | vedotin | | | | | | | | |
| | | infusion | | infusion | | | | | | | | |
| | Days | N/A | Prior to | End of | | | | | | | | X^{G} |
| | 15 to | | Cycle 2 | enfortumab | | | | | | | | |
| | 21 | | dosing | vedotin | | | | | | | | |
| | | | | infusion | | | | | | | | |
| All other | Day 1 | Predose | Within | Start of | X^{B} | X^{B} | X^{B} | X^{B} | X^{C} | X^{H} | | |
| cycles | | | 24 h | infusion | | | | | | | | |

| | | | | | | | | |
|---|---|---|---|---|---|---|---|---|
| End of Treatment | N/A | X | X | X | X | X | X | X^{D} |
| (within 30-37 days of last dose) | | | | | | | | |

ATA = antitherapeutic antibody; cfDNA = cell-free DNA; GE = gene expression; IV = Intravenous; N/A = not applicable; PBMC = peripheral blood mononuclear cells; PK = pharmacokinetics; TME = tumor microenvironment.
Note:
For subjects who permanently discontinued enfortumab vedotin or pembrolizumab, no PK samples were collected. If at some point prospective PK blood sample collection was no longer required, sites were notified.
^{A}Archival tumor tissue acquired within 6 months of enrollment and after receipt of any prior therapies OR a freshly obtained baseline tumor specimen was required prior to receiving study treatment, if feasible. See Section 6.1.7.6(ii).
^{B}Cycles 3 and 4 and every even-numbered cycle thereafter, up to Cycle 12.
^{C}Cycles 3 and 4 only.
^{D}Optional tumor tissue biopsy.
^{E}If nonprotocol mandated biopsies of other tissue were conducted as part of standard care, tissue may have been submitted for further biomarker analysis.
^{F}PK for pembrolizumab was collected for EV + Pembro arm only. ATA for pembrolizumab was collected for EV + Pembro arm only.
^{G}Cycle 1 fresh tissue biopsy collection was optional.
^{H}cfDNA collected on Day 1 of every cycle 3+.
^{I}Required for Cycle 1.

### (i) Biomarkers in Blood

Biomarker assessments in blood samples may have included, but may not have been limited to, markers of immune function, including abundance and phenotype of immune cell subsets, T-cell clonality analyses, abundance of cytokines (e.g., inflammation markers or hyperglycemia associated markers), and circulating tumor DNA as a marker of tumor response or therapy resistance. These assessments were compared to similar assessments conducted in enfortumab vedotin monotherapy trials, and may have provided insight into treatment related changes in activation state of peripheral immune system associated with enfortumab vedotin-induced tumor cell death and immune checkpoint blockade.

### (ii) Biomarkers in Tumor Tissue

To understand possible markers for response, resistance and treatment-induced changes in the tumor cells and TME archival tumor sections, tumor sections after the last systemic therapy or a fresh biopsy, and an optional fresh on-treatment biopsies were requested. If nonprotocol-mandated biopsies were conducted as part of standard care, optional submission of tissue samples was requested.

Pre-treatment fresh tumor biopsy tissue was required and should have been acquired within ≤6 months prior to receiving study treatment, but after completion of the most recent prior systemic therapy. Alternatively, approximately 20 slides of a <6-month-old biopsy or resection were provided, if available. Comparison of archival tumor biomarkers (see below) with those of freshly obtained, pre-treatment tumor tissue enabled an assessment of the durability of these biomarkers.

Combination of enfortumab vedotin and CPI may be expected to both increase the early innate as well as the subsequent adaptive antitumor immune response in the tumor compared to CPI alone. To characterize and correlate treatment-associated changes in the tumor immune microenvironment with patient outcome, an on-treatment biopsy was required between Days 15 to 21 of Cycle 1 for patients with accessible tumors (in the opinion of the investigator). The on-treatment biopsy was performed prior to Cycle 2 Day 1 dosing, and if possible, fresh biopsies should have been obtained from the same lesion as the pre-treatment biopsy.

Biomarker assessments in archival and freshly obtained tumor tissue may have included, but were not limited to:
Tumor expression of Nectin-4 and PD-L1 proteins
Exploratory analysis of GE (e.g., Nectin-4, PD-1 and PD-L1, drug transporters such as ABCB1/P-gp, signatures of diverse immune subpopulations) and markers of disease subtype (eg, TCGA subtypes)
Mutational load and disease relevant genomic changes (e.g., fibroblast growth factor receptor aberrations)
Protein markers of the tumor and tumor immune microenvironment (e.g., CD68, CD8, FOXP3, CD163)

### 6.1.7.6 Biospecimen Repository

For patients in the US who provided additional consent, remaining de-identified unused blood and/or tissue was retained by the sponsors and used for future research, including but not limited to the evaluation of targets for novel therapeutic agents, the biology of ADC sensitivity and resistance mechanisms, and the identification of biomarkers of ADCs. Blood, urine, and tissue samples donated for future research are retained for a period of up to 25 years. If additional consent was not provided, any remaining biological samples were destroyed following study completion.

### 6.1.7.7 Patient Reported Outcomes (PRO)

Patient Reported Outcome (PRO) assessments included the EQ 5D-5L, EORTC QLQ-C30, BPI-SF, and HRU assessments (see Section 6.1.7.8(i) to 6.1.7.8(iii) for details). PRO assessments are completed on Day 1 prior to dosing, once weekly for the first 3 cycles (within 2 days prior to dosing), and once every cycle (within 2 days prior to dosing) for the remainder of the treatment portion of the study. On dosing visit days, assessments should have been completed prior to dosing. PRO assessments were administered at each follow-up visit and each long term follow-up contact (see Table 16).

TABLE 16
Schedule of events

| | | | | | | | | | | | | | | | Long- | |
|---|---|---|---|---|---|---|---|---|---|---|---|---|---|---|---|---|
| | | | | | | | | | | | | | | | term | Survival |
| | | Screening/ | | Enrollment/ | | | | | | | | | | Follow- | Follow- | Follow- |
| | | Baseline | | Randomization | Every 3-Week Cycle | | | | | | | | EOT | up | up^{CC} | up^{CC} |
| | | Day | | | | | | | | | | | | | | |
| | | | | Within 1 to | | D2 | | | | | | | Within 30 | | | |
| | | D −28 | D −7 | 2 d of First | | to | | | | | | | to 37 d of | Every 9 | Every 12 | Every 12 |
| | | to 1 | to 1 | Dose | D1 | D4^{A} | D8 | D9^{A} | D10^{A} | D11^{A} | D15 | D15 to 21 | Last Dose^{B} | Weeks | Weeks | Weeks |
| | | Visit Window | | | | | | | | | | | | | | |
| | | | | | ±2 d2 | | ±1 d | | | | ±1 d | | | ±7 d | ±7 d | ±7 d |
| Screening/ | Inclusion/Exclusion, | X | | Submit | | | | | | | | | | | | |
| Baseline | medical history | | | confirmation | | | | | | | | | | | | |
| Assessments | Informed consent | X | | prior to | | | | | | | | | | | | |
| | Acquire and submit | X^{C} | | treatment | | | | | | | | X^{D} | X^{E} | | | |
| | fresh tumor biopsy tissue | | | | | | | | | | | | | | | |
| | Initiate collection of | X | | | | | | | | | | | | | | |
| | archived tumor specimen^{F} | | | | | | | | | | | | | | | |
| | Complete eye exam^{G} | X | | | | | | | | | | | X | | | |
| | Brain scan^{G} | X | | | | | | | | | | X^{W} | X^{W} | X^{W} | | |
| | Bone scan^{G} | X | | | | | | | | | | X^{W} | X^{W} | X^{W} | | |
| | INR/PT/PTT | | X | | | | | | | | | | | | | |
| | Serology for hepatitis B | X | | | | | | | | | | | | | | |
| | surface antigen and | | | | | | | | | | | | | | | |
| | antihepatitis B | | | | | | | | | | | | | | | |
| | core antibody | | | | | | | | | | | | | | | |
| | Serology for | X | | | | | | | | | | | | | | |
| | antihepatitis C | | | | | | | | | | | | | | | |
| | antibody | | | | | | | | | | | | | | | |
| | HbA1c^{S} | X | | | | | | | | | | | | | | |
| | Urinalysis with reflexive | X | | | | | | | | | | | | | | |
| | microscopic analysis | | | | | | | | | | | | | | | |
| | Pregnancy test | | X^{V} | | X | | | | | | | | X | | | |
| | (urine or serum; | | | | | | | | | | | | | | | |
| | only for females | | | | | | | | | | | | | | | |
| | of childbearing | | | | | | | | | | | | | | | |
| | potential)^{FF} | | | | | | | | | | | | | | | |
| Safety | Physical exam | | X | | X^{H,Y} | | | | | | | | X | X^{I} | | |
| Assessments | (including weight) | | | | | | | | | | | | | | | |
| | Height | | X | | | | | | | | | | | | | |
| | Vital signs | | X | | X^{H,Y} | | X^{Y} | | | | | | X | | | |
| | CBC with differential^{J,T} | | X | | X^{H,Y} | | X^{Y} | | | | X | | X | | | |
| | Serum chemistry panel^{J,T} | | X | | X^{H,X,Y} | | X^{X,Y} | | | | X | | X | | | |
| | CrCl | | X | | | | | | | | | | | | | |
| | Thyroid function tests | X | | | X^{H,Y} | | | | | | | | | | | |
| | ECOG performance | | X | | X^{H,Y} | | | | | | | | | X^{I} | | |
| | status | | X | | | | | | | | | | | | | |
| | ECG^{K} | | X | | | | | | | | | | X | | | |
| | Transthoracic ECHO^{T,EE} | | X | | | | | | | | | | | | | |
| | Slit lamp examination | | | | | | | | | | | | X^{R} | | | |
| | Concomitant medications | Related to study | | | Collect from Day 1 (predose) through 30 days (AEs) or 90 days (SAEs related | | | | | | | | | | | |
| | Adverse event collection | procedures^{L} | | | to pembrolizumab) post last dose or through the EOT visit, whichever is later. | | | | | | | | | | | |
| | | | | | For AECIs, AEs and concomitant medications may be followed until AE | | | | | | | | | | | |
| | | | | | resolution, return to baseline, or withdrawal from study, whichever is later. | | | | | | | | | | | |
| Patient | Quality of Life | | | | X^{BB} | | X | | | | X | | X | X | X | X |
| Reported | (QOL), Pain, and | | | | | | | | | | | | | | | |
| Outcome | HRUAssessment^{BB} | | | | | | | | | | | | | | | |
| Assessments | | | | | | | | | | | | | | | | |
| Treatment^{DD} | Enfortumab vedotin | | | | X | | X | | | | | | | | | |
| | administration^{U} | | | | | | | | | | | | | | | |
| | Pembrolizumab | | | | X^{M} | | | | | | | | | | | |
| PK/ | Blood, tissue sample | See PK, ATA, and biomarker tables for sample collection details^{A} | | | | | | | | | | | | | | |
| Immunogenicity/ | collection | | | | | | | | | | | | | | | |
| Biomarker | | | | | | | | | | | | | | | | |
| Response | Imaging^{N} | X | | | | | | | | | | X^{O} | X^{P} | x^{I,O} | | |
| Assessment | Survival status | | | | | | | | | | | | | | X^{Q} | X^{Q} |
| Randomization^{AA} | | | | X^{AA} | | | | | | | | | | | | |

^{A}See Table 15 for PK, ATA, and biomarker sampling.
^{B}EOT evaluations should have been obtained before the initiation of nonprotocol therapy. If EOT evaluations were completed before 30 days following the last study treatment, a phone screen was conducted 30-37 days following the patient's last study treatment to ensure that no changes in AE profile have occurred.
^{C}Pre-treatment fresh tumor biopsy tissue should have been acquired within ≤6 months prior to receiving study treatment, but after completion of the most recent prior systemic therapy.
^{D}For patients with accessible tumors (in the opinion of the investigator), required on-treatment biopsy were collected between Days 15 to 21 (must be prior to Cycle 2 Day 1 dosing) during Cycle 1 only. If possible, fresh biopsies should have been obtained from the same lesion as the pre-treatment biopsy.
^{E}Optional biopsy was collected at EOT. If possible, fresh biopsies should have been obtained from the same lesion as the pre-treatment biopsy.
^{F}Invasive tumor tissue (from primary or metastatic site) obtained within 6 months of enrollment and after receipt of any prior therapies OR a freshly obtained baseline tumor specimen. Samples must have been available for participation in the study. Tumor block was preferred. If tumor block was not available, slides were acceptable. Not required for retreatment.
^{G}Brain scans and bone scans were required at screening. Repeated as clinically indicated throughout the study.
^{H}Not required for Cycle 1 if baseline assessments performed within 1 day.
^{I}Patients who discontinued study treatment for reasons other than radiographically confirmed disease progression by iRECIST guidelines (Seymour 2017) (Section 6.1.15) had physical exams, ECOG, and disease assessments every 9 weeks (±1-week) according to the original schedule calculated from Cycle 1 Day 1 up to 1 year after first dose, then every 12 weeks (±1 week). Scans were to be done until the patient had radiologically confirmed disease progression per RECIST Version 1.1 (EV Mono arm) or per iRECIST (EV + Pembro arrn) on or following study therapy as determined by the investigator, initiated a subsequent anticancer therapy, died, the study closed, or the patient withdrew consent, whichever occurred first. During study treatment, palliative radiotherapy on a nontarget lesion that was not progressing was not considered a subsequent anticancer therapy; however, radiotherapy on any target lesion was a subsequent anticancer therapy. The investigator made treatment decisions based on site assessments of scans per RECIST Version 1.1 (EV Mono arm) or iRECIST (EV + Pembro arm).
^{J}Abnormal lab values that were clinically significant are confirmed within 1 day during Cycle 1, as clinically indicated.
^{K}Standard Baseline/EOT ECG assessments were performed for all patients.
^{L}From time of informed consent.
^{M}In EV + Pembro arm, pembrolizumab should have been administered approximately 30 minutes after completion of enfortumab vedotin administration (Day 1).
^{N}Computed tomography (CT) (with IV contrast) of the chest, abdomen, and pelvis. If contrast media were contraindicated, please refer to the current Image Acquisition Guidelines Section 6.1.13) for preferred scanning and contrast options. Other regions should have been scanned if the patient had known disease or new symptoms suggestive of disease in that region. Response assessment was evaluated using RECIST Version 1.1 (Eisenhauer 2009) (Table 40) and iRECIST guidelines when applicable. Primary analysis was based on RECIST Version 1.1. For patients in the EV + Pembro arm, secondary analysis of response was based on iRECIST guidelines.
^{O}Response assessment was performed every 9 weeks (±1-week) from Cycle 1 Day 1. Objective responses were confirmed per RECIST Version 1.1 with repeat scans at least 4 weeks after first documentation of response (scans required at 4 to 5 weeks). Tumor imaging should also have been performed whenever disease progression was suspected. Patients in the EV + Pembro arm with unconfirmed progressive disease (iUPD) on therapy per iRECIST guidelines (Seymour 2017) (Section 6.1.15) may have continued on study treatment until progression was confirmed by the investigator (confirmed progressive disease [iCPD]) with a subsequent response scan 4 to 9 weeks after iUPD. Subsequent scans were to be performed every 9 weeks (±1-week) according to the original schedule until 1 year after first dose, then every 12 weeks (±1-week).
^{P}Not required if conducted <4 weeks prior to EOT.
^{Q}Contacted patient for survival status, clinical progression status, and collection of subsequent anticancer treatment information every 12 weeks (±1-week) after radiologically confirmed progressive disease per RECIST Version 1.1 guidelines until death, study closure, or patient withdrew consent, whichever occurred first.
^{R}Only required for patients who experienced corneal AEs during the study; must have been performed >4 weeks after last dose.
^{S}If HbAlc was elevated (≥6.5%), referred patient to appropriate provider during Cycle 1 for glucose management.
^{T}Safety labs may have been collected up to 1 day prior to dosing.
^{U}At least 1 week must have elapsed between doses of enfortumab vedotin.
^{V}Either serum or urine pregnancy test. Baseline assessment Day −3 to Day 1.
^{W}Brain scans should have been repeated at this time point if inactive brain metastases were known at baseline, or as clinically indicated throughout the study. Bone scans should have been repeated at this time point if bone metastases were identified at baseline, or if bone metastasis was known or suspected, or as clinically indicated throughout the study.
^{X}Verify blood glucose is <250 mg/dL prior to dosing. Patients with diabetes must have been tested in the clinic and blood glucose must have been <250 mg/dL prior to dosing. Use of insulin was permitted as part of the standard of care.
^{Y}Procedures should have been conducted pre-dose. Patients who discontinued treatment with enfortumab vedotin and continued treatment with pembrolizumab should have returned to the clinic for Day 8 and Day 15 visits (for safety labs). Day 8 vital signs were not collected for patients who discontinued treatment with enfortumab vedotin permanently.
^{Z}Window applied to Cycle 2+
^{AA}Randominzation to have occurred within 1 to 2 business days of planned first dose.
^{BB}Patient reported outcome (PRO) assessments include QOL measures, both general and oncology specific (EQ 5D-5L and EORTC QLQ-C30, respectively), as well as outcome specific PROs around pain (BPI-SF), and health resource utilization (HRU) assessments. Patient reported outcomes were administered at baseline on Day 1 prior to dosing, once weekly for the first 3 cycles (within 2 days prior to dosing), and once every cycle (within 2 days prior to dosing) for the remainder of the treatment portion of the study, once at each follow-up visit and at each long-term follow-up contact. If a visit occurs out of the assessment window, assessments should still have been completed. Visit window was −2 days for C1D8 through C3D15, and −1 day for C4+.
CCAfter initiation of subsequent anticancer therapy or radiologically confirmed disease progression on or following study therapy (by investigator assessment), patients were followed via imaging per institutional guidelines, but not less frequently than every 12 weeks until PFS2 was documented or the patient started another anticancer treatment, whichever occurred first. All subsequent anticancer therapy including date of progression for PFS2 was recorded on the case report form. In addition, patients were contacted every 12 weeks after EOT for survival status and clinical progression status on or following subsequent therapy. Following PFS2, patients entered the survival follow-up period and were followed every 12 weeks (±1-week) for survival status until death, study closure, or withdrawal of consent, whichever occurred first.
^{DD}The results of all local laboratory tests were reviewed prior to study drug administration in order to determine whether to proceed with dosing or whether dose modification was required.
^{EE}A transthoracic echocardiogram was required for subjects with NYHA Class III heart failure or a history of coronary heart disease, arrhythmia or other significant heart disease
^{FF}Either serum or urine pregnancy test. Not required for Cycle 1 if baseline assessment was performed within 7 days. Repeat every month (±1 week) for 6 months after EOT. The pregnancy test may be performed within 1 day prior to administration of study drug.

Assessments were completed by the patient at home on an electronic device (preferred medium for reporting) that was given to each patient at the first clinic visit. The device has preset reminders for the patient to fill out their assessments at prespecified timepoints. Sites were required to monitor the patients' compliance with the ePRO devices through an online portal. Assessments may have been reported on paper or by clinical staff by telephone (using approved scripts that are made available to sites) only if the use of an electronic device was not feasible. When using paper or interview methods, patients were provided with instructions for filling in the assessments at clinic visits, prior to dosing, and at prespecified timepoints. The investigator or site designee should have reviewed the assessment data for correct completion when collected. The investigator or site designee, and site personnel were trained on how to provide support to each patient on the use of the PRO device (or paper assessment only if the use of the electronic device is not feasible).

### (i) EORTC Core Quality of Life (QLQ-C-30) Assessment

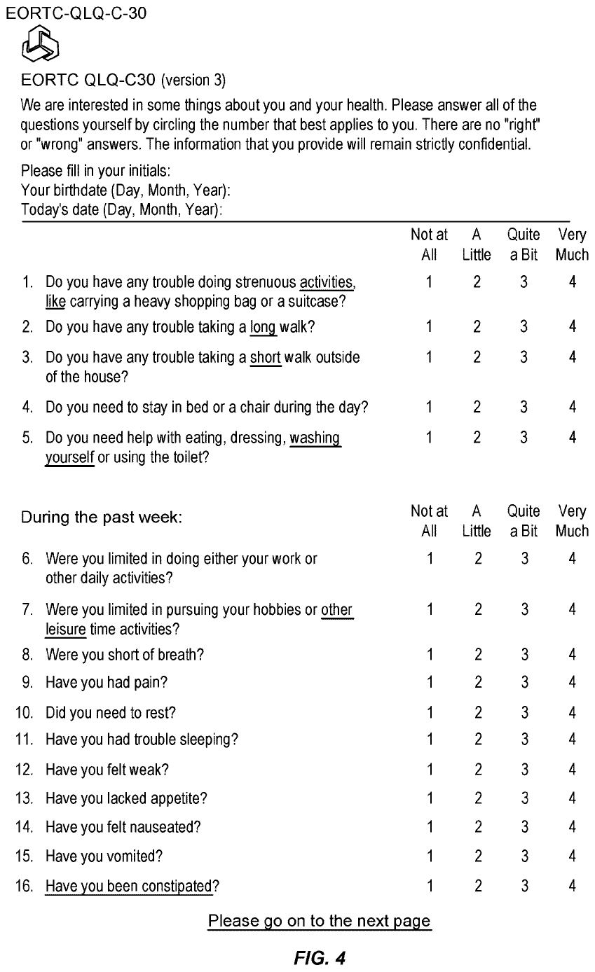

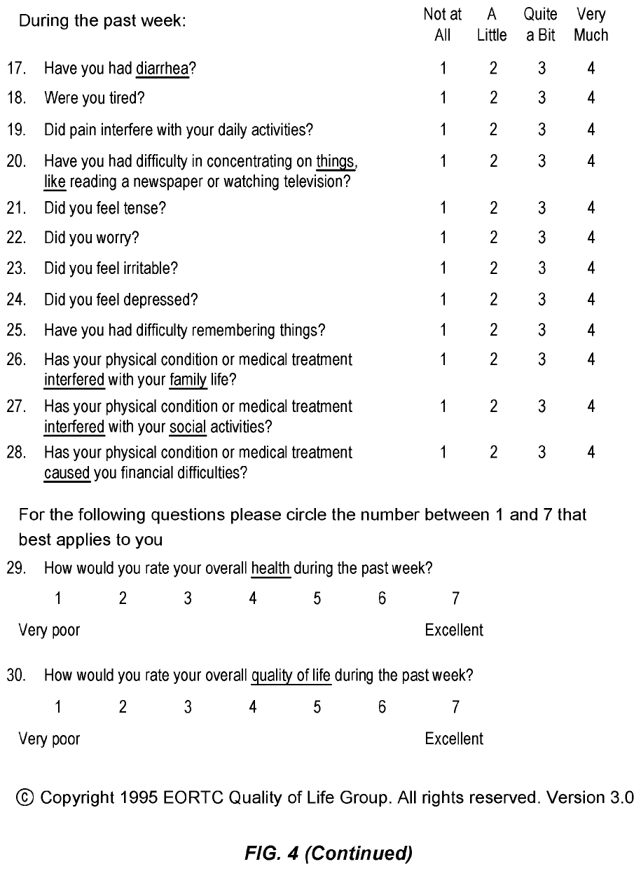

The EORTC Core Quality of Life (QLQ-C30) was developed to measure aspects of QoL pertinent to patients with a broad range of cancers who are participating in clinical trials (Aaronson 1993); (Sneeuw 1998). The current version of the core instrument (QLQ-C30, Version 3) is depicted in FIG. 4 and is a 30-item assessment consisting of the following:
5 functional domains (physical, role, cognitive, emotional, and social);
3 symptoms scales (fatigue, pain, nausea and vomiting);
Single items for symptoms (shortness of breath, loss of appetite, sleep disturbance, constipation, diarrhea) and financial impact of the disease, and;
2 global items (health, overall QoL).

Each domain was scored from 0 to 100. For the global health status/QoL and functional domain scores, higher scores represent better QoL and functioning, respectively. For symptom scales, higher scores represent greater symptomatology (see FIG. 4).

### (ii) EuroQol-5 Dimensions

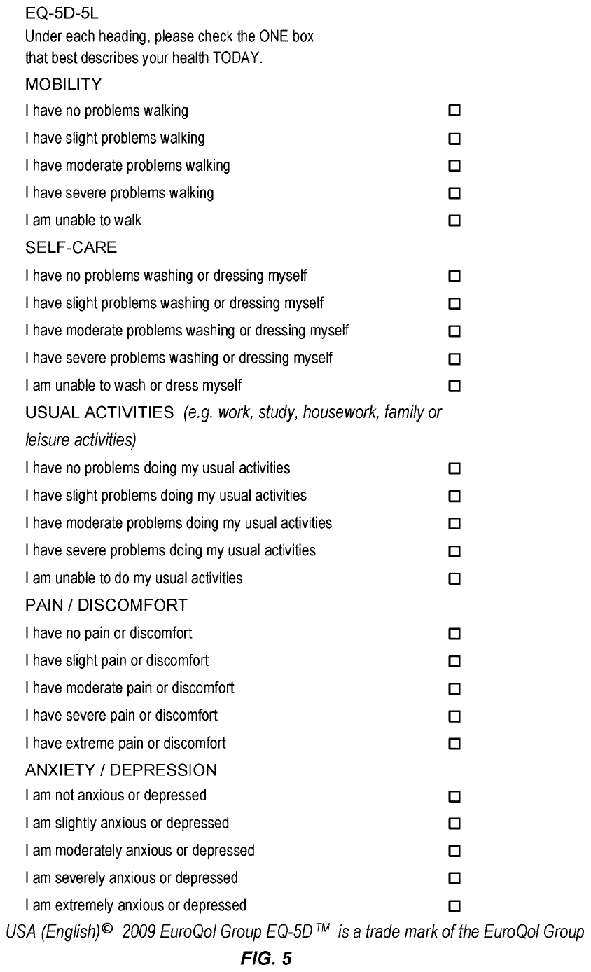

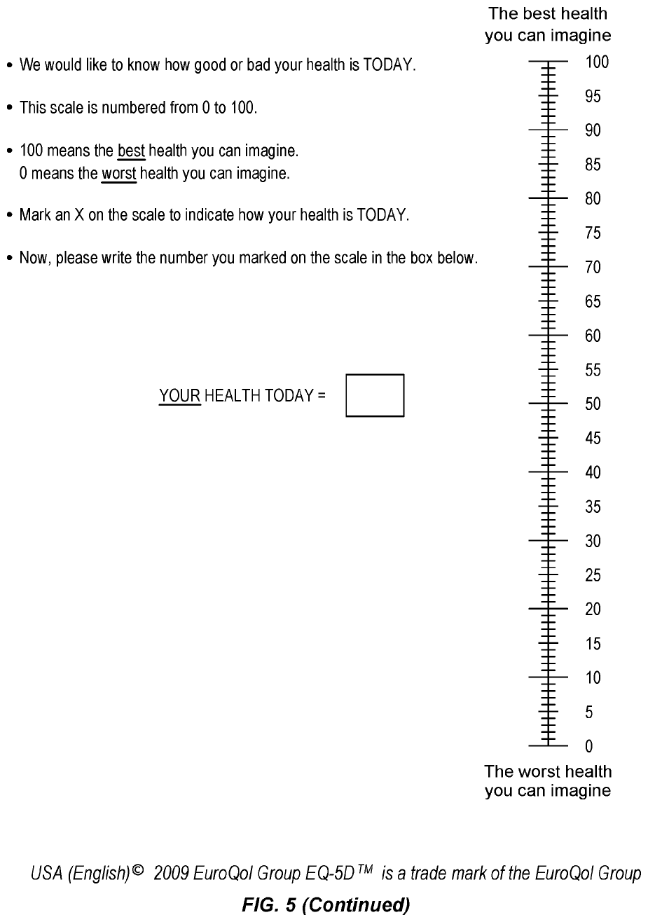

The EQ-5D is a standardized instrument developed by the EuroQol Group for use as a generic, preference-based measure of health outcomes. It is applicable to a wide range of health conditions and treatments and provides a simple descriptive profile and a single index value for health status. The EQ-5D is a 5-item self-reported measure of functioning and well being, which assesses 5 dimensions of health, including mobility, self-care, usual activities, pain/discomfort, and anxiety/depression (see FIG. 5). Each dimension comprises 3 levels (no problems, some/moderate problems, extreme problems). A unique EQ-5D health state is defined by combining 1 level from each of the 5 dimensions. Responses to the 5 items are then converted to a weighted health state index (utility score) based on values derived from general population samples (Herdman 2011). The health utility score is between 0 and 1, where 0 is death and 1 is perfect health. In addition to the utility score, this assessment also records the respondent's self-rated health status on a vertical graduated (0 to 100) visual analogue scale (see FIG. 5).

### 6.1.7.8 Safety Assessments

The assessment of safety during the course of this study consisted of the surveillance and recording of AEs including SAEs, recording of concomitant medication, and measurements of protocol-specified physical examination findings, cardiac monitoring, and laboratory tests. Safety was monitored by a restricted SMC on an ongoing basis as described in Sections 6.1.3 and 6.1.8.3.

All unscheduled laboratory assessments associated with the treatment of bladder cancer disease and AEs under study should have been captured in an unscheduled visit and laboratory reports sent to a central laboratory vendor.

### (i) Adverse Events

### (a) About Adverse Event

### Adverse Event

According to the International Council for Harmonisation (ICH) E2A guideline Definitions and Standards for Expedited Reporting, and 21 Code of Federal Regulations (CFR) 312.32, Investigational New Drug (IND) Safety Reporting, an AE is any untoward medical occurrence in a patient or clinical investigational subject administered a medicinal product and which does not necessarily have a causal relationship with this treatment.

The following information should have been considered when determining whether or not to record a test result, medical condition, or other incident on the AEs and Pre-existing Conditions CRF:
From the time of informed consent through the day prior to study Day 1, only study protocol related AEs should be recorded. A protocol-related AE is defined as an untoward medical event occurring as a result of a protocol mandated procedure.
All medical conditions present or ongoing predose on study Day 1 should be recorded.
All AEs (regardless of relationship to study drug) should be recorded from study Day 1 (predose) through the end of the safety reporting period. Complications that occur in association with any procedure (eg, biopsy) should be recorded as AEs whether or not the procedure was protocol mandated.
Changes in medical conditions and AEs, including changes in severity, frequency, or character, during the safety reporting period should be recorded.
In general, an abnormal laboratory value should not be recorded as an AE unless it is associated with clinical signs or symptoms, requires an intervention, results in a SAE, or results in study termination or interruption/discontinuation of study treatment. When recording an AE resulting from a laboratory abnormality, the resulting medical condition rather than the abnormality itself should be recorded (e.g., record “anemia” rather than “low hemoglobin”).

### Serious Adverse Events

An AE should have been classified as an SAE if it met one of the following criteria:
Fatal: AE resulted in death
Life threatening: The AEs placed the patient at immediate risk of death. This classification does not apply to an AE that hypothetically might cause death if it were more severe
Hospitalization: The AE resulted in hospitalization or prolonged an existing inpatient hospitalization. Hospitalizations for elective medical or surgical procedures or treatments planned before the signing of informed consent in the study or routine check-ups are not SAEs by this criterion. Admission to a palliative unit or hospice care facility is not considered to be a hospitalization. Pre-planned hospitalizations for therapeutic, diagnostic, or surgical procedures of the underlying cancer or study target disease that did not worsen during the clinical trial need not be captured as SAEs.
Disabling/incapacitating: An AE that resulted in a persistent or significant incapacity or substantial disruption of the patient's ability to conduct normal life functions.
Congenital anomaly or birth defect: An adverse outcome in a child or fetus of a patient exposed to the molecule or study treatment regimen before conception or during pregnancy.
Medically significant: The AE did not meet any of the above criteria, but could have jeopardized the patient and might have required medical or surgical intervention to prevent one of the outcomes listed above or involves suspected transmission via a medicinal product of an infectious agent.

### Adverse Event Severity

AE severity should have been graded using the National Cancer Institute's Common Terminology Criteria for Adverse Events (NCI CTCAE), Version 4.03.

AE severity and seriousness were assessed independently. ‘Severity’ characterizes the intensity of an AE. ‘Serious’ is a regulatory definition and serves as a guide to the sponsor for defining regulatory reporting obligations (see definition for SAEs, above).

### Relationship of the Adverse Event to Study Treatment

The relationship of each AE to enfortumab vedotin and/or pembrolizumab and/or carboplatin/cisplatin/gemcitabine should have been evaluated by the investigator using the following criteria:
Related: There is evidence to suggest a causal relationship between the drug and the AE, such as:
   A single occurrence of an event that is uncommon and known to be strongly associated with drug exposure (eg, angioedema, hepatic injury, Stevens-Johnson Syndrome)
   One or more occurrences of an event that is not commonly associated with drug exposure, but is otherwise uncommon in the population exposed to the drug (eg, tendon rupture)
Unrelated: Another cause of the AE is more plausible (e.g., due to underlying disease or occurs commonly in the study population), or a temporal sequence cannot be established with the onset of the AE and administration of the study treatment, or a causal relationship is considered biologically implausible.

### (b) Procedures for Eliciting and Recording Adverse Events

Investigator and study personnel reported all AEs and SAEs whether elicited during patient questioning, discovered during physical examination, laboratory testing and/or other means by recording them on the CRF and/or SAE form, as appropriate.

### Eliciting Adverse Events

An open-ended or non-directed method of questioning should have been used at each study visit to elicit the reporting of AEs.

### Recording Adverse Events

The following information should have been recorded on the AEs and Pre-existing Conditions CRF:
Description including onset and resolution dates
Whether it met SAE criteria
Severity
Relationship to study treatment or other causality
Outcome

### Diagnosis Vs Signs or Symptoms

In general, the use of a unifying diagnosis was preferred to the listing out of individual symptoms. Grouping of symptoms into a diagnosis should only have been done if each component sign and/or symptom was a medically confirmed component of a diagnosis as evidenced by standard medical textbooks. If any aspect of a sign or symptom did not fit into a classic pattern of the diagnosis, the individual symptom was reported as a separate AE.

Important exceptions for this study are adverse reactions associated with the infusion of study drug. For IRRs, record the NCI CTCAE term of ‘infusion related reaction’ with an overall level of severity (per NCI CTCAE). In addition, each sign or symptom of the reaction was recorded as an individual AE. If multiple signs or symptoms occurred with a given infusion related event, each sign or symptom should have been recorded separately with its level of severity.

### Recording Serious Adverse Events

For SAEs, the event(s) was recorded on both the CRF and an SAE form.

The following should be considered when recording SAEs:
Death is an outcome of an event. The event that resulted in the death should have been recorded and reported on both an SAE form and CRF.
For hospitalizations, surgical, or diagnostic procedures, the illness leading to the surgical or diagnostic procedure should have been recorded as the SAE, not the procedure itself. The procedure should have been captured in the narrative as part of the action taken in response to the illness.

### Progression of the Underlying Cancer

Since progression of the underlying malignancy is being assessed as an efficacy variable, it should not have been reported as an AE or SAE. Radiographic signs of disease progression (e.g., ‘tumor progression’ or ‘metastases’) should not have been reported as AEs or SAEs (these data are captured in the efficacy assessment). The clinical symptoms and signs of disease progression (e.g., ‘fatigue’, ‘dyspnea’) may have been reported as AEs or SAEs if the symptom cannot have been determined as being exclusively due to progression of the underlying malignancy or did not fit the expected pattern of progression for the disease under study; disease progression was not reported as the AE term. In addition, complications from progression of the underlying malignancy should have been reported as AEs or SAEs.

Death is an outcome of an event. The event that resulted in the death should have been recorded and reported on both an SAE form and CRF.

### Pregnancy

Notification to Drug Safety: A Pregnancy Report Form was completed for all pregnancies that occurred from the time of first study drug dose until 6 months after the last dose of study drug(s) including any pregnancies that occurred in the partner of a male study patient. Pregnancies that occurred in a male patient's partner were reported if the estimated date of conception was after the male patient's first study drug dose. Email or fax was sent to the Drug Safety Department within 48 hours of becoming aware of a pregnancy (see e-mail or fax number specified on the SAE report form). All pregnancies were monitored for the full duration; all perinatal and neonatal outcomes should have been reported. Infants should have been followed for a minimum of 8 weeks.

Collection of data on the CRF: All pregnancies (as described above) that occurred within 30 days of the last dose of study drug(s) were also recorded on the AEs and Pre Existing Conditions CRF.

Abortion, whether accidental, therapeutic, or spontaneous, should have been reported as an SAE. Congenital anomalies or birth defects, as defined by the ‘serious’ criterion above should have been reported as SAEs.

### Corneal Adverse Events

Corneal ulcer or keratitis AEs ≥Grade 2 should have been graded within their respective NCI CTCAE categories. Grade 1 corneal ulcer or keratitis AEs should have been graded per “Eye disorders—Other, specify” criteria. Other corneal AEs should have been recorded and graded per “Eye disorders—Other, specify” criteria.

### Diabetes and Hyperglycemia

Grading for diabetes should have been based on the NCI CTCAE v4.03 event term of glucose intolerance. Grading for hyperglycemia should have been based on the NCI CTCAE v4.03 event term of hyperglycemia.

### Adverse Events of Possible Hepatic Origin

If an AE was accompanied by increases in LFT values (e.g., AST, ALT, bilirubin, etc.) or was suspected to be due to hepatic dysfunction, see Section 6.1.12 for detailed information on recommended monitoring and assessment of liver abnormalities. See 6.1.6.2(iii)(a) for treatment discontinuation recommendations related to hepatic safety.

Patients with AEs of hepatic origin accompanied by LFT abnormalities should have been carefully monitored.

### (c) Reporting Periods for Adverse Events and Serious Adverse Events

SAEs were followed until significant changes returned to baseline, the event stabilized (recovering/resolving) or was no longer considered clinically significant by the investigator, or the patient died or withdrew consent. All non-SAEs were followed through the safety reporting period. The investigator made every attempt to follow all patients with non serious AE for outcome. Certain non-serious AESIs may have been followed until resolution, return to baseline, or study closure, whichever is later. The reporting timeframe for AE meeting any serious criteria is described in Section (d) below (“Serious Adverse Events Require Immediate Reporting”).

The safety reporting period for all AEs and SAEs (except SAEs related to pembrolizumab) was from study Day 1 (predose) through the EOT visit or 30 days after the last study treatment, whichever was later. The reporting period for SAEs related to pembrolizumab was from study Day 1 (predose) through the EOT visit or 90 days after the last study treatment, whichever was later, or 30 days following cessation of study treatment if the participant initiated new anticancer therapy. All study protocol-related AEs were recorded from the time of informed consent. All SAEs that occurred after the safety reporting period and were considered study treatment-related in the opinion of the investigator should also have been reported to the sponsor. The investigator made every attempt to follow all patients with non serious AE for outcome.

### (d) Serious Adverse Events Require Immediate Reporting

Within 24 hours of observing or learning of an SAE, investigators were to report the event to the sponsor, regardless of the relationship of the event to the study treatment regimen.

For initial SAE reports, available case details were recorded on an SAE form. At a minimum, the following should have been included:
Patient number
Date of event onset
Description of the event
Investigator's causality assessment
Study treatment, if known

The completed SAE form and SAE Fax Cover Sheet were emailed or faxed to the sponsor's Drug Safety Department within 24 hours.

Relevant follow-up information was submitted to the sponsor as soon as it becomes available.

### (e) Adverse Events of Special Interest

Selected AEs were also known as AESIs and must have been reported to the sponsor.

Enfortumab vedotin

Certain non-serious AESIs may have been followed (including collection of concomitant medications) until resolution, return to baseline, or patient withdrawal from study, whichever is later.

AESIs related to enfortumab vedotin or enfortumab vedotin in combination with pembrolizumab for this purpose included the events listed below (*denotes that the event must be ≥Grade 2 or any grade resulting in a dose modification to be considered an AESI):
Rash
Peripheral neuropathy
Corneal events
Hyperglycemia
IRR
imAEs
   neumonitis*
   diarrhea* or colitis*
   epatitis or increased AST/ALT or total bilirubin*
   ndocrinopathy* (defined as hypophysitis, adrenal insufficiency, hypothyroidism, hyperthyroidism)
   nephritis or acute renal failure
   myocarditis
   pancreatitis
   myasthenia gravis or syndrome
   vasculitis
   cholangitis sclerosing
   Investigator-confirmed imAEs consistent with an immune-mediated mechanism of action that require treatment with systemic corticosteroids, other immunosuppressive, or endocrine therapy, and had no clear alternate etiology.

### (f) Adverse Events of Special Interest Requiring Immediate Reporting

For the time period beginning when the consent form is signed until treatment allocation, any AESI listed below, or follow-up to an AESI listed below, which occurs in any patient, must have been reported within 24 hours to the sponsor if it caused the patient to be excluded from the trial, or was the result of a protocol-specified intervention, including but not limited to washout or discontinuation of usual therapy, diet, or a procedure.

For the time period beginning at Cycle 1, Day 1 through 30 days following the last study drug dose, any AESI listed below, or follow-up to an AESI, whether or not related to the sponsor's product, must have been reported within 24 hours to the sponsor, either by electronic media or by paper. Electronic reporting procedures can be found in the EDC data entry guidelines.

AESIs for this purpose include:
An overdose of sponsor's product (for enfortumab vedotin: as defined such that in the event of an overdose >10% of enfortumab vedotin, the site should have notified the sponsor as soon as they were aware of the overdose. The patient should have been closely monitored for adverse reactions. Supportive care per institutional standards should have been administered (enfortumab vedotin) and for pembrolizumab: as defined such that an overdose of pembrolizumab is defined as ≥1000 mg (5 times the dose) of pembrolizumab. No specific information is available on the treatment of overdose of pembrolizumab. In the event of an overdose, the site should have notified the sponsor as soon as they were aware of the overdose. The patient should have been observed closely for signs of toxicity. Appropriate supportive treatment should have been provided if clinically indicated (pembrolizumab)) that was not associated with clinical symptoms or abnormal laboratory results.
Increased LFTs not otherwise explained by an underlying liver condition as defined in Section 6.1.6.2(iii)(a). Additional recommendations for liver safety monitoring and assessment may be found in Section 6.1.12.

### (f) Vital Signs

Vital sign measurements were performed to include heart rate (bpm), diastolic and systolic blood pressure (mmHg), and temperature. Vital sign values were recorded, and any diagnosis associated with clinically significant abnormal vital signs were recorded as an AE or pre-existing condition.

### (ii) Clinical Laboratory Tests

Samples were drawn for local labs.

Local laboratory testing included institutional standard tests for study eligibility, evaluating safety, and making clinical decisions. All local laboratory results must have been reviewed prior to study drug administration in order to determine whether to proceed with dosing or whether dose modification was required.

The following laboratory assessments were performed by the local lab to evaluate safety at scheduled time points (see Table 17) during the course of the study:
The serum chemistry panel is to include the following tests: albumin, alkaline phosphatase, ALT, AST, bicarbonate, blood urea nitrogen, calcium, creatinine, chloride, glucose, lactate dehydrogenase, phosphorus, potassium, sodium, total bilirubin, amylase, lipase, uric acid, and GFR.
   Verify blood glucose is <250 mg/dL prior to dosing (see Section 6.1.6.4(iv)). Patients with diabetes must be tested in the clinic and blood glucose must be <250 mg/dL prior to dosing. Use of insulin is permitted as part of the SOC.
The CBC with differential is to include the following tests: white blood cell count with five part differential (neutrophils, lymphocytes, monocytes, eosinophils, and basophils), platelet count, hemoglobin, and hematocrit.
CrCl at baseline as determined per institutional standards.
Thyroid function tests, including:
   riiodothyronine or free triiodothyronine
   Free thyroxine
   hyroid-stimulating hormone
Standard urinalysis (with reflexive microscopy)
INR/PT/PTT
Serology for hepatitis B surface antigen and antihepatitis B core antibody.
Serology for antihepatitis C antibody. If positive, follow-up with polymerase chain reaction/viral load testing.

SA serum or urine beta human chorionic gonadotropin pregnancy test for females of childbearing potential.
HbA1c. If HbA1c is elevated (6.5%), refer patient to appropriate provider during Cycle 1 for glucose management.
   (iii) Physical Examination Including Weight

Physical examinations should have included assessments ofthe following body parts/systems: skin, abdomen, extremities, head, heart, lungs, neck, and neurological. Height was only be collected at the Baseline visit. Weight was collected at specified time points (see Table 17) and additionally per institutional standards, if applicable, but did not need to be collected at visits following EOT.

### (iv) ECOG Performance Status

ECOG performance status (see Table 17) is evaluated at protocol-specified time points.

TABLE 17
Performance Status Scales Conversion

| | Karnofsky | | Lansky | | ECOG |
|---|---|---|---|---|---|
| Percent | Description | Percent | Description | Score | Description |
| 100 | Normal, no | 100 | Fully active, normal. | 0 | Normal activity. Fully active, |
| | complaints, no | 90 | Minor restrictions in | | able to carry on all pre-disease |
| | evidence of disease. | | physically strenuous | | performance without |
| 90 | Able to carry on | | activity. | | restriction. |
| | normal activity; | | | | |
| | minor signs or | | | | |
| | symptoms of | | | | |
| | disease. | | | | |
| 80 | Normal activity with | 80 | Active, but tires | 1 | Symptoms, but ambulatory. |
| | effort; some signs or | | more quickly. | | Restricted in physically |
| | symptoms of | 70 | Both greater | | strenuous activity, but |
| | disease. | | restriction of, and | | ambulatory and able to carry |
| 70 | Cares for self, unable | | less time spent in, | | out work of a light or sedentary |
| | to carry on normal | | play activity. | | nature (eg, light housework, |
| | activity or to do | | | | office work). |
| | active work. | | | | |
| 60 | Requires occasional | 60 | Up and around, but | 2 | In bed <50% of the time. |
| | assistance, but is | | minimal active play; | | Ambulatory and capable of all |
| | able to care for most | | keeps busy with | | self-care, but unable to carry |
| | of his/her needs. | | quieter activities. | | out any work activities. Up and |
| 50 | Requires | 50 | Gets dressed, but lies | | about more than 50% of |
| | considerable | | around much of the | | waking hours. |
| | assistance and | | day; no active play; | | |
| | frequent medical | | able to participate in | | |
| | care. | | all quiet active play | | |
| | | | and activities. | | |
| 40 | Disabled, requires | 40 | Mostly in bed, | 3 | In bed >50% of the time. |
| | special care and | | participates in quiet | | Capable of only limited self- |
| | assistance. | | activities. | | care, confined to bed or chair |
| 30 | Severely disabled, | 30 | In bed, needs | | more than 50% of waking |
| | hospitalization | | assistance even for | | hours. |
| | indicated. Death not | | quiet play. | | |
| | imminent. | | | | |
| 20 | Very sick, | 20 | Often sleeping, play | 4 | 100% bedridden. Completely |
| | hospitalization | | entirely limited to | | disabled. Cannot carry on any |
| | indicated. Death not | | very passive | | self-care. Totally confined to |
| | imminent. | | activities. | | bed or chair. |
| 10 | Moribund, fatal | 10 | No play, does not get | | |
| | processes | | out of bed. | | |
| | progressing rapidly. | | | | |
| 0 | Dead. | 0 | Dead. | 5 | Dead. |

### Cardiac Monitoring

ECGs were conducted at baseline and at the EOT visit. Additional ECGs should have been conducted if clinically indicated. Routine 12-lead ECGs were performed after the patient was in a supine position for at least 5 minutes. All ECGs conducted under Amendment 6 and earlier amendments of this protocol were performed centrally. All ECGs conducted under Amendment 7 of this protocol were performed locally. The ECG assessments should have been performed prior to obtaining the PK and biomarker samples, if possible. F or subjects with NYHA Class III heart failure (see Section 6.1.11) or a history of coronary heart disease, arrhythmia or other significant heart disease, a transthoracic ECHO was required at baseline. If clinically indicated, future testing should have used the same modality.

### (v) Complete Eye Examination

Patients had a complete eye examination at screening performed by a qualified optometrist or ophthalmologist, including but not limited to: visual acuity, slit lamp, tonometry examination, and dilated fundus examination. Subsequent eye examinations were conducted as clinically indicated. EOT slit lamp examinations were required for patients who experienced corneal AEs during the study. EOT slit lamp examinations (only required for patients who experience corneal AEs during the study) must have been performed at least 4 weeks from last dose.

### 6.1.7.9 Post-treatment Assessments

### (i) Follow-Up Assessments

Patients who discontinued study treatment received physical exams, ECOG assessment, and response assessments every 9 weeks (±I-week) after EOT up to 1 year after first dose, then every 12 weeks (±1 week). PRO assessments (QLQ-C-30, EQ-5D-5L, BPI-SF, HRU) were completed once every cycle. Scans were done until radiologically confirmed disease progression on or following study treatment (per iRECIST guidelines as determined by the investigator for EV+Pembro arm; or per RECIST Version 1.1 for EV Mono arm), initiation of a subsequent anticancer therapy, patient death, study closure, or withdrawal of consent, whichever occurred first. During study treatment, palliative radiotherapy on a nontarget bone lesion that was not progressing (as long as a target lesion is not within the field of radiation) was not considered a subsequent anticancer therapy; however, radiotherapy on any target lesion was a subsequent anticancer therapy.

### (ii) Long-Term Follow-Up Assessments

After radiologically confirmed PD on or following study treatment and discontinuation of study treatment, patients were contacted every 12 weeks (±1 week) to obtain information on subsequent anticancer therapy and survival status. Long-term follow-up continue until patient death, study closure, or withdrawal of consent, whichever occurred first.

After initiation of subsequent anticancer therapy or radiologically confirmed disease progression per RECIST Version 1.1 (EV Mono arm) or per iRECIST (EV+Pembro arm) on or following study treatment, patients were followed via imaging per institutional guidelines, but not less frequently than every 12 weeks until PFS2 was documented or the patient started another anticancer treatment, whichever occurred first. All subsequent anticancer therapy including date of progression for PFS2 were recorded on the CRF. In addition, patients were contacted every 12 weeks after EOT for survival status and clinical progression status on or following subsequent therapy.

Following PFS2, patients entered the survival follow-up period and were followed every 12 weeks (±1 week) for survival status until death, study closure, or withdrawal of consent, whichever occurred first.

PRO assessments (QLQ-C-30, EQ-5D-5L, BPI-SF, HRU) were completed once every cycle.

### 6.1.7.10 Appropriateness of Measurements

The safety measures that were used in this trial were considered standard procedures for evaluating the potential adverse effects of study medications.

The determination of antitumor activity is based on confirmed objective response assessments as defined by RECIST Version 1.1 (Eisenhauer 2009) (Table 40) and treatment decisions by the investigator were based on iRECIST guidelines (Seymour 2017) (Section 6.1.15) for EV+Pembro arm; or per RECIST Version 1.1 for EV Mono arm. RECIST criteria were considered standard in oncological practice for this type of neoplasm, and the intervals of evaluation in this protocol are appropriate for disease management. iRECIST guidelines are newly published criteria that were applied as exploratory criteria for this study. These guidelines allowed consideration that immunotherapeutics, such as pembrolizumab, may result in infiltration of immune cells leading to transient increase in the size in malignant lesions, or undetectable lesions becoming detectable. The guidelines are identical to those of RECIST Version 1.1 in many respects but have been adapted to account for instances where an increase in tumor burden, or the appearance of new lesions, does not reflect true tumor progression.

Immunogenicity is commonly assessed for biologics; therefore, standard tests were performed to detect the possible presence of specific antibodies to enfortumab vedotin.

PK assessments are also common in clinical studies to help characterize dose exposure response relationships.

Exploratory biomarker measurements in peripheral blood samples enable correlation with PK assessments and are common in clinical studies. Assessments conducted on pre-treatment tumor tissue are similarly common. Both peripheral blood and tumor biomarker samples were assessed using commonly employed, standard tests.

### 6.1.8 Data Analysis Methods

### 6.1.8.1 Determination of Sample Size

Approximately 150 patients were randomized in a 1:1 ratio to receive either enfortumab vedotin monotherapy (EV Mono arm) or enfortumab vedotin in combination with pembrolizumab (EV+Pembro arm).

The sample size was not based on power calculations for formal hypothesis testing but was selected based on the precision of the estimate for ORR as characterized by 95% CIs. Formal statistical comparisons between the 2 arms were not performed.

For a sample size of 75 patients per arm, assuming the ORR is between 50% and 70%, the 2 sided 95% CIs are summarized below in Table 18.

TABLE 18
Summary of 2 sided 95% CIs

| | 95% Exact CI |
|---|---|
| ORR | (n = 75) |
| 50% | (39%, 62%) |
| 60% | (48%, 71%) |
| 70% | (59%, 81%) |

The In total, approximately 150 patients were enrolled in the study.

### 6.1.8.2 Study Endpoint Definitions

### (i) Objective Response Rate (ORR)

The primary endpoint was ORR by BICR. ORR by investigator assessment was a secondary endpoint.

The ORR was defined as the proportion of patients with confirmed CR or PR according to RECIST Version 1.1 (Eisenhauer 2009) (Table 40). Patients who did not have at least 2 (initial response and confirmation scan) post-baseline response assessments were counted as non responders.

A separate endpoint of ORR summarizing the proportion of patients with confirmed CR or PR based on iRECIST guidelines was analyzed for arm utilizing pembrolizumab.

### (ii) Duration of Response (DOR)

The DOR according to RECIST Version 1.1 was defined as the time from first documentation of objective response (that is subsequently confirmed) to the first documentation of objective tumor progression, or to death due to any cause, whichever came first.

DOR by BICR and by investigator assessment are secondary endpoints.

DOR is only calculated for the patients achieving a confirmed CR or PR.

DOR data is censored as described below:
Patients who do not have PD and are still on study at the time of an analysis are censored at the date of the last disease assessment documenting absence of PD;
Patients who have started an anticancer therapy other than the study treatment prior to documentation of PD are censored at the date of the last disease assessment prior to start of new therapy;
Patients who are removed from the study prior to documentation of PD are censored at the date of the last disease assessment documenting absence of PD.

For arm utilizing pembrolizumab, a separate endpoint of DOR was analyzed based on iRECIST guidelines, with DOR based on iRECIST guidelines (iDOR) defined as the time from first documentation of objective response (that is subsequently confirmed) by investigator assessment to the first documentation of objective tumor progression (that is subsequently confirmed), or to death due to any cause, whichever came first.

(iii) Disease Control Rate (DCR)

DCR is defined as the proportion of patients with CR, PR, or SD per RECIST Version 1.1 (Eisenhauer 2009) (see Table 40).

DCR by BICR and by investigator assessment were secondary endpoints.

For arm utilizing pembrolizumab, a separate endpoint of DCR was analyzed based on iRECIST guidelines, with iDCR defined as CR based on iRECIST guidelines (iCR)+partial response based on iRECIST guidelines (iPR)+stable disease based on iRECIST guidelines (iSD).

### (iv) Progression-free Survival on Study Therapy (PFS)

PFS is defined as the time from start of study treatment to first documentation of objective tumor progression on or following study therapy per RECIST Version 1.1, or to death due to any cause, whichever comes first.

PFS by BICR and by investigator assessment were secondary endpoints

The same censoring rules outlined in Section 9.2.2 for DOR was applied to PFS. Patients lacking an evaluation of tumor response after their first dose had their event time censored at Day 1.

For arm utilizing pembrolizumab, a separate endpoint of PFS was analyzed based on iRECIST guidelines, with iPFS defined as the time from start of study treatment to the first date at which progression criteria were met (i.e., the date of iUPD) provided that iCPD was confirmed at the next assessment, or to death due to any cause, whichever came first.

### (v) Overall Survival

OS is defined as the time from start of study treatment to date of death due to any cause. OS was a secondary endpoint

### 6.1.8.3 Statistical and Analytical Plans

The statistical and analytical plans presented below summarize the more complete plans detailed in the statistical analytical plan (SAP). A change to the data analysis methods described in the protocol required a protocol amendment only if it altered a principal feature of the protocol. The SAP was finalized prior to database lock. Any changes to the methods described in the final SAP were described and justified in the clinical study report.

### (i) General Considerations

Disease response was summarized per RECIST Version 1.1 by BICR as well as by investigator assessment. In addition, disease response was summarized per the iRECIST guidelines (Seymour 2017) (Section 6.1.15) for the EV+Pembro arm by investigator assessment.

In general, descriptive statistics were presented that include the number of observations, mean, standard deviation, median, minimum and maximum for continuous variables, and the number and percentages (of non-missing) per category for categorical variables.

Unless otherwise specified, CI was calculated at two-sided 95% level.

Unless otherwise specified, CI was calculated at two-sided 95% level.

The two-sided 95% exact CI using Clopper-Pearson methodology (Clopper 1934) was calculated for the response rates where applicable (eg, ORR, DCR, pCRR, and pDSR).

For time-to-event endpoints, the median survival time was estimated using the Kaplan Meier method; the associated 95% CI was calculated based on the complementary log-log transformation (Collett 1994).

### Randomization and Blinding

This was an open-label study.

Approximately 150 patients were randomized in a 1:1 ratio to the EV Mono arm or the EV+Pembro arm based on the following stratification factors: the absence or presence of liver metastases, and ECOG performance status (0 versus 1/2).

The EV Mono arm included approximately 75 patients.

The EV+Pembro arm included approximately 75 patients.

### Data Transformations and Derivations

Time variables based on 2 dates, e.g., Start Date and End Date, were calculated as (End Date−Start Date+1) (in days) unless otherwise specified in the planned analysis section.

Unless otherwise specified in the analysis plan, baseline values used in all analyses were the most recent non-missing measurement prior to the first dose of study drug.

### Analysis Sets

The safety analysis set includes all patients who received any amount of study drug (either enfortumab vedotin or combination agent). The safety analysis set was used for all safety analyses.

The full analysis set (FAS) included all patients who were enrolled in the study and received any amount of study drug (enfortumab vedotin or combination agent). The FAS was used for the analysis of efficacy endpoints.

The efficacy-evaluable analysis set included all patients in the FAS who had at least 2 post-baseline response assessments or discontinued from treatment for any reason. The efficacy-evaluable analysis set was used for additional analyses of efficacy endpoints.

The PK analysis set included all patients who received enfortumab vedotin and from whom at least 1 blood sample was collected and assayed for enfortumab vedotin, MMAE, or TAb concentration. Corresponding records of the time of dosing and sample collection must also be available for all enfortumab vedotin, MMAE, and TAb concentration. The PK analysis set was used for analyses of PK parameters.

Additional analysis sets of patients may have been defined in the SAP.

### Examination of Subgroups

As exploratory analyses, subgroup analyses may have been conducted for selected endpoints.

Subgroups may have included the following:
Age (<65, ≥65 years old)
Sex (female, male)
ECOG performance status (0, 1-2)
Bellmunt risk score (0-1, ≥2)
Visceral metastases vs. lymph nodes only disease
Liver metastases at baseline (yes, no)
PD-L1 expression (CPS <10, ≥10)

### Timing of Analyses

The primary analysis was conducted based on confirmed ORR.

Additional cutoff dates may have been defined and corresponding database locks may have occurred to allow for more precise estimates of time-to-event endpoints.

### (ii) Patient Disposition

An accounting of study patients by disposition was tabulated and the number of patients in each analysis set was summarized. Patients who discontinued study treatment and patients who withdrew from the study were summarized with reason for discontinuation or withdrawal for all enrolled patients.

(iii) Patient Characteristics

Demographics accounting of study patients by disposition were tabulated and the number of patients in each analysis set were summarized. Patients who discontinued study treatment and patients who withdrew from the study were summarized with reason for discontinuation or withdrawal for all enrolled patients.

### (iv) Efficacy Analyses

The primary analysis of efficacy endpoints were analyzed using the FAS as defined in Section 9.4.1.7. Supplemental analyses of efficacy endpoints may have been presented using the efficacy-evaluable analysis sets.

The primary analysis of efficacy endpoints was analyzed using the FAS as defined in Section 6.1.7.11. Supplemental analyses of efficacy endpoints may have been presented using the efficacy-evaluable analysis sets.

The primary endpoint was ORR by BICR. The observed ORR by BICR and the 95% CIs was provided for the FAS population using Clopper-Pearson methodology by arm.

The analyses on secondary endpoints, ORR by investigator assessment, DCR by BICR, and DCR by investigator assessment was summarized, and their exact 2-sided 95% CIs using the Clopper-Pearson method (Clopper 1934) was calculated.

Other secondary endpoints, such as DOR by BICR, DOR by investigator assessment, PFS by BICR, PFS by investigator assessment, OS are time-to-event endpoints, and they were analyzed using Kaplan-Meier methodology and Kaplan-Meier plots were provided. Details on the censoring algorithm were provided in the SAP.

ORR, DCR, DOR, and PFS per iRECIST guidelines (Seymour 2017) (Section 6.1.15) for the EV+Pembro arm by investigator assessment were performed similarly as described above.

Formal statistical comparisons between the 2 arms were not performed.

### (v) Pharmacokinetic and ATA Analyses

Enfortumab vedotin ADC, MMAE, and TAb concentrations were summarized with descriptive statistics at each PK sampling time point. PK parameters, including, but not limited to AUC, Cmax, and Tmax, were estimated by noncompartmental analyses and summarized by descriptive statistics. The relationship between PK and pharmacodynamics endpoints, safety, or efficacy may have been explored.

The incidence of ATA were summarized by visit using the safety analysis set.

### (vi) Patient Reported Outcomes Analyses

Completion and compliance rates for each assessment were summarized. PRO analyses were performed on the FAS population. If any additional analysis was planned, it was detailed in a supplemental SAP.

(vii) Biomarker Analyses

Relationships of biomarker parameters (e.g., pre-treatment values, absolute and relative changes from pre-treatment) to efficacy, safety, and PK parameters were explored. Relationships and associated data that were determined to be of interest were summarized. Details of these analyses were described separately.

(viii) Safety Analyses

The safety analysis set was used to summarize all safety endpoints.

### (a) Extent of Exposure

Duration of treatment, number of cycles, total dose and dose intensity were summarized. Dose modifications were also summarized in similar manner. Details were provided in the SAP.

### (b) Adverse Events

An overview of AEs provided a tabulation of the incidence of all AEs, TEAEs, treatment-related AEs, Grade 3 and higher AEs, SAEs, treatment-related SAEs, deaths, and AEs leading to study treatment discontinuation. AEs are defined as treatment emergent if they are newly occurring or worsen following study treatment.

AEs are listed and summarized by Medical Dictionary for Regulatory Activities (MedDRA), preferred term, severity, and relationship to study drug. In the event of multiple occurrences of the same AE with the same preferred term in 1 patient, the AE was counted once as the occurrence. The incidence of AEs was tabulated by preferred term. AEs leading to premature discontinuation of study drug were summarized and listed in the same manner.

### (c) Deaths and Serious Adverse Events

SAEs were listed and summarized in the same manner as all AEs. Events with a fatal outcome were listed.

### (d) Clinical Laboratory Results

Laboratory values (e.g., chemistry, hematology, and urinalysis with reflexive microscopic analysis) may have been presented graphically by visit. Summary statistics may have been tabulated as appropriate by scheduled visit. Laboratory values were listed with grade per NCI CTCAE Version 4.03 and flagged when values were outside the normal reference range.

### (e) Other Safety Analyses

### Vital Signs

Summary statistics and change from baseline and/or predose to postdose may have been tabulated where appropriate.

### ECOG Status

ECOG status was summarized for each visit. Shifts from baseline to the best and worst post-baseline score may have been tabulated.

### ECG

ECG status (normal, abnormal clinically significant, or abnormal not clinically significant) may have been summarized for each scheduled and unscheduled ECG, and shifts from baseline may have been tabulated.

### 6.1.9 Study Results

The phase 1b/2 study of enfortumab vedotin combined with pembrolizumab as first-line treatment in patients with unresectable locally advanced or metastatic urothelial cancer (la/mUC) who are unable to receive cisplatin-based chemotherapy described in this Example completed enrollment on Oct. 11, 2021 with 151 subjects enrolled. The number of subjects who received any amount of study drug in the EV+Pembro arm of the study was 76. The number of subjects who received any amount of study drug in the EV Mono arm of the study was 73. The results from the dose escalation/cohort A treated with EV+pembro (which completed enrollment on Jan. 28, 2019 with 45 subjects enrolled and 45 who received any amount of study drug) are included as comparison for various analyses as indicated.

Regarding disposition, as shown in Table 19, progressive disease was the most comment reason for treatment discontinuation, with 25 subjects having remained on treatment in the EV+Pembro arm of the study and 8 subjects having remained on treatment in the EV Mono arm of the study.

TABLE 19

| | Dose Esc/Cohort A | Cohort K | Cohort K |
|---|---|---|---|
| | EV + pembro | EV + pembro | EV Mono |
| | (N = 45) | (N = 77) ^{a} | (N = 74) ^{a} |
| Subjects who received any | 45 (100.0) | 76 (98.7) | 73 (98.6) |
| amount of study drug | | | |
| Subjects on treatment | 0 | 25 (32.5) | 8 (10.8) |
| Subjects off treatment | 45 (100.0) | 51 (66.2) | 65 (87.8) |
| Primary reason for | | | |
| treatment discontinuation | | | |
| Progressive disease | 19 (42.2) | 33 (42.9) | 40 (54.1) |
| Adverse event | 15 (33.3) | 12 (15.6) | 18 (24.3) |
| Physician decision | 1 (2.2) | 1 (1.3) | 3 (4.1) |
| Subject decision | 9 (20.0) | 4 (5.2) | 3 (4.1) |
| Other | 1 (2.2) | 1 (1.3) | 1 (1.4) |
| Subjects on study | 18 (40.0) | 54 (70.1) | 46 (62.2) |
| Subjects off study | 27 (60.0) | 23 (29.9) | 28 (37.8) |

^{a} Includes 1 subject in each arm who were enrolled and never received study treatment

Regarding demographics, as shown in Table 20, the demographics and baseline disease characteristics are representative of the 1 L cisplatin ineligible LA or mUC population with high unmet medical need.

TABLE 20

| | | |
|---|---|---|
| Dose Esc/Cohort A | Cohort K | Cohort K |
| EV + pembro | EV + pembro | EV Mono |
| (N = 45) | (N = 76) | (N = 73) |

Age (yr)

| | | | |
|---|---|---|---|
| Median | 69.0 | 71.0 | 74.0 |
| Min, Max | 51, 90 | 51, 91 | 56, 89 |

Age group

| | | | |
|---|---|---|---|
| ≥75 years | 16 (35.6) | 24 (31.6) | 34 (46.6) |

Sex, n (%)

| | | | |
|---|---|---|---|
| Male | 36 (80.0) | 54 (71.1) | 56 (76.7) |
| Female | 9 (20.0) | 22 (28.9) | 17 (23.3) |

Race, n (%)

| | | | |
|---|---|---|---|
| Asian | 0 | 5 (6.6) | 6 (8.2) |
| Black or African American | 1 (2.2) | 5 (6.6) | 5 (6.8) |
| White | 42 (93.3) | 61 (80.3) | 55 (75.3) |
| Other | 1 (2.2) | 1 (1.3) | 0 |
| Unknown or not reportable | 1 (2.2) | 4 (5.3) | 7 (9.6) |

Geographic region, n (%)

| | | | |
|---|---|---|---|
| North America | 45 (100.0) | 73 (96.1) | 66 (90.4) |
| Europe | 0 | 3 (3.9) | 7 (9.6) |

Regarding the baseline characteristics, as shown in Table 21, the study population is an elderly population with male predominance, multiple comorbidities, with known poor prognostic factors, representative of cisplatin-ineligible populations in prior studies.

TABLE 21

| | | |
|---|---|---|
| Dose Esc/Cohort A | Cohort K | Cohort K |
| EV + pembro | EV + pembro | EV Mono |
| (N = 45) | (N = 76) | (N = 73) |

ECOG performance status, n (%)

| | | | |
|---|---|---|---|
| 0 | 15 (33.3) | 33 (43.4) | 28 (38.4) |
| 1 | 22 (48.9) | 33 (43.4) | 35 (47.9) |
| 2 | 8 (17.8) | 10 (13.2) | 10 (13.7) |

Body mass index (kg/m²)

| | | | |
|---|---|---|---|
| Median | 27.9 | 25.8 | 26.4 |
| Min, Max | 20, 55 | 14, 43 | 16, 42 |
| >=30 kg/m² | 11 (24.4) | 20 (26.3) | 16 (21.9) |

Renal function based on CrCL, n (%)

| | | | |
|---|---|---|---|
| Normal: >=90 mL/min | 9 (20.0) | 9 (11.8) | 4 (5.5) |
| Mild decrease: | 12 (26.7) | 14 (18.4) | 13 (17.8) |
| >=60 and <90 mL/min | | | |
| Moderate decrease: | 23 (51.1) | 50 (65.8) | 53 (72.6) |
| >=30 and <60 mL/min | | | |
| Severe decrease: | 1 (2.2) | 3 (3.9) | 3 (4.1) |
| >=15 and <30 mL/min^{a} | | | |

Bajorin risk factors, n (%) ^{b}

| | | | |
|---|---|---|---|
| 0 | 16 (35.6) | 25 (32.9) | 26 (35.6) |
| 1 | 29 (64.4) | 51 (67.1) | 47 (64.4) |

^{a}Patients met the inclusion criteria based on the GFR at screening visit, although baseline CrCL calculated from the last measurement before first dose of study drug was below 30 mL/min.
^{b} Bajorin risk factors include visceral metastases (bone, lung, liver) and ECOG PS >2.
Subjects with ECOG PS >2 are not eligible for the study

Regarding the baseline disease characteristics, as shown in Table 22, the study population is representative of cisplatin-ineligible populations in other clinical trials and with known poor prognostic factors (e.g.; renal impairment, upper tract disease, visceral disease including liver metastases).

TABLE 22

| | | |
|---|---|---|
| Dose Esc/Cohort A | Cohort K | Cohort K |
| EV + pembro | EV + pembro | EV Mono |
| (N = 45) | (N = 76) | (N = 73) |

PD-L1 expression, n (%)

| | | | |
|---|---|---|---|
| n | 33 | 75 | 66 |
| CPS ≥10 | 15/33 (45.5) | 31/75 (41.3) | 28/66 (42.4) |
| CPS <10 | 18/33 (54.5) | 44/75 (58.7) | 38/66 (57.6) |

Time from diagnosis of la/mUC to enrollment (months)

| | | | |
|---|---|---|---|
| Median | 1.84 | 1.38 | 1.58 |
| Min, Max | 0.3, 15.2 | 0.4, 34.8 | 0.5, 86.3 |

Current extent of disease, n (%)

| | | | |
|---|---|---|---|
| Locally advanced | 0 | 3 (3.9) | 1 (1.4) |
| Metastatic | 45 (100.0) | 73 (96.1) | 72 (98.6) |

Primary tumor location, n (%)

| | | | |
|---|---|---|---|
| Upper tract | 15 (33.3) | 30 (39.5) | 21 (28.8) |
| Lower tract | 30 (66.7) | 46 (60.5) | 51 (69.9) |
| Both | 0 | 0 | 1 (1.4) |

Metastasis disease sites at baseline^{a}, n (%)

| | | | |
|---|---|---|---|
| Bone | 8 (17.8) | 19 (25.0) | 21 (28.8) |
| Liver | 14 (31.1) | 13 (17.1) | 13 (17.8) |
| Lung | 19 (42.2) | 37 (48.7) | 30 (41.1) |

Metastasis Category, n (%)

| | | | |
|---|---|---|---|
| Visceral metastases | 38 (84.4) | 64 (84.2) | 60 (82.2) |
| Lymph node only | 7 (15.6) | 10 (13.2) | 12 (16.4) |

^{a}A subject may have metastatic disease in more than one location

Regarding reasons for cisplatin ineligibility, as shown in Table 23, the cisplatin-ineligible population is clearly defined. Renal insufficiency was the most common reason for cisplatin ineligibility, followed by hearing loss.

TABLE 23

| | Dose Esc/Cohort A | Cohort K | Cohort K |
|---|---|---|---|
| | EV + pembro | EV + pembro | EV Mono |
| | (N = 45) | (N = 76) | (N = 73) |
| Subjects meeting at least one of the Galsky criteria^{a} | 44 (97.8) | 76 (100.0) | 72 (98.6) |
| CrCL <60 and ≥30 mL/min | 25 (55.6) | 48 (63.2) | 44 (60.3) |
| ECOG PS of 2 | 6 (13.3) | 6 (7.9) | 9 (12.3) |
| ≥Grade 2 hearing loss | 5 (11.1) | 11 (14.5) | 11 (15.1) |
| CrCL <60 and ≥30 mL/min and ECOG PS of 2 | 2 (4.4) | 4 (5.3) | 1 (1.4) |
| CrCL <60 and ≥30 mL/min and ≥ Grade 2 hearing loss | 5 (11.1) | 7 (9.2) | 7 (9.6) |
| ECOG PS of 2 and ≥ Grade 2 hearing loss | 1 (2.2) | 0 | 0 |
| Subjects considered cisplatin ineligible by the | 1 (2.2) | 0 | 1 (1.4) |
| investigator although not meeting Galsky criteria^{b} | | | |

^{a}Cisplatin-ineligibility categories are mutually exclusive. Each subject is only counted once. Assessments performed at the screening/baseline visit are used for categorization.
^{b}One subject in Cohort A was considered cisplatin ineligible by the investigator due to solitary kidney, and 1 subject in Cohort K (EV Mono) was considered cisplatin ineligible by the investigator due to age and Grade 1 hearing loss.

According to the blinded independent central review (BICR) (per RECIST 1.1), overall response rate (ORR) for the Cohort K EV+pembro arm was 64.5% (95% CI: 52.7%, 75.1%) with a complete response (CR) of 10.5%, as shown in Table 24. The ORR per investigator assessment was consistent with the BICR assessment. The concordance between investigator and BICR was 86.7% in the Cohort K EV+pembro arm.

TABLE 24

| | | | |
|---|---|---|---|
| Dose Esc/Cohort A | Cohort K | Dose Escal/A/K | Cohort K |
| EV + pembro | EV + pembro | EV + pembro | EV Mono |
| (N = 45) | (N = 76) | (N = 121) | (N = 73) |

Best Overall Response^{a}, n (%)

| | | | | |
|---|---|---|---|---|
| Complete Response (CR) | 7 (15.6) | 8 (10.5) | 15 (12.4) | 3 (4.1) |
| Partial Response (PR) | 26 (57.8) | 41 (53.9) | 67 (55.4) | 30 (41.1) |
| Stable Disease (SD) | 5 (11.1) | 17 (22.4) | 22 (18.2) | 25 (34.2) |
| Progressive Disease (PD) | 5 (11.1) | 6 (7.9) | 11 (9.1) | 7 (9.6) |
| Not Evaluable (NE) | 0 | 3 (3.9) | 3 (2.5) | 5 (6.8) |
| No Assessment | 2 (4.4) | 1 (1.3) | 3 (2.5) | 3 (4.1) |
| ORR (CR or PR), n (%)^{b} | 33 (73.3) | 49 (64.5) | 82 (67.8) | 33 (45.2) |
| 95% CI | (58.1, 85.4) | (52.7, 75.1) | (58.7, 76.0) | (33.5, 57.3) |
| Median time to response | 1.94 | 2.07 | 2.07 | 2.07 |
| (range) | (1.1, 13.2) | (1.1, 6.6) | (1.1, 13.2) | (1.9, 15.4) |

^{a}CR or PR was confirmed with repeat scans ≥4 weeks after initial response.
^{b}Confirmed ORR.

Table 25 shows a side-by-side comparison of the Cohort K EV+pembro arm and Cohort K EV monotherapy arm that includes data presented in Table 24, supra, to illustrate the differences between the two arms. As indicated above, the ORR for the Cohort K EV+pembro arm was 64.5% (95% CI: 52.7%, 75.1%) with a complete response (CR) of 10.5%. In contrast, the ORR for the Cohort K EV monotherapy arm was 45.2% (95% CI: 33.5%, 57.3%) with a complete response (CR) of 4.1%.

TABLE 25

| | EV + P | EV mono |
|---|---|---|
| | (N = 76) | (N = 73) |
| Confirmed ORR, % (95% CI) | 64.5 (52.7, 75.1) | 45.2 (33.5, 57.3) |
| Best overall response per BICR, % | | |
| Complete Response | 10.5 | 4.1 |
| Partial Response | 53.9 | 41.1 |
| Stable Disease | 22.4 | 34.2 |
| Progressive Disease | 7.9 | 9.6 |
| Not Evaluable | 3.9 | 6.8 |
| No Assessment | 1.3 | 4.1 |
| Median (range) time to ORR, mos | 2.1 (1.1, 6.6) | 2.1 (1.9, 15.4) |
| Median (range) treatment duration, mos | 9.0 (0.6, 26.1+) | 5.5 (0.5, 26.9+) |
| Median (95% CI) follow-up, mos | 14.8 (12.9, 17.3) | 15.0 (12.7, 17.4) |

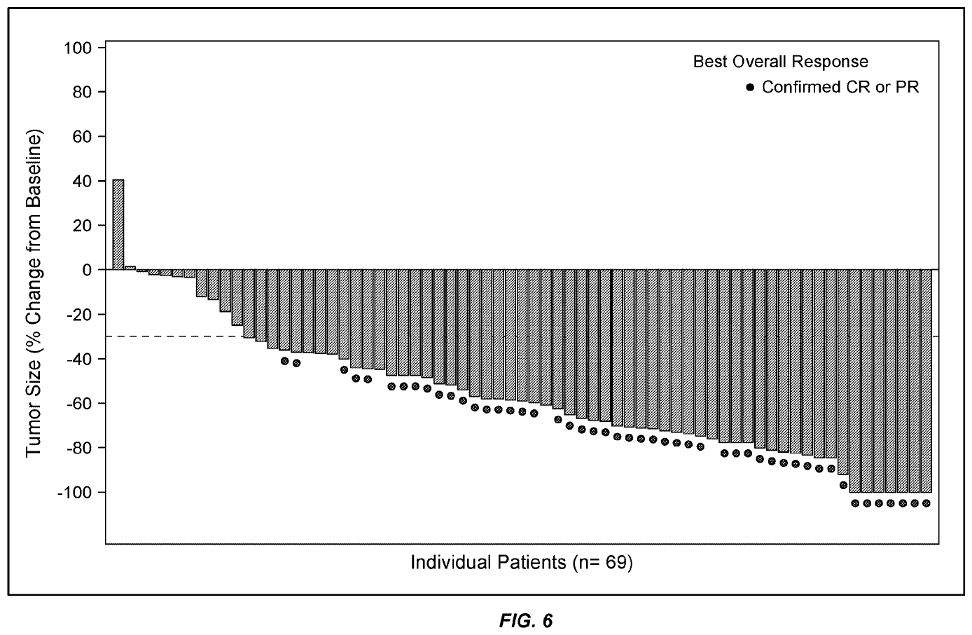

Significant tumor reduction in target lesions was observed, where 97.1% of assessable Cohort K EV+pembro subjects had tumor reduction by blinded independent central review (see FIG. 6).

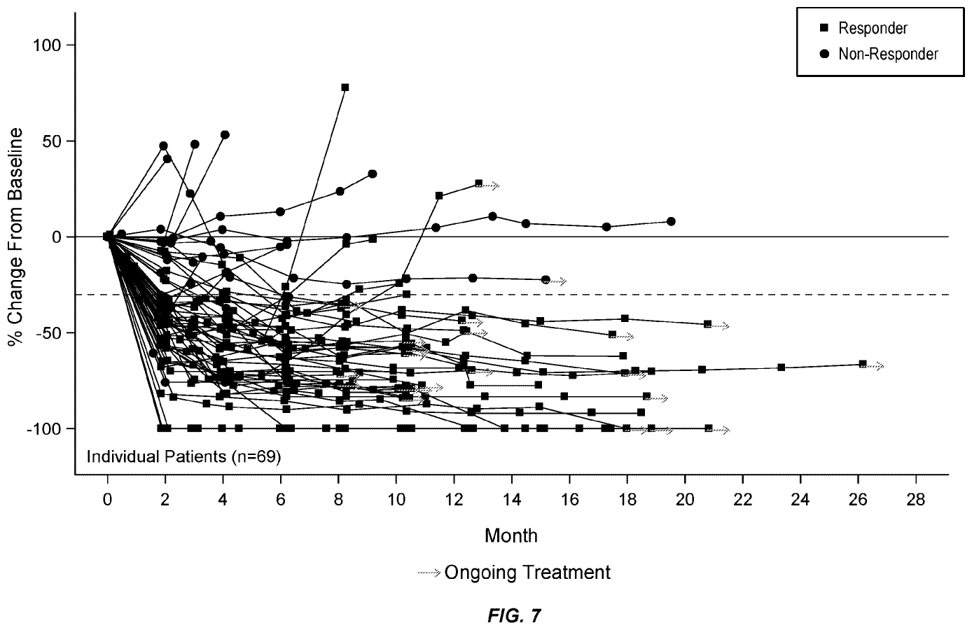

The percent change from baseline in sum of diameters of target lesions over time by BICR is shown in FIG. 7. 85.7% of responses were observed at first assessment (Week 9+/−1 week) (see FIG. 7). The median time to response was 2.07 months (range: 1.1 to 6.6). The median time to response was 2.07 months (range: 1.1 to 6.6) (see FIG. 7).

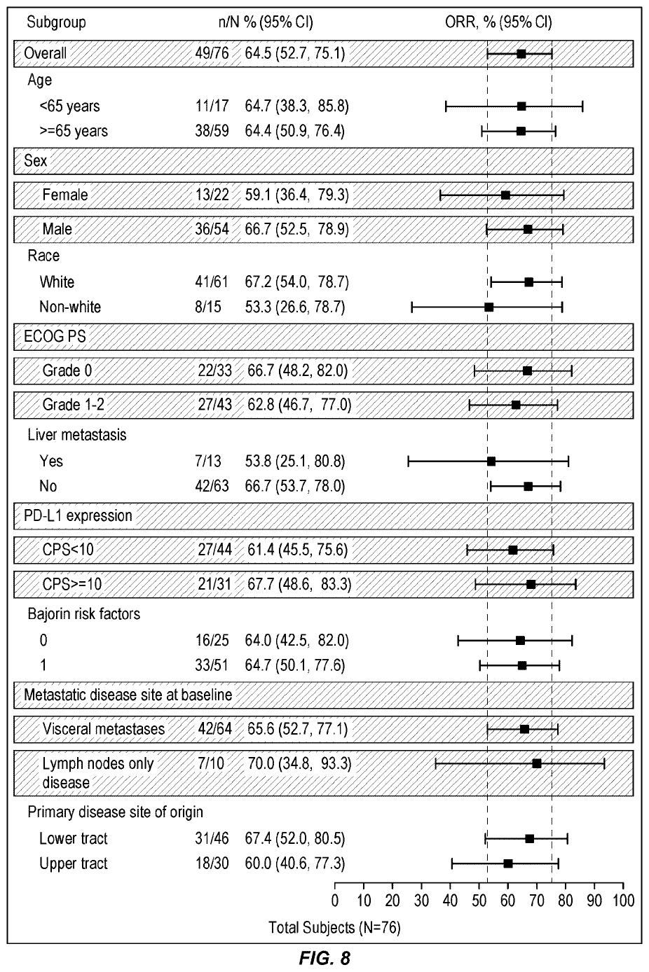

In the ORR subgroup analysis of patients in the Cohort K EV+pembro arm as shown in FIG. 8, ORRs were consistent across all pre-specified subgroups. Responses were observed regardless of PD-L1 expression status at baseline. As indicated supra, the ORR for the Cohort K EV+pembro arm was 64.5% (95% CI: 52.7%, 75.1%). 53.8% confirmed ORR was observed in patients with liver metastases. In addition, ORR point estimates were >50% for all subgroups.

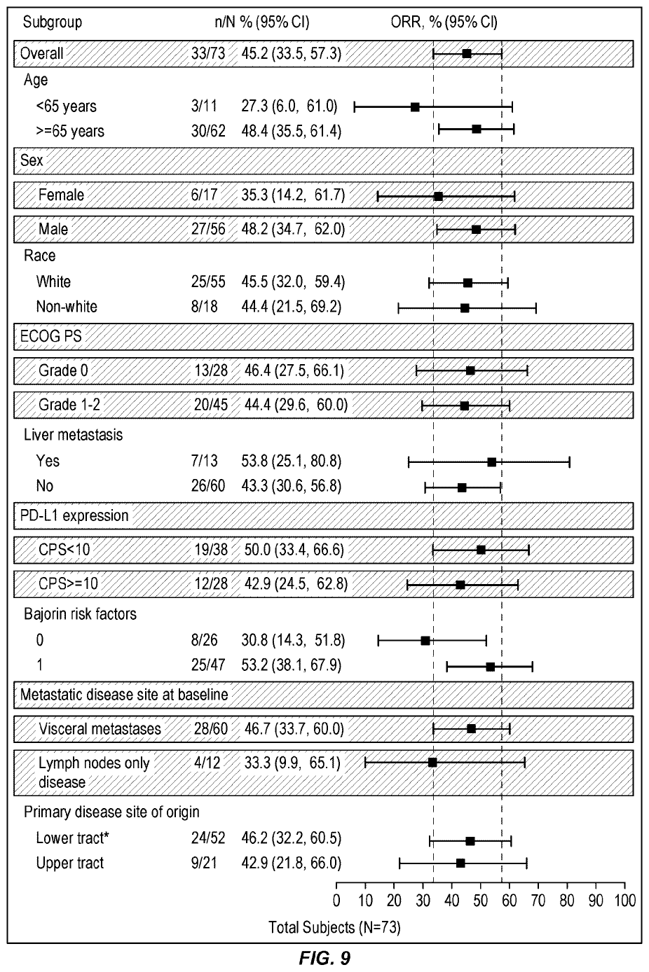

In the ORR subgroup analysis of patients in the Cohort K EV monotherapy arm as shown in FIG. 9, ORRs were consistent across all pre-specified subgroups. Responses were observed regardless of PD-L1 expression status at baseline. As indicated supra, the ORR for the Cohort K EV monotherapy arm was 45.2% (95% CI: 33.5%, 57.3%). 53.8% confirmed ORR was observed in patients with liver metastases.

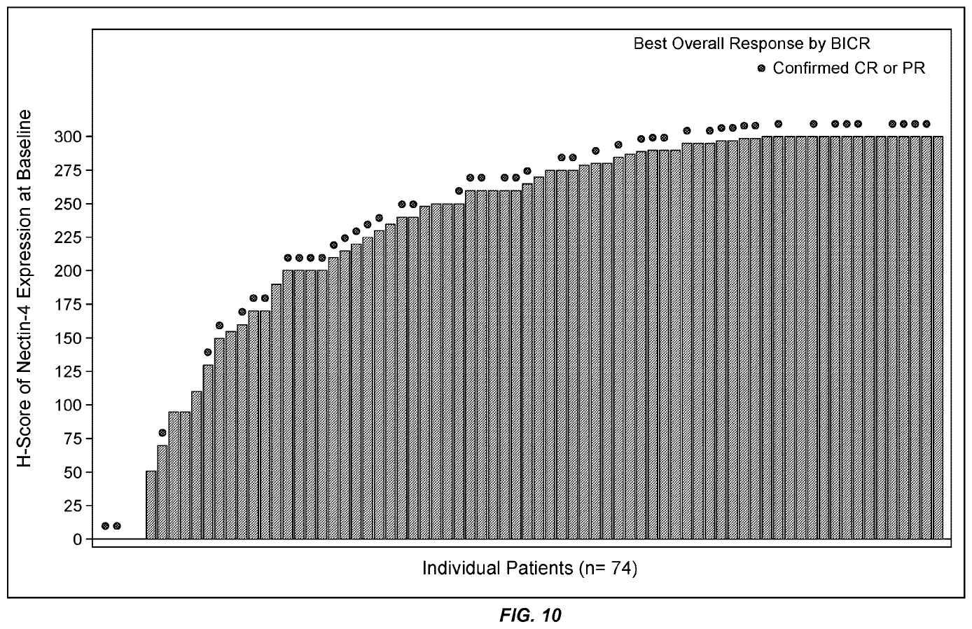

FIG. 10 shows the H-score of Nectin-4 expression at baseline and best overall response by BICR. Nectin-4 was detected in tumor tissue from 94.6% of subjects who had adequate tissue for testing. Activity was observed regardless of Nectin-4 expression at baseline.

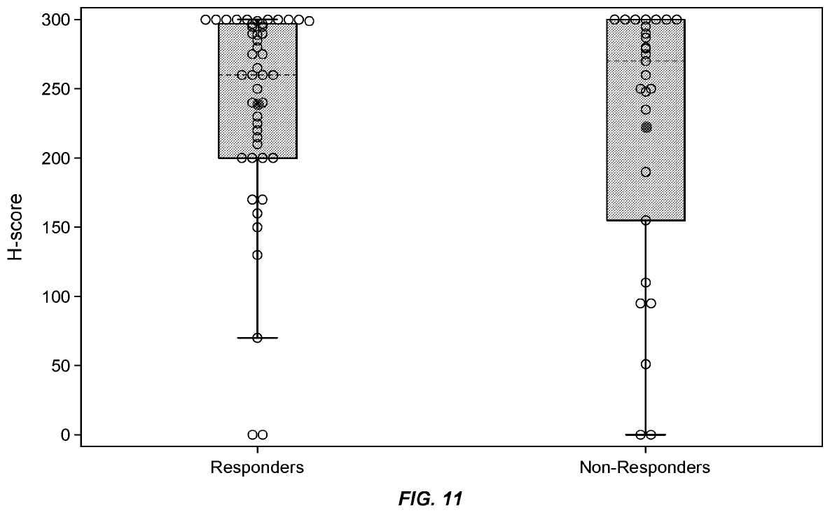

FIG. 11 shows the H-Score of Nectin-4 expression at baseline by best overall response per BICR. There was no evidence to support an expression-response relationship between Nectin-4 and study treatment (see FIG. 11 and Table 26). Responses were observed in patients with low Nectin-4 expression.

TABLE 26

| | N | MEAN | STD | MIN | Q1 | MEDIAN | Q3 | MAX |
|---|---|---|---|---|---|---|---|---|
| Responders | 47 | 239.4 | 74.72 | 0 | 200.0 | 260.0 | 297.0 | 300 |
| Non- | 27 | 222.8 | 98.15 | 0 | 155.0 | 270.0 | 300.0 | 300 |
| Responders | | | | | | | | |

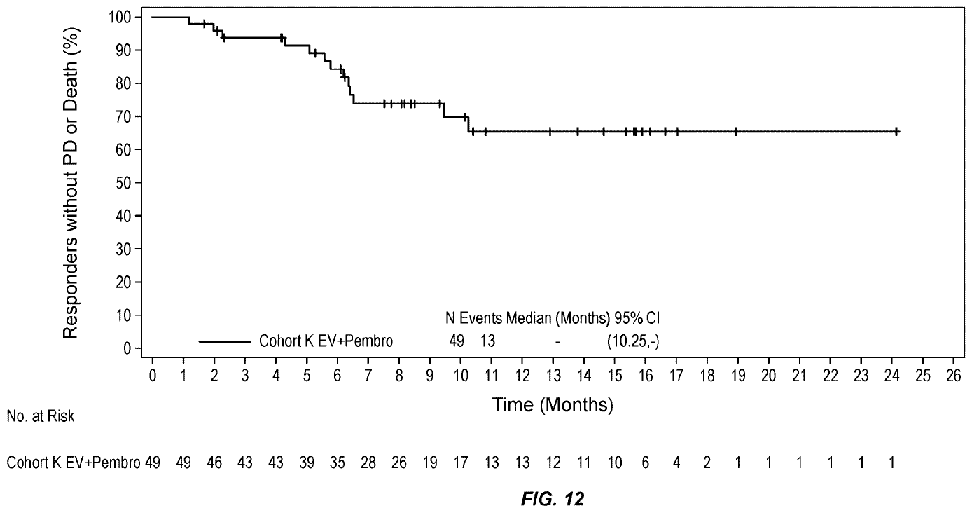

According to the blinded independent central review, median DOR for the Cohort K EV+pembro arm was not reached (see FIG. 12 and Table 27). 84.2% and 65.4% of responders had DOR ≥6 months and DOR ≥12 months respectively (see FIG. 12 and Table 27).

TABLE 27

| | Dose Esc/ | | |
|---|---|---|---|
| | Cohort A | Cohort K | Cohort K |
| | EV + pembro | EV + pembro | EV Mono |
| | (N = 45) | (N = 76) | (N = 73) |
| No. of responders | 33 | 49 | 33 |
| No. of events | 15 | 13 | 14 |
| mDOR (95% CI), | 22.1 | — | 13.2 |
| months | (8.38, —) | (10.25, —) | (6.14, 15.97) |
| % with DOR ≥6 months | 74.1% | 84.2% | 71.2% |
| % with DOR ≥12 months | 63.9% | 65.4% | 56.3% |

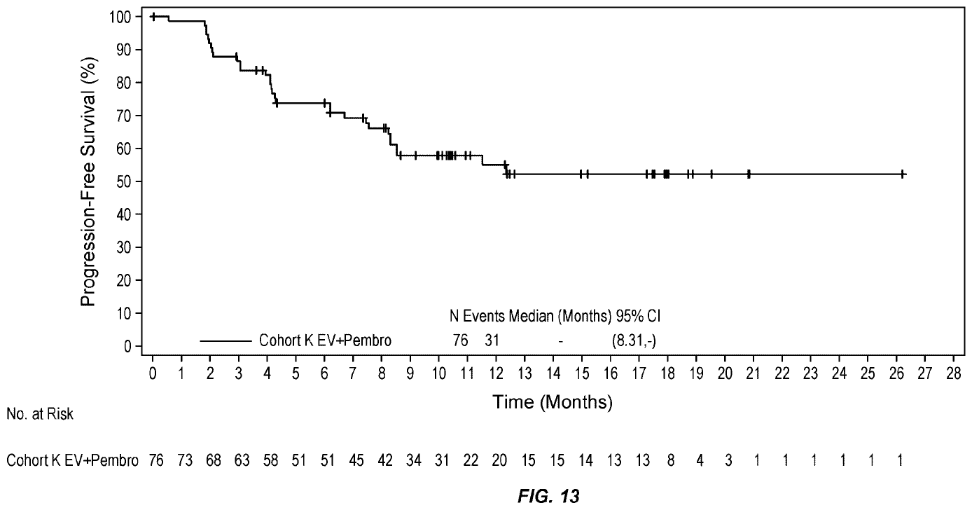

According to the blinded independent central review, median PFS for the Cohort K EV+pembro arm was not reached (see FIG. 13 and Table 28). At 6-months, the PFS was 73.8% and at 12-months the PFS was 55.1% (see FIG. 13 and Table 28).

TABLE 28

| | Dose Esc/ | | |
|---|---|---|---|
| | Cohort A | Cohort K | Cohort K |
| | EV + pembro | EV + pembro | EV Mono |
| | (N = 45) | (N = 76) | (N = 73) |
| No. of events | 25 | 31 | 38 |
| mPFS (95% CI), | 12.7 | — | 8.0 |
| months | (6.11, —) | (8.31, —) | (6.05, 10.35) |
| PFS at 6 months | 72.4% | 73.8% | 64.2% |
| PFS at 12 months | 55.0% | 55.1% | 35.8% |

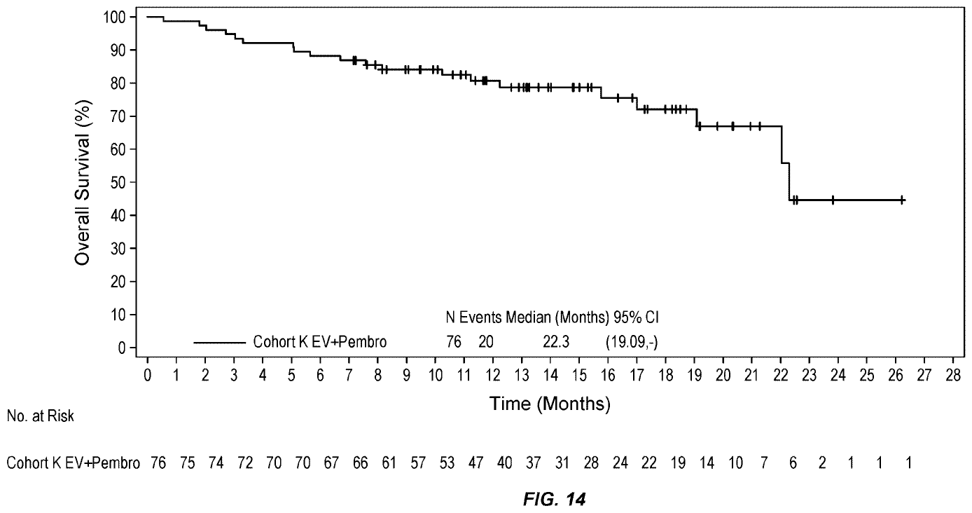

According to the blinded independent central review at this stage of the study, median OS for the Cohort K EV+pembro arm was 22.3 months (95% CI 19.09, −), with 6-month OS at 88.2% and 12-month OS at 80.7% (see FIG. 14 and Table 29).

TABLE 29

| | Dose Esc/ | | |
|---|---|---|---|
| | Cohort A | Cohort K | Cohort K |
| | EV + pembro | EV + pembro | EV Mono |
| | (N = 45) | (N = 76) | (N = 73) |
| No. of events | 22 | 20 | 26 |
| mOS (95% CI), | 26.1 | 22.3 | 21.7 |
| months | (15.51, —) | (19.09, —) | (15.21, —) |
| OS at 6 months | 95.4% | 88.2% | 83.6% |
| OS at 12 months | 83.4% | 80.7% | 70.7% |
| Median follow-up | 44.7 | 14.8 | 15.0 |
| time (months) | | | |

Using duration of treatment as a parameter for summary of exposure, as shown in Table 30, median treatment duration was 9.03 months with a median number of cycles of 11 (1, 29). The relative dose intensity for EV in the Cohort K EV+Pembro arm was 77.23%

TABLE 30

| | Dose Esc/Cohort A | Cohort K | Cohort K |
|---|---|---|---|
| | EV + pembro | EV + pembro | EV Mono |
| | (N = 45) | (N = 76) | (N = 73) |
| Duration of treatment (months) | | | |
| Median | 7.00 | 9.03 | 5.52 |
| Min, Max | 0.7, 32.9 | 0.6, 26.1 | 0.5, 26.9 |
| ≥0 month | 45 (100.0) | 76 (100.0) | 73 (100.0) |
| ≥1 month | 43 (95.6) | 74 (97.4) | 67 (91.8) |
| ≥3 months | 38 (84.4) | 65 (85.5) | 52 (71.2) |
| ≥6 months | 29 (64.4) | 52 (68.4) | 35 (47.9) |
| ≥12 months | 15 (33.3) | 18 (23.7) | 8 (11.0) |
| Number of cycles per subject | | | |
| Median | 9.0 | 11.0 | 8.0 |
| Min, Max | 1, 36 | 1, 29 | 1, 33 |
| Median relative dose intensity for EV | 75.12% | 77.23% | 92.91% |

Cohort K has a longer median EV treatment exposure than Dose Escalation/Cohort A, as shown in Table 31.

TABLE 31

| | Dose Esc/Cohort A | Cohort K | Dose Esc/A/K | Cohort K |
|---|---|---|---|---|
| | EV + pembro | EV + pembro | EV + pembro | EV Mono |
| | (n = 45) | (n = 76) | (n = 121) | (n = 73) |
| | n (%) | n (%) | n (%) | N (%) |
| Patients on treatment, n (%) | 0 | 25 (32.5) | 25 (20.5) | 8 (10.8) |
| Median duration of exposure | | | | |
| (range), months | | | | |
| EV and/or pembro | 7.00 | 9.03 | 8.97 | 5.52 |
| | (0.7, 32.9) | (0.6, 26.1) | (0.6, 32.9) | (0.5, 26.9) |
| EV | 6.44 | 7.05 | 6.90 | 5.52 |
| | (0.7, 32.9) | (0.6, 26.1) | (0.6, 32.9) | (0.5, 26.9) |
| pembro | 6.47 | 7.13 | 6.90 | — |
| | (0.7, 28.1) | (0.6, 26.1) | (0.6, 28.1) | |

Regarding the overall summary of treatment-emergent adverse events, the safety profile was consistent, manageable, and tolerable, as shown in Table 32.

TABLE 32

| | Dose | | | |
|---|---|---|---|---|
| | Esc/Cohort A | Cohort K | Dose Esc/A/K | Cohort K |
| | EV + pembro | EV + pembro | EV + pembro | EV Mono |
| | (n = 45) | (n = 76) | (n = 121) | (n = 73) |
| | n (%) | n (%) | n (%) | N (%) |
| TEAEs | 45 (100.0) | 76 (100.0) | 121 (100.0) | 73 (100.0) |
| Treatment-related TEAEs | 43 (95.6) | 76 (100.0) | 119 (98.3) | 68 (93.2) |
| Grade 3-5 TEAEs | 37 (82.2) | 65 (85.5) | 102 (84.3) | 57 (78.1) |
| Treatment-related grade 3-5 TEAEs | 29 (64.4) | 48 (63.2) | 77 (63.6) | 35 (47.9) |
| Serious TEAEs | 24 (53.3) | 36 (47.4) | 60 (49.6) | 30 (41.1) |
| Treatment-related serious TEAEs | 7 (15.6) | 18 (23.7) | 25 (20.7) | 11 (15.1) |
| TEAEs leading to death | 2 (4.4) | 5 (6.6) | 7 (5.8) | 5 (6.8) |
| Treatment-related TEAEs leading | 1 (2.2) | 3 (3.9) | 4 (3.3) | 2 (2.7) |
| to death | | | | |
| TEAEs leading to treatment | | | | |
| discontinuation | | | | |
| EV only | 8 (17.8) | 19 (25.0) | 27 (22.3) | 18 (24.7) |
| Pembro only | 7 (15.6) | 17 (22.4) | 24 (19.8) | — |
| Both EV and Pembro | 9 (20.0) | 6 (7.9) | 15 (12.4) | — |
| EV or Pembro | 21 (46.7) | 38 (50.0) | 59 (48.8) | 18 (24.7) |

Most common treatment-related TEAEs (all grades) occurring in ≥20% subjects are shown in Table 33. The most common TEAEs in Cohort K EV+Pembro arm combination included fatigue, peripheral sensory neuropathy, and alopecia.

TABLE 33

| | Dose Esc/Cohort A | Cohort K | Dose Esc/A/K | Cohort K |
|---|---|---|---|---|
| | EV + pembro | EV + pembro | EV + pembro | EV Mono |
| | (n = 45) | (n = 76) | (n = 121) | (n = 73) |
| Preferred Term | n (%) | n (%) | n (%) | N (%) |
| Overall | 45 (100.0) | 76 (100.0) | 121 (100.0) | 73 (100.0) |
| Fatigue | 27 (60.0) | 46 (60.5) | 73 (60.3) | 36 (49.3) |
| Peripheral sensory | 26 (57.8) | 40 (52.6) | 66 (54.5) | 33 (45.2) |
| neuropathy | | | | |
| Alopecia | 25 (55.6) | 37 (48.7) | 62 (51.2) | 29 (39.7) |
| Weight decreased | 17 (37.8) | 41 (53.9) | 58 (47.9) | 29 (39.7) |
| Rash maculo-popular | 19 (42.2) | 38 (50.0) | 57 (47.1) | 23 (31.5) |
| Diarrhoea | 23 (51.1) | 31 (40.8) | 54 (44.6) | 30 (41.1) |
| Pruritus | 19 (42.2) | 30 (39.5) | 49 (40.5) | 23 (31.5) |
| Decreased appetite | 20 (44.4) | 26 (34.2) | 46 (38.0) | 39 (53.4) |
| Nausea | 18 (40.0) | 25 (32.9) | 43 (35.5) | 29 (39.7) |
| Dysgeusia | 19 (42.2) | 23 (30.3) | 42 (34.7) | 26 (35.6) |
| Anaemia | 14 (31.1) | 22 (28.9) | 36 (29.8) | 18 (24.7) |
| Urinary tract | 14 (31.1) | 22 (28.9) | 36 (29.8) | 18 (24.7) |
| infection | | | | |
| Constipation | 18 (40.0) | 15 (19.7) | 33 (27.3) | 26 (35.6) |
| Oedema peripheral | 13 (28.9) | 19 (25.0) | 32 (26.4) | 19 (26.0) |
| Dry eye | 11 (24.4) | 18 (23.7) | 29 (24.0) | 9 (12.3) |
| Arthralgia | 13 (28.9) | 14 (18.4) | 27 (22.3) | 14 (19.2) |
| Dizziness | 12 (26.7) | 15 (19.7) | 27 (22.3) | 10 (13.7) |
| Dry skin | 13 (28.9) | 12 (15.8) | 25 (20.7) | 18 (24.7) |

Regarding the treatment-related TEAEs with greater than or equal to Grade 3 occurring in greater than or equal to 5% of subjects, anaemia, rash maculo-papular, increased lipase, and urinary tract infection were the most common, as shown in Table 34.

TABLE 34

| | Dose Esc/Cohort A | Cohort K | Dose Esc/A/K | Cohort K |
|---|---|---|---|---|
| | EV + pembro | EV + pembro | EV + pembro | EV Mono |
| | (n = 45) | (n = 76) | (n = 121) | (n = 73) |
| Preferred Term | n (%) | n (%) | n (%) | N (%) |
| Overall | 37 (82.2) | 65 (85.5) | 102 (84.3) | 57 (78.1) |
| Anaemia | 6 (13.3) | 12 (15.8) | 18 (14.9) | 8 (11.0) |
| Rash maculo-popular | 5 (11.1) | 13 (17.1) | 18 (14.9) | 1 (1.4) |
| Lipase increased | 8 (17.8) | 7 (9.2) | 15 (12.4) | 1 (1.4) |
| Urinary tract infection | 8 (17.8) | 7 (9.2) | 15 (12.4) | 7 (9.6) |
| Hyperglycaemia | 7 (15.6) | 7 (9.2) | 14 (11.6) | 7 (9.6) |
| Fatigue | 6 (13.3) | 7 (9.2) | 13 (10.7) | 6 (8.2) |
| Neutropenia | 4 (8.9) | 7 (9.2) | 11 (9.1) | 6 (8.2) |
| Acute kidney injury | 5 (11.1) | 5 (6.6) | 10 (8.3) | 8 (11.0) |
| Diarrhoea | 2 (4.4) | 6 (7.9) | 8 (6.6) | 5 (6.8) |
| Haematuria | 1 (2.2) | 7 (9.2) | 8 (6.6) | 2 (2.7) |
| Hyponatraemia | 3 (6.7) | 5 (6.6) | 8 (6.6) | 3 (4.1) |
| Amylase increased | 4 (8.9) | 3 (3.9) | 7 (5.8) | 1 (1.4) |
| Chronic kidney disease | 2 (4.4) | 5 (6.6) | 7 (5.8) | 2 (2.7) |
| Hypotension | 4 (8.9) | 3 (3.9) | 7 (5.8) | 2 (2.7) |
| Syncope | 3 (6.7) | 4 (5.3) | 7 (5.8) | 2 (2.7) |

Serious adverse events (SAEs) occurring in greater than or equal to 3% of subjects are shown in Table 35.

TABLE 35

| | Dose Esc/Cohort A | | Cohort K | | Dose Esc/A/K | | Cohort K | |
|---|---|---|---|---|---|---|---|---|
| | EV + pembro | | EV + pembro | | EV + pembro | | EV Mono | |
| | (n = 45) | | (n = 76) | | (n = 121) | | (n = 73) | |
| | n (%) | | n (%) | | n (%) | | N (%) | |
| Preferred Term | All | Related | All | Related | All | Related | All | Related |
| Overall | 24 (53.3) | 7 (15.6) | 36 (47.4) | 18 (23.7) | 60 (49.6) | 25 (20.7) | 30 (41.1) | 11 (15.1) |
| Acute kidney injury | 5 (11.1) | 1 (2.2) | 4 (5.3) | 1 (1.3) | 9 (7.4) | 2 (1.7) | 6 (8.2) | 0 |
| Urinary tract infection | 4 (8.9) | 0 | 4 (5.3) | 0 | 8 (6.6) | 0 | 5 (6.8) | 0 |
| Urosepsis | 3 (6.7) | 0 | 3 (3.9) | 0 | 6 (5.0) | 0 | 0 | 0 |
| Haematuria | 0 | 0 | 4 (5.3) | 0 | 4 (3.3) | 0 | 2 (2.7) | 0 |
| Pneumonia | 2 (4.4) | 0 | 2 (2.6) | 1 (1.3) | 4 (3.3) | 1 (0.8) | 1 (1.4) | 0 |
| Pneumonitis | 0 | 0 | 4 (5.3) | 4 (5.3) | 4 (3.3) | 4 (3.3) | 1 (1.4) | 1 (1.4) |
| Sepsis | 1 (2.2) | 0 | 3 (3.9) | 1 (1.3) | 4 (3.3) | 1 (0.8) | 3 (4.1) | 0 |

Table 36 shows TEAEs leading to death. TEAEs leading to death were heavily confounded by disease progression, pre-existing comorbidities, and/or advanced age.

TABLE 36

| | Dose Esc/Cohort A | Cohort K | Dose Esc/A/K | Cohort K |
|---|---|---|---|---|
| | EV + pembro | EV + pembro | EV + pembro | EV Mono |
| | (n = 45) | (n = 76) | (n = 121) | (n = 73) |
| Preferred Term | n (%) | n (%) | n (%) | N (%) |
| Overall | 2 (4.4) | 5 (6.6) | 7 (5.8) | 5 (6.8) |
| Cardiac arrest | 1 (2.2) | 0 | 1 (0.8) | 0 |
| Multiple organ dysfunction | 1 (2.2) | 0 | 1 (0.8) | 1 (1.4) |
| syndrome | | | | |
| Hydrocephalus | 0 | 1 (1.3) | 1 (0.8) | 0 |
| Pneumonitis | 0 | 1 (1.3) | 1 (0.8) | 0 |
| Respiratory failure | 0 | 1 (1.3) | 1 (0.8) | 1 (1.4) |
| Sepsis | 0 | 1 (1.3) | 1 (0.8) | 1 (1.4) |
| Septic shock | 0 | 1 (1.3) | 1 (0.8) | 0 |
| Embolism | 0 | 0 | 0 | 1 (1.4) |
| Pulmonary sepsis | 0 | 0 | 0 | 1 (1.4) |

A summary of dose modifications is shown in Table 37. Relative dose intensity is consistent in the EV+P combination across cohorts, regardless of dose modifications.

TABLE 37

| | Dose Esc/Cohort A | Cohort K | Dose Esc/A/K | Cohort K |
|---|---|---|---|---|
| | EV + pembro | EV + pembro | EV + pembro | EV Mono |
| | (n = 45) | (n = 76) | (n = 121) | (n = 73) |
| | n (%) | n (%) | n (%) | N (%) |
| TEAEs leading to EV dose | 16 (35.6) | 39 (51.3) | 55 (45.5) | 28 (38.4) |
| reduction | | | | |
| TEAEs leading to dose | | | | |
| interruption | | | | |
| EV only | 17 (37.8) | 30 (39.5) | 47 (38.8) | 34 (46.6) |
| Pembro only | 15 (33.3) | 30 (39.5) | 45 (37.2) | — |
| Both EV and Pembro | 25 (55.6) | 36 (47.4) | 61 (50.4) | — |
| EV or Pembro | 37 (82.2) | 60 (78.9) | 97 (80.2) | 34 (46.6) |
| Median relative dose | 75.12% | 77.23% | 77.2% | 92.91% |
| intensity for EV | | | | |

EV adverse events of special interest (AESI) are shown in Table 38.

TABLE 38

| | Dose Esc/Cohort A | | Cohort K | | Dose Esc/A/K | | Cohort K | |
|---|---|---|---|---|---|---|---|---|
| | EV + pembro | | EV + pembro | | EV + pembro | | EV Mono | |
| | (n = 45) | | (n = 76) | | (n = 121) | | (n = 73) | |
| | All Grade | ≥Gr 3 | All Grade | ≥Gr 3 | All Grade | ≥Gr 3 | All Grade | ≥Gr 3 |
| | n (%) | n (%) | n (%) | n (%) | n (%) | n (%) | n (%) | n (%) |
| Peripheral Neuropathy | 28 (62.2) | 2 (4.4) | 49 (64.5) | 2 (2.6) | 77 (63.6) | 4 (3.3) | 43 (58.9) | 3 (4.1) |
| Skin reactions | 34 (75.6) | 9 (20.0) | 53 (69.7) | 16 (21.1) | 87 (71.9) | 25 (20.7) | 38 (52.1) | 6 (8.2) |
| Hyperglycemia | 9 (20.0) | 7 (15.6) | 13 (17.1) | 7 (9.2) | 22 (18.2) | 14 (11.6) | 10 (13.7) | 7 (9.6) |
| Ocular disorders | 22 (48.9) | 0 | 28 (36.8) | 0 | 50 (41.3) | 0 | 27 (37.0) | 0 |
| Dry eye | 21 (46.7) | 0 | 25 (32.9) | 0 | 46 (38.0) | 0 | 26 (35.6) | 0 |
| Corneal disorders | 1 (2.2) | 0 | 0 | 0 | 1 (0.8) | 0 | 4 (5.5) | 0 |
| Blurred vision | 7 (15.6) | 0 | 9 (11.8) | 0 | 16 (13.2) | 0 | 10 (13.7) | 0 |
| Infusion-related reactions | 3 (6.7) | 1 (2.2) | 4 (5.3) | 0 | 7 (5.8) | 1 (0.8) | 4 (5.5) | 0 |

Pembrolizumab adverse events of special interest (AEOSI) are shown in Table 39. Pembrolizumab AEOSI in the EV+Pembro combination arm were generally consistent with those observed in pembrolizumab monotherapy.

TABLE 39

| | Dose Esc/Cohort A | | Cohort K | | Dose Esc/A/K | |
|---|---|---|---|---|---|---|
| | EV + pembro | | EV + pembro | | EV + pembro | |
| | (n = 45) | | (n = 76) | | (n = 121) | |
| | All Grade | ≥Gr 3 | All Grade | ≥Gr 3 | All Grade | ≥Gr 3 |
| | n (%) | n (%) | n (%) | n (%) | n (%) | n (%) |
| Adrenal Insufficiency | 1 (2.2) | 0 | 3 (3.9) | 0 | 4 (3.3) | 0 |
| Colitis | 3 (6.7) | 1 (2.2) | 3 (3.9) | 1 (1.3) | 6 (5.0) | 2 (1.7) |
| Hepatitis | 0 | 0 | 2 (2.6) | 2 (2.6) | 2 (1.7) | 2 (1.7) |
| Hyperthyroidism | 2 (4.4) | 0 | 3 (3.9) | 0 | 5 (4.1) | 0 |
| Hypophysitis | 1 (2.2) | 0 | 1 (1.3) | 0 | 2 (1.7) | 0 |
| Hypothyroidism | 3 (6.7) | 0 | 10 (13.2) | 0 | 13 (10.7) | 0 |
| Infusion reactions | 2 (4.4) | 0 | 3 (3.9) | 0 | 5 (4.1) | 0 |
| Myasthenic syndrome | 1 (2.2) | 1 (2.2) | 2 (2.6) | 2 (2.6) | 3 (2.5) | 3 (2.5) |
| Myocarditis | 1 (2.2) | 0 | 1 (1.3) | 0 | 2 (1.7) | 0 |
| Myositis | 2 (4.4) | 2 (4.4) | 2 (2.6) | 0 | 4 (3.3) | 2 (1.7) |
| Nephritis | 1 (2.2) | 1 (2.2) | 1 (1.3) | 1 (1.3) | 2 (1.7) | 2 (1.7) |
| Pancreatitis | 0 | 0 | 2 (2.6) | 1 (1.3) | 2 (1.7) | 1 (0.8) |
| Pneumonitis | 4 (8.9) | 1 (2.2) | 7 (9.2) | 4 (5.3) | 11 (9.1) | 5 (4.1) |
| Severe skin reactions | 11 (24.4) | 10 (22.2) | 21 (27.6) | 15 (19.7) | 32 (26.4) | 25 (20.7) |
| Thyroiditis | 0 | 0 | 1 (1.3) | 0 | 1 (0.8) | 0 |

### Skin Reactions (AESI)/Severe Skin Reactions (AEOSI)

Skin reactions are an anticipated overlapping toxicity for enfortumab vedotin treatment and pembrolizumab treatment. Pembrolizumab AEOSI ‘severe skin reactions’ were observed more frequently in the enfortumab vedotin plus pembrolizumab group of Cohort K (27.6%) compared with the pembrolizumab monotherapy (KEYTRUDA USPI). Enfortumab vedotin AESI ‘skin reactions’ were observed more frequently in the enfortumab vedotin plus pembrolizumab arm (69.7%) compared with the enfortumab vedotin monotherapy arm of Cohort K (52.1%).

### Summary of Clinical Outcomes

### Efficacy

The enfortumab vedotin (EV) and Pembrolizumab combination therapy offers a clinically meaningful therapeutic option to cisplatin ineligible patients. The current available options for cisplatin ineligible patients are: (1) gemcitabine/carboplatin followed by avelumab maintenance for patients with disease control after platinum-based chemotherapy (4-6 cycles), (2) gemcitabine/carboplatin, and (3) CPI monotherapy for PD-L1 high expressors or platinum ineligible patients regardless of PD-L1 status. The EV and pembrolizumab combination therapy demonstrates a high magnitude of benefits regardless of Nectin-4 and PD-L1 expression level, with rapid and durable responses. For example, for the Cohort K EV+Pembro arm, a high ORR of 64.5% was observed, with activity regardless of Nectin-4 and PD-L1 expression level. In addition, response rate compared favorably when compared to historical data with gemcitabine/carboplatin. The EV and Pembrolizumab combination therapy demonstrated consistently remarkable efficacy across subgroups of patients (i.e., PD-L1 expression/liver metastases/upper tract disease, etc.). Clinically meaningful overall response rates were observed. The ORR estimate exceeds that for historical benchmark (i.e., gemcitabine/carboplatin). The DOR rate at 6 months was 84.2% and the majority of responders continue to respond to study treatment in Cohort K. A rapid time to response was observed, with the majority of responses (85.7%) seen at first assessment (~week 9). DOR, PFS, and OS are encouraging in this study. Moreover, the EV and Pembrolizumab combination therapy demonstrated greater activity than its individual components.

### Safety

The safety profile for the EV and Pembrolizumab combination therapy is manageable and tolerable in a predominantly elderly, frail population with multiple comorbidities. The safety profile for the EV and Pembrolizumab combination therapy remains generally consistent with the profiles for EV monotherapy and Pembro monotherapy. Adverse events of interest were consistent with those previously reported for enfortumab vedotin and pembrolizumab monotherapies. The rate of dose discontinuation suggests that the EV and Pembrolizumab combination is tolerable. The rate of dose reductions suggests that the EV and Pembrolizumab combination is manageable. EV AESI (e.g., peripheral neuropathy, hyperglycemia, ocular disorders, and infusion reactions) in EV+Pembro are consistent with EV monotherapy. No new safety issues were observed. With the exception of severe skin reactions, the pembrolizumab AEOSI observed in EV+Pembro are consistent with those observed in Pembrolizumab monotherapy. AEOSI are generally manageable. No new safety issues were observed. Skin reactions/severe skin reactions are an anticipated overlapping toxicity for enfortumab vedotin and pembrolizumab therapy. Treatment-related deaths occurred in elderly, frail patients and were confounded by comorbidities.

### 6.1.10 List of Abbreviations and Descriptions of Terms

ACS American Cancer Society
ADC antibody-drug conjugate
AE adverse event
AESI adverse event of special interest
ALT alanine aminotransferase
ANC absolute neutrophil count
aPTT activated partial thromboplastin time
ASCO American Society of Clinical Oncology
AST aspartate aminotransferase
ASTRO American Society for Radiation Oncology
ATA antitherapeutic antibodies
AUA American Urological Association
AUC area under the curve
BCG Bacillus Calmette-Guerin
BICR Blinded independent central review
BPI-SF Brief Pain Inventory Short Form
BSA body surface area
carboplatin cis-Diammine (cyclobutane 1,1 dicarboxylato) platinum
CBC complete blood count
cCR clinical complete response
CFR Code of Federal Regulations
CI confidence interval
cisplatin cis-diamminedichloroplatinum (II)
CNS central nervous system
CPI checkpoint inhibitor
CR complete response
CrCl creatinine clearance
CRF case report form
CPS combined positive score
CT computed tomography
CTLA-4 cytotoxic T-lymphocyte-associated protein
DE DLT-evaluable
DCR disease control rate
DFS disease free survival
DLT dose-limiting toxicity
DOR duration of response
ECD extracellular domain
ECG electrocardiogram
ECHO transthoracic echocardiogram
ECOG Eastern Cooperative Oncology Group
eCRF electronic case report form
EFS event-free survival
ELISA enzyme-linked immunosorbent assay
EORTC European Organization for the Research and Treatment of Cancer
EOT end of treatment
EQ-5D-5L EuroQOl-5 dimensions
EV Enfortumab vedotin
EV Mono Enfortumab vedotin monotherapy
EV+Pembro Enfortumab vedotin in combination with pembrolizumab
FAS full analysis set
GE gene expression
GFR glomerular filtration rate
HbA1c hemoglobin A1c
HIV human immunodeficiency virus
HRU health resource utilization
ICH International Council for Harmonisation
iCPD confirmed progressive disease based on iRECIST guidelines
IEC independent ethics committee
Ig immunoglobulin
IHC immunohistochemistry
imAE immune-mediated adverse event
IND investigational new drug
INR international normalized ratio
IRB institutional review board
iRECIST modified RECIST 1.1 for immune-based therapeutics
IRR infusion-related reaction
iUPD unconfirmed progressive disease (based on iRECIST guidelines)
IV intravenous
la/mUC locally advanced or metastatic urothelial cancer
LFT liver function test
M-CAVI methotrexate/carboplatin/vinblastine
MDRD Modification of Diet in Renal Disease
MMAE monomethyl auristatin E
MRI magnetic resonance imaging
MTD maximum tolerated dose
MVAC methotrexate, vinblastine, doxorubicin, and cisplatin
NAC neoadjuvant chemotherapy
NCI CTCAE National Cancer Institute's Common Terminology Criteria for
Adverse Events
NGS Next Generation Sequencing
NMIBC non-muscle invasive bladder cancer
NYHA New York Heart Association
ORR objective response rate
OS overall survival
PBMC peripheral blood mononuclear cell
pCR pathological complete response
pCRR pathological complete response rate
PD progressive disease
PD-1 programmed cell death 1
PD-L1 programmed death-ligand 1
PD-L2 programmed death-ligand 2
pDS pathological downstaging
pDSR pathological downstaging rate
Pembro pembrolizumab
PFS progression-free survival
PFS2 progression-free survival on subsequent therapy
PK pharmacokinetics
PLND pelvic lymph node dissection
PR partial response
PRO patient reported outcomes
PSA prostate specific antigen
PT prothrombin time
PTT partial thromboplastin time
QLQ-C30 Core QoL Assessment
QoL quality of life
Q2W every 2 weeks
Q3W every 3 weeks
RC radical cystectomy
RECIST Response Evaluation Criteria in Solid Tumors Version
SAE serious adverse event
SAP statistical analysis plan
SD stable disease
SDRIFE symmetrical drug-related intertriginous and flexural exanthema
SJS Stevens-Johnson Syndrome
SMC Safety Monitoring Committee
SOC standard of care
SUO Society of Urologic Oncology
TAb total antibody
T1DM Type 1 diabetes mellitus
TEAE treatment-emergent adverse event
TEN toxic epidermal necrolysis
TME tumor microenvironment
ULN upper limit of normal
USP United States Pharmacopeia

TABLE 40
RECIST Criteria Summary (Version 1.1)
Response Evaluation Criteria in Solid Tumors*

| Term | Definition |
|---|---|
| Complete response (CR) | Disappearance of all target lesions. Any pathological lymph nodes |
| | must have reduction in short axis to <10 mm. |
| Partial response (PR) | A ≥30% decrease in the sum of diameters of target lesions, taking |
| | as reference the baseline sum diameters. |
| Progressive disease (PD) | At least a 20% increase in the sum of diameters of target lesions, |
| | taking as reference the smallest sum of diameters on study (this |
| | includes the baseline sum if that is the smallest on study). In |
| | addition to the relative increase of 20%, the sum must also |
| | demonstrate an absolute increase of at least 5 mm. The appearance |
| | of one or more new lesions is also considered progression. |
| Stable disease (SD) | Neither sufficient shrinkage to qualify for PR nor sufficient |
| | increase to qualify for PD, taking as reference the smallest sum of |
| | diameters while on study. |
| Measurable lesion(s) | Must be accurately measured in at least 1 dimension (longest |
| | diameter in the plane of measurement is to be recorded) with a |
| | minimum size of 10 mm by CT/MRI scan (CT slice thickness no |
| | greater than 5 mm) (maximum of 5 lesions, no more than 2 per |
| | site, at least 10 mm in long axis [or 15 mm in short axis for nodal |
| | lesions]). |
| | A lymph node must be ≥15 mm in short axis when assessed by |
| | CT/MRI scan to be considered measurable. |

From RECIST Version 1.1 (Eisenhauer 2009).

A response (CR or PR) was considered confirmed if the following disease assessment (at least 4-5 weeks after the initial response) still showed response (CR or PR). In cases where the initial response is followed by SD, it was considered as confirmed if the SD was later followed by PR or CR. For example, if a patient had PR in Week 9, SD in Week 14, and PR in Week 18, this PR was considered as confirmed.

Baseline lesion identification: When more than one measurable lesion was present at baseline all lesions up to a maximum of five lesions total (and a maximum of two lesions per organ) representative of all involved organs were identified as target lesions and were recorded and measured at baseline. Target lesions were selected on the basis of their size (lesions with the longest diameter), were representative of all involved but in addition should be those that lend themselves to reproducible repeated measurements (bone lesions should not be selected as a target lesion). It may be the case that, on occasion, the largest lesion did not lend itself to reproducible measurement in which circumstance the next largest lesion which can be measured reproducibly was selected. Lesions in the urinary bladder that were detected on CT/MRI scans were selected as target lesions, provided the radiologist considered the lesion(s) lended to reproducible measurements at post-baseline timepoints and lesions were measured with adequate bladder distension. The same imaging modality was used throughout the study treatment. * Pathological lymph nodes which are defined as measurable and may be identified as target lesions must have met the criterion of a short axis of ≥15 mm by CT or MRI scan. All other pathological nodes (those with short axis ≥10 mm but <15 mm) were considered nontarget lesions. Lymph nodes that have a short axis <10 mm were considered non-pathological and were not be recorded or followed. All other lesions (or sites of disease) including pathological lymph nodes were identified as nontarget lesions and were also be recorded at baseline.

### 6.1.11 New York Heart Association Classification

A Functional and Therapeutic Classification for Prescription of Physical Activity for Cardiac Patients

Class I: patients with no limitation of activities; they suffer no symptoms from ordinary activities.

Class II: patients with slight, mild limitation of activity; they are comfortable with rest or with mild exertion.

Class III: patients with marked limitation of activity; they are comfortable only at rest.

Class IV: patients who should be at complete rest, confined to bed or chair; any physical activity brings on discomfort and symptoms occur at rest.

On-line source: http://www.heart.org/HEARTORG/Conditions/HeartFailure/AboutHeartFailure/Classes-of-Heart-Failure_UCM_306328_Article.jsp

### 6.1.12 Liver Safety Monitoring and Assessment

The following recommendations are from the Food and Drug Administration (FDA) Guidance for Industry titled “Drug-Induced Liver Injury: Premarketing Clinical Evaluation” issued July 2009.

Any patient with an increase of serum aminotransferases to >3×upper limit of normal (ULN) or bilirubin >2×ULN should undergo detailed testing for liver enzymes (including at least alanine aminotransferase (ALT), aspartate aminotransferase (AST), alkaline phosphatase (ALP), and total bilirubin). To confirm the abnormality, testing should be repeated within 72 hours of notification of the test results.

### Description of Liver Abnormalities:

Confirmed liver abnormalities are characterized as Moderate and Severe:

### Moderate:

ALT or AST>3×ULN OR Total Bilirubin >2×ULN

Severe:
ALT or AST>3×ULN AND Total Bilirubin >2×ULN (*See Hy's Law Definition)
ALT or AST >8×ULN
ALT or AST >5×ULN for more than 2 weeks
ALT or AST >3×ULN and International Normalized Ratio (INR) >1.5 (if INR testing is applicable/evaluated)
ALT or AST >3×ULN with the appearance of symptoms suggestive of liver injury (e.g., right upper quadrant pain or tenderness) and/or eosinophilia (>5%)

The investigator may have determined that abnormal liver function results, other than as described above, may have qualified as moderate or severe abnormalities and required additional monitoring and follow-up.

*Hy's Law Definition: Drug-induced jaundice caused by hepatocellular injury, without a significant obstructive component, has a high rate of bad outcomes, from 10 to 50% mortality (or transplant). The 2 “requirements” for Hy's Law are: 1) Evidence that a drug can cause hepatocellular-type injury, generally shown by an increase in transaminase elevations higher 3×ULN (“2×ULN elevations are too common in treated and untreated patients to be discriminating”). 2) Cases of increased bilirubin (at least 2×ULN) with concurrent transaminase elevations at least 3×ULN and no evidence of intra- or extra-hepatic bilirubin obstruction (elevated ALP) or Gilbert's syndrome (Temple et al., Pharmacoepidemiol Drug Saf (2006); 15(4): 241-3.).

### Follow-Up Procedures

Confirmed moderate and severe abnormalities in hepatic functions should have been thoroughly characterized by obtaining appropriate expert consultations, detailed pertinent history, physical examination and laboratory tests. Patients with confirmed abnormal liver function testing should have been followed as described below.

Confirmed moderately abnormal liver function tests (LFTs) should have been repeated 2 to 3 times weekly then weekly or less if abnormalities stabilized or the study drug was discontinued and the patient was asymptomatic.

Severe hepatic liver function abnormalities as defined above, in the absence of another etiology, may have been considered an important medical event and may have been reported as a SAE. The sponsor should have been contacted and informed of all patients for whom severe hepatic liver function abnormalities possibly attributable to study drug were observed.

To further assess abnormal hepatic laboratory finding, it was recommended that the investigator:
Obtain a more detailed history of symptoms and prior or concurrent diseases. Illnesses and conditions such as hypotensive events, and decompensated cardiac disease that may lead to secondary liver abnormalities should be noted. Nonalcoholic steatohepatitis is seen in obese hyperlipoproteinemic and/or diabetic patients, and may be associated with fluctuating aminotransferase levels.
Obtain a history of concomitant drug use (including nonprescription medication, complementary and alternative medications), alcohol use, recreational drug use and special diets
Obtain a history of exposure to environmental chemical agents.
Based on the patient's history, other testing may be appropriate including:
   Acute viral hepatitis (A, B, C, D, E or other infectious agents),
   Ultrasound or other imaging to assess biliary tract disease,
   ther laboratory tests including international normalized ratio (INR), direct bilirubin.
Consider gastroenterology or hepatology consultations.

Additional testing was conducted as determined by the investigator to further evaluate possible etiology. See Section 6.1.6.2(iii)(a) for treatment discontinuation recommendations related to hepatic safety.

### 6.1.13 Image Acquisition Guidelines

Chest-Abdomen-Pelvis Scans (in decreasing order of preference of the following):
1. CT Chest-Abdomen-Pelvis with IV contrast
2. CT Chest without IV contrast and MRI Abdomen-Pelvis with IV gadolinium contrast (if iodinated contrast is medically contraindicated)
3. CT chest without IV contrast and MRI Abdomen-Pelvis without IV gadolinium contrast (if iodinated contrast and gadolinium contrast medically contraindicated)
4. MRI Chest-Abdomen and Pelvis with IV gadolinium contrast
5. Chest-Abdomen-Pelvis CT without IV contrast (oral contrast is recommended) (if patient has contraindication to iodinated contrast and MRI scan)

### Brain Scan (if Clinically Indicated) (in Decreasing Order of Preference):

1. Brain MRI with IV gadolinium
2. Brain CT with IV contrast (if gadolinium and/or MRI is medically contraindicated)
3. Brain MRI without IV gadolinium (if gadolinium is medically contraindicated)
4. Brain CT without IV contrast (if gadolinium is medically contraindicated)

### CT Oral Contrast

1. Radio opaque agents (eg, iodine and barium based agents)
2. Radio-lucent agents (whole milk, VoLumen®, water)

Important: Imaging modality, anatomical coverage, and acquisition parameters should have remained consistent across all imaging visits for each patient.

### 6.1.14 Guidance on Contraception

For the purposes of this guidance, complete abstinence, if consistent with the patient's preferred lifestyle, was an acceptable form of contraception. Complete abstinence is defined as abstinence starting from the time of informed consent and continuing throughout the study and until the end of systemic exposure (at least 6 months after the final dose of study drug).

### Acceptable Methods for Highly Effective Birth Control (Preventing Conception)

Patients who are of childbearing potential or whose partners are of childbearing potential and who are sexually active in a way that could lead to pregnancy may have chosen any TWO of the following methods (please see acceptable combinations below):
Hormonal
   (excluding progestin-only pills; method must be associated with inhibition of ovulation), unless contraindicated
Intrauterine device with failure rate <1%
Tubal ligation
Vasectomy (at least 90 days from the date of surgery with a semen analysis documenting azoospermia)
Barrier method (male or female condom with or without spermicide, cervical cap with or without spermicide, diaphragm with or without spermicide)
A person of childbearing potential is defined as anyone born female who has experienced menarche and who has not undergone surgical sterilization (eg, hysterectomy, bilateral salpingectomy, bilateral oophorectomy) or has not completed menopause. Menopause is defined clinically as 12 months of amenorrhea in a person born female over age 45 in the absence of other biological, physiological, or pharmacological causes.
A barrier method should only be used with a highly effective birth control method that is not a barrier method. Barrier methods alone, including a double-barrier method, are not considered highly effective contraceptive measures (see unacceptable methods of contraception).

Acceptable combinations of contraceptive methods include hormonal method and vasectomy, hormonal method and barrier method, intrauterine device and vasectomy, intrauterine device and barrier method, tubal ligation and vasectomy, and tubal ligation and barrier method.

Acceptable methods for preventing secondary exposure to seminal fluid are as follows: option 1—male condom (with or without spermicide) and cervical cap and option 2—male condom (with or without spermicide) and diaphragm. Subjects born male and who were sexually active with a pregnant or breastfeeding person must have used the contraceptives in Options 1 or 2.

Unacceptable methods of contraception include periodic abstinence, spermicide only, no method, progestin-only pills, withdrawal, concomitant use of female and male condoms, rhythm, and barrier methods alone (including double-barrier methods).

6.1.15 iRECIST: Guidelines for Response Criteria for Use in Trials Testing Immunotherapeutics

Response was also be assessed using modified RECIST 1.1 for immune-based therapeutics (iRECIST) guidelines (Seymour 2017). Immunotherapeutics may have resulted in infiltration of immune cells leading to transient increase in the size in malignant lesions, or undetectable lesions becoming detectable. The criteria were identical to those of Response Evaluation Criteria in Solid Tumors (RECIST) Version 1.1 in many respects but have been adapted to account for instances where an increase in tumor burden, or the appearance of new lesions, did not reflect true tumor progression.

Key differences are described below. All responses defined using iRECIST guidelines are designated with a prefix. iRECIST time point and best responses were recorded separately.

### Confirming Disease Progression

Unlike RECIST Version 1.1, the iRECIST guidelines require the confirmation of progression and uses the terms iUPD (unconfirmed immune progressive disease) and iCPD (confirmed immune progressive disease). Confirmatory scans should have been performed at least 4 weeks, but no longer than 9 weeks after iUPD for the purpose of this trial.

iCPD was confirmed if further increase in tumor burden, compared to the last assessment, was seen as evidenced by 1 or more of the following:
Continued increase in tumor burden (from iUPD) where RECIST Version 1.1 definitions of progression had been met (from nadir) in target, nontarget disease or new lesions
Progression in target disease worsens with an increase of at least 5 mm in the absolute value of the sum
Continued unequivocal progression in nontarget disease with an increase in tumor burden
Increase in size of previously identified new lesion (s) (an increase of at least 5 mm in the absolute value of the sum of those considered to be target new lesions) or additional new lesions.
RECIST Version 1.1 criteria are met in lesions types (target and/or nontarget and/or new lesions) where progression was not previously identified, including the appearance of additional new lesions

If i iUPD was not confirmed at the next assessment, then the appropriate response was assigned (iUPD if the criteria were still met, but no worsening, or iSD [immune stable disease], iPR [immune partial response] or iCR [immune complete response] if those criteria were met compared to baseline). Please refer to the iRECIST guidelines (Seymour 2017) for more details.

### New Lesions

New lesions should have been assessed and measured as they appeared using RECIST Version 1.1 criteria (maximum of 5 lesions, no more than 2 per site, at least 10 mm in long axis or 15 mm in short axis for nodal lesions), and recorded as New Lesions-Target (NLT) and New Lesion-Non-Target (NLNT) to allow clear differentiation from baseline target and nontarget lesions.

New lesions may either meet the criteria of NLT or NLNT to drive iUPD (or iCPD). However, the measurements of target lesions should NOT have been included in the sum of measures of original target lesions identified at baseline. Rather, these measurements were collected separately in the case report form.

### 6.2 Example 2—Enfortumab Vedotin (EV) with or without Pembrolizumab (P) in Cisplatin-Ineligible Patients (Pts) With Previously Untreated Locally Advanced or Metastatic Urothelial Cancer (la/mUC); Additional 3-Month Follow-up on Cohort K Data

### 6.2.1 Background

As discussed supra, despite available therapeutic options, a significant unmet need remains for cisplatin-ineligible patients with locally advanced or metastatic urothelial cancer (la/mUC) in the first-line setting. Gem-carbo (i.e., a combination of gemcitabine and carboplatin) followed by avelumab maintenance is only available to patients who do not progress after platinum-based chemotherapy. PD-1/L1 inhibitor monotherapy is only available to select patients.

The Enfortumab Vedotin (EV)+Pembrolizumab (P) combination has rapid, durable responses (68% confirmed ORR [95% CI: 58.7 to 76.0]) (see Table 24, supra) and a manageable safety profile in patients with previously untreated la/mUC.

After a median follow-up of 18 months, the following Example is an update on the results of EV-103 Cohort K shown in Example 1 (i.e., 3 additional months of follow-up relative to Example 1).

6.2.2 EV-103 Cohort K'Part of an Open-Label, Multiple Cohort, Phase 1b/2 Study in Patients with Urothelial Carcinoma.

The dosing for EV was 1.25 mg/kg IV on days 1 and 8, and pembrolizumab (P) 200 mg IV on day 1 of every 3-week cycle. The primary endpoint was confirmed ORR by RECIST v1.1 per BICR. The key secondary endpoints were confirmed ORR per RECIST v1.1 by investigator, DOR, DCR, PFS by BICR and by investigator, OS, safety/tolerability, and laboratory abnormalities.

The sample size was based on precision of the estimate for ORR characterized by 95% CIs. No formal statistical comparisons were made between the 2 treatment arms.

The stratification factors were liver metastases (present/absent) and ECOG PS (0, 1, or 2). The exploratory endpoints were pharmacokinetics, antitherapeutic, antibody, biomarkers of activity including baseline PD-L1 status and Nectin-4 expression, progression-free survival on subsequent therapy by investigator, and patient reported outcomes.

### 6.2.3 Methods

Untreated ineligible patients with la/mUC were randomized 1:1 to EV (1.25 mg/kg, day 1, day 8) as monotherapy (EV Mono) or in combination with P (200 mg, day 1) using 3-week cycles. The primary endpoint was confirmed objected response rate (cORR) per RECIST v1.1 by blinded independent central review (BICR). Secondary endpoints included duration of response (DOR), disease control rate (DCR), progression-free survival (PFS), overall survival (OS), and safety. No formal statistical comparisons were planned between the treatment arms.

### 6.2.4 Results

The phase 1b/2 study of enfortumab vedotin combined with pembrolizumab as first-line treatment in patients with unresectable locally advanced or metastatic urothelial cancer (la/mUC) who are unable to receive cisplatin-based chemotherapy described in this Example completed enrollment with 149 subjects enrolled. The number of subjects who received any amount of study drug in the EV+Pembrolizumab arm of the study was 76. The number of subjects who received any amount of study drug in the EV Mono arm of the study was 73.

Regarding demographics, as shown in Table 41, the demographics and baseline disease characteristics are representive of the 1L cisplatin-ineligible LA/mUC population.

TABLE 41

| | EV + pembro | EV Mono |
|---|---|---|
| | (N = 76) | (N = 73) |
| Male sex, n (%) | 54 (71.1) | 56 (76.7) |
| Age (yrs), median (range) | 71 (51, 91) | 74 (56, 89) |
| White race, n (%) | 61 (80.3) | 55 (75.3) |

ECOG PS, n (%)

| | | |
|---|---|---|
| 0 | 33 (43.4) | 28 (38.4) |
| 1 | 33 (43.4) | 35 (47.9) |
| 2 | 10 (13.2) | 10 (13.7) |

Primary tumor location,a n (%)

| | | |
|---|---|---|
| Lower tract | 46 (60.5) | 51 (69.9) |
| Upper tract | 30 (39.5) | 21 (28.8) |

Metastasis disease site(s)^{a}, n (%)

| | | |
|---|---|---|
| Bone | 19 (25.0) | 21 (28.8) |
| Liver | 13 (17.1) | 13 (17.8) |
| Lung | 37 (48.7) | 30 (41.1) |

Metastasis category, n (%)

| | | |
|---|---|---|
| Lymph node only | 10 (13.2) | 12 (16.4) |
| Visceral disease | 64 (84.2) | 60 (82.2) |
| Not applicable^{b} | 2 (2.6) | 1 (1.4) |

^{a}Only key sites are identified. Patients may have had metastatic disease in more than 1 location
^{b}Patients had locally advanced disease without metastasis to lymph nodes or distant organs

Table 42 provides a summary of patient disposition. A total of 149 patients were treated (EV+pembro, N=77, and EV Mono, N=74, for a total of 149 patients treated). The median follow-up for EV+Pembrolizumab (95% CI) was 17.6 (16.03, 20.37) months. The median follow-up for EV mono (95% CI) was 18.2 (15.90, 20.07) months.

TABLE 42

| | EV + pembro | EV Mono |
|---|---|---|
| | (N = 77) | (N = 74) |
| Patients treated, n | 76 | 73 |
| Patients on treatment, n (%) | 19 (24.7) | 6 (8.1) |
| Patients off treatment, n (%) | 57 (74.0) | 67 (90.5) |

Reason for treatment discontinuation, n (%)

| | | |
|---|---|---|
| Progressive disease | 35 (45.5) | 40 (54.1) |
| Adverse event | 13 (16.9) | 20 (27.0) |
| Patient decision | 4 (5.2) | 3 (4.1) |
| Physician decision | 3 (3.9) | 3 (4.1) |
| Other | 2 (2.6) | 1 (1.4) |
| Patients off study, n (%) | 26 (33.8) | 33 (44.6) |

Reason for study discontinuation, n (%)

| | | |
|---|---|---|
| Death | 23 (29.9) | 30 (40.5) |
| Patient withdrawal of consent | 2 (2.6) | 2 (2.7) |
| Other | 1 (1.3) | 1 (1.4) |

According to the BICR, the EV+Pembrolizumab confirmed ORR with rapid response was 64.5% (95% CI, 52.7%, 75.1%), as shown in Table 43. For the EV+Pembrolizumab group, 42/49 (85.7%) of responses were observed at first assessment (week 9±1 wk). cORRs were consistent across all pre-specified subgroups. Also, for the EV+Pembrolizumab group, 7/13 (53.8%) cORR was observed in patients with liver metastases. For the EV Mono group, the activity observed was consistent with prior results in 2L+la/mUC.

TABLE 43

| | EV + pembro | EV Mono |
|---|---|---|
| | (N = 76) | (N = 73) |
| Confirmed ORR, n (%) | 49 (64.5) | 33 (45.2) |
| (95% CI) | (52.7, 75.1) | (33.5, 57.3) |

Best overall response, n (%)

| | | |
|---|---|---|
| Complete response | 8 (10.5) | 4 (5.5) |
| Partial response | 41 (53.9) | 29 (39.7) |
| Stable disease | 17 (22.4) | 25 (34.2) |
| Progressive disease | 6 (7.9) | 7 (9.6) |
| Not evaluable | 3 (3.9) | 5 (6.8) |
| No Assessment | 1 (1.3) | 3 (4.1) |
| Median time to objective response, | 2.07 | 2.07 |
| mos (range) | (1.1, 6.6) | (1.9, 15.4) |
| Median number of treatment | 12.0 (1, 34) | 8.0 (1, 33) |
| cycles (range) | | |

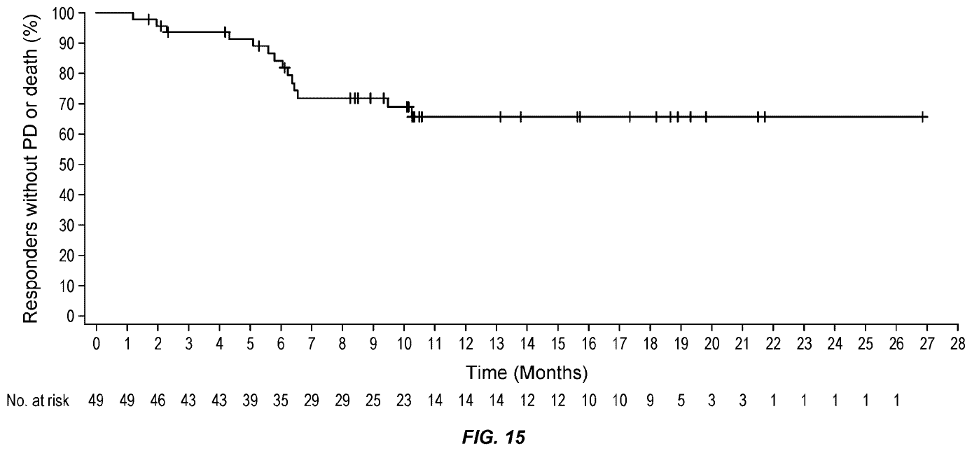

According to the BICR, median DOR for the EV+Pembrolizumab group was not reached (see FIG. 15 and Table 44). At 12 months, 65.6% of responders were still responding. For the EV Mono group, median DOR was 13.2 months (6.14, −) (see Table 44).

TABLE 44

| | EV + pembro | EV Mono |
|---|---|---|
| | (N = 76) | (N = 73) |
| Responders, n | 49 | 33 |
| Progression events, n | 14 | 14 |
| mDOR (95% CI), mos | — | 13.2 |
| | (10.25, —) | (6.14, —) |
| DOR ≥12 mos, % | 65.6 | 59.7 |

Disease Control Rate (DCR) was 86.6% (77.1, 93.5) for the EV+Pembrolizumab group [data not shown]. For the EV Mono group, DCR was 79.5% (68.4, 88.0) [data not shown].

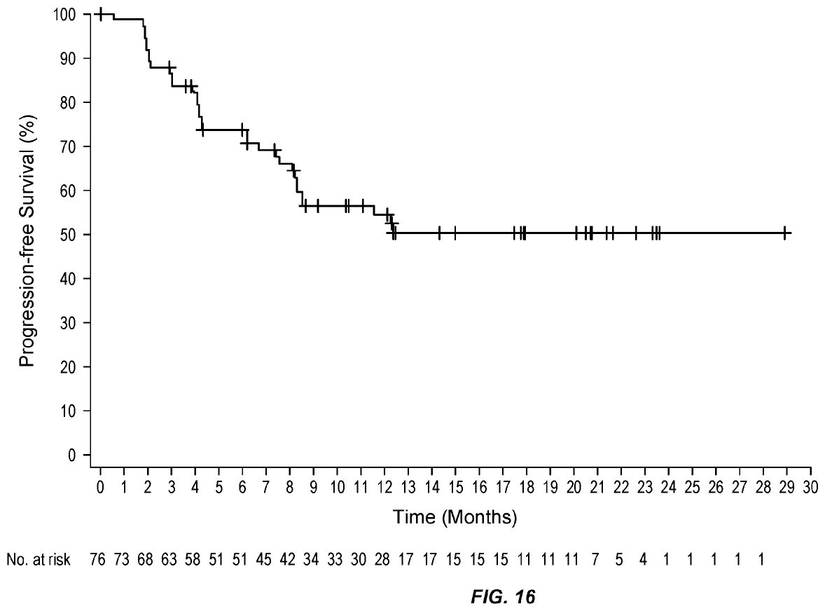

According to the BICR, median PFS for the EV+Pembrolizumab group was not reached (see FIG. 16 and Table 45). For the EV+Pembrolizumab group, the PFS rate at 6 and 12 months (95% CI) was 73.8% (62.01, 82.42) and 54.5% (41.74, 65.61), respectively (see FIG. 16 and Table 45). For the EV Mono group, median PFS was 8.2 (6.05, 15.28) months (see Table 45). In addition, for the EV Mono group, PFS rate at 6 and 12 months was 64.2% (51.09, 74.69) and 40.3% (26.62, 53.60), respectively [data not shown].

TABLE 45

| | EV + pembro | EV Mono |
|---|---|---|
| | (N = 76) | (N = 73) |
| PFS events, n | 33 | 37 |
| mPFS (95% CI), mos | — | 8.2 |
| | (8.31, —) | (6.05, 15.28) |
| PFS at 12 mos, % | 54.5 | 40.3 |

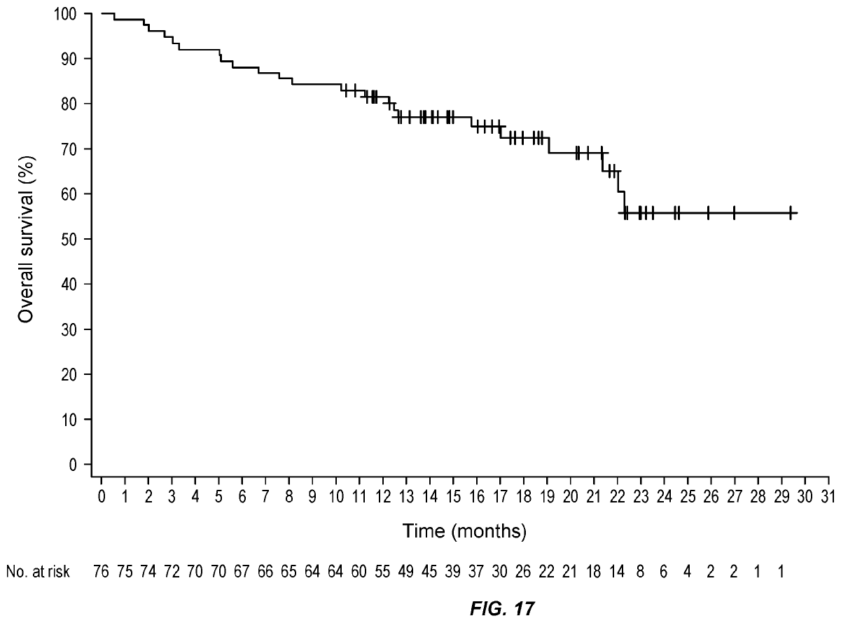

According to the BICR, median OS for the EV+Pembrolizumab group was not reached (see FIG. 17 and Table 46). For the EV+Pembrolizumab group, the OS rate at 6 and 12 months (95% CI) was 88.2% (78.48, 93.65) and 81.5% (70.78, 88.61) respectively (see FIG. 17 and Table 46). For the EV Mono group, median OS was 21.7 (15.47, −) (see Table 46). Also, for the EV Mono group, OS rate at 6 and 12 months was 83.6% (72.87, 90.31) and 69.7% (57.68, 78.87), respectively [data not shown].

TABLE 46

| | EV + pembro | EV Mono |
|---|---|---|
| | (N = 76) | (N = 73) |
| OS events, n | 23 | 30 |
| mOS (95% CI), mos | — | 21.7 |
| | (21.39, —) | (15.47, —) |
| OS at 12 mos, % | 81.5 | 69.7 |
| Median follow-up time, mos | 17.6 | 18.2 |

Table 47 provides treatment-related adverse events (TRAE's) for the EV+pembrolizumab and EV mono treatment groups. The most common TRAE's with EV+Pembrolizumab were fatigue, peripheral sensory neuropathy, maculopapular rash, and alopecia (see Table 47). For the EV+Pembrolizumab group, 19 (25%) of patients had serious TRAE's. For the EV Mono group, 11 (15.1%) of the patients had serious TRAE's. For the EV+Pembrolizumab group, 3 (3.9%) of the patients had TRAE's (pneumonitis, respiratory failure, or sepsis) that led to death. For the EV Mono group, 2 (2.7%) of the patients had treatment-related adverse events (multiple organ dysfunction or respiratory failure) that led to death.

TABLE 47

| | EV + pembro | | EV Mono | |
|---|---|---|---|---|
| TRAE's any grades by | (n = 76) | | (n = 73) | |
| preferred term ≥20% | n (%) | | n (%) | |
| of patients | Any Grade | Grade ≥3 | Any Grade | Grade ≥3 |
| Overall | 76 (100.0) | 49 (64.5) | 68 (93.2) | 34 (46.6) |
| Fatigue | 43 (56.6) | 7 (9.2) | 29 (39.7) | 6 (8.2) |
| Peripheral sensory | 41 (53.9) | 1 (1.3) | 32 (43.8) | 2 (2.7) |
| neuropathy | | | | |
| Rash maculopapular | 36 (47.4) | 13 (17.1) | 21 (28.8) | 1 (1.4) |
| Alopecia | 35 (46.1) | 0 | 27 (37.0) | 0 |
| Pruritus | 30 (39.5) | 3 (3.9) | 19 (26.0) | 1 (1.4) |
| Dysgeusia | 23 (30.3) | 0 | 25 (34.2) | 0 |
| Weight decreased | 23 (30.3) | 3 (3.9) | 22 (30.1) | 1 (1.4) |
| Diarrhea | 22 (28.9) | 5 (6.6) | 20 (27.4) | 4 (5.5) |
| Decreased appetite | 20 (26.3) | 0 | 28 (38.4) | 0 |
| Nausea | 21 (27.6) | 0 | 26 (35.6) | 1 (1.4) |
| Dry eye | 16 (21.1) | 0 | 8 (11.0) | 0 |

Table 48 provides TRAE's of special interest for EV. The majority of TRAE's of special interest for EV were low grade (see Table 48). Skin reactions were observed more frequently with EV+Pembrolizumab. Peripheral neuropathy remains the most common reason for study treatment discontinuation.

TABLE 48

| | EV + pembro | | EV Mono | |
|---|---|---|---|---|
| | (n = 76) | | (n = 73) | |
| | n (%) | | n (%) | |
| | Any Grade | Grade ≥3 | Any Grade | Grade ≥3 |
| Skin reactions | 51 (67.1) | 16 (21.1) | 33 (45.2) | 5 (6.8) |
| Peripheral neuropathy | 48 (63.2) | 2 (2.6) | 40 (54.8) | 2 (2.7) |
| Ocular disorders | 20 (26.3) | 0 | 21 (28.8) | 0 |
| Dry eye | 20 (26.3) | 0 | 21 (28.8) | 0) |
| Blurred vision | 2 (2.6) | 0 | 5 (6.8) | 0 |
| Corneal disorders | 0 | 0 | 4 (5.5) | 0 |
| Hyperglycemia | 11 (14.5) | 5 (6.6) | 8 (11.0) | 7 (9.6) |
| Infusion-related reactions | 3 (3.9) | 0 | 4 (5.5) | 0 |

The TRAE's of special interest for pembrolizumab were consistent with previously observed results with pembrolizumab monotherapy, except for severe skin reactions, which were reported with a higher incidence in this study (see Table 49).

TABLE 49

| | EV + pembro | |
|---|---|---|
| | (n = 76) | |
| | n (%) | |
| | Any Grade | Grade ≥3 |
| Severe skin reactions^{a} | 21 (27.6) | 15 (19.7) |
| Hypothyroidism | 10 (13.2) | 0 |
| Pneumonitis | 7 (9.2) | 4 (5.3) |
| Adrenal insufficiency | 3 (3.9) | 0 |
| Colitis | 3 (3.9) | 1 (1.3) |
| Hyperthyroidism | 3 (3.9) | 0 |
| Infusion reactions | 3 (3.9) | 0 |
| Hepatitis | 2 (2.6) | 2 (2.6) |
| Myasthenic syndrome | 2 (2.6) | 2 (2.6) |
| Myositis | 2 (2.6) | 0 |
| Pancreatitis | 2 (2.6) | 1 (1.3) |
| Hypophysitis | 1 (1.3) | 0 |
| Myocarditis | 1 (1.3) | 0 |
| Nephritis | 1 (1.3) | 1 (1.3) |
| Thyroiditis | 1 (1.3) | 0 |

^{a}There are differences in the rates of skin reactions reported for treatment-related AESIs for EV and TEAEs of special interest for pembrolizumab because the analyses for reporting these events were conducted using different methodologies developed for EV and pembrolizumab monotherapies.

### 6.2.5 Conclusion

At a median follow-up of 18 months, EV+pembrolizumab continues to show a high cORR with rapid and durable responses as a first line treatment in cisplatin-ineligible patients with la/mUC. Notably, median PFS and OS have not been yet been reached. In addition, adverse events for the combination of EV+pembrolizumab shows a manageable safety profile with no new safety concerns after the extended treatment exposure. EV monotherapy results were generally consistent with prior results in 2L+la/mUC. All in all, the data in this example shows efficacy, safety, and tolerability of the EV+pembrolizumab combination therapy for the treatment of cisplatin-ineligible patients with la/mUC in the first-line setting, a population of patients for which a significant unmet need exists.

What is claimed is:

1. A method of treating cancer in a human subject, comprising administering to the subject:
(a) an effective amount of an antibody drug conjugate (ADC) comprising an anti-191P4D12 antibody or antigen binding fragment thereof and
(b) an effective amount of an anti-PD-1 antibody;
wherein the anti-191P4D12 antibody or antigen binding fragment thereof binds to 191P4D12 and is conjugated to one or more units of monomethyl auristatin E (MMAE);
wherein the anti-PD-1 antibody comprises: (i) a light chain variable region comprising light chain CDR1, CDR2 and CDR3 of SEQ ID NOs: 24, 25 and 26, respectively and (ii) a heavy chain variable region comprising heavy chain CDR1, CDR2 and CDR3 of SEQ ID NOs: 29, 30 and 31, respectively;
wherein the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23;
wherein the subject has urothelial or bladder cancer;
wherein the subject has not received an immune checkpoint inhibitor (CPI) therapy; and
wherein the subject is ineligible to receive cisplatin treatment (cisplatin ineligible).

2. The method of claim 1, wherein the subject has visceral metastases.

3. The method of claim 1, wherein the subject has lymph nodes only disease.

4. The method of any one of claims 1 to 3, wherein the disease site of origin is in the upper tract.

5. The method of any one of claims 1 to 3, wherein the disease site of origin is in the lower tract.

6. The method of any one of claims 1 to 5, wherein the subject has a PD-L1 expression combined positive score (CPS) greater than or equal to 10.

7. The method of any one of claims 1 to 5, wherein the subject has a PD-L1 expression CPS less than 10.

8. The method of any one of claims 1 to 6, wherein the subject has a Nectin-4H-score between 0 and 300.

9. The method of any one of claims 1 to 7, wherein the subject has a Nectin-4H-score between 0 and 200.

10. The method of any one of claims 1 to 9, wherein the subject has an ECOG performance status score of 1 to 2.

11. The method of any one of claims 1 to 9, wherein the subject has one or more of the conditions selected from the group consisting of: ECOG performance status score of 2, impaired renal function, and no less than Grade 2 hearing loss.

12. The method of any one of claims 1 to 9, wherein the subject has NYHA Class III heart failure.

13. The method of claim 11, wherein the subject has an ECOG performance status score of 2, and
wherein the subject (i) has hemoglobin ≥10 g/dL; (ii) has GFR ≥50 mL/min; and (iii) does not have NYHA Class III heart failure.

14. The method of claim 11, wherein the impaired renal function is determined by creatinine clearance (CrCl) less than 60 mL/min.

15. The method of claim 11, wherein the impaired renal function is determined by CrCl less than 60 but no less than 30 mL/min.

16. The method of claim 11, wherein the impaired renal function is determined by CrCl less than 30 but no less than 15 mL/min.

17. The method of any one of claims 1 to 16, wherein the subject has one or more of the conditions selected from the group consisting of:
(i) absolute neutrophil count no less than 1500/μL;
(ii) platelet count no less than 100,000/μL;
(iii) hemoglobin no less than 9 g/dL;
(iv) serum bilirubin no more than either of 1.5 times of upper limit of normal (ULN) or 3 times ULN for patients with Gilbert's disease;
(v) CrCl no less than 30 mL/min, and
(vi) alanine aminotransferase and aspartate aminotransferase no more than 3 fold of ULN.

18. The method of claim 17, wherein the subject has all of conditions (i) to (vi) of claim 15.

19. The method of any one of claims 14 to 18, wherein the CrCl is measured by 24 hour urine collection or estimated by the Cockcroft-Gault criteria.

20. The method of any one of claims 1 to 19, wherein the subject has no more than Grade 2 sensory or motor neuropathy.

21. The method of any one of claims 1 to 20, wherein the subject has no active central nervous system metastases.

22. The method of any one of claims 1 to 21, wherein the subject has no uncontrolled diabetes.

23. The method of claim 22, wherein the uncontrolled diabetes is determined by hemoglobin A1c (HbA1c) no less than 8% or HbA1c between 7 and 8% with associated diabetes symptoms that are not otherwise explained.

24. The method of claim 23, wherein the associated diabetes symptoms comprise or consist of polyuria, polydipsia, or both polyuria and polydipsia.

25. The method of any one of claims 1 to 24, wherein the subject has locally advanced or metastatic urothelial cancer.

26. The method of any one of claims 1 to 25, wherein the subject has locally advanced or metastatic bladder cancer.

27. The method of any one of claims 1 to 26, wherein the anti-191P4D12 antibody or antigen binding fragment thereof comprises CDR-H1 comprising the amino acid sequence of SEQ ID NO:9, CDR-H2 comprising the amino acid sequence of SEQ ID NO:10, CDR-H3 comprising the amino acid sequence of SEQ ID NO:11; CDR-L1 comprising the amino acid sequence of SEQ ID NO:12, CDR-L2 comprising the amino acid sequence of SEQ ID NO:13, and CDR-L3 comprising the amino acid sequence of SEQ ID NO:14, or wherein the anti-191P4D12 antibody or antigen binding fragment thereof comprises CDR-H1 comprising the amino acid sequence of SEQ ID NO:16, CDR-H2 comprising the amino acid sequence of SEQ ID NO:17, CDR-H3 comprising the amino acid sequence of SEQ ID NO:18; CDR-L1 comprising the amino acid sequence of SEQ ID NO:19, CDR-L2 comprising the amino acid sequence of SEQ ID NO:20, and CDR-L3 comprising the amino acid sequence of SEQ ID NO:21.

28. The method of any one of claims 1 to 26, wherein the anti-191P4D12 antibody or antigen binding fragment thereof comprises CDR-H1 consisting of the amino acid sequence of SEQ ID NO:9, CDR-H2 consisting of the amino acid sequence of SEQ ID NO:10, CDR-H3 consisting of the amino acid sequence of SEQ ID NO:11; CDR-L1 consisting of the amino acid sequence of SEQ ID NO:12, CDR-L2 consisting of the amino acid sequence of SEQ ID NO:13, and CDR-L3 consisting of the amino acid sequence of SEQ ID NO:14, or
wherein the anti-191P4D12 antibody or antigen binding fragment thereof comprises CDR-H1 consisting of the amino acid sequence of SEQ ID NO:16, CDR-H2 consisting of the amino acid sequence of SEQ ID NO:17, CDR-H3 consisting of the amino acid sequence of SEQ ID NO:18; CDR-L1 consisting of the amino acid sequence of SEQ ID NO:19, CDR-L2 consisting of the amino acid sequence of SEQ ID NO:20, and CDR-L3 consisting of the amino acid sequence of SEQ ID NO:21.

29. The method of any one of claims 1 to 28, wherein the anti-191P4D12 antibody or antigen binding fragment thereof comprises a heavy chain variable region comprising the amino acid sequence of SEQ ID NO:22 and a light chain variable region comprising the amino acid sequence of SEQ ID NO:23.

30. The method of any one of claims 1 to 29, wherein the anti-191P4D12 antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

31. The method of any one of claims 1 to 30, wherein the anti-191P4D12 antigen binding fragment is an Fab, F(ab′)2, Fv or scFv.

32. The method of any one of claims 1 to 30, wherein the anti-191P4D12 antibody is a fully human antibody.

33. The method of any one of claims 1 to 30 and 32, wherein the anti-191P4D12 antibody is an IgG1 and light chain is a kappa light chain.

34. The method of any one of claims 1 to 33, wherein the anti-191P4D12 antibody or antigen binding fragment thereof is recombinantly produced.

35. The method of any one of claims 1 to 34, wherein the anti-191P4D12 antibody or antigen binding fragment is conjugated to each unit of MMAE via a linker.

36. The method of claim 35, wherein the linker is an enzyme-cleavable linker, and wherein the linker forms a bond with a sulfur atom of the antibody or antigen binding fragment thereof.

37. The method of claim 35 or 36, wherein the linker has a formula of: -Aa-Ww-Yy-; wherein -A- is a stretcher unit, a is 0 or 1; —W— is an amino acid unit, w is an integer ranging from 0 to 12; and —Y— is a spacer unit, y is 0, 1, or 2.

38. The method of claim 37, wherein the stretcher unit has the structure of Formula (1) below; the amino acid unit is valine-citrulline; and the spacer unit is a PAB group comprising the structure of Formula (2) below:

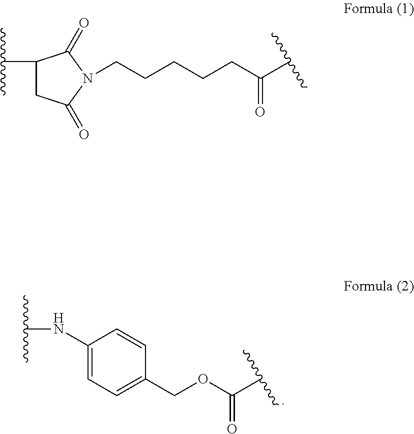 [SMILES: *C(=O)CCCCCN1C(=O)CC(*)C1=O ; *Nc1ccc(COC(*)=O)cc1]

39. The method of claim 37 or 38, wherein the stretcher unit forms a bond with a sulfur atom of the antibody or antigen binding fragment thereof, and wherein the spacer unit is linked to MMAE via a carbamate group.

40. The method of any one of claims 1 to 39, wherein the ADC comprises from 1 to 20 units of MMAE per antibody or antigen binding fragment thereof.

41. The method of any one of claims 1 to 40, wherein the ADC comprises from 1 to 10 units of MMAE per antibody or antigen binding fragment thereof.

42. The method of any one of claims 1 to 41, wherein the ADC comprises from 2 to 8 units of MMAE per antibody or antigen binding fragment thereof.

43. The method of any one of claims 1 to 42, wherein the ADC comprises from 3 to 5 units of MMAE per antibody or antigen binding fragment thereof.

44. The method of any one of claims 1 to 43, wherein the ADC has the following structure: 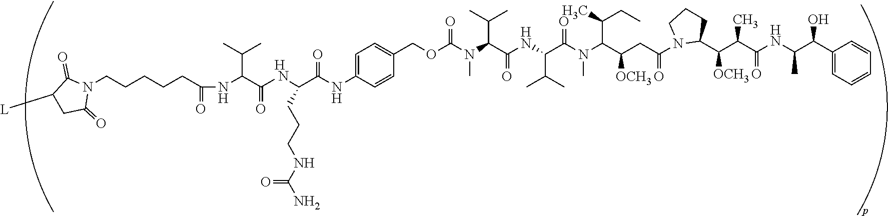
wherein L- represents the anti-191P4D12 antibody or antigen binding fragment thereof and p is from 1 to 10.

45. The method of claim 44, wherein p is from 2 to 8.

46. The method of claim 44 or 45, wherein p is from 3 to 5.

47. The method of any one of claims 44 to 46, wherein p is from 3 to 4.

48. The method of any one of claims 44 to 47, wherein p is about 4.

49. The method of any one of claims 44 to 47, wherein the average p value of the effective amount of the antibody drug conjugates is about 3.8.

50. The method of any one of claims to 49, wherein the ADC is administered to the subject at a dose of about 1 to about 10 mg/kg of the subject's body weight, about 1 to about 5 mg/kg of the subject's body weight, about to about 2.5 mg/kg of the subject's body weight, or about 1 to about 1.25 mg/kg of the subject's body weight.

51. The method of any one of claims 1 to 50, wherein the ADC is administered to the subject at a dose of about 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, about 2.25 mg/kg, or about 2.5 mg/kg of the subject's body weight.

52. The method of any one of claims 1 to 51, wherein the ADC is administered to the subject at a dose of about 1 mg/kg of the subject's body weight.

53. The method of any one of claims 1 to 51, wherein the ADC is administered to the subject at a dose of about 1.25 mg/kg of the subject's body weight.

54. The method of any one of claims 1 to 53, wherein the ADC is administered to the subject by an intravenous (IV) injection or infusion.

55. The method of any one of claims 1 to 54, wherein the ADC is administered to the subject by an IV injection or infusion up to 2 days of a 21-day treatment cycle.

56. The method of any one of claims 1 to 55, wherein the ADC is administered to the subject by an IV injection or infusion on days 1 and 8 of a 21-day treatment cycle.

57. The method of any one of claims 1 to 56, wherein the ADC is administered to the subject by an IV injection or infusion over about 30 minutes up to 2 days of a 21-day treatment cycle.

58. The method of any one of claims 1 to 57, wherein the ADC is administered by an IV injection or infusion over about 30 minutes on days 1 and 8 of a 21-day treatment cycle.

59. The method of any one of claims 1 to 58, wherein the ADC is formulated in a pharmaceutical composition comprising L-histidine, polysorbate-20 (TWEEN-20), and trehalose dehydrate.

60. The method of any one of claims 1 to 59, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.

61. The method of any one of claims 1 to 59, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

62. The method of any one of claims 1 to 61, wherein the ADC has the following structure: 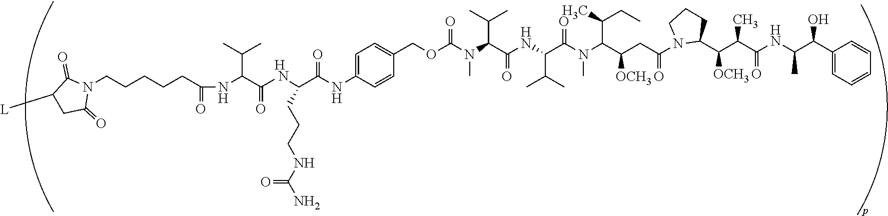
wherein L- represents the antibody or antigen binding fragment thereof and p is from about 3 to about 4, the anti-191P4D12 antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered at a dose of about 1.25 mg/kg of the subject's body weight, and wherein the dose is administered by an IV injection or infusion over about 30 minutes on days 1 and 8 of a 21-day treatment cycle.

63. The method of any one of claims 1 to 60, wherein the anti-PD-1 antibody is administered to the subject at a dose of about 100 mg to about 400 mg.

64. The method of any one of claims 1 to 61, wherein the anti-PD-1 antibody is administered to the subject at a dose of about 200 mg.

65. The method of claim 62, wherein:
(a) the anti-PD-1 antibody is administered to the subject at a dose of about 200 mg; and
(b) after step (a), the anti-PD-1 antibody is administered to the subject at a dose of about 400 mg every 42 days.

66. The method of any one of claims 1 to 65, wherein the anti-PD-1 antibody is administered to the subject by an IV infusion on 1 day of the 21-day treatment cycle.

67. The method of any one of claims 1 to 66, wherein the anti-PD-1 antibody is administered to the subject by an IV infusion on day 1 in each treatment cycle of 21 days.

68. The method of any one of claims 1 to 67, wherein the anti-PD-1 antibody is administered to the subject by an IV infusion over about 30 minutes on 1 day of the 21-day treatment cycle.

69. The method of any one of claims 1 to 66, wherein the anti-PD-1 antibody is administered to the subject by an IV infusion over about 30 minutes on day 1 in each treatment cycle of 21 days.

70. The method of any one of claims 1 to 69, whereby the subject has a complete response following the treatment.

71. The method of any one of claims 1 to 69, wherein the subject has a partial response following the treatment.

72. The method of any one of claims 1 to 69, wherein the subject has a complete response or a partial response following the treatment.

73. The method of any one of claims 1 to 69, wherein the subject has a stable disease following the treatment.

74. The method of any one of claims 1 to 69, wherein the subject has a duration of response of at least or about 6 months following the treatment.

75. The method of any one of claims 1 to 69, wherein the subject has a duration of response of at least or about 12 months following the treatment.

76. The method of any one of claims 1 to 69, wherein the subject has a duration of response of at least or about 24 months following the treatment.

77. The method of any one of claims 1 to 69, wherein the subject has a duration of response of at least or about 27 months following the treatment.

78. The method of any one of claims 1 to 69, wherein the subject has a progression free survival of at least or about 6 months following the treatment.

79. The method of any one of claims 1 to 69, wherein the subject has a progression free survival of at least or about 8 months following the treatment.

80. The method of any one of claims 1 to 69, wherein the subject has a progression free survival of at least or about 12 months following the treatment.

81. The method of any one of claims 1 to 69, wherein the subject has a progression free survival of at least or about 20 months following the treatment.

82. The method of any one of claims 1 to 69, wherein the subject has a progression free survival of at least or about 29 months following the treatment.

83. The method of any one of claims 1 to 69, wherein the subject has an overall survival of at least or about 22 months following the treatment.

84. The method of any one of claims 1 to 69, wherein the subject has an overall survival of at least or about 27 months following the treatment.

85. The method of any one of claims 1 to 69, wherein the subject has a overall survival of at least or about 30 months following the treatment.

86. The method of any one of claims 1 to 69, wherein the subject has an overall survival ranging from 19 to 25 months following the treatment.

87. The method of any one of claims 1 to 69, wherein the subject has an overall survival ranging from 28 to 32 months following the treatment.

88. The method of any one of claims 1 to 69, wherein a population of the subjects is treated by the methods, and wherein percentage of the subjects having complete response in the treated population is at least or about 10%.

89. The method of any one of claims 1 to 69, wherein a population of the subjects is treated by the methods, and wherein percentage of the subjects having partial response in the treated population is at least or about 54%.

90. The method of any one of claims 1 to 69, wherein a population of the subjects is treated by the methods, and wherein objective response rate in the treated population is at least or about 65%.

91. The method of any one of claims 1 to 69, wherein a population of the subjects is treated by the methods, and wherein objective response rate in the treated population ranges from 53% to 75%.

92. The method of any one of claims 1 to 69, wherein a population of the subjects is treated by the methods, and wherein percentage of the subjects having stable disease in the treated population is at least or about 22%.

93. The method of any one of claims 1 to 69, wherein a population of the subjects is treated by the methods, and wherein duration of response in the treated population is at least or about 6 months.

94. The method of any one of claims 1 to 69, wherein a population of the subjects is treated by the methods, and wherein duration of response in the treated population is at least or about 12 months.

95. The method of any one of claims 1 to 69, wherein a population of the subjects is treated by the methods, and wherein duration of response in the treated population is at least or about 24 months.

96. The method of any one of claims 1 to 69, wherein a population of the subjects is treated by the methods, and wherein duration of response in the treated population is at least or about 27 months.

97. The method of any one of claims 1 to 69, wherein a population of the subjects is treated by the methods, and wherein progression free survival in the treated population is at least or about 6 months.

98. The method of any one of claims 1 to 69, wherein a population of the subjects is treated by the methods, and wherein progression free survival in the treated population is at least or about 12 months.

99. The method of any one of claims 1 to 69, wherein a population of the subjects is treated by the methods, and wherein progression free survival in the treated population is at least or about 20 months.

100. The method of any one of claims 1 to 69, wherein a population of the subjects is treated by the methods, and wherein progression free survival in the treated population is at least or about 29 months.

101. The method of any one of claims 1 to 69, wherein a population of the subjects is treated by the methods, and wherein median overall survival in the treated population is at least or about 22 months.

102. The method of any one of claims 1 to 69, wherein a population of the subjects is treated by the methods, and wherein median overall survival in the treated population is at least or about 27 months.

103. The method of any one of claims 1 to 69, wherein a population of the subjects is treated by the methods, and wherein median overall survival in the treated population is at least or about 30 months.

104. The method of any one of claims 1 to 69, wherein a population of the subjects is treated by the methods, and wherein overall survival in the treated population ranges from 19 to 25 months.

105. The method of any one of claims 1 to 69, wherein a population of the subjects is treated by the methods, and wherein overall survival in the treated population ranges from 30 to 32 months.

106. The method of any one of claims 1 to 70 and 72, wherein the complete response rate is at least or about 10% for a population of subjects treated with the method.

107. The method of any one of claims 1 to 69, 71, and 72, wherein the partial response rate is at least or about 54% for a population of subjects treated with the method.

108. The method of any one of claims 1 to 72, wherein objective response rate is at least or about 65% for a population of subjects treated with the method.

109. The method of any one of claims 1 to 72, wherein objective response rate is from 53% to 75% for a population of subjects treated with the method.

110. The method of any one of claims 1 to 69 and 73, wherein the stable disease rate is at least or about 22% for a population of subjects treated with the method.

111. The method of any one of claims 1 to 69 and 74 to 77, wherein the duration of response is at least or about 6 months for a population of subjects treated with the method.

112. The method of any one of claims 1 to 69 and 74 to 77, wherein the duration of response is at least or about 12 months for a population of subjects treated with the method.

113. The method of any one of claims 1 to 69 and 74 to 77, wherein the duration of response is at least or about 24 months for a population of subjects treated with the method.

114. The method of any one of claims 1 to 69 and 74 to 77, wherein the duration of response is at least or about 27 months for a population of subjects treated with the method.

115. The method of any one of claims 1 to 69 and 78 to 82, wherein the progression free survival is at least or about 6 months for a population of subjects treated with the method.

116. The method of any one of claims 1 to 69 and 78 to 82, wherein the progression free survival is at least or about 12 months for a population of subjects treated with the method.

117. The method of any one of claims 1 to 69 and 78 to 82, wherein the progression free survival is at least or about 20 months for a population of subjects treated with the method.

118. The method of any one of claims 1 to 69 and 78 to 82, wherein the progression free survival is at least or about 29 months for a population of subjects treated with the method.

119. The method of any one of claims 1 to 69 and 83 to 87, wherein the median overall survival is at least or about 22 months for a population of subjects treated with the method.

120. The method of any one of claims 1 to 69 and 83 to 87, wherein the median overall survival is at least or about 27 months for a population of subjects treated with the method.

121. The method of any one of claims 1 to 69 and 83 to 87, wherein the median overall survival is at least or about 30 months for a population of subjects treated with the method.

122. The method of any one of claims 1 to 69, 79, and 80, wherein the overall survival is from 19 to 25 months for a population of subjects treated with the method.

123. The method of any one of claims 1 to 69, 79, and 80, wherein the overall survival is from 30 to 32 months for a population of subjects treated with the method.


SEQUENCE LISTING (47 sequences)

SEQ ID NO: 1 - DNA, 3464 bp, Homo sapiens
  source 1..3464: mol_type = other DNA; note = misc_feature - cDNA of 191P4D12
  CDS 264..1796: protein_id = 2; translation = MPLSLGAEMWGPEAWLLLLLLLASFTGRCPAGELETSDVVTVVL GQDAKLPCFYRGDSGEQVGQVAWARVDAGEGAQELALLHSKYGLHVSPAYEGRVEQPP PPRNPLDGSVLLRNAVQADEGEYECRVSTFPAGSFQARLRLRVLVPPLPSLNPGPALE EGQGLTLAASCTAEGSPAPSVTWDTEVKGTTSSRSFKHSRSAAVTSEFHLVPSRSMNG QPLTCVVSHPGLLQDQRITHILHVSFLAEASVRGLEDQNLWHIGREGAMLKCLSEGQP PPSYNWTRLDGPLPSGVRVDGDTLGFPPLTTEHSGIYVCHVSNEFSSRDSQVTVDVLD PQEDSGKQVDLVSASVVVVGVIAALLFCLLVVVVVLMSRYHRRKAQQMTQKYEEELTL TRENSIRRLHSHHTDPRSQPEESVGLRAEGHPDSLKDNSSCSVMSEEPEGRSYSTLTT VREIETQTELLSPGSGRAEEEEDQDEGIKQAMNHFVQENGTLRAKPTGNGIYINGRGH LV
ggccgtcgttgttggccacagcgtgggaagcagctctgggggagctcggagctcccgatcacggcttcttgggggtagctacggctgggtgtgtagaacggggccggggctggggctgggtcccctagtggagacccaagtgcgagaggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtggctcagacccaggtgtgcgggcatagctggagctggaatctgcctccggtgtgagggaacctgtctcctaccacttcggagccatgggggcaagtgtgaagcagccagtccctgggtcagccagaggcttgaactgttacagaagccctctgccctctggtggcctctgggcctgctgcatgtacatattttctgtaaatatacatgcgccgggagcttcttgcaggaatactgctccgaatcacttttaatttttttcttttttttttcttgccctttccattagttgtattttttatttatttttatttttatttttttttagagatggagtctcactatgttgctcaggctggccttgaactcctgggctcaagcaatcctcctgcctcagcctccctagtagctgggactttaagtgtacaccactgtgcctgctttgaatcctttacgaagagaaaaaaaaaattaaagaaagcctttagatttatccaatgtttactactgggattgcttaaagtgaggcccctccaacaccagggggttaattcctgtgattgtgaaaggggctacttccaaggcatcttcatgcaggcagccccttgggagggcacctgagagctggtagagtctgaaattagggatgtgagcctcgtggttactgagtaaggtaaaattgcatccaccattgtttgtgataccttagggaattgcttggacctggtgacaagggctcctgttcaatagtggtgttggggagagagagagcagtgattatagaccgagagagtaggagttgaggtgaggtgaaggaggtgctgggggtgagaatgtcgcctttccccctgggttttggatcactaattcaaggctcttctggatgtttctctgggttggggctggagttcaatgaggtttatttttagctggcccacccagatacactcagccagaatacctagatttagtacccaaactcttcttagtctgaaatctgctggatttctggcctaagggagaggctcccatccttcgttccccagccagcctaggacttcgaatgtggagcctgaagatctaagatcctaacatgtacattttatgtaaatatgtgcatatttgtacataaaatgatattctgtttttaaataaacagacaaaacttgaaaaa

SEQ ID NO: 2 - protein, 510 aa, Homo sapiens
  source 1..510: note = misc_feature - 191P4D12
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 3 - DNA, 1432 bp, Homo sapiens
  source 1..1432: mol_type = other DNA; note = misc_feature - cDNA of Ha22-2(2,4)6.1 heavy chain
  CDS 32..1432: protein_id = 4; translation = MELGLCWVFLVAILEGVQCEVQLVESGGGLVQPGGSLRLSCAAS GFTFSSYNMNWVRQAPGKGLEWVSYISSSSSTIYYADSVKGRFTISRDNAKNSLSLQM NSLRDEDTAVYYCARAYYYGMDVWGQGTTVTVSSASTKGPSVFPLAPSSKSTSGGTAA LGCLVKDYFPEPVTVSWNSGALTSGVHTFPAVLQSSGLYSLSSVVTVPSSSLGTQTYI CNVNHKPSNTKVDKRVEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSREEMTKNQVSLTCLVKGFY PSDIAVEWESNGQPENNYKTTPPVLDSDGSFFLYSKLTVDKSRWQQGNVFSCSVMHEA LHNHYTQKSLSLSPGK
ggtgatcagcactgaacacagaggactcaccatggagttggggctgtgctgggttttccttgttgctattttagaaggtgtccagtgtgaggtgcagctggtggagtctgggggaggcttggtacagcctggggggtccctgagactctcctgtgcagcctctggattcaccttcagtagctataacatgaactgggtccgccaggctccagggaaggggctggagtgggtttcatacattagtagtagtagtagtaccatatactacgcagactctgtgaagggccgattcaccatctccagagacaatgccaagaactcactgtctctgcaaatgaacagcctgagagacgaggacacggctgtgtattactgtgcgagagcatactactacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 4 - protein, 466 aa, Homo sapiens
  source 1..466: note = misc_feature - Ha22-2(2,4)6.1 heavy chain
MELGLCWVFLVAILEGVQCEVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 735 bp, Homo sapiens
  source 1..735: mol_type = other DNA; note = misc_feature - cDNA of Ha22-2(2,4)6.1 light chain
  CDS 25..735: protein_id = 6; translation = MDMRVPAQLLGLLLLWFPGSRCDIQMTQSPSSVSASVGDRVTIT CRASQGISGWLAWYQQKPGKAPKFLIYAASTLQSGVPSRFSGSGSGTDFTLTISSLQP EDFATYYCQQANSFPPTFGGGTKVEIKRTVAAPSVFIFPPSDEQLKSGTASVVCLLNN FYPREAKVQWKVDNALQSGNSQESVTEQDSKDSTYSLSSTLTLSKADYEKHKVYACEV THQGLSSPVTKSFNRGEC
agtcagacccagtcaggacacagcatggacatgagggtccccgctcagctcctggggctcctgctgctctggttcccaggttccagatgcgacatccagatgacccagtctccatcttccgtgtctgcatctgttggagacagagtcaccatcacttgtcgggcgagtcagggtattagcggctggttagcctggtatcagcagaaaccagggaaagcccctaagttcctgatctatgctgcatccactttgcaaagtggggtcccatcaaggttcagcggcagtggatctgggacagatttcactctcaccatcagcagcctgcagcctgaagattttgcaacttactattgtcaacaggctaacagtttccctcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - protein, 236 aa, Homo sapiens
  source 1..236: note = misc_feature - Ha22-2(2,4)6.1 light chain
MDMRVPAQLLGLLLLWFPGSRCDIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 466 aa, Homo sapiens; identical to SEQ ID NO: 4
  source 1..466: note = misc_feature - Ha22-2(2,4)6.1 heavy chain

SEQ ID NO: 8 - protein, 236 aa, Homo sapiens; identical to SEQ ID NO: 6
  source 1..236: note = misc_feature - Ha22-2(2,4)6.1 light chain

SEQ ID NO: 9 - protein, 5 aa, Homo sapiens
  source 1..5: note = CDR1 of heavy chain
SYNMN

SEQ ID NO: 10 - protein, 17 aa, Homo sapiens
  source 1..17: note = CDR2 of heavy chain
YISSSSSTIYYADSVKG

SEQ ID NO: 11 - protein, 8 aa, Homo sapiens
  source 1..8: note = CDR3 of heavy chain
AYYYGMDV

SEQ ID NO: 12 - protein, 11 aa, Homo sapiens
  source 1..11: note = CDR1 of light chain
RASQGISGWLA

SEQ ID NO: 13 - protein, 7 aa, Homo sapiens
  source 1..7: note = CDR2 of light chain
AASTLQS

SEQ ID NO: 14 - protein, 9 aa, Homo sapiens
  source 1..9: note = CDR3 of light chain
QQANSFPPT

SEQ ID NO: 15 - protein, 4 aa, synthetic construct
  source 1..4: note = linker
GFLG

SEQ ID NO: 16 - protein, 8 aa, synthetic construct
  source 1..8: note = VH CDR1 according to IMGT
GFTFSSYN

SEQ ID NO: 17 - protein, 8 aa, synthetic construct
  source 1..8: note = VH CDR2 according to IMGT
ISSSSSTI

SEQ ID NO: 18 - protein, 10 aa, synthetic construct
  source 1..10: note = VH CDR3 according to IMGT
ARAYYYGMDV

SEQ ID NO: 19 - protein, 6 aa, synthetic construct
  source 1..6: note = VL CDR1 according to IMGT
QGISGW

SEQ ID NO: 20 - skipped

SEQ ID NO: 21 - protein, 9 aa, synthetic construct
  source 1..9: note = VL CDR3 according to IMGT
QQANSFPPT

SEQ ID NO: 22 - protein, 117 aa, synthetic construct
  source 1..117: note = heavy chain variable region (VH), the 20th amino acid (glutamic acid) to the 136th amino acid (serine) of SEQ ID NO:7
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 23 - protein, 108 aa, synthetic construct
  source 1..108: note = light chain variable region (VL), the 23rd amino acid (aspartic acid) to the 130th amino acid (arginine) of SEQ ID NO:8
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKR

SEQ ID NO: 24 - protein, 15 aa, synthetic construct
  source 1..15: note = VL CDR1 of Pembrolizumab
RASKGVSTSGYSYLH

SEQ ID NO: 25 - protein, 7 aa, synthetic construct
  source 1..7: note = VL CDR2 of Pembrolizumab
LASYLES

SEQ ID NO: 26 - protein, 9 aa, synthetic construct
  source 1..9: note = VL CDR3 of Pembrolizumab
QHSRDLPLT

SEQ ID NO: 27 - protein, 111 aa, synthetic construct [sequence omitted]

SEQ ID NO: 28 - protein, 218 aa, synthetic construct
  source 1..218: note = Light chain of Pembrolizumab
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 5 aa, synthetic construct
  source 1..5: note = VH CDR1 of Pembrolizumab
NYYMY

SEQ ID NO: 30 - protein, 17 aa, synthetic construct
  source 1..17: note = VH CDR2 of Pembrolizumab
GINPSNGGTNFNEKFKN

SEQ ID NO: 31 - protein, 11 aa, synthetic construct
  source 1..11: note = VH CDR3 of Pembrolizumab
RDYRFDMGFDY

SEQ ID NO: 32 - protein, 120 aa, synthetic construct [sequence omitted]

SEQ ID NO: 33 - protein, 447 aa, synthetic construct
  source 1..447: note = Heavy chain of Pembrolizumab
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 15 aa, synthetic construct
  source 1..15: note = VL CDR1 of hPD-1.08A
RASKSVSTSGFSYLH

SEQ ID NO: 35 - protein, 7 aa, synthetic construct
  source 1..7: note = VL CDR2 of hPD-1.08A
LASNLES

SEQ ID NO: 36 - protein, 9 aa, synthetic construct
  source 1..9: note = VL CDR3 of hPD-1.08A
QHSWELPLT

SEQ ID NO: 37 - protein, 5 aa, synthetic construct
  source 1..5: note = VH CDR1 of hPD-1.08A
SYYLY

SEQ ID NO: 38 - protein, 16 aa, synthetic construct
  source 1..16: note = VH CDR2 of hPD-1.08A
GVNPSNGGTNFSEKFK

SEQ ID NO: 39 - protein, 11 aa, synthetic construct
  source 1..11: note = VH CDR3 of hPD-1.08A
RDSNYDGGFDY

SEQ ID NO: 40 - protein, 139 aa, synthetic construct
  source 1..139: note = Mature h109A heavy chain variable region
MDWTWSILFLVAAPTGAHSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 41 - protein, 130 aa, synthetic construct
  source 1..130: note = Mature K09A light chain variable region
MAPVQLLGLLVLFLPAMRCEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 42 - protein, 130 aa, synthetic construct
  source 1..130: note = Mature K09A light chain variable region
MAPVQLLGLLVLFLPAMRCEIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 43 - protein, 130 aa, synthetic construct
  source 1..130: note = Mature K09A light chain variable region
MAPVQLLGLLVLFLPAMRCDIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 44 - protein, 466 aa, synthetic construct
  source 1..466: note = Mature 409 heavy chain
MAVLGLLFCLVTFPSCVLSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 237 aa, synthetic construct
  source 1..237: note = Mature K09A light chain
MAPVQLLGLLVLFLPAMRCEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 237 aa, synthetic construct
  source 1..237: note = Mature K09A light chain
MAPVQLLGLLVLFLPAMRCEIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 237 aa, synthetic construct
  source 1..237: note = Mature K09A light chain
MAPVQLLGLLVLFLPAMRCDIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC